SEQUENCE LISTING (12 sequences)

SEQ ID NO: 1 - DNA, 22 bp, Artificial Sequence
  Description of Artificial Sequence Synthetic primer
tggacgggactattgaaggctg

SEQ ID NO: 2 - DNA, 21 bp, Artificial Sequence
  Description of Artificial Sequence Synthetic primer
cgccttttcagtagatggagg

SEQ ID NO: 3 - DNA, 24 bp, Artificial Sequence
  Description of Artificial Sequence Synthetic primer
gcggattgaccgtaatgggatagg

SEQ ID NO: 4 - DNA, 11 bp, Artificial Sequence
  Description of Artificial Sequence Synthetic probe
rtgaynnngcr

SEQ ID NO: 5 - DNA, 20 bp, Artificial Sequence
  Description of Artificial Sequence Synthetic primer
tctcctcgctggaaaaagaa

SEQ ID NO: 6 - DNA, 20 bp, Artificial Sequence
  Description of Artificial Sequence Synthetic primer
atttcgtgtcggtcgtgtaa

SEQ ID NO: 7 - DNA, 21 bp, Artificial Sequence
  Description of Artificial Sequence Synthetic primer
cctggcaaggttacgaagtga

SEQ ID NO: 8 - DNA, 21 bp, Artificial Sequence
  Description of Artificial Sequence Synthetic primer
cagtttcatcccgtcgatctc

SEQ ID NO: 9 - DNA, 21 bp, Artificial Sequence
  Description of Artificial Sequence Synthetic probe
ctgatgttccagcaagtgccc

SEQ ID NO: 10 - DNA, 21 bp, Artificial Sequence
  Description of Artificial Sequence Synthetic probe
gttgaggggactttcccaggc

SEQ ID NO: 11 - protein, 4 aa, Artificial Sequence
  Description of Artificial Sequence Synthetic peptide
DEVD

SEQ ID NO: 12 - DNA, 15 bp, Artificial Sequence
  Description of Artificial Sequence Synthetic oligonucleotide
ttttttttttttttt

(19) United States
(12) Patent Application Publication
(10) Pub. No.: US 2015/0080462 A1
(43) Pub. Date: Mar. 19, 2015

(54) COMPOSITIONS AND METHODS FOR THE TREATMENT OR PREVENTION OF DISORDERS RELATING TO OXIDATIVE STRESS

(71) Applicant: THE JOHNS HOPKINS UNIVERSITY, Baltimore, MD (US)

(72) Inventors: Shyam Biswal, Ellicott City, MD (US); Sylvain Dore, Baltimore, MD (US); Rajesh Kumar Thimmulappa, Baltimore, MD (US); Tirumalai Rangasamy, Baltimore, MD (US); Yoshihito Sakata, Baltimore, MD (US); Zahoor Ahmad Shah, Owings Mills, MD (US); Hean Zhuang, Ellicot City, MD (US); Anju Singh, Baltimore, MD (US)

(21) Appl. No.: 14/451,415

(22) Filed: Aug. 4, 2014

Related U.S. Application Data
(63) Continuation of application No. 12/780,605, filed on May 14, 2010.
Application No. 12/780,605 is a continuation of application No. 12/556,503, filed on Sep. 9, 2009, now abandoned.
Application No. 12/556,503 is a continuation of application No. 11/988,185, now abandoned.
(60) Provisional application No. 60/800,975, filed on May 17, 2006.
(60) Provisional application No. 60/696,485, filed on Jul. 1, 2005.

(51) Int. Cl. A61K 31/353; A61K 45/06

(52) U.S. Cl. CPC A61K 31/353; A61K 45/06
USPC 514/456; 549/399; 435/375

(57) ABSTRACT

The present invention features methods for treating or preventing conditions, diseases, or disorders related to oxidative stress. In one embodiment, the method increases Nrf2 biological activity or expression. In particular, the invention provides for the treatment or prevention of diseases relating to oxidative stress including emphysema, sepsis, septic shock, ischemic injury, cerebral ischemia and neurodegenerative disorders, meningitis, encephalitis, hemorrhage, cerebral ischemia, heart ischemia, cognitive deficits and neurodegenerative disorders.

### CROSS-REFERENCE TO RELATED APPLICATION

This application claims the benefit of the following U.S. Provisional Application Nos. 60/696,485, which was filed on Jul. 1, 2005, and 60/800,975, which was filed on May 17, 2006, the entire disclosures of which are hereby incorporated in its entirety.

### STATEMENT OF RIGHTS TO INVENTIONS MADE UNDER FEDERALLY SPONSORED RESEARCH

This work was supported by the following grants from the National Institutes of Health, Grant Nos: AT001836, AA014911, AT002113, NS046400, and HL081205. The government may have certain rights in the invention.

### BACKGROUND OF THE INVENTION

Oxidative Stress describes the level of oxidative damage caused by reactive oxygen species in a cell, tissue, or organ. Reactive oxygen species (e.g., free radicals, reactive anions) are generated in endogenous metabolic reactions. Exogenous sources of reactive oxygen species include exposure to cigarette smoke and environmental pollutants. Reactions between free radicals and cellular components results in the alteration of macromolecules, such as polyunsaturated fatty acids in membrane lipids, essential proteins, and DNA. Where the formation of free radicals exceeds antioxidant activity, oxidative stress results. Oxidative stress is implicated in a variety of disease states, including Alzheimer's disease, Parkinson's disease, inflammatory diseases, neurodegenerative diseases, heart disease, HIV disease, chronic fatigue syndrome, hepatitis, cancer, autoimmune diseases cancer, and aging. Methods of preventing or treating pathologies associated with oxidative damage are urgently required.

### SUMMARY OF THE INVENTION

As described below, the present invention features methods for treating or preventing oxidative stress.

In one aspect, the invention generally features a method for increasing an antioxidant response in a cell (e.g., a pulmonary epithelial cell, a pulmonary endothelial cell, an alveolar cell, or a neuronal cell). The method involves contacting a cell expressing Nrf2 with an agent; and increasing (e.g., by at least about 10%, 25%, 50%, 75%, 85%, 95%) Nrf2 expression or biological activity in the cell relative to a control cell, thereby increasing an antioxidant response in the cell. In one embodiment, the method prevents or ameliorates a disease or disorder selected from the group consisting of pulmonary inflammatory conditions, pulmonary fibrosis, asthma, chronic obstructive pulmonary disease, emphysema, sepsis, septic shock, ischemic injury, cerebral ischemia and neurodegenerative disorders, meningitis, encephalitis, hemorrhage, cerebral ischemia, heart ischemia, cognitive deficits and neurodegenerative disorders. In another embodiment, Nrf2 expression reduces (e.g., by at least about 5%, 10%, 25%, 50%, 75%, 85%, 95%) subepithelial fibrosis, mucus metaplasia, or a structural alteration associated with airway remodeling. In another embodiment, the agent is a compound (e.g., Triterpenoid-155, Triterpenoid-156, Triterpenoid-162, Triterpenoid-225, or tricyclic bis-enones, a flavenoid, epicatechin, Egb-761, bilobalide, ginkgolide, or tert-butyl hydroperoxide) listed in Table 1A.

In another aspect, the invention features a method of preventing or ameliorating in a subject in need thereof a pulmonary inflammatory condition selected from the group consisting of pulmonary fibrosis, asthma, chronic obstructive pulmonary disease, and emphysema. The method involves contacting a pulmonary cell (e.g., pulmonary epithelial cell, a pulmonary endothelial cell, an alveolar cell) with an agent that increases by at least 10% an Nrf2 biological activity in the cell, thereby preventing or ameliorating the pulmonary inflammatory condition.

In yet another aspect, the invention features a method of preventing or ameliorating sepsis or septic shock in a subject (e.g., a human patient) in need thereof. The method involves contacting a cell of the subject with an agent that increases by at least 10% an Nrf2 biological activity in the cell, thereby preventing or ameliorating sepsis or septic shock.

In yet another aspect, the invention provides a method of preventing or ameliorating in a subject in need thereof a neurodegenerative disease that is any one or more of Alzheimer's disease (AD) Creutzfeldt-Jakob disease, Huntington's disease, Lewy body disease, Pick's disease, Parkinson's disease, amyotrophic lateral sclerosis (ALS), and neurofibromatosis. The method involves contacting a neuronal cell with an agent listed in Table 1A, where the agent increases by at least 10% an Nrf2 biological activity in the cell, and the agent is not a triterpenoid, thereby preventing or ameliorating the neurodegenerative condition.

In yet another aspect, the invention features a method of preventing or reducing cell death following an ischemic injury. The method involves contacting a cell at risk of cell death with an agent that increases by at least about 10% an Nrf2 biological activity in the cell, thereby preventing or reducing (e.g., by at least about 10%, 25%, 50%, 75%, 85% or more) cell death relative to an untreated control cell. In one embodiment, the method reduces apoptosis in a neural tissue of the subject.

In yet another aspect, the invention features a method increasing an antioxidant response in a cell. The method involves contacting the cell with a Nrf2 activating compound, thereby increasing an antioxidant response.

In yet another aspect, the invention features a method for protecting a neuronal cell from ischemic injury. The method involves contacting the neuronal cell with a Keap1 inhibitor, thereby protecting the neuronal cell from ischemic injury.

In yet another aspect, the invention features a method for ameliorating in a subject a condition related to oxidative stress. The method involves administering to the subject a vector containing an Nrf2 nucleic acid molecule positioned for expression in a mammalian cell; and expressing a Nrf2 polypeptide, or fragment thereof, in a cell of the subject, thereby ameliorating the condition in the subject.

In yet another aspect, the invention features a method for ameliorating a condition related to oxidative stress in a subject. The method involves administering to the subject a vector containing a Keap1 inhibitory nucleic acid molecule positioned for expression in a mammalian cell; and expressing the inhibitory nucleic acid molecule in a cell of the subject, thereby treating the subject.

In yet another aspect, the invention features a vector containing an Nrf2 nucleic acid molecule operably linked to a promoter suitable for expression in a pulmonary or neuronal cell.

In yet another aspect, the invention features a pulmonary host cell containing the vector of a previous aspect.

In yet another aspect, the invention features a vector containing a Keap1 inhibitory nucleic acid molecule operably linked to a promoter suitable for expression in a pulmonary or neuronal cell.

In yet another aspect, the invention features a Keap1 inhibitory nucleic acid molecule selected from the group consisting of an antisense oligonucleotide, siRNA, snRNA, or a ribozyme.

In yet another aspect, the invention features host cell containing the vector of a previous aspect or the inhibitory nucleic acid molecule of a previous aspect.

In yet another aspect, the invention features a pharmaceutical composition for the treatment or prevention of a pulmonary inflammatory condition, pulmonary fibrosis, asthma, chronic obstructive pulmonary disease, emphysema, sepsis, septic shock, containing a therapeutically effective amount of an agent that increases a Nrf2 biological activity or Nrf2 expression.

In yet another aspect, the invention features a pharmaceutical composition for the treatment or prevention of a pulmonary inflammatory condition, pulmonary fibrosis, asthma, chronic obstructive pulmonary disease, emphysema, sepsis, septic shock, cerebral ischemia or a neurodegenerative disorder containing a therapeutically effective amount of an agent that inhibits a Keap1 biological activity or Keap1 expression. In one embodiment, the agent reduces Keap1 inhibition of Nrf2. In another embodiment, the agent is an inhibitory nucleic acid molecule that decreases the expression of a Keap1 polypeptide or nucleic acid molecule.

In another aspect, the invention provides a pharmaceutical composition containing a Keap-1 inhibitory molecule in a pharmaceutically acceptable excipient.

In yet another aspect, the invention provides a packaged pharmaceutical containing a therapeutically effective amount of an agent that inhibits the expression or activity of Keap-1, and instructions for use in treating or preventing a pulmonary inflammatory condition, pulmonary fibrosis, asthma, chronic obstructive pulmonary disease, emphysema, sepsis, septic shock, cerebral ischemia, or a neurodegenerative disease.

In yet another aspect, the invention provides a packaged pharmaceutical containing a therapeutically effective amount of a Nrf-2 activating agent, and instructions for use in treating or preventing pulmonary inflammatory conditions, pulmonary fibrosis, asthma, chronic obstructive pulmonary disease, emphysema, sepsis, or septic shock.

In yet another aspect, the invention provides a method for identifying a subject as having or having a propensity to develop a pulmonary inflammatory conditions, pulmonary fibrosis, asthma, chronic obstructive pulmonary disease, emphysema, sepsis, or septic shock. The method involves detecting an alteration in a Keap1 or Nrf2 nucleic acid molecule present in a biological sample of the subject relative to a reference. In one embodiment, the alteration is a mutation in the nucleic acid sequence or an alteration in the polypeptide expression of Keap1 or Nrf2.

In yet another aspect, the invention provides a kit for the amelioration of a pulmonary inflammatory condition, pulmonary fibrosis, asthma, chronic obstructive pulmonary disease, emphysema, sepsis, or septic shock in a subject, the kit containing a nucleic acid molecule selected from the group consisting of: Keap-1 and Nrf-2 and written instructions for use of the kit for detection of the aforementioned conditions, diseases or disorders in a biological sample.

In yet another aspect, the invention provides a method of identifying an agent for the treatment or prevention of oxidative stress. The method involves contacting a cell that expresses a Keap-1 polypeptide with an agent; and comparing the expression of the Keap1 polypeptide in the cell contacted by the agent with the level of expression in a control cell not contacted by the agent, where a decrease in the expression of the Keap-1 polypeptide identifies the agent as treating or preventing oxidative stress.

In yet another aspect, the invention provides a method of identifying an agent for the treatment or prevention of oxidative stress. The method involves contacting a cell that expresses a Keap-1 nucleic acid molecule with an agent; and comparing the expression of the Keap1 nucleic acid molecule in the cell contacted by the agent with the level of expression in a control cell not contacted by the agent, where a decrease in the expression of the Keap-1 nucleic acid molecule thereby identifies the agent as treating or preventing oxidative stress.

In yet another aspect, the invention provides a method of identifying an agent for the treatment or prevention of oxidative stress. The method involves contacting a cell that expresses a Keap-1 polypeptide with an agent; and comparing the biological activity of the Keap1 polypeptide in the cell contacted by the agent with the level of biological activity in a control cell not contacted by the agent, where a decrease in the biological activity of the Keap-1 polypeptide thereby identifies the agent as treating or preventing oxidative stress.

In yet another aspect, the invention provides a method of identifying an agent for the treatment or prevention of oxidative stress. The method involves contacting a cell that expresses a Nrf2 polypeptide with an agent; and comparing the biological activity of the Nrf2 polypeptide in the cell contacted by the agent with the level of biological activity in a control cell not contacted by the agent, where an increase in the biological activity of the Nrf2 polypeptide thereby identifies the agent as treating or preventing oxidative stress.

In yet another aspect, the invention provides a method of identifying an agent for the treatment or prevention of oxidative stress. The method involves contacting a cell that expresses a Nrf2 polypeptide with an agent; and comparing the expression of the Nrf2 polypeptide in the cell contacted by the agent with the level of expression in a control cell not contacted by the agent, where an increase in the expression of the Nrf2 polypeptide identifies the agent as treating or preventing oxidative stress.

In yet another aspect, the invention provides a method of identifying an agent for the treatment or prevention of oxidative stress. The method involves contacting a cell that expresses a Nrf2 nucleic acid molecule with an agent; and comparing the expression of the Nrf2 nucleic acid molecule in the cell contacted by the agent with the level of expression in a control cell not contacted by the agent, where an increase in the expression of the Nrf2 nucleic acid molecule thereby identifies the agent as treating or preventing oxidative stress.

In yet another aspect, the invention provides a method of identifying an agent for the treatment or prevention of oxidative stress. The method involves contacting a cell containing a vector containing a Keap-1 nucleic acid molecule operably linked to a detectable reporter; detecting the level of reporter gene expression in the cell contacted with the candidate compound with a control cell not contacted with the candidate compound, where a decrease in the level of the reporter gene expression identifies the candidate compound as a candidate compound that treats or prevents oxidative stress.

In yet another aspect, the invention provides a method of identifying an agent for the treatment or prevention of oxidative stress. The method involves contacting a cell containing an expression vector containing a Nrf2 nucleic acid molecule operably linked to a detectable reporter; detecting the level of reporter gene expression in the cell contacted with the candidate compound with a control cell not contacted with the candidate compound, where an increase in the level of the reporter gene expression identifies the candidate compound as a candidate compound that treats or prevents oxidative stress.

In various embodiments of any of the above aspects, the compound is a compound listed in Table 1A or otherwise described herein. Exemplary compounds include, but are not limited to, Triterpenoid-155, Triterpenoid-156, Triterpenoid-162, Triterpenoid-225, or tricyclic bis-enones, flavonoids, epicatechin, Egb-761, bilobalide, ginkgolide, or tert-butyl hydroperoxide, and their derivatives. In still other embodiments of any of the above aspects, the method increases Nrf2 transcription, translation, or biological activity, or decreases Keap1 transcription, translation, or biological activity. In still other embodiments of any of the above aspects, the agent increases a Nrf2 biological activity that is any one or more of binding to an antioxidant-response element (ARE), nuclear accumulation, or the transcriptional induction of target genes (e.g., HO-1, NQO1, GCLm, GST α1, TrxR, Pxr 1, GSR, G6PDH, γGCLm, GCLc, G6PD, GST α3, GST p2, SOD2, SOD 3 and GSR). In still other embodiments, the agent reduces Keap1 inhibition of Nrf2 or the agent is an inhibitory nucleic acid molecule (e.g., an siRNA, an antisense oligonucleotide, a ribozyme, or a shRNA or a modified derivative thereof) that decreases the expression of a Keap1 polypeptide or nucleic acid molecule. In still other embodiments, the agent (e.g., antibody or an Nrf2 peptide fragment) disrupts Keap1 binding to Nrf2. In still other embodiments, the cell is in vivo or in vitro. In still other embodiments of the above aspects, the condition, disease or disorder is any one or more of pulmonary inflammatory conditions, pulmonary fibrosis, asthma, chronic obstructive pulmonary disease, emphysema, sepsis, septic shock, meningitis, encephalitis, hemorrhage, ischemic injury, cerebral ischemia, heart ischemia, cognitive deficits and neurodegenerative disorders. In still other embodiments, the neurodegenerative disorder is selected from the group consisting of Alzheimer's disease (AD) Creutzfeldt-Jakob disease, Huntington's disease, Lewy body disease, Pick's disease, Parkinson's disease, amyotrophic lateral sclerosis (ALS), and neurofibromatosis. In still other embodiments, the agent is administered in an aerosol composition.

Other features and advantages of the invention will be apparent from the detailed description, and from the claims.

### DEFINITIONS

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “ameliorate” is meant decrease, suppress, attenuate, diminish, arrest, or stabilize the development or progression of a disease.

By “antioxidant response” is meant an increase in the expression or activity of a Nrf2 regulated gene. Exemplary Nrf2 regulated genes are described herein.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease or disorder related to oxidative stress” is meant any pathology characterized by an increase in oxidative stress. Exemplary diseases or disorders related to oxidative stress include one or more of the following: pulmonary inflammatory conditions, pulmonary fibrosis, asthma, chronic obstructive pulmonary disease, emphysema, sepsis, septic shock, meningitis, encephalitis, hemorrhage, ischemic injury, cerebral ischemia, heart ischemia, cognitive deficits and neurodegenerative disorders

By “Nrf2 expression or biological activity” is meant binding to an antioxidant-response element (ARE), nuclear accumulation, the transcriptional induction of target genes, or binding to a Keap1 polypeptide.

By “Keap1 polypeptide” is meant a polypeptide comprising an amino acid sequence having at least 85% identity to GenBank Accession No. AAH21957.

By “Keap1 nucleic acid molecule” is meant a nucleic acid molecule that encodes a Keap1 polypeptide or fragment thereof.

By “neurodegenerative disorder” is meant any disease or disorder characterized by increased neuronal cell death, including neuronal apoptosis or neuronal necrosis.

By “pulmonary inflammatory condition” is meant any disease or disorder characterized by characterized by an increase in airway inflammation, intermittent reversible airway obstruction, airway hyperreactivity, excessive mucus production, or an increase in cytokine production (e.g., elevated levels of immunoglobulin E and Th2 cytokines).

By “ischemic injury” is meant any negative alteration in the function of a cell, tissue, or organ in response to hypoxia.

By “reperfusion injury” is meant any negative alteration in the function of a cell, tissue, or organ in response restore of blood flow following transient occlusion.

By “oxidative stress” is meant cellular damage or a molecular alteration in response to a reactive oxygen species.

By “protect a cell” is meant prevent or ameliorate an undesirable change in a cell or in a cellular component (e.g., molecular component). Typically, the undesirable change is in the function, structure, or physiology of the cell.

By “Nrf2 polypeptide” is meant a protein or protein variant, or fragment thereof, that comprises an amino acid sequence substantially identical to at least a portion of GenBank Accession No. NP_{—}006164 (human nuclear factor (erythroid-derived 2)-like 2) and that has a Nrf2 biological activity (e.g., activation of target genes through binding to antioxidant response element (ARE), regulation of expression of antioxidants and xenobiotic metabolism genes).

By “Nrf2 nucleic acid molecule” is meant a polynucleotide encoding an Nrf2 polypeptide or variant, or fragment thereof.

The phrase “in combination with” is intended to refer to all forms of administration that provide the inhibitory nucleic acid molecule and the chemotherapeutic agent together, and can include sequential administration, in any order.

The term “subject” is intended to include vertebrates, preferably a mammal. Mammals include, but are not limited to, humans.

By “marker” is meant any protein or polynucleotide having an alteration in expression level or activity that is associated with a disease or disorder.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibitory nucleic acid” is meant a single or double-stranded RNA, siRNA (short interfering RNA), shRNA (short hairpin RNA), or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises or corresponds to at least a portion of a target nucleic acid molecule, or an ortholog thereof, or comprises at least a portion of the complementary strand of a target nucleic acid molecule.

By “antisense nucleic acid”, it is meant a non-enzymatic nucleic acid molecule that binds to target RNA by means of RNA-RNA or RNA-DNA interactions and alters the activity of the target RNA (for a review, see Stein et al. 1993; Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk N A et al., 1999; Delihas N et al., 1997; Aboul-Fadi T, 2005.)

By “small molecule” inhibitor is meant a molecule of less than about 3,000 daltons having Nrf2 antagonist activity.

The term “siRNA” refers to small interfering RNA; a siRNA is a double stranded RNA that “corresponds” to or matches a reference or target gene sequence. This matching need not be perfect so long as each strand of the siRNA is capable of binding to at least a portion of the target sequence. SiRNA can be used to inhibit gene expression, see for example Bass, 2001, Nature, 411, 428 429; Elbashir et al., 2001, Nature, 411, 494 498; and Zamore et al., Cell 101:25-33 (2000).

By “corresponds to an Nrf2 gene” is meant comprising at least a fragment of the double-stranded gene, such that each strand of the double-stranded inhibitory nucleic acid molecule is capable of binding to the complementary strand of the target Nrf2 gene.

The term “microarray” is meant to include a collection of nucleic acid molecules or polypeptides from one or more organisms arranged on a solid support (for example, a chip, plate, or bead).

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant protein of the invention, or an RNA molecule).

By “reference” is meant a standard or control condition.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

By “promoter” is meant a polynucleotide sufficient to direct transcription.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

The term “pharmaceutically-acceptable excipient” as used herein means one or more compatible solid or liquid filler, diluents or encapsulating substances that are suitable for administration into a human.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and still more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Therapeutic compound” means a substance that has the potential of affecting the function of an organism. Such a compound may be, for example, a naturally occurring, semi-synthetic, or synthetic agent. For example, the test compound may be a drug that targets a specific function of an organism. A test compound may also be an antibiotic or a nutrient. A therapeutic compound may decrease, suppress, attenuate, diminish, arrest, or stabilize the development or progression of disease, disorder, or infection in a eukaryotic host organism.

By “transformed cell” is meant a cell into which (or into an ancestor of which) has been introduced, by means of recombinant DNA techniques, a polynucleotide molecule encoding (as used herein) a protein of the invention.

### BRIEF DESCRIPTION OF THE DRAWINGS

FIGS. 1 (A-L) Increased susceptibility of nrf2−/− mice to cigarette smoke (CS)-induced emphysema. FIG. 1 panels a-l show H&E stained lung sections from the air-exposed nrf2+/+ and nrf2−/− mice show normal alveolar structure (n=5 per group). Lung sections from the CS-treated (6 months) nrf2−/− mice show increased air space enlargement when compared with the lung sections from the CS-treated nrf2+/+ mice. Original magnification, 20×.

FIG. 2 (A-C) Cigarette smoke exposure causes lung cell apoptosis as assessed by TUNEL in nrf2−/− lungs. FIG. 2A consists of 12 panels showing TUNEL-stained, DAPI-stained, and merged images. Lung sections (n=5 per group) of room air-exposed or cigarette smoke (CS)-exposed (6 months) nrf2+/+ or nrf2−/− mice were subjected to TUNEL (right column) and DAPI stain (middle column). Merged images are shown in the right column. CS-exposed nrf2−/− mice show abundant TUNEL-positive cells (arrows) in the alveolar septa. Magnification, 20×. FIG. 2B is a graph showing quantification of TUNEL positive cells/total number of cells (DAPI). The numbers of TUNEL positive cells were significantly (*) higher in the CS exposed nrf2−/− mice when compared to its wild-type counterpart. mo, months. Values represent mean±SEM. FIG. 2C consists of 6 panels showing the identification of apoptotic (TUNEL-positive) type II epithelial cells (left column), endothelial cells (middle column), and alveolar macrophages (right column) in the lungs of CS-exposed (6 months) nrf2+/+ and nrf2−/− mice. Type II epithelial cells, endothelial cells, and alveolar macrophages were detected with anti-SpC, anti-CD 34 and Mac-3 antibodies respectively, as outlined in the Methods section. Nuclei were detected with DAPI. Shown are the merged images, with co-localization of cell specific markers and apoptosis (arrows indicate colocalization); non-apoptotic (TUNEL negative) cells with positive cell specific marker are highlighted with arrows. TUNEL-positive apoptotic cells lacking a cell specific marker are highlighted by arrowheads. The majority of TUNEL positive cells consisted of endothelial and type II epithelial cells, whereas most of alveolar macrophages were TUNEL negative.

FIGS. 3 (A-E) CS treatment leads to activation of caspase 3 in nrf2−/− lungs. FIG. 3A consists of four panels showing active caspase 3 expression in lung sections from the CS-exposed (6 months) nrf2+/+ and nrf2−/− mice. CS-exposed nrf2−/− mice show increased numbers of caspase 3-positive cells in the alveolar septa (n=5 per group). Magnification, 40×. FIG. 3B is a graph showing the number of caspase 3-positive cells in the lungs of air- and CS-exposed mice. Caspase 3-positive cells were significantly higher in the lungs of CS-exposed nrf2−/− mice. FIG. 3C shows the results of Western blot analysis. There is increased expression of the 18 kDa active form of caspase 3 in lungs of CS-exposed (6 months) nrf2−/− mice (lanes 1 and 3: air- and CS-exposed nrf2+/+ mice; lanes 2 and 4: air- and CS-exposed Nrf2−/− mice, respectively). FIG. 3D is a graph showing the quantification of procaspase 3 and active caspase 3 obtained in Western blots of air- or CS-exposed nrf2+/+ and −/− lungs. Values are represented as mean±SEM. FIG. 3E is a graph showing Caspase 3 activity in the lungs of air- or CS-exposed (6 months) nrf2+/+ and nrf2−/− mice. Caspase 3 activity was significantly higher in the lungs of CS-exposed nrf2−/− mice than in the lungs of wild-type counterpart (n=3 per group). Values (relative fluorescence units) are represented as mean±SEM.*, significantly greater than the CS-exposed nrf2+/+ mice. P≦0.05.

FIGS. 4 (A-C) Increased sensitivity of nrf2−/− mice to oxidative stress after CS exposure. FIG. 4A is one panel showing immunohistochemical staining for 8-oxo-dG in lung sections from the mice exposed to CS (6 months) (n=5 per group). Lung sections from the CS-exposed nrf2−/− mice show increased staining for 8-oxo-dG (indicated by arrows) when compared to lung sections from CS-exposed nrf2+/+ mice and the respective air-exposed control mice. Magnification, 40×. FIG. 4B is a graph showing quantification of 8-oxo-dG positive alveolar septal cells in lungs after 6 months of CS exposure. The number of anti-8-oxo-dG antibody-reactive cells was significantly higher in the lung tissues of the CS-exposed nrf2−/− mice than in the lung tissues of the CS-exposed nrf2+/+ mice and air-exposed control mice. Values (positive cells/mm alveolar length) represent mean±SEM. *, significantly greater than the CS exposed nrf2+/+ mice. P≦0.05. FIG. 4C is four panels showing immunohistochemical staining with normal mouse-IgG1 antibody in sections of lungs of air or CS-exposed nrf2+/+ and −/− mice. Magnification, 40×.

FIGS. 5 (A-C) Increased inflammation in the lungs of CS-exposed nrf2−/− mice. FIG. 5A is a graph showing lavaged inflammatory cells from control and CS-exposed mice. The number of macrophages in BAL fluid collected from CS-exposed nrf2−/− mice (1.5 months and 6 months of age) was significantly higher than in the BAL fluid from CS-exposed nrf2+/+ mice and the respective age-matched control mice. Values represent mean±SEM (n=8). *, significantly greater than control group of the same genotype; †, significant across the genotypes in CS-exposed group. P, ≦0.05. FIG. 5B is a series of four panels showing immunohistochemical detection of macrophages (arrows) in lungs of nrf2+/+ and nrf2−/− mice exposed to CS for 6 months. Magnification, 40×. FIG. 5C is a graph showing the quantification of macrophages in lungs after 6 months CS exposure. Lung sections from the CS-exposed nrf2−/− mice showed a significantly increased number of macrophages than wild-type counterpart exposed to CS (P≦0.025). There was no significant difference in the number of alveolar macrophages between the air-exposed nrf2+/+ and −/− mice (P≦0.9).

FIGS. 6 (A & B) Activation of Nrf2 in CS-exposed nrf+/+ lungs. FIG. 6A shows the results of EMSA to determine the DNA binding activity of Nrf2. For gel shift analysis, 10 μg of nuclear proteins from the lungs of air-and CS-exposed mice was incubated with the labeled human NQO1 ARE sequence and analyzed on a 5% non-denaturing polyacrylamide gel. For supershift assays, the labeled NQO1 ARE was first incubated with 10 μg of nuclear extract and then with 4 μg of anti-Nrf2 antibody for 2 h. Nuclear protein of nrf2+/+ lungs display increased binding to the ARE-containing sequence (lower arrow, [major band) after CS exposure, with a supershifted band caused by preincubation with anti-Nrf2 antibody, thus confirming the binding of Nrf2 to the ARE sequence (upper arrow, super shifted band). Ra-IgG₁: rabbit IgG₁. FIG. 6B shows the results of Western blot analysis. Western blot analysis with anti-Nrf2 antibody showed the nuclear accumulation of the transcription factor Nrf2 in the lungs of nrf2+/+ mice in response to CS exposure. Lanes 1 and 3: air-exposed nrf2−/− and +/+ mice, lanes 2 and 4: CS-exposed nrf2−/− and +/+ mice, respectively; lamin 1: loading control. Western blot analysis was carried out three times with the nuclear proteins isolated from the lungs of three different air or CS exposed nrf2+/+ and −/− mice.

FIGS. 7 (A & B) Validation of microarray data by Northern blot and enzyme assays. FIG. 7A is two panels showing analysis of mRNA levels of NQO1, GCLm, GST al, HO-1, TrxR, Pxr 1, GSR, and G6PDH in the lungs of nrf2+/+ and nrf2−/− mice exposed to either air or CS, n=3 per group. FIG. 7B is a series of five graphs that show the effect of CS on the specific activities of selected enzymes in the lungs of nrf2+/+ and nrf2−/− mice. Values represent mean±SE (n=3 per group). *, significantly greater than control group of the same genotype. P≦0.05.

FIGS. 8 (A-G) Increased allergen-driven asthmatic inflammation in OVA challenged Nrf2^{-/-} mice. The graphs shown in panels A-E represent total number of cells×104/ml in BAL fluid following OVA challenge. (A) Total and differential inflammatory cell populations [(B) 1^{st} challenge with OVA; (C), 2^{nd} challenge with OVA; (D) and (E), 3rd challenge with OVA] in the BAL fluid of OVA and saline challenged Nrf2^{+/+} and Nrf2^{-/-} mice (n=8/group). There was a progressive increase in the total number of inflammatory cells in the BAL fluid of both OVA challenged Nrf2^{+/+} and Nrf2 mice from the 1^{st} to 3^{rd} challenges. The number of inflammatory cells in the BAL fluid of Nrf2^{-/-} OVA mice was significantly higher than in the BAL fluid of Nrf2^{+/+} OVA mice as well as the respective saline challenged mice. The number of eosinophils, lymphocytes, neutrophils and epithelial cells were significantly (*) higher in the BAL fluid of Nrf2^{-/-} OVA mice compared to Nrf2^{+/+} OVA mice. As shown in FIGS. 9 A-9D, Nrf2^{-/-} mice had increased infiltration of inflammatory cells into the lungs following OVA challenge. Pretreatment with NAC significantly (*) reduced the inflammatory cells (F), predominantly eosinophils (G) in the BAL fluid of Nrf2^{-/-} OVA mice (n=6 mice in each group). Data are mean±SEM. P≦0.05. The figure is representative of three experiments (n=6 mice per group).

FIGS. 9 (A-D) Increased infiltration of inflammatory cells into lungs of OVA challenged Nrf2−/− mice. FIGS. 9 (A-D) shows H & E staining of lung sections. Lung tissues from the saline and OVA challenged (3^{rd} challenge) Nrf2^{+/+} and Nrf2^{-/-} mice (n=6) were stained with H&E and examined by light microscopy (20×). FIG. 9 (A) consists of four panels of stained lung sections. A higher number of inflammatory cells was observed in the perivascular, peribronchial and parenchymal tissues of the Nrf2^{-/-} OVA mice as compared to a few inflammatory cell infiltrates observed in the Nrf2^{+/+} OVA mice. FIGS. 9 (B) and 9 (C) consist of four panels of stained lung sections. Immunohistochemical staining with anti-major basophilic protein (anti-MBP) antibody showed numerous eosinophils around the blood vessels (BV) and airways (AW) (FIG. 9 B) and in the parenchymal tissues (FIG. 9 C) of Nrf2^{-/-} OVA mice compared to the Nrf2^{+/+} OVA mice. FIG. 9 (D) consists of four panels of stained lung sections from the saline or NAC treated (7 days before 1^{st} OVA challenge) Nrf2-deficient mice. Widespread peribronchial and perivascular inflammatory infiltrates were observed in OVA sensitized mice after antigen provocation (FIG. 9D, bottom right panel). Pretreatment of Nrf2-deficient mice with NAC resulted in significant reduction in the infiltration of inflammatory cells in the peribronchial and perivascular region (D, bottom left panel).

FIGS. 10 (A-F) increased oxidative stress markers, eotaxin and enhanced activation of NF-κB in the lungs of Nrf2^{-/-} OVA mice. Panels 10A and 10B are graphs that show increased levels of lipid hydroperoxides and protein carbonyls, respectively, in the lungs of OVA challenged Nrf2 mice. Values are mean±SEM. *, significantly higher than the Nrf2^{+/+} OVA mice. n=6 mice in each group. FIG. 10C is a graph showing eotaxin level in the BAL fluid. When compared to OVA challenged Nrf2^{+/+} mice, the BAL eotaxin level was markedly higher in OVA challenged (both 1^{st} and 3^{rd} challenge) Nrf2^{-/-} mice (P≦0.05). n=6 mice in each group. Activation of NF-κB in the lungs is shown in FIGS. 10D-F. Western blot was used to determine the activation of p50 and p65 subunits of NF-κB in the lungs (FIG. 10D). Lanes 1 and 2: saline challenged Nrf2^{+/+} and Nrf2^{-/-} mice, respectively. Lanes 3 and 4: OVA challenged Nrf2^{+/+} and Nrf2^{-/-} mice, respectively. Quantification of p50 and p65 subunits of NF-κB obtained in Western blots is shown in panel (E). Values are mean±SEM of three experiments. FIG. 10F shows an ELISA measurement of p65/Rel A subunit of NF-κB using Mercury TransFactor kit. *, P≦0.05 versus OVA challenged Nrf2 wild-type mice. Data are mean±SEM of three experiments.

FIGS. 11 (A & B) Nrf2-deficient mice show increased mucus cell hyperplasia in response to allergen challenge. FIG. 11 (A) is a panel of 4 lung sections (72 h after the final OVA challenge) stained with PAS. Epithelial cells are shown with arrows in the proximal airways of OVA challenged mice. Pronounced mucus cell hyperplasia is found in Nrf2^{-/-} OVA mice (40×). FIG. 11 (B) is a graph showing the percentage of airway epithelial cells positive for mucus glycoproteins as determined by PAS staining. Lung sections from the Nrf2^{-/-} OVA mice showed significantly higher numbers of PAS positive cells than the lung sections from the Nrf2^{+/+} OVA mice (*). Data are mean±SEM. P≦0.05.

FIGS. 12 (A-D) Nrf2-deficent mice show increased airway responsiveness to acetylcholine challenge. FIG. 12 shows 4 graphs, (A-D). OVA challenged Nrf2^{+/+} and Nrf2^{-/-} mice (3^{rd} challenge) were challenged with acetylcholine aerosol by nebulization with an Aeroneb Pro-nebulizer (n=7 mice per group). Lung resistance and compliance were measured. The percent increase in elastance (C) and resistance (D) to acetylecholine challenge were significantly higher (*) in the Nrf2^{-/-} OVA mice when compared to Nrf2^{+/+} OVA mice and the respective saline challenged mice. No significant difference in baseline elastance (A) and resistance (B) was observed in either the saline and OVA challenged Nrf2^{+/+} and Nrf2^{-/-} mice in the absence of acetylcholine challenge. Data are mean±SEM. P≦0.05.

FIGS. 13 (A & B) Th2 cytokine levels in the BAL fluid of Nrf2^{+/+} and Nrf2^{-/-} mice challenged with ovalbumin. FIGS. 13 (A & B) are graphs. BAL fluids collected 48 h after the 2^{nd} OVA challenge were used for cytokine assays using ELISA. Graphs show that the amounts of both IL-4 (A) and IL-13 (B) were significantly higher (*) in the BAL fluid of Nrf2^{-/-} OVA mice than Nrf2^{+/+} OVA mice (n=8/group). Data are mean±SEM. P≦0.05.

FIGS. 14 (A & B) Activation of Nrf2 in the lungs of OVA challenged Nrf2^{+/+} mice FIG. 14 (A) shows the results of EMSA. EMSA was used to determine the activation of Nrf2 in the lungs of Nrf2^{+/+} OVA mice. Equal amounts of nuclear extracts (10 μg) prepared from lungs were incubated with radio-labeled ARE from the hNQO1 promoter and analyzed by EMSA. EMSA analysis showed the increased binding of nuclear proteins isolated from the lungs of OVA challenged Nrf2^{+/+} mice to ARE sequence. The super-shifted band is indicated by the arrow. FIG. 14 (B) shows the result of immunoblot analysis with anti-Nrf2 antibody. Lanes 1 and 2: saline challenged Nrf2^{-/-} and Nrf2^{+/+} mice, respectively; Lanes 3 and 4: OVA challenged Nrf2^{-/-} and Nrf2^{+/+} mice, respectively. The figure is representative of three experiments.

FIG. 15 Real Time RT-PCR analysis of selected antioxidant genes in the lungs of OVA challenged Nrf2^{+/+} and Nrf2^{-/-} mice. FIG. 15 is a panel of 9 graphs quantifying the results of RT-PCR analysis. Real Time RT-PCR analysis showed increased levels of mRNA for genes including γ GCLm, GCLc, G6PD, GST α3, GST p2, HO-1, SOD2, SOD 3 and GSR in the lungs of Nrf2^{+/+} OVA as compared to gene levels in the lungs of Nrf2^{-/-} OVA mice and saline challenged mice. Solid bar, Nrf2^{+/+} mice; open bar, Nrf2^{-/-} mice.

FIGS. 16 (A & B) Redox status in the lungs of Nrf2^{+/+} and Nrf2^{-/-} mice. FIGS. 16 (A & B) are graphs showing the % GSH increase and GSH/GSSG ratios in the lungs of saline and OVA challenged Nrf2^{+/+} and Nrf2^{-/-} mice. FIG. 16 (A) shows GSH levels in the lungs of Nrf2 wild-type and knock out mice. OVA challenged (1^{st} and 3^{rd} challenge) Nrf2^{+/+} mice showed a significant increase in GSH level in the lungs when compared with the OVA challenged Nrf2^{-/-} mice. The endogenous total GSH was 15% higher in the saline challenged Nrf2^{+/+} than the Nrf2^{-/-} mice. Furthermore, there was greater increase in GSH in the OVA challenged wild-type mice [54% vs 14.8% (1^{st} challenge); 40% vs 17% (3^{rd} challenge)] than the Nrf2 challenged with OVA. FIG. 16 (B) shows the GSH/GSSG ratio in the lungs of OVA challenged Nrf2^{+/+} mice. In response to OVA challenge, there was a dramatic increase in the GSH/GSSG ratio in the lungs of Nrf2^{+/+} mice [8.6 (saline), 15.9 (1^{st} challenge); 8.3 (saline), 14.3 (3^{rd} challenge)]. There was a smaller increase in the GSH/GSSG ratio in Nrf2 OVA mice [4.8 (saline), 6.5 (1^{st} challenge); 4.9 (saline), 6.2 (3^{rd} challenge)]. GSH/GSSG ratio was also significantly higher (*) in the lungs of saline challenged Nrf2^{+/+} mice than Nrf2^{-/-} mice. n=6 mice per group. Data are mean±SEM. P≦0.05.

FIGS. 17 (A-C) Expression of Nrf2-dependent antioxidant genes in CD4⁺ T cells and macrophages. FIG. 17A shows the results of RT-PCR, showing the expression of Nrf2 and Nrf2 dependent antioxidant genes (HO-1, GCLc and GCLm) in CD4⁺ T cells in the lung (lanes 1 and 2), and macrophages (lanes 3 and 4), isolated from the OVA challenged Nrf2^{+/+} and Nrf2^{-/-} mice. Lanes 1 and 3 are Nrf2^{-/-} OVA lung CD4⁺ T cells and macrophages, respectively; Lanes 2 and 4 are Nrf2^{+/+} OVA lung CD4⁺ T cells and macrophages, respectively. β actin was used as the internal control. FIGS. 17 (B) and (C) are graphs showing that the message levels of the antioxidant genes HO-1, GCLc and GCLm were significantly higher in the CD4⁺ T cells (B) and macrophages (C) isolated from the lungs of OVA challenged Nrf2 wild-type than the knock out counterpart.

FIGS. 18 (A-D). Transient transfection in mouse Hepa cells and human Jurkat T cells. (A) is a graph showing Nrf2 overexpression in mouse Hepa cells, (B) is a graph showing overexpression of Nrf2 in Jurkat cell line and the analysis of Nrf2 dependent antioxidant genes, (C) is a graph showing the effect of Nrf2 overexpression on IL-13 promoter activity and (D) is a graph showing IL-13 protein level in the Jurkat cell line. Nrf2-pUB6 construct was transfected into mouse Hepa cells stably transfected with HO-1 ARE. Transfection of Hepa cells with Nrf2-pUB6 construct enhanced the HO-1 ARE luciferase activity, suggesting the activation of HO-1 promoter activity by the transcription factor Nrf2 (A). Jurkat T cells were transiently transfected with Nrf2 overexpressing-pUB6 vector or empty pUB6 vector and stimulated with or without PMA and calcium ionophore A23187 (B-D). (B) Real Time RT-PCR analysis revealed a significantly increased expression of Nrf2 and Nrf2-regulated antioxidant genes, GCLc, and NQO1 in Jurkat cells transfected with Nrf2 overexpressing vector and stimulated with PMA plus A23187, as compared to Jurkat cells transfected with pUB6 control vector and stimulated with PMA plus A23187, and Jurkat cells stimulated with PMA plus A23187 or control Jurkat cells. (*P≦0.05). The results are mean±SEM of three independent experiments. Jurkat PMA, Jurkat cells stimulated with PMA plus A23187; pUB6 PMA, Jurkat cells transfected with pUB6 empty vector and stimulated with PMA plus A23187; Nrf2-pUB6 PMA, Jurkat cells transfected with Nrf2-pUB6 vector and stimulated with PMA plus A23187. (C) Nrf2 overexpression did not affect transcriptional activation of the proximal IL-13 or IL-4 promoters. Data are the average of n=2 independent experiments, and are expressed relative to the activity of the promoter in unstimulated cells which was set equal to 1. The shaded triangle indicates increasing amounts of Nrf2 or empty expression vectors (0 to 5 μg). In contrast to the robust secretion of IL-13, the Jurkat T cells used in these experiments do not secrete abundant levels of IL-4 protein, and there was no effect of Nrf2 overexpression on IL-4 secretion. A23+PMA, Jurkat cells stimulated with A23187 plus PMA. The protein level of the Th2 cytokine IL-13 (D) in the culture supernatants was measured using ELISA. No significant difference was observed in the level of secreted IL-13 protein in cells overexpressing Nrf2. Data are expressed as mean±SEM of three independent experiments. (P≦0.05).

FIGS. 19 (A & B) Nrf2−/− mice are more sensitive to LPS and septic peritonitis-induced septic shock. FIGS. 19 (A and B) are graphs showing mortality after LPS administration. Age-matched male nrf2+/+ (n=10) and nrf2−/− mice (n=10) were intraperitoneally injected with LPS (0.75 and 1.5 mg per mouse). FIG. 19 (C) is a graph showing the results of experiments wherein acute septic peritonitis was induced by CLP. CLP and sham operation were performed on age-matched male nrf2+/+(n=10) and nrf2−/− mice (n=10) as described in methods. Mortality was assessed every 12 h for 5 days. *, Nrf2+/+ had improved survival compared to nrf2−/− mice (P<0.05).

FIG. 20 Non-lethal dose of LPS induced greater lung inflammation in nrf2-deficient lungs. FIGS. 20 (A and B) are graphs showing BAL fluid analysis of nrf2−/− and nrf2+/+ mice after 6 and 24 h of ip injection of LPS (60 μg per mouse). FIG. 20 (C) is a graph showing BAL fluid analysis of nrf2−/− and nrf2+/+ mice after 6 h and 24 h of LPS instillation (10 μg per mouse). FIG. 20 (D) consists of four panels showing histopathological analysis of lungs by H&E staining 24 h after instillation of LPS. Arrows indicate accumulation of inflammatory cells in the alveolar spaces. Magnification, ×20. FIG. 20 (E) consists of four panels showing results of immunohistology of lungs of both genotypes using anti-mouse neutrophil antibody 24 h after LPS instillation. Sections were counterstained with hematoxylin. Arrows indicate neutrophils; Magnification, ×40. FIG. 20 (F) is a graph showing myeloperoxidase activity in lung homogenates of both genotypes 6 and 24 h after LPS instillation. FIG. 20 (G) is a graph wherein pulmonary edema was assessed by the ratio of wet to dry lung weight 24 h after LPS instillation. Data are presented as mean±SE (n=5). * Differs from vehicle control of the same genotype; †, differs from LPS treated wild-type type mice. P<0.05.

FIGS. 21 (A-C) LPS and CLP induces greater secretion of TNF-α in nrf2-deficient mice. (A-C) are graphs showing serum concentrations of TNF-α. (A) Serum concentration of TNF-α in nrf2+/+ and nrf2−/− mice 1.5 h after LPS injection (1.5 mg per mouse). (B) Serum concentration of TNF-α in nrf2+/+ and nrf2−/− mice 6 h after CLP. (C) TNF-α levels in the BAL fluid at 2 h after LPS delivery either by ip injection (60 μg per mouse) and or intratracheal instillation (10 μg per mouse). TNF-α in the BAL fluid of vehicle treated mice was not detectable. Data are presented as mean±SE. * Differs from vehicle control of the same genotype; †, differs from LPS treated wild-type mice. P<0.05. ND, Not detected.

FIGS. 22 (A-C) Greater expression of pro-inflammatory genes associated with innate immune response in the lungs of nrf2-deficient mice. (A-C) are graphs showing the expression of Cytokines (A), Chemokines (B) and Adhesion molecules/receptors (C) 30 min after non-lethal ip injection of LPS (60 μg per mouse) in nrf2-deficient and wild-type mice obtained from microarray analysis. Data is represented as mean fold change obtained from comparing LPS challenge to vehicle treated lungs of the same genotype on a semilog scale. All the represented fold change values of LPS treated lungs of nrf2−/− mice is significant compared to wild-type mice at P<0.05.

FIGS. 23 (A-C) TNF-α stimulus induced greater lung inflammation in nrf2-deficient mice. FIG. 23 (A) is a graph showing BAL fluid analysis at 6 h after ip injection of TNF-α (10 μg per mouse). FIG. 23 (B) consists of two panels showing histopathological analysis of lungs of nrf2+/+ and nrf2−/− mice by H&E staining 24 h after ip injection of TNF-α (10 μg per mouse). Vehicle treated lungs are not shown. Magnification, ×20. FIG. 23 (C) is a panel of three graphs showing expression analysis of TNF-α, IL-1β and IL-6 by real time PCR in the lungs of nrf2−/− and nrf2+/+ mice 30 min after TNF-α challenge. Data are presented as mean±SE. * Differs from vehicle control of the same genotype; †, differs from LPS treated wild-type mice.

FIGS. 24 (A-D) LPS induced greater NF-κB activation in nrf2-deficient mice lungs. FIG. 24(A) shows the results of EMSA. Lung nuclear extracts from nrf2−/− and nrf2+/+ mice were assayed for NF-κB-DNA binding activity by EMSA 30 min after instillation of LPS (10 μg per mouse). The major NF-κB bands contained p65 and p55 subunits, as determined by the supershift obtained by p65 and p50 antibody. Lanes: 1, vehicle Nrf2+/+; 2, LPS Nrf2+/+; 3, vehicle Nrf2−/−; 4, LPS Nrf2−/−; 5, LPS, Nrf2+/+ with p65 antibody, 6, LPS, Nrf2+/+ with p50 antibody. SS, supershift. FIG. 24 (B) is a graph showing quantification of NF-κB-DNA binding as performed by densitometric analysis. All values are mean±SE obtained from three animals per treatment group and are represented as relative to respective vehicle control. FIG. 24 (C) shows the results of Western blot analysis. The blot shows nuclear accumulation of p65 by western blot in the nuclear extracts derived from lungs of nrf2+/+ and nrf2−/− mice 30 min after instillation of LPS (10 μg per mouse). Lamin B1 was used as loading control. FIG. 24 (D) is a graph showing densitometric analysis of western blot of RelA relative to wild-type vehicle control. All values are mean±SE (n=3). * Differs from vehicle control of the same genotype, †, differs from LPS treated wild-type type mice. P<0.05.

FIGS. 25 (A-C) Lack of nrf2 augments NF-κB activation in macrophages. FIG. 25 (A) shows results of EMSA experiments. Nuclear extracts of nrf2+/+ and nrf2−/− peritoneal macrophages were assayed for NF-κB-DNA binding by EMSA 20 min after LPS treatment (1 ng/ml). Oct1 was used as loading control. FIG. 25 (B) is a graph showing densitometric analysis of NF-κB-DNA binding relative to wild-type vehicle control. Values are mean±SE (n=3). FIG. 25 (C) is a graph showing TNF-α levels in the culture media from nrf2+/+ and nrf2−/− peritoneal macrophages after 0.5 h, 1 h and 3 h of LPS treatment (1 ng/ml). * Differs from vehicle control of the same genotype; †, Differs from wild-type treatment group. P<0.05

FIGS. 26 (A-H) LPS and or TNF-α stimulus induces greater NF-κB activation in nrf2-deficient MEFs. FIG. 26 (A) shows the results of EMSA experiments. Nuclear extracts from nrf2+/+ and nrf2−/− MEFs were assayed for NF-κB-DNA binding activity by EMSA 30 min after LPS (0.5 μg/ml) and or TNF-α (10 ng/ml). The major NF-κB bands contained p65 and p55 subunits, as determined by the supershift analysis using p65 and p55 antibody. FIG. 26 (B) is a graph showing the quantification of NF-κB-DNA binding. Quantification was performed by densitometric analysis. All values are mean±SE (n=3) and are represented relative to respective vehicle control. FIG. 26 (C) is a graph showing the results of experimentation wherein NF-κB mediated reporter activity in MEFs of both genotypes challenged with LPS (0.5 μg/ml) and TNF-α (10 ng/ml). At 24 h after transfection with pNF-κB-luc vector, cells were treated with either LPS and or TNF-α for 3 h and then luciferase activity was measured. Data are mean SE from 3 independent experiments (n=3). FIG. 26 (D) is an immunoblot of IκB-α and P-IκB-α protein in nrf2+/+ and nrf2−/− MEFs after LPS (0.5 μg/ml) or TNF-α (10 ng/ml) stimulus. FIGS. 26 (E and F) are graphs showing the quantification of IκB-α (E) and P-IκB-α (F) protein in nrf2+/+ and nrf2−/− MEFs by densitometric analysis. Data are mean±SE (n=3). FIG. 26 (G) are the results of [Western analysis showing IKK activity in nrf2+/+ and nrf2−/− MEFs after LPS (0.5 μg/ml) or TNF-α (10 ng/ml) stimulus. FIG. 26 (H) is a graph showing quantification of IKK activity in nrf+/+ and nrf2−/− MEFs by densitometric analysis. Data are mean±SE from (n=3). * Differs from vehicle control of the same genotype; †, Differs from wild-type treatment group. P<0.05

FIG. 27 Nrf2 deficiency increases LPS and or poly(I:C) induced IRF3 mediated luciferase reporter activity in MEFs. FIG. 27 is a graph showing relative fold change in luciferase activity. At 24 h after transfection with ISRE-Tk-Luc vector, cells were treated with LPS and or poly(I:C) for 6 h and luciferase assays were performed 6 h after treatment. For poly(I:C) stimulation, MEFs were transfected with 6 μg of poly(I:C) in 8 μl of Lipofectamine2000. Data are mean±SE from 3 independent experiments (n=3). * Differs from vehicle control of the same genotype; †, Differs from wild-type treatment group. P<0.05

FIGS. 28 (A-D) Lower levels of GSH in the lungs and MEFs of nrf2-deficient mice. FIG. 28 (A) is a graph showing the constitutive expression of GCLC in lungs and MEFs of nrf2+/+ and nrf2−/− mice. FIG. 28 (B) is a graph showing GSH levels in the lungs of mice of both genotypes 24 h after LPS instillation (10 μg per mouse). Data are mean±SE from 3 independent experiments and are expressed as percent increase relative to vehicle-treated nrf2+/+ group. FIG. 28 (C) is a graph showing the ratio of GSH to GSSG measured 24 h after LPS instillation in the lung of nrf2+/+ and nrf2−/− mice. Data are mean±SE from 3 independent experiments FIG. 28 (D) is a graph showing GSH levels in nrf2+/+ and nrf2−/− MEFs at 1 h after LPS (0.5 μg/ml) stimulus. Data are presented as mean±SE (n=4). * Differs from vehicle control of the same genotype; j, Differs from wild-type treatment group. P<0.05

FIGS. 29 (A-D) Pretreatment with exogenous antioxidants alleviate inflammation in nrf2-deficient mice. FIG. 29 (A) is a graph showing NF-κB mediated luciferase reporter activity in nrf2−/− MEFs pretreated for 1 h with NAC (10 mM) and or GSH-MEE (GSH) (1 mM) after 3 h of LPS (0.5 μg/ml) and or TNF-α (10 ng/ml) stimulus. Data are presented as mean±SE (n=4). * Differs from vehicle control; †, differs from group that was treated with LPS or TNF-α only, P<0.05. FIG. 29 (B) is a graph showing expression of TNF-α, IL-1β and IL-6 by real time PCR at 30 mM in the lungs of nrf2−/− mice pretreated with NAC after LPS (ip, 60 μg per mouse) challenge. FIG. 29 (C) is a graph showing results of BAL fluid analysis at 6 h in lungs of nrf2−/− mice pretreated with NAC after LPS (ip, 60 μg per mouse) challenge. Nrf2−/− mice were pretreated with three doses of NAC (500 mg/kg body weight, ip, every 4 h). Data are presented as mean±SE (n=4). * Differs from vehicle control; †, Differs from only LPS treatment. P<0.05. FIG. 29 (D) is a graph showing LPS induced mortality in nrf2−/− and nrf2+/+ mice pretreated with NAC. Age-matched male nrf2−/− (n=10) and nrf2+/+ mice (n=10) were either pretreated with NAC (ip, 500 mg/kg body weight) and or saline every day for 4 days followed by LPS challenge (1.5 mg per mouse). Mortality (% survival) was assessed every 12 h for 5 days. *, Mice pretreated with NAC had improved survival compared to vehicle-pretreated mice (P<0.05).

FIG. 30 p55 and p75 levels are increased with LPS treatment. FIG. 30 is a graph showing serum levels of p55 and p75 as analyzed by ELISA (R & D Systems). Nrf2-deficient and wild-type mice after 6 h of treatment with either vehicle and or LPS (1.5 mg/mouse). *, differs from vehicle control of the same genotype; P<0.05. ND, Not detected.

FIG. 31 Protein levels of TLR4 and CD14. FIG. 31 shows two panels of results from Western blot analysis. Constitutive protein levels of TLR4 are shown in the left panel, and protein levels of CD14 are shown in the right panel. Protein levels were determined from whole cell extracts obtained from peritoneal macrophages of nrf2−/− and nrf2+/+ mice by immunoblot. Immunoblot analysis was performed as described in the methods section using antibodies specific for the TLR4 and CD14.

FIGS. 32 (A & B) Increased binding of p65/Rel A subunit in LPS treated Nrf2−/− mice. FIG. 32 (A) is a graph showing the results of a DNA binding activity assay. The graph shows that there is increased binding of p65/Rel A subunit from the lung nuclear extracts obtained from LPS treated Nrf2−/− mice to an NF-κB binding sequence compared with its wild-type counterpart. FIG. 32 (B) is a graph showing that in response to LPS or TNF-α treatment, nuclear extracts from nrf2−/− MEFs demonstrated increased binding of p65/Rel A subunit to NF-103 binding sequence when compared to wild-type MEFs.

FIG. 33 Rigid and Flexible probes. FIG. 33 is a photo showing examples of rigid and flexible probes. The probe on the left is a 6-0 monofilament preheated and coated with methyl methacrylate glue (rigid probe). The probe on the right is an 8-0 monofilament coated with silicone (flexible probe).

FIG. 34 Middle cerebral artery occlusion technique. FIG. 34 is a schematic diagram showing the technique of middle cerebral artery occlusion with 8-0 monofilament coated with silicone (flexible probe) is shown. CCA, common carotid artery; ECA, external carotid artery; ICA, internal carotid artery; MCA, middle cerebral artery.

FIG. 35 Comparison of infarction volume: rigid and flexible probe. FIG. 35 consists of two panels, top and bottom. The top panel shows representative images of brain slices showing infarction after 90 minutes of ischemia and 22 hours of reperfusion. The middle cerebral artery was occluded with a rigid probe (left) or a flexible probe (right). The horizontal line represents 1 mm distance. The bottom panel is a graph that shows no significant difference was observed in infarction volume obtained by the two techniques.

FIG. 36 No difference in cerebral infarction volume between WT and HO-1^{-/-} mice using a rigid probe. FIG. 36 consists of two panels, top and bottom. The top panel shows representative images of brain slices from WT (left) and HO-1^{-/-} (right) mice after 90 minutes of middle cerebral artery occlusion with a rigid probe and 22 hours of reperfusion. The horizontal line represents 1 mm distance. FIG. 36, bottom panel, is a graph showing cerebral infarction volume was similar in the HO-1^{-/-} and WT mice.

FIG. 37 No difference in cerebral infarction volume between WT and HO-1^{-/-} mice using a flexible probe. FIG. 37 consists of two panels, top and bottom. The top panel shows representative images of brain slices from WT (left) and HO-1^{-/-} (right) mice after 90 minutes of middle cerebral artery occlusion with a flexible probe and 22 hours of reperfusion. The horizontal line represents 1 mm distance. FIG. 37, bottom panel, is a graph showing cerebral infarction volume was similar in the HO-1^{-/-} and WT mice.

FIG. 38 Corrected infarct volume is greater in Nrf^{-/-} (30.8±6.1%) mice. FIG. 38 is a graph showing representative photographs of infarcted brains from WT and Nrf2^{-/-} mice (n=8/group), subjected to 90 minutes MCAO and 24 hours of reperfusion. Scale bar represents 1 mm. The graph represents corrected infarct volume, which was significantly larger in the Nrf2^{-/-} (30.8±6.1%) mice than in the WT mice (17.0±5.1%); *P<0.01.

FIG. 39 Neurological deficit score is greater in Nrf2^{-/-} mice. FIG. 39 is a graph showing the neurological deficit scores of mice 1, 2, and 24 hours after ischemia is shown. Neurological dysfunction was significantly greater in the Nrf2^{-/-} mice (3.1±0.3) than in the WT mice (2.5±0.2) 24 hours after ischemia; *P<0.04. (Rep), reperfusion.

FIG. 40 Relative cerebral blood flow in WT and Nrf2^{-/-} mice is not different. FIG. 40 is a graph showing relative cerebral blood flow (CBF) in WT and Nrf2^{-/-} mice (n=5/group), determined using laser-Doppler flowery is shown. Mice underwent 90 minutes MCAO, and 1 hour reperfusion. CBF was monitored from 15 minutes before MCAO through 1 hour of reperfusion. No significant differences in CBF were observed between WT and Nrf2^{-/-} mice at any time during the experiment.

FIGS. 41 (A-D) Effect of t-BuOOH, NMDA or glutamate treatments on Nrf2 location. This figure consists of four panels (A) through (D) that show the results of Western analysis. Primary cortical neurons were incubated for the times shown (minutes) with serum-free B27 minus antioxidant supplement media alone or that containing (A) t-BuOOH (60 (B) NMDA (100 μM), or (C) glutamate (300 μM). Nuclear and cytoplasmic samples were analyzed by Western blotting using antibodies to Nrf2 and actin. The actin expression level was unchanged. FIG. 41 (D) consists of three histograms that show the ratio of chemiluminescence emitted from the Nrf2 to chemiluminescence emitted from the actin of each sample. Values shown are means±SE for three independent blots. *P<0.001 vs control.

FIGS. 42 (A & B) Effect of t-BuOOH, NMDA, or glutamate in the presence of BHQ. FIGS. 42 A and B are graphs depicting the results of (A) MTT assay and (B) caspase 3/7 assay. Neurons were grown for 24 hours in culture medium alone (control), or in the presence of t-BuOOH (60 μM), NMDA (100 μM), or glutamate (300 μM) with or without t-BHQ (20 μM). FIG. 42 (A) is a graph assessing neuronal viability. Neuronal viability was assessed by MTT assay, and the absorbance at 570 nm is shown (expressed as percent of control). *P<0.001 vs control; #P<0.05 vs t-BuOOH, NMDA, or glutamate, respectively. FIG. 42 (B) is a graph showing caspase-3 activity. Caspase-3 activity was determined and shown as the amount of fluorescent substrate formed *P<0.001 vs control; #P<0.05 vs t-BuOOH, NMDA, or glutamate, respectively.

FIGS. 43 (A & B) Effect of EGb 761 pretreatment on stroke outcome. This figure is two graphs showing the effect of EGb 761 pretreatment on stroke outcome. Panel (a) is a graph showing neurological deficit scores and panel (b) is a graph showing percent corrected infarct volume after 2 h of middle cerebral artery occlusion and 22 h of reperfusion are shown. Data are expressed as mean±sem; n=10-12. **P<0.01 vs. vehicle-treated control.

FIG. 44 Quantification of regional cerebral blood flow. This figure shows the quantification of regional cerebral blood flow (CBF). Regional CBF was determined by [14C]-IAP autoradiography within six regions of contralateral nonischemic cortex, ipsilateral ischemic cortex, and caudate putamen, subdivided into parietal, lateral and medial areas, at 60 min of middle cerebral artery occlusion. The top panel shows [14C]-IAP autoradiographic digitalized images of an vehicle treated wildtype (WT) mouse (left) and a WT mouse that received 100 mg/kg Egb 761 (right). The lower panel is a graph representing mean CBF of each group of mice. Abbreviations: ACA CTX, anterior cerebral artery cortex, CACA, contralateral anterior cerebral artery; P1, parietal 1; CP1, contralateral parietal 1; P2, parietal 2; CP2, contralateral parietal 2; LAT CTX, lateral cortex; CLAT CTX, contralateral lateral cortex; DM CP, dorsomedial caudate putamen; CDM CP, contralateral dorsomedial caudate putamen; VL CP, ventrolateral caudate putamen; CVL CP, contralateral ventrolateral caudate putamen; *P<0.05; **P<0.01.

FIGS. 45 (A-D) Effects of Ginko biloba components on neuronal HO-1 protein expression. Panel (a) shows results of Western Blot analysis. Mouse cortical neuronal cells were treated for 8 h with EGb 761, bilobalide, or ginkgolides before being harvested and analyzed by Western blot. The top panel of the Western Blot shows that neurons treated with EGb 761 expressed HO-1 more intensely than neurons treated with bilobalide or ginkgolides. The bottom panel shows actin expression in the same blot to indicate similar protein loading in all lanes. Panels (b, c) are graphs showing that EGb 761 increased HO-1 protein expression in a (b) dose and (c) time-dependent manner. The data were calculated as a ratio of the HO-1 and actin band intensities in each lane. Panel (d) shows the results of Western analysis. Cultured neurons were pretreated for 1 h with cycloheximide (CHX) or actinomycin D (ATD) in the concentrations shown before having 100 μg/ml EGb 761 added to the culture medium for an additional 3, 5, or 6 h. The top panel of the blot shows the effect of the various drug regimens HO-1 protein expression. The bottom panel of the blot shows actin expression in the same blot to indicate similar protein loading in all lanes.

FIG. 46 Effects of Ginko biloba components on the expression of HO-2 and NADPH-cytochrome P₄₅₀ reductase. FIG. 46 are the results of Western blot analysis showing the effects of Ginko biloba components on the expression of HO-2 and NADPH-cytochrome P₄₅₀ reductase (CP₄₅₀R) proteins in neurons. Mouse cortical neuronal cultures were treated for 8 h with EGb761, bilobalide, or ginkgolides in the concentrations shown before being harvested for Western blot analysis. Actin expression is shown to indicate that protein loading was similar in all lanes.

FIG. 47 Effect of Egb 761 on the minimal HO-1 promoter. FIG. 47 is a graph showing the dose response effect of EGb 761 on the minimal HO-1 promoter is shown. Hepa pARE-luc cells were treated for 18 h with various concentrations of EGb 761 before being harvested for luminescence measurement. *P<0.05, **P<0.01 when compared with the control group.

FIGS. 48 (A-C) Egb 761 is neuroprotective against H₂O₂— and glutamate-induced toxicity. FIG. 48 (a, b) are graphs showing cell viability (% of control) of primary neurons treated and cultured in different conditions. Primary neurons cultured for 14 d were pre-treated for 6 h with 100 μg/ml EGb 761 or vehicle before being exposed to fresh medium containing H₂O₂ (20), glutamate (30 μM), or vehicle (Control) with or without 5 μM SnPPIX for an additional 18 h. FIG. 48(c) is a graph reporting cell viability (% of control) of primary neurons cultured for 14 d that were pre-treated with 10 μM of the protein synthesis inhibitor cycloheximide (CHX) or vehicle for 1 h before being exposed to 100 μg./ml EGb 761 or vehicle for 6 h. Cells were rinsed and incubated with fresh medium containing glutamate (30 μM) or vehicle for an additional 18 h. Each experiment was conducted in quadruplicate and repeated three times with different primary culture batches. Cell survival was estimated by the MTT assay and expressed as a percent of control viability. *P<0.05. **P<0.01 compared with control groups.

FIG. 49 Protective effect of EC. FIG. 49 is a graph showing the protective effect of EC against MCAO in HO1 WT mice. EC dose-dependently protected MCAO induced brain injury, and infarct volumes (corrected infarct volume,%) were observed to be significantly smaller at doses of 30 mg/kg (20.1±2.7%; p<0.007); 15 mg/kg 24.9±3.8%; p<0.01); 5 mg/kg (28.8±2.9%; p<0.04) as compared to the vehicle treated group (Normal saline) (34.2±3.4%). No significant difference in infarct volumes was observed at 2.5 mg/kg (33.8±3.3%). Drug was given 90 mins before MCAO. MCA was occluded for 90 mins, and reperfusion was allowed for 24 h. After 24 h of reperfusion, animals were killed and TTC was done on brain sections. 8-12 animals were used per group.

FIG. 50 Effects of treatment of EC on the 4-point neurological severity score. FIG. 50 is a graph showing the effects of EC treatment on the 4-point neurological severity score (neurological deficit score). There was a significant difference of neurological deficit observed at 30 mg/kg (2.5±0.25; p<0.01); 15 mg/kg (2.7±0.39; p<0.01) and 5 mg/kg (3±0.35; p<0.03), as compared to the vehicle treatment. No differences in neurological deficit score were observed at the dose of 2.5 mg/kg (3.3±0.29).

FIGS. 51 (A & B) Effect of EC on cerebral blood flow. FIG. 51 panel (a) is a graph showing the results of 4 different EC treatments (30 mg/kg, 15 m/kg, 5 mg/kg and 2.5 mg/kg) on cerebral blood flow. No significant differences were observed in cerebral blood flow as monitored by Laser Doppler (b).

FIG. 52 Corrected infarct volume in vehicle-treated and EC treated HO1^{-/-} mice. FIG. 52 is a graph showing infarct volume (%) when HO1^{-/-} mice were treated with either normal saline or EC (30 mg/kg) 90 minutes before MCAO. 24 h after reperfusion, animals were sacrificed and TTC done on brain sections. There was no significant difference observed in infarct volumes between the vehicle treated HO1^{-/-} (37.1±3.9%) and EC treated HO1^{-/-} (33.8±3.2%) mice.

FIG. 53 Neurological score after EC treatment. Neurological score in HO1^{-/-} mice is shown. No significant differences were observed between the normal saline and EC (30 mg/kg) treated HO1^{-/-} mice.

FIG. 54 Corrected infarct volume after treatment with EC. FIG. 54 is a graph showing the results of treatment with EC or vehicle control in another cohort of experiments. 2 groups of Nrf2 WT mice (12 each) were treated with EC (30 mg/kg) or vehicle, 90 minutes before MCAO. Following 24 h of reperfusion, animals were sacrificed and TTC done on brain sections. Nrf2WT mice demonstrated a significant difference (p<0.04) in infarct volumes between the EC (24.1±1.8%) and vehicle (31.3±1.9%) treated group.

FIG. 55 Neurological deficit score after treatment with EC. FIG. 55 is a graph showing neurological deficit scores in Nrf2 WT mice treated with EC (30 mg/KG) or vehicle, 90 minutes before MCAO is shown. Neurological deficit scores were observed at 24 h. These scores were observed to be significantly (p<0.02) low in EC (2.3±0.1) treated group as compared to the vehicle (3.1±0.26) group.

FIG. 56 Corrected infarct volume. FIG. 56 is a graph showing the results of a separate cohort of experiments in which 2 groups of Nrf2^{-/-} mice (12 mice each) were treated with EC (30 mg/Kg) or vehicle, 90 minutes before MCAO. After 24 h of reperfusion, brains were dissected out and TIC was done on brain sections. EC treated (43.0±2.4) mice were not observed to have significant protective effect as compared to the vehicle (44.8±4.6) treated group.

FIG. 57 Neurological deficit scores after treatment with EC. FIG. 57 is a graph showing neurological deficit scores of Nrf2^{-/-} mice treated with either EC (30 mg/kg) or vehicle, 90 minutes before MCAO. 24 h later mice were observed for neurological deficit scores and no significant difference between EC (3.4±0.17) and vehicle (3.5±0.1) treated groups was found.

FIG. 58 Corrected infarct volume after treatment with EC. FIG. 58 is a graph showing post-treatment paradigms. 12 HO1 WT mice in each group were subjected to 90 minutes MCAO. After 2 h or 4.5 h of reperfusion, mice were treated with either single dose of EC (30 mg/kg) or vehicle (Normal saline). Mice were survived for 72 h. All 12 mice in both 2 and 4.5 h EC treatment groups survived. 10 mice survived in the vehicle treatment group. There was a significant difference (p<0.03) observed in the infarct volume between 2 h EC post-treatment group (33.5±3.2) as compared to the vehicle post-treatment group (46.6±5.3). The protective trend was not observed to be statistically significant at 6 h EC post-treatment and in the vehicle groups.

FIG. 59 Neurological Deficit scores after treatment with EC. FIG. 59 is a graph showing neurological deficit scores in HO1 WT mice after 2 and 4.5 h EC (30 mg/kg), or Vehicle treatment is shown. At 24 h of reperfusion, animals were observed for neurological deficit scores, which were found to be statistically significant at 3.5 h (2.8±0.3), but not at 6 h (1.8±0.1), as compared to vehicle (3.5±0.26) groups.

FIG. 60 Corrected infarct volume after treatment with EC. FIG. 60 is a graph showing corrected infarct volume. In a separate cohort of experiments, 2 groups of Nrf2^{-/-} mice (12 mice each) were treated with EC (30 mg/Kg) or vehicle, 90 minutes before MCAO. After 24 h of reperfusion, brains were dissected out and TTC done. EC treated (43.0±2.4) mice were not observed to have significant protective effect as compared to the vehicle (44.8±4.6) treated group.

FIG. 61 Neurological Deficit scores after treatment with EC. FIG. 61 is a graph showing the neurological deficit scores of Nrf2^{-/-} mice treated with either EC (30 mg/kg) or vehicle before 90 minutes if MCAO. 24 h later mice were observed for neurological deficit scores and no significant difference between EC (3.4±0.17) and vehicle (3.5±0.1) treated groups were found

FIG. 62 Screening for Nrf2 inhibitors by high throughput screening of chemical libraries. FIG. 62 is a schematic showing the method for screening for Nrf2 inhibitors. Liquid handlers are used, including one Tekbench™Work Station, two Cybi-Well™ systems, and BioMek2000™ workstation. The machines are capable of handling 96- and 384-well plates in a variety of formats including high throughput liquid handling, cherrypicking and volume dispensing. The detection modules include the Tecan Safire 2 reader, ICR-8000™ atomic absorption spectrometer, SpectraMax™ 340 reader, and LAS-3000 Fuji imaging station. The liquid handling and detection module are highly integrated by a Mitsubishi RV-2AJ robotic arm and Zymark Twister™ II arm. In addition, both liquid handling modules and detection modules are robotically linked to accessory units including a Kendro Cytomat 6070 automated incubator, Elx-405 plate washers, and Multidrop dispensers.

FIG. 63 Compounds identified from the Spectrum 2000 library. FIG. 63 is a graph showing the relative luciferase activity produced by cells treated with the indicated compounds. The Soectrum 2000 library was used.

FIG. 64 Compounds identified from the Sigma Lopac library. FIG. 64 is a graph showing the relative luciferase activity produced by cells treated with the indicated compounds. The Sigma Lopac library was used.

### DETAILED DESCRIPTION OF THE INVENTION

The invention generally features therapeutic compositions and methods useful for the treatment and diagnosis of a disease associated with oxidative stress. The invention is based, at least in part, on the discoveries that mammals having reduced levels of Nrf2 are particularly susceptible to tissue damage associated with oxidative stress, including pulmonary inflammatory conditions, sepsis, and neuronal cell death associated with ischemic injury. Importantly, Nrf2 provides protection against oxidative stress and reduces neuronal cell death associated with ischemic injury. Accordingly, agents that increase the expression or biological activity of Nfr2 are useful for the prevention and treatment of diseases or disorders associated with increased levels of oxidative stress or reduced levels of antioxidants, including pulmonary inflammatory conditions, pulmonary fibrosis, asthma, chronic obstructive pulmonary disease, emphysema, sepsis, septic shock, cerebral ischemia and neurodegenerative disorders.

Nuclear Factor E2p45-Related Factor (Nrf2)

Nuclear factor erythroid-2 related factor 2 (NRF2), a cap-and-collar basic leucine zipper transcription factor, regulates a transcriptional program that maintains cellular redox homeostasis and protects cells from oxidative insult (Rangasamy T, et al., J Clin Invest 114, 1248 (2004); Thimmulappa R K, et al. Cancer Res 62, 5196 (2002); So H S, et al. Cell Death Differ (2006)). NRF2 activates transcription of its target genes through binding specifically to the antioxidant-response element (ARE) found in those gene promoters. The NRF2-regulated transcriptional program includes a broad spectrum of genes, including antioxidants, such as γ-glutamyl cysteine synthetase modifier subunit (GCLm), γ-glutamyl cysteine synthetase catalytic subunit (GCLc), heme oxygenase-1, superoxide dismutase, glutathione reductase (GSR), glutathione peroxidase, thioredoxin, thioredoxin reductase, peroxiredoxins (PRDX), cysteine/glutamate transporter (SLC7A11) (7, 8)], phase II detoxification enzymes [NADP(H) quinone oxidoreductase 1 (NQO1), GST, UDP-glucuronosyltransferase (Rangasamy T, et al. J Clin Invest 114: 1248 (2004); Thimmulappa R K, et al. Cancer Res 62: 5196 (2002)), and several ATP-dependent drug efflux pumps, including MRP1, MRP2 (Hayashi A, et al. Biochem Biophy Res Commun 310: 824 (2003)); Vollrath V, et al. Biochem J (2006)); Nguyen T, et al. Annu Rev Pharmacol Toxicol 43: 233 (2003)).

### KEAP1

KEAP1 is a cytoplasmic anchor of NRF2 that also functions as a substrate adaptor protein for a Cul3-dependent E3 ubiquitin ligase complex to maintain steady-state levels of NRF2 and NRF2-dependent transcription (Kobayashi et al., Mol Cell Biol 24: 7130 (2004); Zhang D D et al. Mol Cell Biol 24: 10491 (2004)). The Keap1 gene is located at human chromosomal locus 19p13.2. The KEAP1 polypeptide has three major domains: (1) an N-terminal Broad complex, Tramtrack, and Bric-a-brac (BTB) domain; (2) a central intervening region (IVR); and (3) a series of six C-terminal Kelch repeats (Adams J, et al. Trends Cell Biol 10:17 (2000)). The Kelch repeats of KEAP1 bind the Neh2 domain of NRF2, whereas the IVR and BTB domains are required for the redox-sensitive regulation of NRF2 through a series of reactive cysteines present throughout this region (Wakabayashi N, et al. Proc Natl Acad Sci USA 101: 2040 (2004)). KEAP1 constitutively suppresses NRF2 activity in the absence of stress. Oxidants, xenobiotics and electrophiles hamper KEAP1-mediated proteasomal degradation of NRF2, which results in increased nuclear accumulation and, in turn, the transcriptional induction of target genes that ensure cell survival (Wakabayashi N, et al. Nat Genet 35: 238 (2003)). Germline deletion of the KEAP1 gene in mice results in constitutive activation of NRF2 (Wakabayashi N, et al Nat Genet 35: 238 (2003)). Recently, a somatic mutation (G430C) in KEAP1 in one lung cancer patient and a small-cell lung cancer cell line (G364C) have been described (Padmanabhan B, et al. Mol Cell 21: 689 (2006)). Prothymosin α, a novel binding partner of KEAP1, has been shown to be an intranuclear dissociator of NRF2-KEAP1 complex and can upregulate the expression of Nrf2 target genes (Karapetian R N, et al. Mol Cell Biol 25: 1089 (2005)).

### Oxidative Stress and Pulmonary Disorders

As reported herein, oxidative stress is involved in the pathogenesis of pulmonary diseases, including asthma, COPD, and emphysema. In particular, increased Nrf2 activation is associated with a decrease in airway remodeling (Rangasamy et al., J Exp Med. 2005; 202:47). Airway remodeling occurs as a result of the proliferation of fibroblasts. Increased remodeling is associated with several pulmonary diseases such as COPD, asthma and interstitial pulmonary fibrosis (IPF). Compounds and strategies that increase Nrf2 biological activity or expression are useful for preventing or decreasing fibrosis and airway remodeling in lungs as a result of COPD, Asthma and IPF. The lungs of Nrf2^{-/-} mice exhibit a defective antioxidant response that leads to worsened asthma, exacerbates airway inflammation and increases airway hyperreactivity (AHR). Critical host factors that protect the lungs against oxidative stress determine susceptibility to asthma or act as modifiers of risk by inhibiting associated inflammation. Nrf2-regulated genes in the lungs include almost all of the relevant antioxidants, such as heme oxygenase-1 (HO-1), γ-glutamyl cysteine synthase (γ-GCS), and several members of the GST family. Methods for increasing Nrf-2 expression or biological activity are, therefore, useful for treating pulmonary diseases associated with oxidative stress, inflammation, and fibrosis. Such diseases include, but are not limited to, chronic bronchitis, emphysema, inflammation of the lungs, pulmonary fibrosis, interstitial lung diseases, and other pulmonary diseases or disorders characterized by subepithelial fibrosis, mucus metaplasia, and other structural alterations associated with airway remodeling.

### Ischemia and Neurodegenerative Disease

Nrf2 protects cells and multiple tissues by coordinately up-regulating ARE-related detoxification and antioxidant genes and molecules required for the defense system in each specific environment. As reported herein, a role has been identified for Nrf2 as a neuroprotectant molecule that reduces apoptosis in neural tissues following transient ischemia. Accordingly, the invention provides compositions and methods for the treatment of a variety of disorders involving cell death, including but not limited to, neuronal cell death. In one embodiment, agents that increase Nrf2 expression or biological activity are useful for the treatment or prevention of virtually any disease or disorder characterized by increased levels of cell death, including ischemic injury (caused by, e.g., a myocardial infarction, a stroke, or a reperfusion injury, brain injury, stroke, and multiple infarct dementia, a secondary exsaunguination or blood flow interruption resulting from any other primary diseases), as well as neurodegenerative disorders (e.g., Alzheimer's disease (AD) Creutzfeldt-Jakob disease, Huntington's disease, Lewy body disease, Pick's disease, Parkinson's disease, amyotrophic lateral sclerosis (ALS), and neurofibromatosis).

### Nrf2 Activating Agents

Given that increased Nrf2 expression or activity is useful for the treatment or prevention of virtually any disease or disorder associated with oxidative stress, agents that activate Nrf2 are useful in the methods of the invention. Such agents are known in the art and are described herein. Exemplary Nrf2 activating compounds include the class of compounds known as tricyclic bis-enones (TBEs) that are structurally related to synthetic triterpenoids, including RTA401 and RTA 402. Compounds useful in the methods of the invention include those described in U.S. Patent Publication No. 2004/002463, as well as those listed in Table 1A (below).

TABLE 1A

| Nrf2 activator | Year | Reference |
|---|---|---|
| 1,2,3,4,6-Penta-O-Galloyl- | 2006 | Mol Pharmacol. 2006 May; 69(5): 1554-63. Epub 2006 |
| Beta-D-Glucose | | Jan 31. |
| 1,2-Diphenol (Catechol) | 2000 | J Biol Chem, Vol. 275, Issue 15, 11291-11299, Apr. 14, |
| | | 2000 |
| 1,2-Dithiole-3-Thione | 2002 | J Biol Chem. 2003 Jan 10; 278(2): 703-11. Epub 2002 |
| | | Oct 4. |
| 1,4-Diphenols (P- | 2000 | J Biol Chem, Vol. 275, Issue 15, 11291-11299, Apr. 14, |
| Hydroquinone) | | 2000 |
| 1-[2-Cyano-3-,12- | 2005 | Cancer Res. 2005 Jun 1; 65(11): 4789-98. |
| Dioxooleana-1,9(11)-Dien- | | |
| 28-Oyl]Imidazole (CDDO- | | |
| Im) | | |
| 15-Deoxy-12,14-Pgj2 | 2000 | J Biol Chem, Vol. 275, Issue 15, 11291-11299, Apr. 14, |
| | | 2000 |
| 1-Chloro-2,4- | 2000 | J Biol Chem. 2000 May 26; 275(21): 16023-9. |
| Dinitrobenzene | | |
| 2,3,7,8- | 2003 | Cancer Res. 2003 Sep 1; 63(17): 5636-45. |
| Tetrachlorodibenzo-P- | | |
| Dioxin | | |
| 2-Cyano-3,12- | 2005 | Biochem Biophys Res Commun. 2005 Jun |
| Dioxooleana-1,9(11)-Dien- | | 17; 331(4): 993-1000. |
| 28-Oic Acid (CDDO) | | |
| 2-Indol-3-Yl- | 2003 | Biochem Biophys Res Commun. 2003 Aug |
| Methylenequinuclidin-3-Ols | | 8; 307(4): 973-9. |
| 3-Hydroxyanthranilic Acid | 2006 | Drug Metab Dispos. 2006 Jan; 34(1): 152-65. Epub 2005 |
| | | Oct 21. |
| 3-Methylcholanthrene | 2006 | Febs J. 2006 Jun; 273(11): 2345-56. |
| 4-Hydroxyestradiol | 2003 | Mol Cell Biol. 2003 Oct; 23(20): 7198-209. |
| 4-Hydroxynonenal | 2005 | J Immunol. 2005 Oct 1; 175(7): 4408-15. |
| 6-Methylsulfinylhexyl | 2002 | J. Biol. Chem., Vol. 277, Issue 5, 3456-3463, Feb. |
| Isothiocyanate | | 1, 2002 |
| 7-Oh Cmrn | 2001 | Cancer Research 61, 3299-3307, Apr. 15, 2001 |
| 9-Cis-Retinoic Acid | 2004 | Proc Natl Acad Sci USA. 2004 Mar 9; 101(10): 3381-6. |
| | | Epub 2004 Feb 25. |
| Acetaminophen | 2001 | Toxicol Sci. 2001 Jan; 59(1): 169-77. |
| Acetylcarnitine | 2004 | J Nutr. 2004 Dec; 134(12 Suppl): 3499s-3506s |
| Acrolein | 2002 | Free Radical Biology & Medicine, Vol. 32, No. 7, Pp. |
| | | 650-662, 2002 |
| Allyl Isothiocyanate | 2005 | J Invest Dermatol. 2005 Apr; 124(4): 825-32. |
| Alpha-Lipoic Acid | 2005 | Chem Res Toxicol. 2005 Aug; 18(8): 1296-305. |
| Apomorphine | 2006 | Ann N Y Acad Sci. 2006 May; 1067: 420-4. |
| Arsenic | 1999 | J. Biol. Chem., Vol. 274, Issue 37, 26071-26078, |
| | | Sep. 10, 1999 |
| AUR ((2,3,4,6-Tetra-O)- | 2001 | J. Biol. Chem., Vol. 276, Issue 36, 34074-34081, |
| Acetyl-1-Thio-D- | | Sep. 7, 2001 |
| Glucopyranosato- | | |
| S)(Triethylphosphine) | | |
| Gold(I) | | |
| Autg ((1-Thio-D- | 2001 | J. Biol. Chem., Vol. 276, Issue 36, 34074-34081, |
| Glucopyranosato) Gold(I) | | Sep. 7, 2001 |
| Autm (Sodium | 2001 | J. Biol. Chem., Vol. 276, Issue 36, 34074-34081, |
| Aurothiomalate | | Sep. 7, 2001 |
| Avicins | 2004 | J Biol Chem. 2004 Mar 5; 279(10): 8919-29. Epub 2003 |
| | | Dec 19. |
| Bis(2- | 2006 | Cell Death Differ. 2006 Feb 17 |
| Hydroxybenzylidene)Acetone | | |
| Bleomycin | 2004 | Cancer Res. 2004 May 15; 64(10): 3701-13. |
| B-Naphthoflavone | 1998 | Oncogene (1998) 17, 3145 ± 3156 |
| Broccoli Seeds | 2004 | Free Radic Biol Med. 2004 Nov 15; 37(10): 1578-90. |
| Bucillamine | 2006 | Biomaterials. 2006 Jun 24; |
| Butylated Hydroxyanisole | 1997 | Biochemical And Biophysical Research Communications |
| | | 236, 313-322 (1997) |
| Butylated Hydroxytoulene | 1999 | PNAS U Oct. 26, 1999 U Vol. 96 U No. 22 U 12731-12736 |
| Cadmuim Chloride | 2000 | J Biol Chem. 2000 May 26; 275(21): 16023-9. |
| Cafestol | 2001 | Cancer Research 61, 3299-3307, Apr. 15, 2001 |
| Carbon Monoxide | 2006 | Cancer Lett. 2006 Mar 3; |
| Carnosol | 2004 | J Clin Invest. 2004 Nov; 114(9): 1248-59. |
| Catechol | 2000 | J Biol Chem. 2000 May 26; 275(21): 16023-9. |
| Chlorogenic Acid | 2005 | Cancer Res. 2005 Jun 1; 65(11): 4789-98. |
| Cigarette Smoke | 2003 | Pharm Res. 2003 Sep; 20(9): 1351-6. |
| Cobalt (Cobalt Chloride) | 2001 | J Biol Chem. 2001 Jul 20; 276(29): 27018-25. Epub 2001 |
| | | May 16. |
| Copper | 2006 | Drug Metab Dispos. 2006 Jan; 34(1): 152-65. Epub 2005 |
| | | Oct 21. |
| Coumarin | 2001 | Cancer Research 61, 3299-3307, Apr. 15, 2001 |
| Curcumin | 2003 | J Biol Chem. 2003 Feb 14; 278(7): 4536-41. Epub 2002 |
| | | Nov 22. |
| Deprenyl (Selegiline) | 2006 | Carcinogenesis. 2006 May; 27(5): 1008-17. Epub 2005 |
| | | Nov 23. |
| Dexamethasone 21- | 2002 | Toxicol Lett. 2002 Jun 7; 132(1): 27-36. |
| Mesylate | | |
| Diallyl Disulfide | 2004 | Free Radic Biol Med. 2004 Nov 15; 37(10): 1578-90. |
| Diallyl Sulfide | 2004 | Febs Lett. 2004 Aug 13; 572(1-3): 245-50. |
| Diallyl Trisulfide (DATS) | 2004 | Free Radic Biol Med. 2004 Nov 15; 37(10): 1578-90. |
| Diesel Exhaust | 2001 | J. Biol. Chem., Vol. 276, Issue 36, 34074-34081, |
| | | Sep. 7, 2001 |
| Diethylmaleate | 2000 | J Biol Chem. 2000 May 19; 275(20): 15370-6. |
| Epicatechin-3-Gallate | 2001 | Drug Metabolism Reviews Volume 33, Number 3-4/ |
| | | 2001 |
| Epigallocatechin-3-Gallate | 2005 | Arterioscler Thromb Vasc Biol. 2005 Oct; 25(10): 2100-5. |
| | | Epub 2005 Aug 25. |
| Eriodictyo | 2006 | Invest Ophthalmol Vis Sci. 2006 Jul; 47(7): 3164-77. |
| Ethoxyquin. | 2000 | Biochem Soc Trans. 2000 Feb; 28(2): 33-41. |
| Ferulic Acid (Trans-4- | 2006 | Carcinogenesis. 2006 May; 27(5): 1008-17. Epub 2005 |
| Hydroxy-3- | | Nov 23. |
| Methoxycinnamic Acid, | | |
| 99% Purity) | | |
| Fisetin | 2006 | Invest Ophthalmol Vis Sci. 2006 Jul; 47(7): 3164-77. |
| Flunarizine | 2006 | World J Gastroenterol. 2006 Jan 14; 12(2): 214-21. |
| Gallic Acid (3,4,5- | 2006 | Carcinogenesis. 2006 May; 27(5): 1008-17. Epub 2005 |
| Trihydroxybenzoic Acid, | | Nov 23. |
| Gentisic Acid | 2006 | Carcinogenesis. 2006 May; 27(5): 1008-17. Epub 2005 |
| | | Nov 23. |
| Glucose Oxidase | 2000 | J Biol Chem. 2000 May 26; 275(21): 16023-9. |
| Glycosides From Digitalis | 2006 | Food Chem Toxicol. 2006 Aug; 44(8): 1299-307. Epub |
| Purpurea | | 2006 Mar 6. |
| Heme | 1999 | J. Biol. Chem., Vol. 274, Issue 37, 26071-26078, |
| | | September 10, 1999 |
| Hemin | 2001 | J Biol Chem. 2001 May 25; 276(21): 18399-406. Epub |
| | | 2001 Mar 1. |
| Hydrogen Peroxide | 2000 | J Biol Chem. 2000 May 26; 275(21): 16023-9. |
| Hyerpoxia | 2005 | Free Radic Biol Med. 2005 Feb 1; 38(3): 325-43. |
| Indole-3-Carbinol | 2001 | Cancer Research 61, 3299-3307, Apr. 15, 2001 |
| Indomethacin | 2002 | Drug Metabolism Reviews Volume 33, Number 3-4/ |
| | | 2002 |
| Insulin | 2006 | Pharmazie. 2006 Apr; 61(4): 356-8. |
| Iodoacetic Acid | 2000 | J Biol Chem. 2000 May 26; 275(21): 16023-9. |
| Kahweol Palmitate | 2001 | Cancer Research 61, 3299-3307, Apr. 15, 2001 |
| Laminar Flow | 2002 | Proc Natl Acad Sci USA. 2002 Sep 3; 99(18): 11908-13. |
| | | Epub 2002 Aug 22. |
| Lead | 2006 | Drug Metab Dispos. 2006 Jan; 34(1): 152-65. Epub 2005 |
| | | Oct 21. |
| Limettin (LMTN) | 2001 | Cancer Research 61, 3299-3307, Apr. 15, 2001 |
| Lipoic Acid. | 2004 | J Clin Invest. 2004 Jan; 113(1): 65-73. |
| Lipopolysacharide | 2005 | Pharm Res. 2005 Nov; 22(11): 1805-20. Epub 2005 Aug |
| | | 16. |
| Luteolin | 2006 | J Neurosci Res. 2006 Jun 26 |
| Lycopene | 2005 | J Neurosci Res. 2005 Feb 15; 79(4): 509-21. |
| Menadione | 2000 | J Biol Chem. 2000 May 26; 275(21): 16023-9. |
| Mercury | 2006 | Am J Respir Cell Mol Biol. 2006 Feb; 34(2): 174-81. Epub |
| | | 2005 Sep 29. |
| Nickel (II) | 2006 | Biochem Biophys Res Commun. 2006 Jul |
| | | 14; 345(4): 1350-7. Epub 2006 May 15. |
| Nitric Oxide-Donating | 2005 | Atherosclerosis. 2005 Oct 20; |
| Aspirin | | |
| Oltipraz | 2001 | Proc Natl Acad Sci USA. 2001 Mar 13; 98(6): 3410-5 |
| Oxidized Low-Density | 2004 | Circ Res. 2004 Mar 19; 94(5): 609-16. Epub 2004 Jan 29. |
| Lipoproteins | | |
| Paraquat | 2000 | J Biol Chem. 2000 May 26; 275(21): 16023-9. |
| Parthenolide | 2005 | J Biochem Mol Biol. 2005 Mar 31; 38(2): 167-76. |
| P-Coumaric Acid (Trans-4- | 2006 | Carcinogenesis. 2006 May; 27(5): 1008-17. Epub 2005 |
| Hydroxycinnamic Acid), | | Nov 23. |
| Pgj2 | 2000 | J Biol Chem, Vol. 275, Issue 15, 11291-11299, Apr. 14, |
| | | 2000 |
| Phenethyl Isothiocyanate | 2003 | Biochim Biophys Acta. 2003 Oct 1; 1629(1-3): 92-101. |
| Phorbol 12-Myristate 13- | 2000 | Proc Natl Acad Sci USA. 2000 Nov 7; 97(23): 12475-80. |
| Acetate (PMA) | | |
| P-Hydroxybenzoic Acid | 2006 | Carcinogenesis. 2006 Apr; 27(4): 803-10. Epub 2005 Nov |
| | | 2. |
| Proteasome Inhibitor MG- | 2006 | Biochem Pharmacol, 2006 Jun 24; |
| 132 | | |
| Proteasome Inhibitors | 2003 | J Biol Chem. 2003 Jan 24; 278(4): 2361-9. Epub 2002 |
| (Lactacystin Or MG-132) | | Nov 14. |
| Pyrrolidine | 2003 | Circ Res. 2003 Mar 7; 92(4): 386-93. Epub 2003; Feb 6. |
| Dithiocarbamate | | |
| Quercetin | 2006 | Invest Ophthalmol Vis Sci. 2006 Jul; 47(7): 3164-77. |
| Quercetin 3-O-Beta-L- | 2006 | Biochem Biophys Res Commun. 2006 May |
| Arabinopyranoside | | 12; 343(3): 965-72. Epub 2006 Mar 29. |
| Resveratrol | 2005 | J Biochem Mol Biol. 2005 Mar 31; 38(2): 167-76. |
| Sodium Arsenite | 2000 | J Biol Chem. 2000 May 26; 275(21): 16023-9. |
| Spermidine | 2003 | Toxicol Sci. 2003 May; 73(1): 124-34. Epub 2003 Mar 25. |
| Spermine | 2003 | Biochem Biophys Res Commun. 2003 Jun 6; 305(3): 662-70. |
| Spermine Nonoate | 2003 | Biochem Biophys Res Commun. 2003 Jun 6; 305(3): 662-70. |
| Sulforaphane | 2002 | Cancer Res. 2002 Sep 15; 62(18): 5196-203. |
| Sulforaphane | 2002 | J. Biol. Chem., Vol. 277, Issue 5, 3456-3463, Feb. |
| | | 1, 2003 |
| Tert-Butylhydroquinone | 1998 | Oncogene (1998) 17, 3145 ± 3156 |
| (T-BHQ) | | |
| TNF-Alpha | 2005 | J Biol Chem. 2005 Jul 29; 280(30): 27888-95. Epub 2005 |
| | | Jun 8. |
| Trans-Stilbene Oxide | 2006 | Biochem Biophys Res Commun. 2006 Jan |
| | | 20; 339(3): 915-22. Epub 2005 Nov 28. |
| Triterpenoid-155 | 2005 | Proc Natl Acad Sci USA. 2005 Mar 22; 102(12): 4584-9. |
| | | Epub 2005 Mar 14. |
| Triterpenoid-156 | 2005 | Proc Natl Acad Sci USA. 2005 Mar 22; 102(12): 4584-9. |
| | | Epub 2005 Mar 14. |
| Triterpenoid-162 | 2005 | Proc Natl Acad Sci USA. 2005 Mar 22; 102(12): 4584-9. |
| | | Epub 2005 Mar 14. |
| Triterpenoid-225 | 2005 | Mol Cancer Ther. 2005 Jan; 4(1): 177-86. |
| Tunicamycin | 2004 | Biochem J. 2004 Jan 1; 377 (Pt 1): 205-13. |
| Ultraviolet A Irradiation | 2005 | Proc Natl Acad Sci USA. 2005 Mar 22; 102(12): 4584-9. |
| | | Epub 2005 Mar 14. |
| Wasabi Extract | 2001 | Toxicol Appl Pharmacol. 2001 Jun 15; 173(3): 154-60. |
| Xanthohumol (XH) | 2005 | Biochem J. 2005 Oct 15; 391(Pt 2): 399-408. |
| Zerumbone | 2004 | Faseb J. 2004 Aug; 18(11): 1258-60. Epub 2004 Jun 18. |
| Zinc | 1999 | J. Biol. Chem., Vol. 274, Issue 37, 26071-26078, |
| | | Sep. 10, 1999 |

Library Screened: Spectrum 2000 and Sigma Lopac 1280
List of Activators

| | |
|---|---|
| 1 | Patulin |
| 2 | Methosyvone |
| 3 | Dehydrovariabilin |
| 4 | Biochanin A |
| 5 | Pdodfilox |
| 6 | 8-2′-Dimethoxyflavone |
| 7 | 6,3′-Dimethoxyflavone |
| 8 | Pinosylvin |
| 9 | Gentian Violet |
| 10 | Gramicidin |
| 11 | Thimerosal |
| 12 | Cantharidin |
| 13 | Fenbendazole |
| 14 | Mebendazole |
| 15 | Triacetylresveratrol |
| 16 | Resveratrol |
| 17 | Tetrachloroisopthalonitrile |
| 18 | Simvastatin |
| 19 | Valdecoxib |
| 20 | beta-Peltatin |
| 21 | 4,6-Dimethoxy-5-methylsioflavone |
| 22 | Nocodazole |
| 23 | Pyrazinecarboxamide |
| 24 | (±)-thero-1-Phenyl-2-decanoylamino-3-morpholino-1-propanol hydrochloride |
| 25 | SU4132 |

### Keap1 RNA Interference

Keap1 is a known inhibitor of Nrf2. Agents that reduce Keap1 expression are useful for the treatment of diseases and disorders associated with oxidative stress. RNA interference (RNAi) is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of shRNAs using a plasmid-based expression system is currently being used to create loss-of-function phenotypes in mammalian cells. siRNAs that target Keap1 decrease Keap1 expression thereby activating Nrf2.

### Keap1 Inhibitory Nucleic Acid Molecules

Keap1 inhibitory nucleic acid molecules are essentially nucleobase oligomers that may be employed as single-stranded or double-stranded nucleic acid molecule to decrease Keap1 expression. In one approach, the Keap1 inhibitory nucleic acid molecule is a double-stranded RNA used for RNA interference (RNAi)-mediated knock-down of Keap1 gene expression. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to an Keap1 gene comprises at least a fragment of the double-stranded gene, such that each strand of the double-stranded inhibitory nucleic acid molecule is capable of binding to the complementary strand of the target Keap1 gene. The inhibitory nucleic acid molecule need not have perfect correspondence to the reference Keap1 sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

The inhibitory nucleic acid molecules provided by the invention are not limited to siRNAs, but include any nucleic acid molecule sufficient to decrease the expression of an Keap1 nucleic acid molecule or polypeptide. Each of the DNA sequences provided herein may be used, for example, in the discovery and development of therapeutic antisense nucleic acid molecule to decrease the expression of Keap1. The invention further provides catalytic RNA molecules or ribozymes. Such catalytic RNA molecules can be used to inhibit expression of an Keap1 nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient receives a dosage between about 50 and 300 mg/m²/day (e.g., 50, 75, 100, 125, 150, 175, 200, 250, 275, and 300).

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with an Keap1 nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

### Nrf2 and Keap1 Polynucleotides

In general, the invention includes any nucleic acid sequence encoding an Nrf2 polypeptide or a Keap1 inhibitory nucleic acid molecule. Also included in the methods of the invention are any nucleic acid molecule containing at least one strand that hybridizes with such a Keap1 nucleic acid sequence (e.g., an inhibitory nucleic acid molecule, such as a dsRNA, siRNA, shRNA, or antisense molecule). The Keap1 inhibitory nucleic acid molecules of the invention can be 19-21 nucleotides in length. In some embodiments, the inhibitory nucleic acid molecules of the invention comprises 20, 19, 18, 17, 16, 15, 14, 13, 12, 11, 10, 9, 8, or 7 identical nucleotide residues. In yet other embodiments, the single or double stranded antisense molecules are 60%, 65%, 70%, 75%, 80%, 85%, 90%, 95%, 96%, 97%, 98%, 99% complementary to the Keap1 target sequence. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

Further embodiments can include any of the above inhibitory polynucleotides, directed to Keap1, Phase II genes, including glutathione —S-transferases (GSTs), antioxidants (GSH), and Phase II drug efflux proteins, including the multidrug resistance proteins (MRPs), or portions thereof.

### Delivery of Nucleobase Oligomers

Naked oligonucleotides are capable of entering tumor cells and inhibiting the expression of Keap1. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Nrf2 Polynucleotide Therapy

Methods for expressing Nrf2 in a cell of a subject are useful for increasing the expression of downstream antioxidant genes. Polynucleotide therapy featuring a polynucleotide encoding a Nrf2 nucleic acid molecule or analog thereof is one therapeutic approach for treating or preventing a disease or disorder associated with oxidative stress and cellular damage in a subject. Expression vectors encoding nucleic acid molecules can be delivered to cells of a subject having a disease or disorder associated with oxidative stress and cellular damage. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a Nrf2 nucleic acid molecule, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of an Nrf2 nucleic acid molecule therapeutic to a cell of a patient diagnosed as having a disease or disorder associated with oxidative stress and cellular damage. For example, a Nrf2 nucleic acid molecule can be introduced into a cell (e.g., a lung cell, a neuronal cell, or a cell at risk of undergoing cell death, including apoptosis) by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the Nrf2 nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Nrf2 nucleic acid molecule expression for use in polynucleotide therapy methods can be directed from any suitable promoter (e.g., the human cytomegalovirus (CMV), simian virus 40 (SV40), or metallothionein promoters), ubiquitin promoter and regulated by any appropriate mammalian regulatory element. In one embodiment, a promoter that directs expression in a pulmonary tissue, a neuronal tissue, a myocardial tissue, pulmonary tissue or any other tissue susceptible to oxidative stress is used, forexample, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, increased Nrf2 expression or biological activity is useful for the treatment or prevention of a disease or disorder associated with oxidative stress and cellular damage. Accordingly, the invention provides therapeutic compositions that increase Nrf2 expression to enhance antioxidant activity in a tissue, such as a lung tissue for the treatment or prevention of a pulmonary inflammatory condition (e.g., pulmonary fibrosis, asthma, chronic obstructive pulmonary disease, emphysema, sepsis, septic shock), or a neural tissue for the treatment of cerebral ischemia or a neurodegenerative disorder. In one embodiment, the present invention provides a pharmaceutical composition comprising a Keap1 inhibitory nucleic acid molecule (e.g., an antisense, siRNA, or shRNA polynucleotide) that decreases the expression of a Keap1 nucleic acid molecule or polypeptide. If desired, the Keap1 inhibitory nucleic acid molecule is administered in combination with an agent that activates Nrf2 or with an antioxidant. In various embodiments, the Keap1 inhibitory nucleic acid molecule is administered prior to, concurrently with, or following administration of the agent that activates Nrf2 or with the antioxidant. Without wishing to be bound by theory, administration of a Keap1 inhibitory nucleic acid molecule enhances the biological activity of Nrf2. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

A nucleic acid molecule encoding Nrf2, an inhibitory nucleic acid molecule of the invention, together with an antioxidant, may be administered within a pharmaceutically-acceptable diluents, carrier, or excipient, in unit dosage form. Conventional pharmaceutical practice may be employed to provide suitable formulations or compositions to administer the compounds to patients suffering from a disease that is associated with oxidative stress. Administration may begin before the patient is symptomatic. Any appropriate route of administration may be employed, for example, administration may be by inhalation, or parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for nucleic acid molecules encoding Nrf2 or Keap1 inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase composition of the invention is likely to depend on such variables as the type and extent of the disorder, the overall health status of the particular patient, the formulation of the compound excipients, and its route of administration.

With respect to a subject having a disease or disorder characterized by oxidative stress, an effective amount is sufficient to increase antioxidant activity or reduce oxidative stress. With respect to a subject having a neurodegenerative disease or other disease associated with excess cell death, an effective amount is sufficient to stabilize, slow, reduce, or reverse the cell death. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Kits

The invention provides kits for preventing, treating, or monitoring a disease associated with oxidative stress, such as pulmonary inflammatory conditions, pulmonary fibrosis, asthma, chronic obstructive pulmonary disease, emphysema, sepsis, septic shock, cerebral ischemia and neurodegenerative disorders. In one embodiment, the kit detects an alteration in the expression of a Marker (e.g., Nrf2, Keap1, Phase II genes, including glutathione —S-transferases (GSTs), antioxidants (GSH)) nucleic acid molecule or polypeptide relative to a reference level of expression. In another embodiment, the kit detects an alteration in the sequence of a Nrf2 nucleic acid molecule derived from a subject relative to a reference sequence. In related embodiments, the kit includes reagents for monitoring the expression of a Nrf2 nucleic acid molecule, such as primers or probes that hybridize to a Nrf2 nucleic acid molecule. In other embodiments, the kit includes an antibody that binds to a Nrf2 polypeptide.

Optionally, the kit includes directions for monitoring the nucleic acid molecule or polypeptide levels of a Marker in a biological sample derived from a subject. In other embodiments, the kit comprises a sterile container that contains the primer, probe, antibody, or other detection regents; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in treating or preventing oxidative stress or cellular damage associated with pulmonary inflammatory conditions, pulmonary fibrosis, asthma, chronic obstructive pulmonary disease, emphysema, sepsis, septic shock, cerebral ischemia and neurodegenerative disorders. Preferably, the kit further comprises any one or more of the reagents described in the assays described herein. In other embodiments, the instructions include at least one of the following: description of the primer or probe; methods for using the enclosed materials for the treatment or prevention of a pulmonary inflammatory condition, pulmonary fibrosis, asthma, chronic obstructive pulmonary disease, emphysema, sepsis, septic shock, cerebral ischemia and neurodegenerative disorders; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a pulmonary inflammatory condition, pulmonary fibrosis, asthma, chronic obstructive pulmonary disease, emphysema, sepsis, septic shock, cerebral ischemia or neurodegenerative disorder can be monitored using the methods and compositions of the invention. In one embodiment, the treatment of oxidative stress in a patient can be monitored using the methods and compositions of the invention. Such monitoring may be useful, for example, in assessing the efficacy of a particular drug in a patient. Therapeutics that enhance the expression or biological activity of a Nrf2 nucleic acid molecule or Nrf2 polypeptide or increase the expression or biological activity of an antioxidant are taken as particularly useful in the invention. Other nucleic acids or polypeptides according to the invention that are useful for monitoring or in aspects of the invention include Nrf2, Keap1, Phase II genes, including glutathione —S-transferases (GSTs), and antioxidants (GSH)).

Screening Assays

One embodiment of the invention encompasses a method of identifying an agent that activates Nrf2 and increases the expression of a downstream antioxidant or that decreases the expression of Keap1. Accordingly, compounds that enhance the expression or activity of a Nrf2 nucleic acid molecule, polypeptide, variant, or portion thereof are useful in the methods of the invention for the treatment or prevention of pulmonary inflammatory conditions, pulmonary fibrosis, asthma, chronic obstructive pulmonary disease, emphysema, sepsis, septic shock, cerebral ischemia and neurodegenerative disorders. The method of the invention may measure an increase in Nrf2 transcription or translation. Any number of methods are available for carrying out screening assays to identify such compounds. In one approach, the method comprises contacting a cell that expresses Nrf2 nucleic acid molecule with an agent and comparing the level of Nrf2 nucleic acid molecule or polypeptide expression in the cell contacted by the agent with the level of expression in a control cell, wherein an agent that increases Nrf2 expression thereby treats or prevents a pulmonary inflammatory condition, pulmonary fibrosis, asthma, chronic obstructive pulmonary disease, emphysema, sepsis, septic shock, cerebral ischemia and neurodegenerative disorders. In another approach, candidate compounds are identified that specifically bind to and enhance the activity of a polypeptide of the invention (e.g., a Nrf2 cytoprotective activity). Methods of assaying such biological activities are known in the art and are described herein. The efficacy of such a candidate compound is dependent upon its ability to interact with a Nrf2 nucleic acid molecule, Nrf2 polypeptide, a variant, or portion. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate an Nrf2 or Keap1 biological activity. Standard methods for decreasing Keap1 expression include mutating or deleting an endogenous Keap1 sequence, interfering with Keap1 expression using RNAi, or microinjecting an Keap1-expressing cell with an antibody that binds Keap1 and interferes with its function. Alternatively, chromosomal nondysjunction can be assayed in vivo, for example, in a mouse model in which Keap1 has been knocked out by homologous recombination, or any other standard method. In another example, a high throughput approach can be used to screen different chemicals for their potency to activate Nrf2. A cell based reporter assay approach can be used for identification of agents that can activate Nrf2 mediated transcription. For example, cells that are stably transfected with a luciferase reporter vector are plated and incubated overnight. Cells are then pretreated with different agents, and luciferase activity is measured, wherein an increase in luciferase activity correlates with an increase in Nrf2 expression. Agents that increase Nrf2 expression or activity by at least about 5%, 10%, or 20% or more (e.g., 25%, 50%, 75%, 85%, or 95%) are identified as useful in the methods of the invention.

Exemplary libraries useful in screening methods include the following:

CB01 (ChemBridge 1) and CB02 (ChemBridge 2):

Library CB01 and CB02 were purchased from ChemBridge Corporation (San Diego, Calif.). It contains 10,000 compounds on 125 plates, 80 compounds per plate.

MSSP (Spectrum 1):

Library MSSP was purchased from MicroSource Discovery Inc. (Groton, Conn.). It contains 2,000 compounds on 25 plates, 80 compounds per plate. The library contains known bioactive compounds and natural products and their derivatives.

Sigma LOPAC 1280:

Library LOPAC 1280 was purchased from Sigma-Aldrich. It contains 1,280 compounds on 16 96-well plates, 80 compounds per plate. The library contains pharmacologically active compounds for all major target classes, such as GPCRs and kinases. Some of them are marketed drugs.

ChemBridge CNS-Set:

The CNS-Set library (50,000 compounds) was developed to facilitate the exploration of compounds which would be more likely to pass the blood brain barrier. The library has a log P between 0-5, a lower molecular weight limit (190-500 instead of 170-700). This library is useful not only for CNS therapeutic targets, where a compound's ability to pass the blood brain barrier is critical, but also for general screening conditions

ChemBridge Divert-SET:

The DIVER Set library (50,000 compounds) is designed as a universal screening library, covering the broadest part of pharmacophore diversity space with the minimum number of compounds. This substantially cuts discovery timescales and cost by reducing the number of compounds that need to be tested. DIVER Set is particularly useful for primary screening against a wide range of biological targets, including those where no structural information is available.

BIOMOL Collection:

This collection consists of three sub-libraries: protein kinase or phosphatase inhibitors (84 compounds (link to 2831.xls), ion channel collection (70 compounds, link to 2805 file) and natural product collection (502 compounds, link to 2865.xls).

Potential antagonists of a Keap1 polypeptide or agonists of Nrf2 include organic molecules, peptides, peptide mimetics, polypeptides, nucleic acid molecules (e.g., double-stranded RNAs, siRNAs, antisense polynucleotides), and antibodies that bind to a Keap1 nucleic acid sequence or polypeptide of the invention and thereby inhibit or extinguish its activity. Potential antagonists also include small molecules that bind to the Keap1 polypeptide thereby preventing binding to a Nrf2 polypeptide with which the Keap1 polypeptide normally interacts, such that the normal biological activity of the Keap1 polypeptide is reduced or inhibited. Small molecules of the invention preferably have a molecular weight below 2,000 daltons, more preferably between 300 and 1,000 daltons, and still more preferably between 400 and 700 daltons. It is preferred that these small molecules are organic molecules.

Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized to identify compounds that interact with Nrf2 or Keap1 nucleic acid molecules or polypeptides. Interacting compounds isolated by this method (or any other appropriate method) may, if desired, be further purified (e.g., by high performance liquid chromatography). Compounds isolated by any approach described herein may be used as therapeutics to treat pulmonary inflammatory conditions, pulmonary fibrosis, asthma, chronic obstructive pulmonary disease, emphysema, sepsis, septic shock, cerebral ischemia and neurodegenerative disorders in a human patient.

In addition, compounds that inhibit the expression of an Keap1 nucleic acid molecule whose expression is increased in a subject, are also useful in the methods of the invention. Any number of methods are available for carrying out screening assays to identify new candidate compounds that alter the expression of a Keap1 nucleic acid molecule.

In one approach, the effect of candidate compounds can be measured at the level of polypeptide production to identify those that promote a decrease in a Keap1 polypeptide level or an increase in Nrf2 polypeptide level. The level of Nrf2 or Keap1 polypeptide can be assayed using any standard method. Standard immunological techniques include Western blotting or immunoprecipitation with an antibody specific for a Keap1 or Nrf2 polypeptide. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of an Nrf2 polypeptide is considered particularly useful. Again, such a molecule may be used, for example, as a therapeutic to delay, ameliorate, or treat pulmonary inflammatory conditions, pulmonary fibrosis, asthma, chronic obstructive pulmonary disease, emphysema, sepsis, septic shock, cerebral ischemia and neurodegenerative disorders in a human patient.

Each of the DNA sequences listed herein may also be used in the discovery and development of a therapeutic compound for the treatment of pulmonary inflammatory conditions, pulmonary fibrosis, asthma, chronic obstructive pulmonary disease, emphysema, sepsis, septic shock, cerebral ischemia and neurodegenerative disorders. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

The invention also includes novel compounds identified by the above-described screening assays. Optionally, such compounds are characterized in one or more appropriate animal models to determine the efficacy of the compound for the treatment of pulmonary inflammatory conditions, pulmonary fibrosis, asthma, chronic obstructive pulmonary disease, emphysema, sepsis, septic shock, cerebral ischemia and neurodegenerative disorders. Desirably, characterization in an animal model can also be used to determine the toxicity, side effects, or mechanism of action of treatment with such a compound. Furthermore, novel compounds identified in any of the above-described screening assays may be used for the treatment of a pulmonary inflammatory conditions, pulmonary fibrosis, asthma, chronic obstructive pulmonary disease, emphysema, sepsis, septic shock, cerebral ischemia and neurodegenerative disorders in a subject. Such compounds are useful alone or in combination with other conventional therapies known in the art.

Table 1A lists compounds that are likely to be useful as Nrf2 activators.

### Test Compounds and Extracts

In general, compounds capable of reducing oxidative stress by increasing the expression or biological activity of a Nrf2 nucleotide or a Nrf2 polypeptide or decreasing the expression or activity of Keap1 are identified from large libraries of either natural product or synthetic (or semi-synthetic) extracts or chemical libraries according to methods known in the art. Methods for making siRNAs are known in the art and are described in the Examples. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptide libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their antioxidant activity should be employed whenever possible.

In an embodiment of the invention, a high throughput approach can be used to screen different chemicals for their potency to affect Nrf2 activity. A cell based transcriptional reporter approach, for example, can be used to identify agents that increase Nrf2 transcription.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of a Nrf2 polypeptide, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, compounds shown to be useful agents for the treatment of a neoplasm are chemically modified according to methods known in the art.

### Combination Therapies

Compositions and methods of the invention may be used in combination with any conventional therapy known in the art. In one embodiment, an agent that activates Nrf2 is used in combination with anti-oxidants known in the art. Exemplary anti-oxidants include, for example, enzymatic antioxidants, such as the families of superoxide dismutase (SOD), catalase, glutathione peroxidase, glutathione S-transferase (GST), and thioredoxin; as well as nonenzymatic antioxidants, including glutathione, ascorbate, α-tocopherol, urate, bilirubin and lipoic acid, vitamin C and β-carotene.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The following non-standard abbreviations are used: Cigarette smoke (CS); nuclear factor erythroid-derived 2-related factor 2 (Nrf2); antioxidant response element (ARE); terminal deoxynucleotidyl transferase-mediated dUTP end-labeling (TUNEL); 8-oxo-7,8-dihydro-2′-deoxyguanosine (8-oxo-dG); bronchoalveolar lavage (BAL); airway hyperreactivity (AHR); electrophoretic mobility shift assay (EMSA); OVA challenged Nrf2^{+/+} mice (Nrf2^{+/+} OVA mice); OVA challenged Nrf2^{-/-} mice (Nrf2^{-/-} OVA mice); mouse embryonic fibroblasts (MEFs); TLR, toll-like receptor (TLR); Epicatechin (EC); common carotid artery (CCA); external carotid artery (ECA); internal carotid artery (ICA), middle cerebral artery (MCA). MCA occlusion (MCAO), Carbon Monoxide (CO), cerebral blood flow (CBF), heme oxygenase (HO), 2,3,5-triphenyltetrazolium chloride (TTC), anterior cerebral artery cortex (ACA CTX); contralateral anterior cerebral artery, (CACA); parietal 1 (P1); contralateral parietal 1 (CP1); parietal 2 (P2); contralateral parietal 2 (CP2); lateral cortex; (LAT CTX); contralateral lateral cortex (CLAT CTX); dorsomedial caudate putamen (DM CP); contralateral dorsomedial caudate putamen (CDM CP); ventrolateral caudate putamen (VL CP); contralateral ventrolateral caudate putamen (CVL CP). CVL CP; AW, airways;

### Example 1

### nrf2−/− Mice have Increased Susceptibility to CS-Induced Emphysema

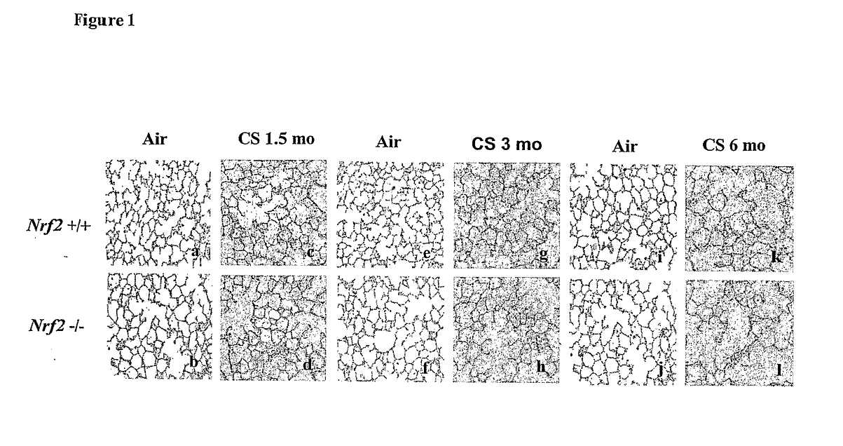

The lungs from air-exposed nrf2-disrupted and wild-type (nrf2+/+) mice showed normal alveolar structure when examined using hemotoxylin and eosin (H&E) staining (FIG. 1). Because the alveolar diameter of air-exposed nrf2−/− mice was slightly smaller than in the wild-type counterpart (Table 1, below), detailed lung morphometric measurements and light microscopic and ultrastructural studies were performed to rule out that nrf2−/− lung had delayed development or structural integrity when maintained at room air.

TABLE 1
Effect of chronic exposure to cigarette smoke on lung morphometry. Values
shown are the mean ± SEM for groups of 5 mice each. *, significantly
greater than the CS exposed (6 months) Nrf2+/+ mice. P ≦ 0.05

| | Time of | Alveolar diameter (μm) | | | Mean linear intercept (μm) | | |
|---|---|---|---|---|---|---|---|
| | exposure | | | % | | | % |
| Groups | (months) | Air | CS | Increase | Air | CS | Increase |
| Nrf2+/+ | 1.5 | 37.2 ± 1.3 | 39.1 ± 1.5 | 5.1 | 51.9 ± 2.3 | 52.3 ± 1.8 | 1.9 |
| | 3 | 37.5 ± 1.6 | 40.5 ± 1.4 | 7.9 | 51.8 ± 2.7 | 53.6 ± 1.6 | 3.3 |
| | 6 | 38.9 ± 1.5 | 42.2 ± 1.7 | 8.5 | 52.6 ± 2.1 | 57.0 ± 1.5 | 8.3 |
| Nrf2−/− | 1.5 | 34.5 ± 1.3 | 37.0 ± 1.6 | 7.2 | 50.0 ± 2.0 | 52.1 ± 2.0 | 4.3 |
| | 3 | 34.9 ± 1.2 | 41.8 ± 1.4 | 19.5 | 52.1 ± 1.8 | 58.0 ± 2.1 | 11.2 |
| | 6 | 35.8 ± 1.4 | 47.7 ± 1.5* | 33.1 | 53.5 ± 1.7 | 67.5 ± 2.3* | 26.1 |

There were no significant differences in alveolar diameter and mean linear intercept between nrf2+/+ and 1-lungs at 3 days, 10 days, 2 months and 6 months of age. Histochemical staining for reticulin and elastin showed similar alveolar architecture in the wild-type and knockout lungs, with progressive attenuation of alveolar septa occurring between day 10 and 2 months of age in both genetic backgrounds. There was no significant difference in the total lung capacity of the air exposed (2 months old) nrf2+/+[(1.19±0.16 ml for 23±1.4 g mice) and −/− mice (1.12±0.19 ml for 23±1.2 g mice)] and the proliferation rate was similar in nrf2+/+ and nrf2−/− lungs. Further, nrf2+/+ and −/− lungs had similar ultrastructural alveolar organization with alveolar-capillary membranes lined by type I epithelial cells, and normal alveolar type II cell population. Histological examination of the lung sections did not reveal any tumors in air or CS-exposed mice. Further, H&E stained lung sections did not show any significant inflammation in the lungs of air-exposed nrf2+/+ and −/− mice (FIG. 1).

To determine the role of Nrf2 in susceptibility to CS-induced emphysema, nrf2-disrupted and wild-type nrf2 (ICR strain) mice were exposed to CS for 1.5 to 6 months, and CS-induced lung damage was assessed by computer-assisted morphometry. There was an increase in alveolar destruction in the lungs of nrf2-disrupted mice when compared to wild-type ICR mice after 6 months of exposure to CS. Both the alveolar diameter (increased by 33.1% in nrf2−/− vs. 8.5% in nrf2+/+ mice) and mean linear intercept (increased by 26.1% in nrf2−/− vs. 8.3% in nrf2+/+ mice) were significantly higher in CS-exposed nrf2-disrupted mice (Table 1, FIG. 1). Alveolar enlargement was detected in the lungs of nrf2−/− mice as early as 3 months of exposure to CS (Table 1, FIG. 1), suggesting an earlier onset of emphysema in nrf2-disrupted mice. Long-term exposure of nrf2+/+ mice to CS for 6 months resulted in an increase of <10% in the mean linear intercept and alveolar diameter (Table 1), highlighting the intrinsic resistance of nrf2+/+ ICR mice to CS-induced pulmonary emphysema. These results show that nrf2−/− mice have increased susceptibility to CS-induced emphysema.

### Example 2

### CS Induced Lung Cell Apoptosis Following CS Treatment and Activated Caspase-3 in nrf2−/− Lungs

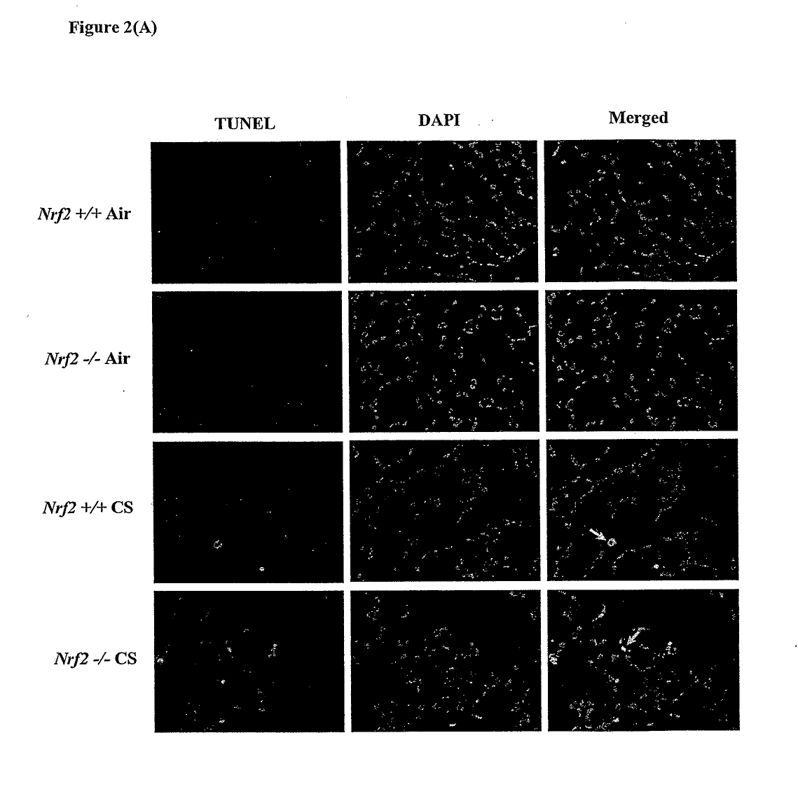

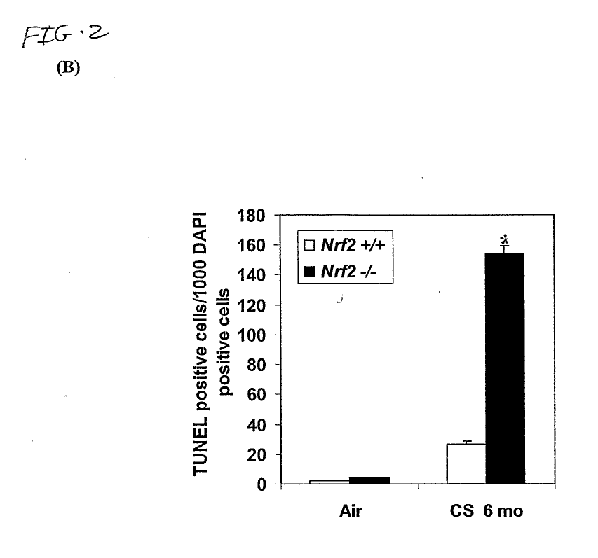

To determine whether chronic exposure to CS (6 months) induced apoptosis of alveolar septal cells in vivo, terminal deoxynucleotidyl transferase-mediated dUTP end-labeling (TUNEL) was conducted on lung sections from air and CS exposed mice. Labeling of DNA strand breaks in situ by the fluorescent TUNEL assay demonstrated a higher number of TUNEL-positive cells in the alveolar septa of CS-exposed nrf2−/− mice (154.27 TUNEL-positive cells/1000 DAPI positive cells) than in CS-exposed nrf2+/+ mice (26.42 TUNEL-positive cells/1000 DAPI positive cells) or air-exposed nrf2−/− or +/+ mice (FIGS. 2A and B). Double staining of the TUNEL-labeled lung sections (FIG. 2C) with anti-SpC (type II epithelial cells), anti-CD34 (endothelial cells) and Mac-3 (macrophages) antibodies revealed the occurrence of apoptosis, predominantly in endothelial cells (nrf2−/−=52±3.6 vs. nrf2+/+=8±1.8 TUNEL-positive CD34-positive cells/1000 DAPI-positive alveolar cells) and type II epithelial cells (nrf2−/−=43±4.3 vs. nrf2+/+=6±0.96 TUNEL-positive SpC-positive cells/1000 DAPI-positive alveolar cells) in the lungs of CS-exposed nrf2−/− mice, when compared with nrf2+/+ mice. Most alveolar macrophages in CS-exposed lungs did not show evidence of apoptosis (nrf2−/−=5±0.42 Mac-3-positive cells/1000 DAPI positive cells vs. nrf2+/+=3±0.96 Mac-3 positive cells/1000 DAPI-positive cells).

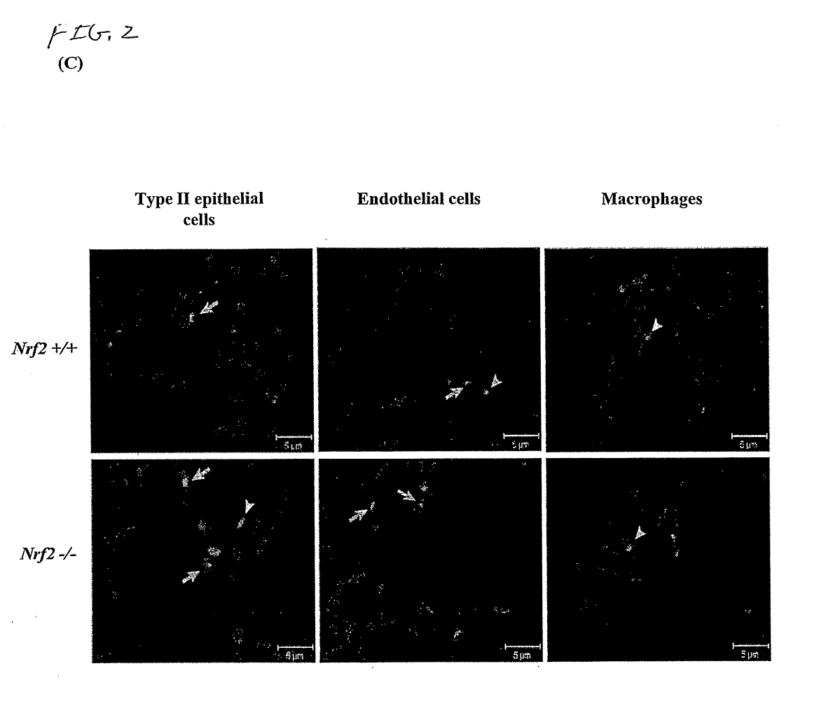

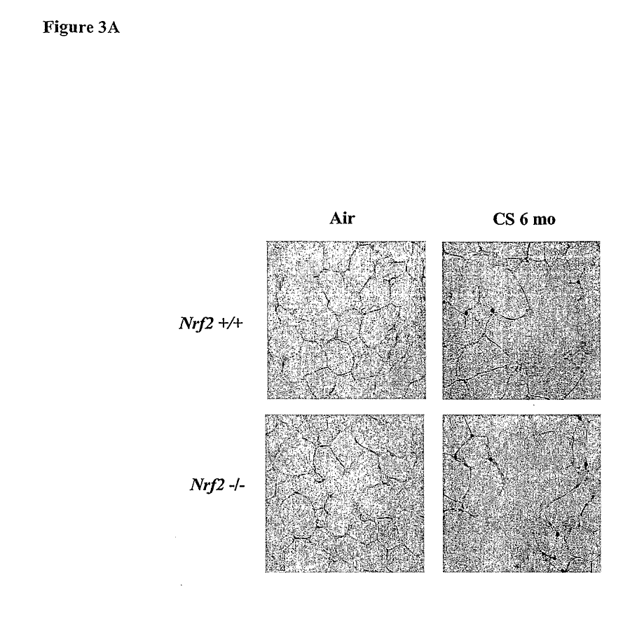

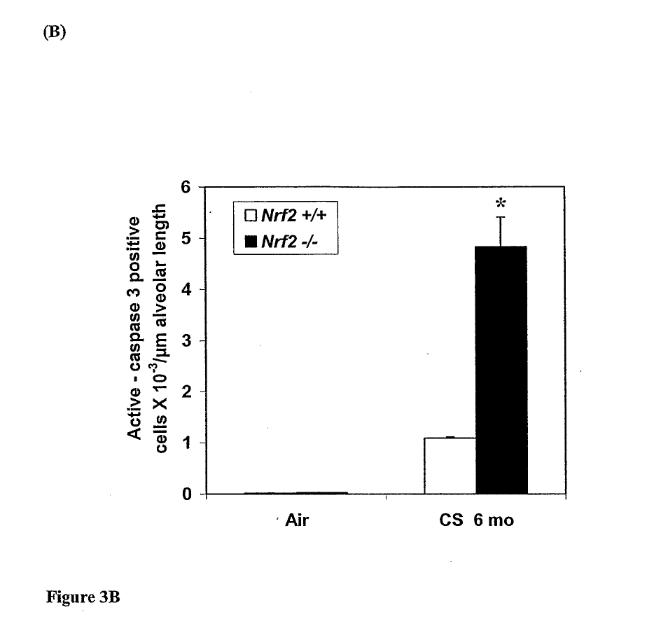

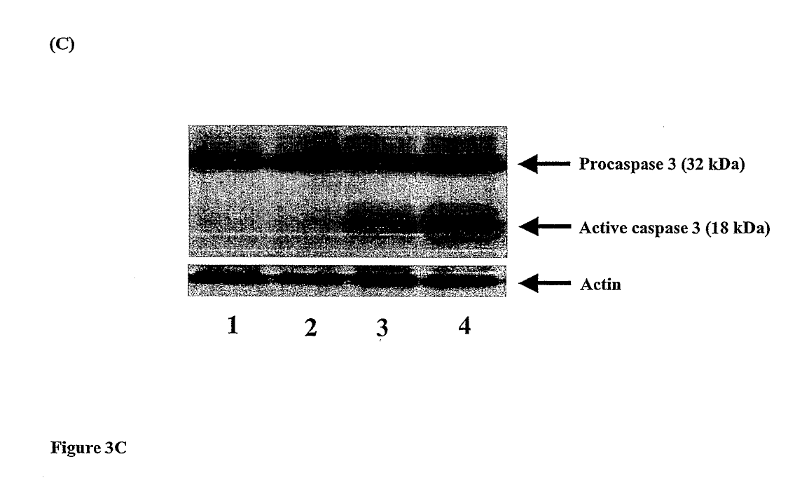

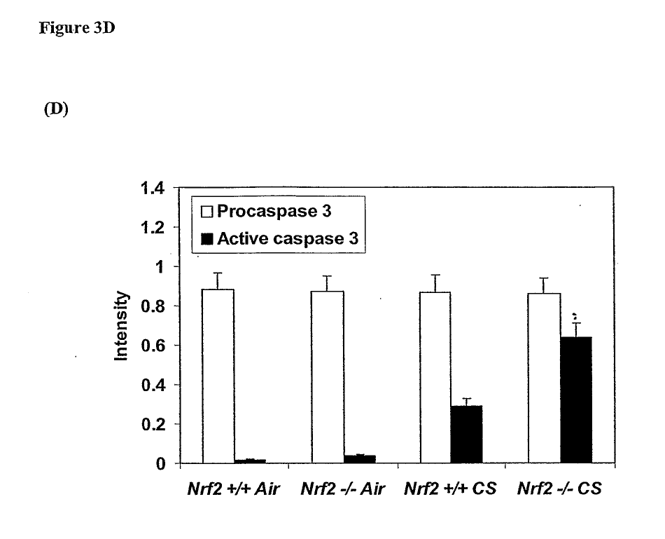

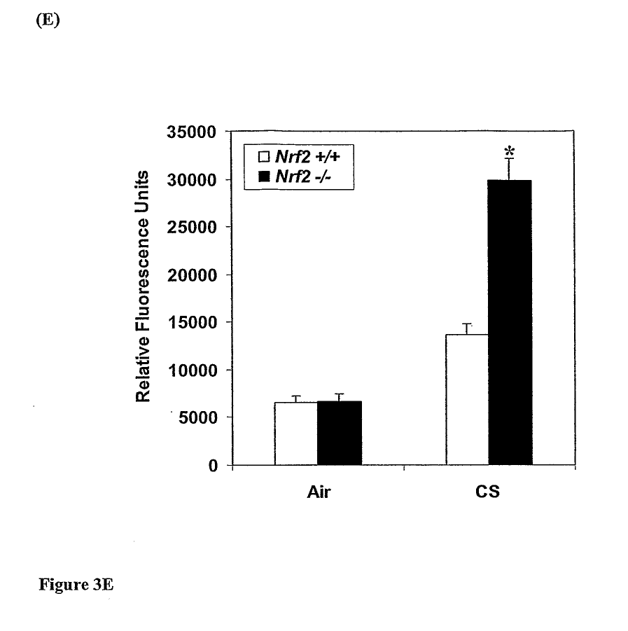

Immunohistochemical analysis showed a higher number of caspase 3-positive cells in the alveolar septa of CS-exposed nrf2−/− mice (4.83 active-caspase 3-positive cells/mm alveolar length) than in CS-exposed nrf2+/+ mice (1.09 active-caspase 3-positive cells/mm alveolar length). Lung sections from the air-exposed control nrf2−/− and wild-type mice showed few or no caspase 3-positive cells (FIGS. 3A and B). Enhanced activation of caspase 3 in nrf2−/− lungs exposed to CS for 6 months was further documented by the increased detection of the 18 kDa active caspase 3 cleaved product in whole lung lysates (2.3 fold increase in nrf2−/− vs. CS-exposed nrf2+/+ mice) (FIGS. 3C and D), and increased caspase 3 enzymatic activity (2.1 fold increased activity in nrf2−/− mice vs. CS-exposed nrf2+/+ mice) (FIG. 3E). These results demonstrate that CS causes lung cell apoptosis, and further that CS treatment leads to activation of caspase-3 in nrf2−/− lungs.

### Example 3

### nrf2−/− Mice have Increased Sensitivity to Oxidative Stress after CS Exposure

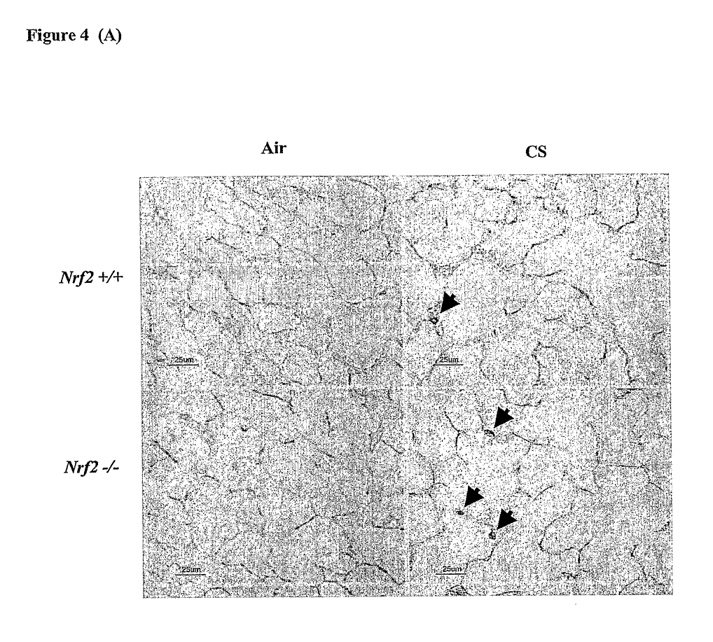

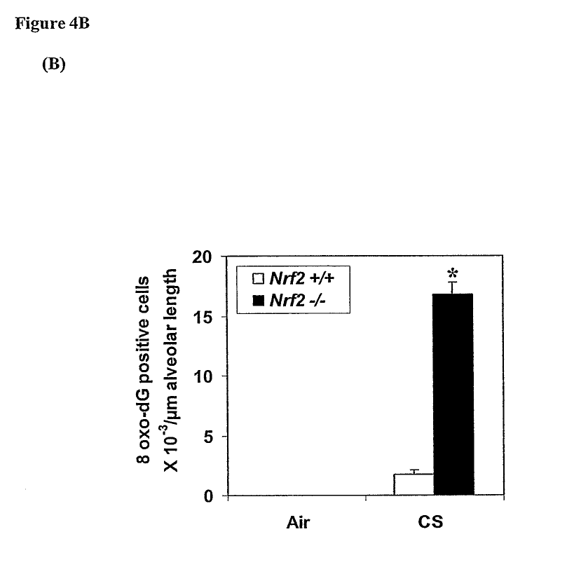

Immunohistochemical staining with anti-8-oxo-dG antibody was used to assess oxidative stress in both nrf2−/− and +/+ lungs after inhalation of CS. A number of alveolar septal cells exhibited staining for 8-oxo-dG in lung sections from nrf2+/+ mice (1.78 positive cells/mm alveolar length) than in CS-exposed nrf2−/− mice (16.8 positive cells/mm alveolar length) (FIGS. 4A and B). Lung sections from air-exposed nrf2+/+ and −/− mice showed few or no 8-oxo-dG-positive cells. Immunostaining with normal mouse IgG antibody did not show any IgG reactive cells in the lungs of air or CS exposed mice (FIG. 4C). These results indicate that exposure to CS for 6 months enhanced oxidative damage to the lungs of the nrf2-disrupted mice. Further, the results show an increased sensitivity of nrf2−/− mice to oxidative stress after CS exposure.

### Example 4

### CS-Exposed nrf2−/− Mice have Increased Inflammation in the Lungs

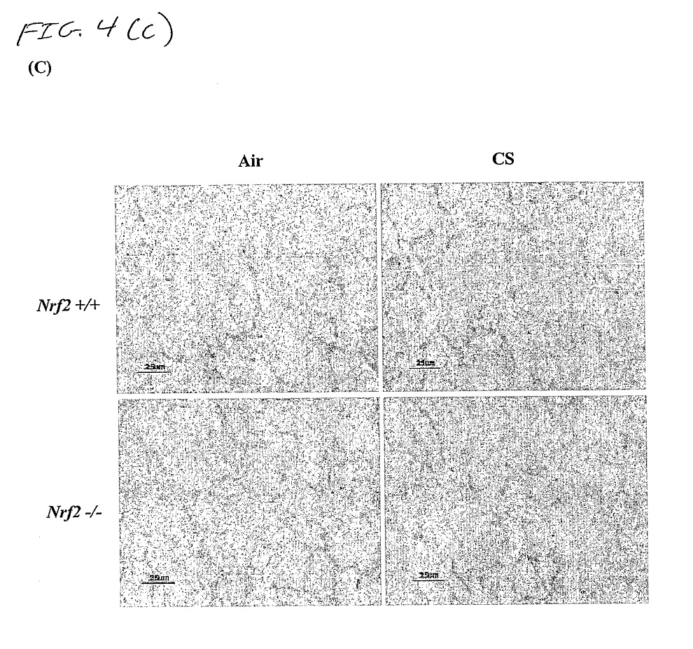

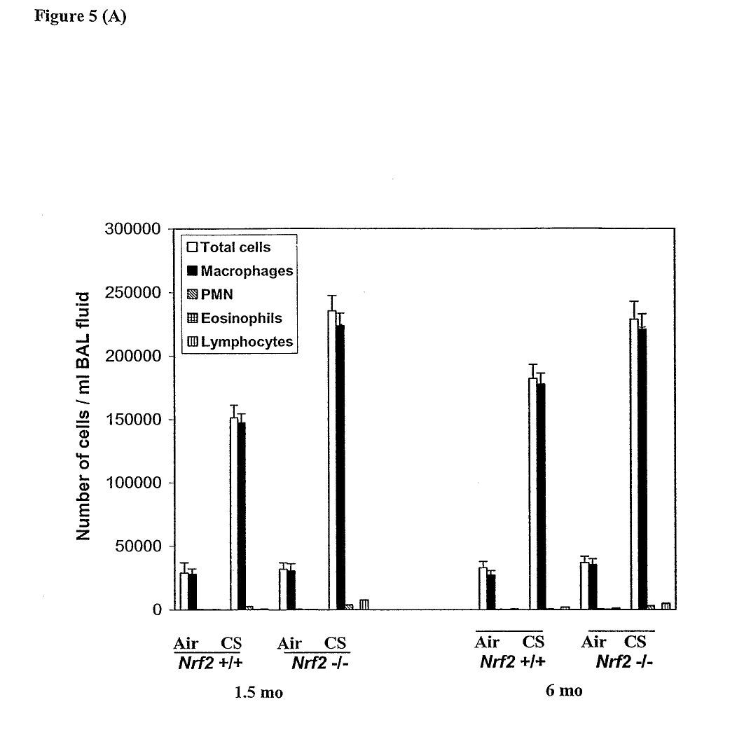

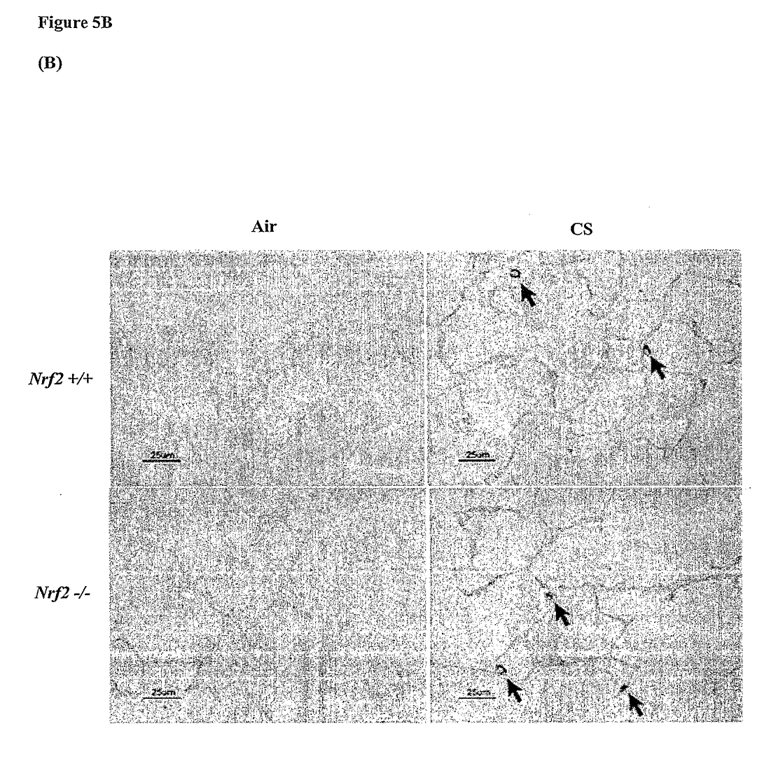

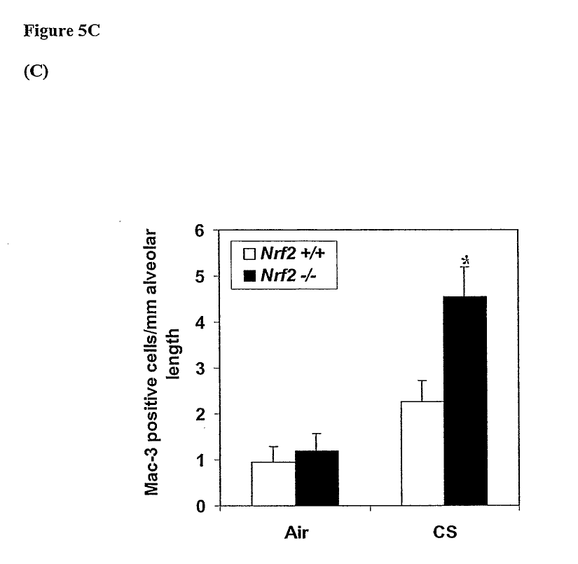

Analysis of differential cell counts of bronchioalveolar fluid (BAL) revealed a significant increase in the number of total inflammatory cells in the lungs of CS-exposed (1.5 or 6 months) nrf2+/+ and −/− mice, when compared to their respective air-exposed control littermates (FIG. 5A). However, the total number of inflammatory cells in BAL fluid from the CS-exposed nrf2−/− mice was significantly higher than in CS-exposed wild-type mice. Among the inflammatory cell population, macrophages were the predominant cell type, constituting as much as 87-90% of the total inflammatory cell population in the BAL fluid of both genotypes exposed to CS. Other inflammatory cells such as polymorphonuclear leukocytes (PMN), eosinophils and lymphocytes constituted 10-13% of the total inflammatory cells in the BAL fluid of both genotypes. Immunohistochemical staining of the lung sections with Mac-3 antibody revealed the presence of increased number of macrophages (FIGS. 5B and C) in the lungs of CS-exposed nrf2−/− mice at 6 months (4.54 Mac-3 positive cells/mm alveolar length) when compared with lungs of their wild-type counterparts (2.27 Mac-3 positive cells/mm alveolar length). Immunohistochemical staining did not show any significant difference in the number of alveolar macrophages in the lungs of air-exposed nrf2+/+ (0.96 Mac-3 positive cells/mm alveolar length) and nrf2−/− mice (1.18 Mac-3 positive cells/mm alveolar length). Further, the number of neutrophils and lymphocytes were significantly smaller than that of macrophages. There were 0.92 vs. 0.49 neutrophils and 0.78 vs 0.43 lymphocytes/mm alveolar length in CS-exposed nrf2−/− and wild-type mice respectively. These results demonstrate that there is increased inflammation in the lungs of CS-exposed nrf2−/− mice.

### Example 5

### Nrf2 is Activated in the Lungs of nrf2+/+ Mice

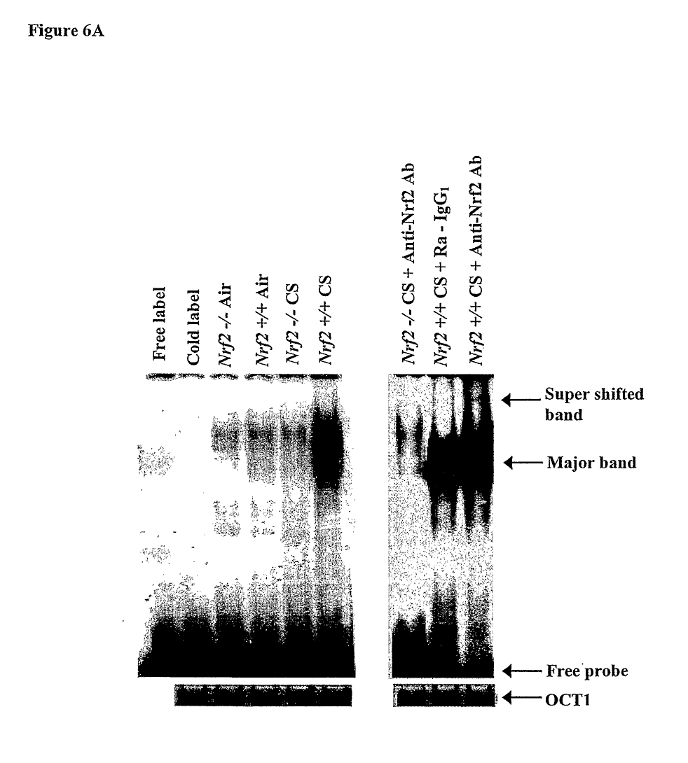

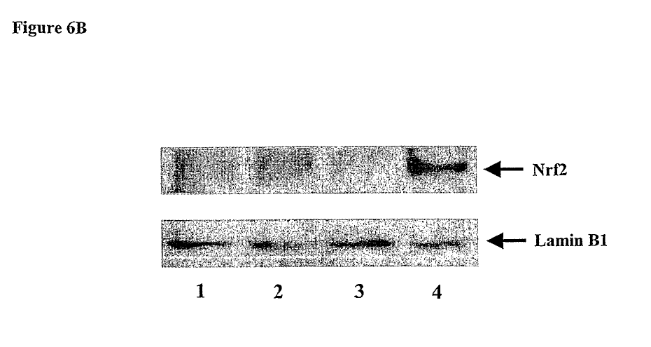

Electrophoretic mobility shift assay (EMSA) was used to determine the activation and DNA binding activity of Nrf2, in the lungs in response to acute exposure of the mice to CS (5 hours). In response to CS, there was an increased binding of nuclear proteins isolated from the lungs of CS-exposed nrf2+/+ mice to an oligonucleotide probe containing the ARE consensus sequence, as compared to the binding of nuclear proteins isolated from CS-exposed nrf2−/− mice or air-exposed control mice. Supershift analysis with anti-Nrf2 antibody also showed the binding of Nrf2 to the ARE consensus sequence, suggesting the activation of Nrf2 in the lungs of nrf2+/+ mice in response to CS exposure (FIG. 6A). However, supershift analysis of the nuclear proteins from the lungs of CS-exposed nrf2−/− mice with anti-Nrf2 antibody did not show any super-shifted band, consistent with the absence of Nrf2 in the ARE-nuclear protein complex.

Western blot analysis was performed to determine the nuclear accumulation of Nrf2 in the lungs in response to CS exposure. Immunoblot analysis (FIG. 6B) demonstrated increased level of Nrf2 in the nuclei isolated from the lungs of CS-exposed nrf2+/+ mice, suggesting the nuclear accumulation of Nrf2 in the lungs of wild-type mice in response to CS exposure. Increases in nuclear Nrf2 are needed for the activation of ARE and the transcriptional induction of various antioxidant genes. These results demonstrate an activation of Nrf2 in CS-exposed lungs in wild type mice with functional Nrf2 (nrf2+/+ mice).

### Example 6

### Nrf2-Dependent Protective Genes were Induced by CS

To determine Nrf2-dependent genes that may account for the emphysema-sensitive phenotype of the nrf2−/− background, the pulmonary expression profile of air-exposed and CS-exposed (5 hours) mice was examined by oligonucleotide microarray analysis using the Affymetrix mouse gene chip U74A. Table 2 (below) lists the genes that were significantly upregulated only in the lungs of nrf2+/+ mice but not in nrf2−/− lungs in response to CS.

TABLE 2
Nrf2-dependent protective genes induced by CS in the lungs of nrf2 wild-type mice.

| | | Fold | |
|---|---|---|---|
| Functional classification | | change ± | |
| and gene accession No. | Gene | SE | ARE position |
| Antioxidants | | | |
| X56824 (X06985) | Heme oxygenase 1^{A} | 4.7 ± 0.4 | −3928, −3992, −6007, |
| | | | −7103, −8978, −9007, |
| | | | −9036, −9065, −9500 |
| U38261 (U10116) | Superoxide dismutase 3^{B} | 1.7 ± 0.4 | −2362, −3171, −5282 |
| X91864 (X68314) | Glutathione peroxidase 2^{B} | 2.7 ± 0.4 | −44, −3600 |
| U13705 (X58295) | Glutathione peroxidase 3^{B} | 1.4 ± 0.4 | −7144, −9421 |
| U85414 (M90656) | Gamma glutamylcysteine | 7.6 ± 0.5 | −3479, −3524, |
| | synthase (catalytic)^{A} | | −5421 |
| U95053 (L35546) | Gamma glutamylcysteine | 7.3 ± 0.5 | −44 |
| | synthase (regulatory)^{A} | | |
| AF090686 (M60396) | Transcobalamine II^{B} | 1.6 ± 0.3 | −3751, −6382, −8236 |
| L39879 (BC004245) | Ferritin light chain 1^{A} | 1.5 ± 0.3 | −1379 |
| AI118194 (X67951) | Peroxiredoxin 1^{B} | 1.5 ± 0.3 | −78, −8413, −9652 |
| AI851983 (X15722) | Glutathione reductase^{B} | 3.3 ± 0.4 | −115, −9433 |
| AB027565 (X91247) | Thioredoxin reductase 1^{B} | 4.3 ± 0.4 | −121, −4326, −9521 |
| Z11911 (X03674) | Glucose-6-phosphate | 2.0 ± 0.3 | −2504, −2109 |
| | dehydrogenase^{B} | | |
| AW120625 (U30255) | Phosphogluconate | 2.1 ± 0.4 | −757, −3963 |
| | dehydrogenase^{B} | | |
| Detoxification enzymes | | | |
| L06047(AF025887) | Glutathione-S-transferase, | 2.0 ± 0.3 | NF |
| | alpha 1^{B} | | |
| J03958 (M16594) | Glutathione-S- | 2.6 ± 0.3 | −6662, −6961, |
| | transferase, alpha 2^{A} | | −7751 |
| X65021 | Glutathione-S- | 1.5 ± 0.3 | No human |
| | transferase, alpha 3^{B} | | homolog |
| AI843119 (U90313) | Glutathione-S- | 2.0 ± 0.3 | −255 |
| | transferase, omega 1^{B} | | |
| X53451 (X06547) | Glutathione-S- | 3.1 ± 0.3 | −71 |
| | transferase, pi 2^{B} | | |
| J03952 (J03817) | Glutathione-S-transferase | 1.6 ± 0.3 | −1209 |
| | GT8.7^{B} | | |
| U12961 (J03934) | NADPH: quinone | 9.3 ± 0.5 | −527 |
| | reductase 1^{A} | | |
| U20257 (U09623) | Alcohol dehydrogenase 7 | 2.8 ± 0.3 | −2894 |
| | (class IV)^{B} | | |
| AV089850 (M74542) | Aldehyde dehydrogenase family | 11.1 ± 0.8 | −4223 |
| | 3, subfamily A1^{B} | | |
| U04204 | Aldo-keto reductase1, | 5.4 ± 0.5 | No human |
| | member B8^{B} | | homolog |
| AB017482 (AH005616) | Retinol oxidase/Aldehyde | 2.3 ± 0.4 | −8579 |
| | oxidase^{B} | | |
| AB025408 (AF112219) | Esterase 10^{B} | 3.4 ± 0.4 | −4105, −4264 |
| U16818 (J04093) | UDP-glucuronosyl transferase^{B} | 1.4 ± 0.3 | −5431, −6221 |
| AF061017 (AF061016) | UDP-glucose dehydrogenase^{B} | 1.5 ± 0.6 | −3438 |
| Protective proteins | | | |
| M64086 (AH002551) | α1-antitrypsin proteinase | 4.7 ± 0.3 | −4117 |
| | inhibitor^{B} | | |
| AB034693 (AB034695) | Endomucin-1^{B} | 1.5 ± 0.3 | −2565 |
| AW120711 (AF087870) | Dnaj (HSP 40) homolog^{B} | 1.9 ± 0.4 | −155, −2797, −5320 |
| D17666 (AU130219) | Mitochondrial stress-70 | 1.6 ± 0.3 | −2675, −3302 |
| | protein^{B} | | |
| AF055638 (AF265659) | GADD45G^{B} | 2.4 ± 0.3 | −327 |
| U08210 (M16983) | Tropoelastin^{B} | 2.8 ± 0.9 | NF |
| X04647 (X05562) | Procollagen type IV, alpha 2^{B} | 1.9 ± 0.4 | NF |
| Transcription factors | | | |
| AB009694 (AJ010857) | mafF^{B} | 2.6 ± 0.4 | −3894, −6537, −8279, |
| | | | −8301, −8445 |
| AF045160 (U81984) | HIF-1 alpha related factor^{B} | 2.0 ± 0.4 | −3855, −5091 |
| Protein degradation | | | |
| AV305832 (M26880) | Ubiquitin C^{B} | 1.8 ± 0.4 | −1393, −3755, −4481 |
| AW121693 (AA020857) | Proteasome (prosome, | 1.7 ± 0.3 | NF |
| | macropain) 26S subunit, non- | | |
| | ATPase, 1^{B} | | |
| U40930 (BC017222) | Seqestosome 1^{B} | 2.9 ± 0.4 | −360, −1328 |
| Transporters | | | |
| M22998* (K03195) | Solute carrier family 2^{B} | 2.9 ± 0.2 | −3351, −5111, −9304 |
| X67056 (S70612) | Glycine transporter^{B} | 1.8 ± 0.3 | −387, −8451 |
| U75215 (BC026216) | Neutral amino acid transporter | 3.8 ± 0.3 | −3695, −8547 |
| | mASCT1^{B} | | |
| Phosphatases | | | |
| M97590 (AH003242) | Tyrosine phosphatase (PTP1)^{B} | 1.6 ± 0.3 | −6045, −3232, −7029, |
| | | | −9884 |
| X58289 (X5431) | Protein tyrosine phosphatase, | 1.7 ± 0.4 | −8166, −9561, −9662 |
| | receptor type B^{B} | | |
| Receptor | | | |
| AJ250490 (AJ001015) | Receptor activity modifying | 1.6 ± 0.3 | −5023, −3455 |
| | protein 2^{B} | | |

^{A}Genes have already been reported to have ARE(s) and regulated by Nrf2;
^{B}Genes with the newly identified AREs using Genamics expression 1.1 pattern finder tool software; ARE(s) reported in the table are for human genes homologous to the respective mouse gene; the number in parenthesis refers to human accession number. To locate the ARE (s) in each gene, 10 kb sequences upstream of the transcription start site (TSS) in both the strands were scanned using the ARE consensus sequence RTGAYNNNGCR as probe; TSS for all the genes was determined by following the Human Genome build 34, version 1 of the NCBI database.
NF, not found.

The regions upstream of the transcription start site of these Nrf2-dependent genes were analyzed for the presence of putative ARE(s) using the Genamics Expression 1.1 Pattern Finder Tool software. The location of the ARE(s) in these Nrf2-dependent genes are presented in Table 2. Nrf2 regulates about 50 antioxidant and cytoprotective genes. The majority of these Nrf2-regulated genes contain possible functional ARE(s) in the genomic sequences upstream of their transcription start sites.

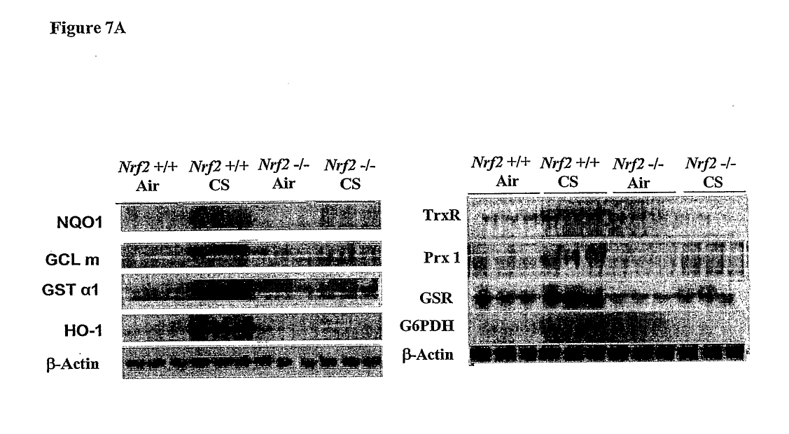

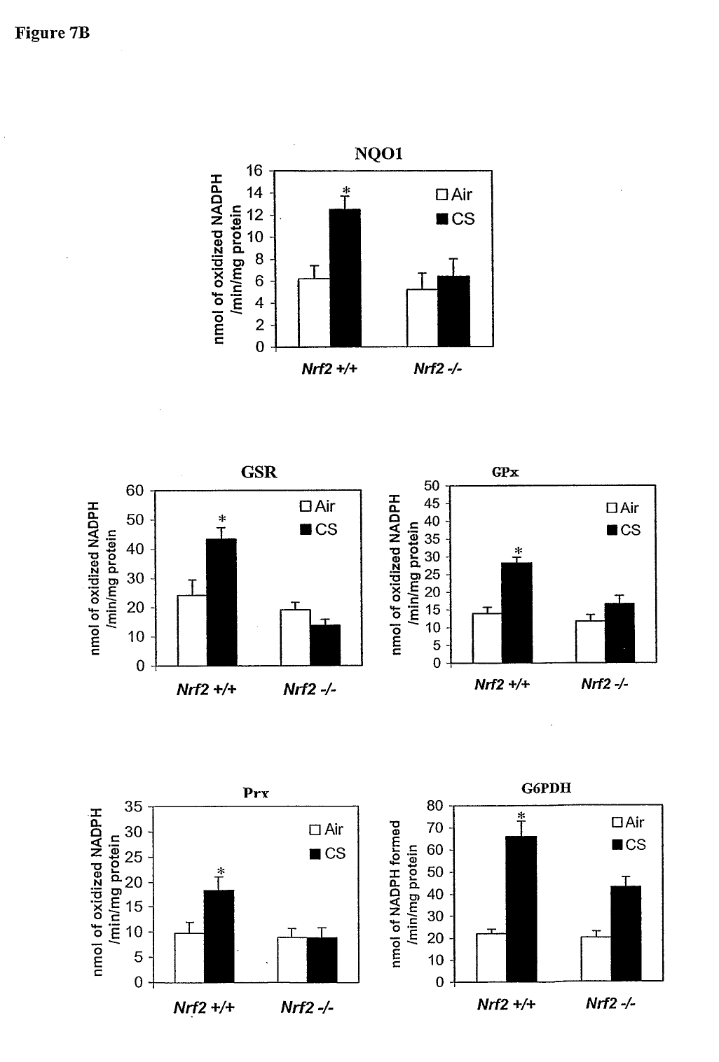

Validation of the microarray data was performed using the samples used in the arrays. Northern hybridization confirmed the transcriptional induction of genes involved in glutathione synthesis (GCLm), NADPH regeneration [glucose 6 phosphate dehydrogenase (G6PDH)], detoxification of oxidative stress inducing components of CS [NADPH: quinine oxidoreductase 1 (NQO1), GST α1, HO-1, thioredoxin reductase (TrxR) and peroxiredoxin 1 (Prx 1)] in the lungs of CS-exposed nrf2+/+ but not nrf2−/− mice (FIG. 7A). Glutathione reductase (GSR) was also induced in CS-exposed nrf2−/− mice; however, the magnitude of the induction was significantly higher in nrf2 wild-type mice than in nrf2-disrupted mice. The increases in these induced genes (NQO1, 7.2-fold; GST α1, 2-fold; γ-GCS(h), 4.8-fold; TrxR, 4.8-fold; G6PDH, 2.2-fold; HO-1, 3.4-fold; GSR, 1.8 fold; Prx 1, 1.6-fold) as measured by Northern analysis were comparable to those determined by microarray.

Enzyme assays of selected gene products [NQO1, GSR, Prx, glutathione peroxidase (GPx) and G6PDH] were carried out to determine the extent to which their transcriptional induction in the lung paralleled changes in their activities (FIG. 7B). There was a significant increase in the activities of all the enzymes in the lungs of CS-exposed nrf2+/+ mice when compared to CS-exposed nrf2−/− mice, as well as in the respective air-exposed control mice. Moreover, the basal activities of these enzymes were significantly lower in the air-exposed nrf2-disrupted mice than in the air-exposed wild-type mice. Taken together, this data demonstrated that Nrf2-dependent protective genes were induced by CS in the lungs of nrf2 wild-type mice.

### Example 7

### Nrf2−/− Mice Had Increased Asthmatic Inflammation Following OVA Challenge

Oxidative stress has been postulated to play an important role in the pathogenesis of asthma. Nrf2 is a redox-sensitive basic-leucine zipper transcription factor that is involved in the transcriptional regulation of many antioxidant genes. As described herein, disruption of the Nrf2 gene leads to severe allergen-driven airway inflammation and hyperresponsiveness in mice sensitized with ovalbumin, termed “OVA challenged”. Thus, the responsiveness of Nrf2-directed antioxidant pathways likely acts as a major determinant of susceptibility to allergen mediated asthma.

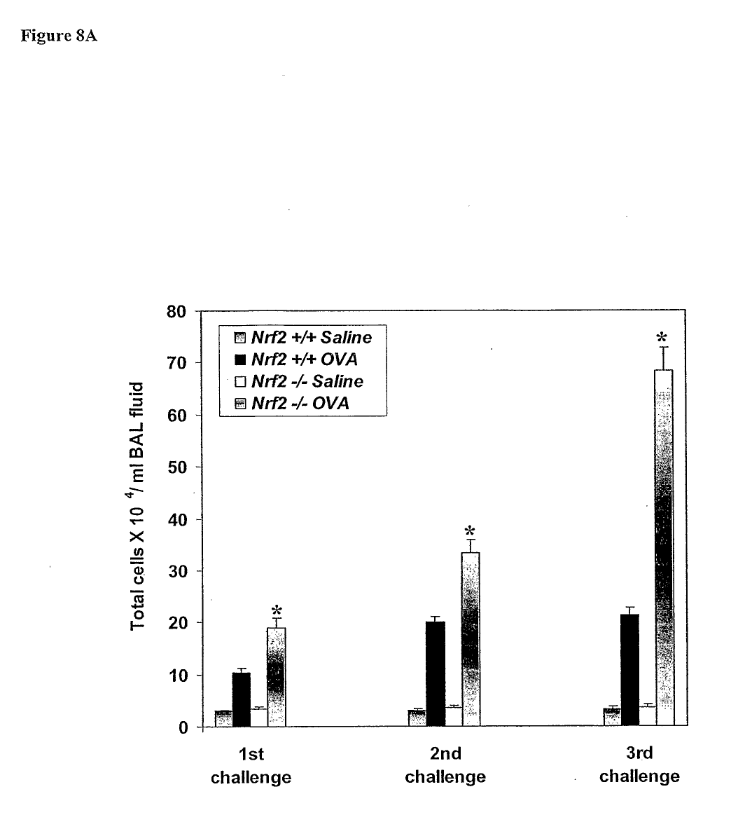

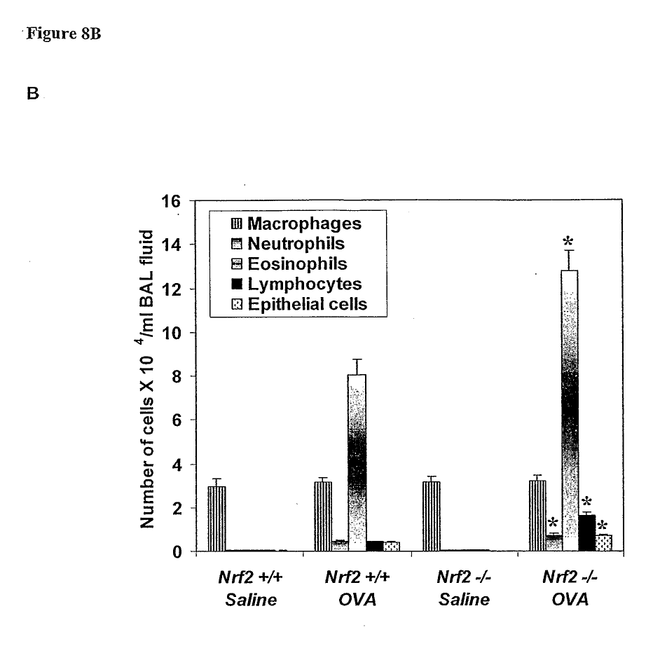

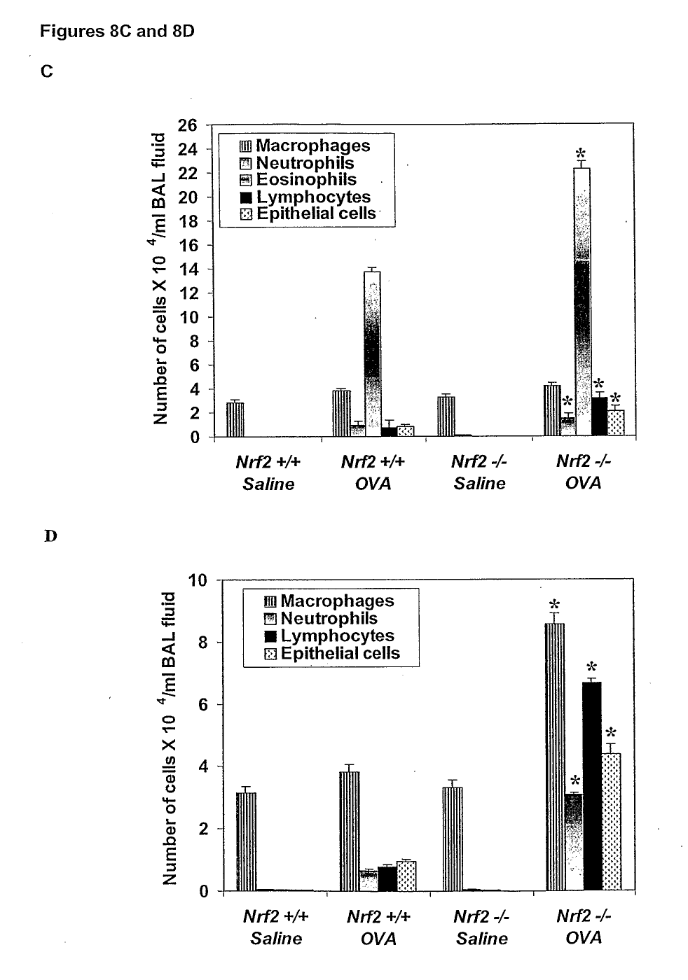

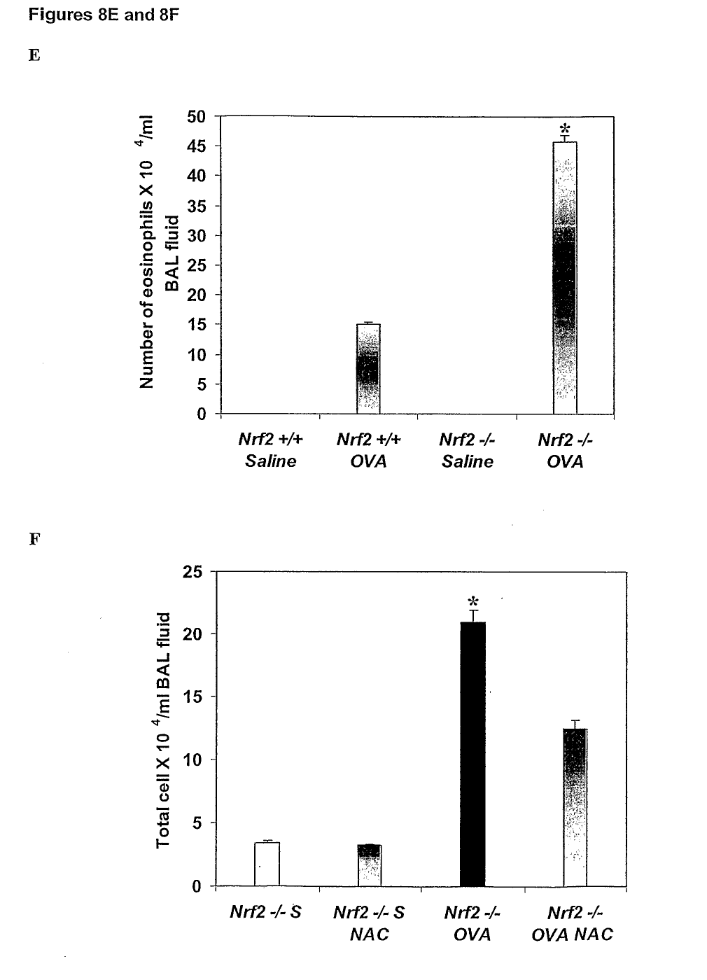

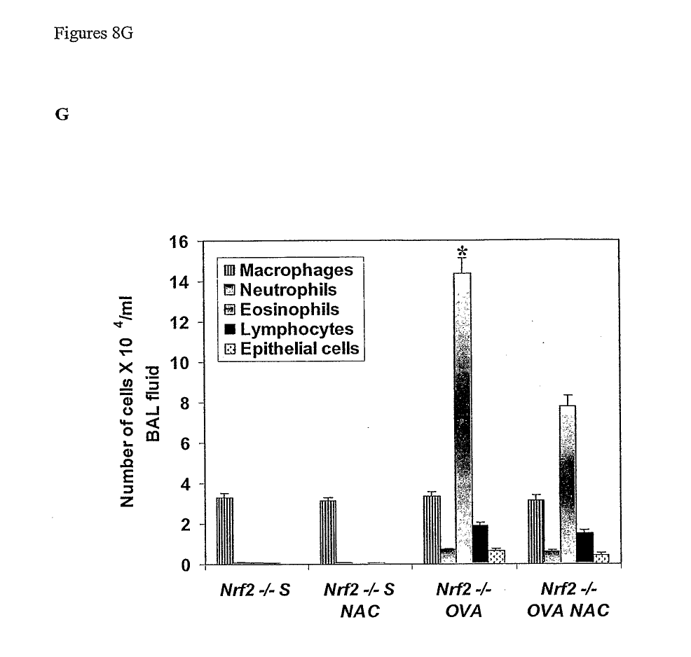

The total number of inflammatory cells in the BAL fluid of all OVA challenged (1^{st} to 3rd) Nrf2-deficient mice (Nrf2^{-/-} OVA mice) was significantly higher than OVA challenged Nrf2 wild-type mice (Nrf2^{+/+} OVA mice) (FIG. 8A). The number of inflammatory cells in the BAL fluid of Nrf2^{-/-} OVA mice (3^{rd} challenge) was 2.9 fold higher (0.67 million/ml BAL) than its level (0.23 million/ml BAL) in Nrf2^{+/+} OVA mice. The increase in inflammation was progressive from the 1^{st} to the 3^{rd} OVA challenge. Differential cell count analysis showed a significantly higher number of eosinophils, lymphocytes and neutrophils as well as epithelial cells in the BAL fluid of Nrf2 OVA mice (FIGS. 8 B, C, D, and E). Seventy two hours after the 3^{rd} challenge, there were 2.3-, 3-, 4.5-, 4.8- and 8.5-fold more macrophages, eosinophils, epithelial cells, neutrophils and lymphocytes respectively in the BAL fluid of Nrf2^{-/-} OVA mice than Nrf2^{+/+} OVA mice (FIGS. 8 D and E). Among the inflammatory cell populations, eosinophils were the predominant cell population, followed by macrophages, lymphocytes and neutrophils at each time point (FIGS. 8 B, C, D, and E). These results demonstrate increased allergen-driven asthmatic inflammation in OVA challenged Nrf2−/− mice.

### Example 8

### OVA Challenged Nrf2−/− Mice Had Increased Infiltration of Inflammatory Cells

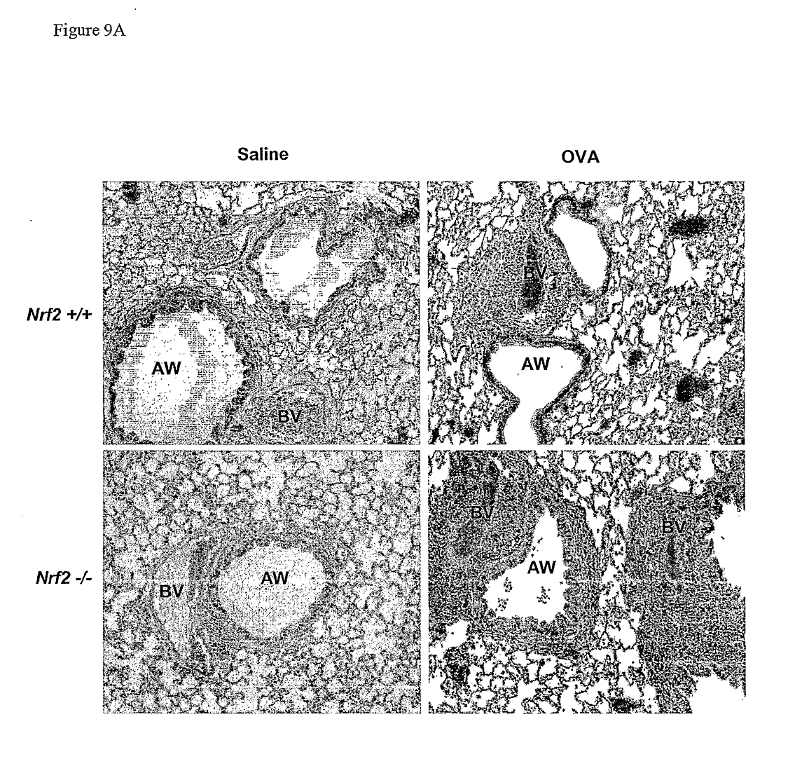

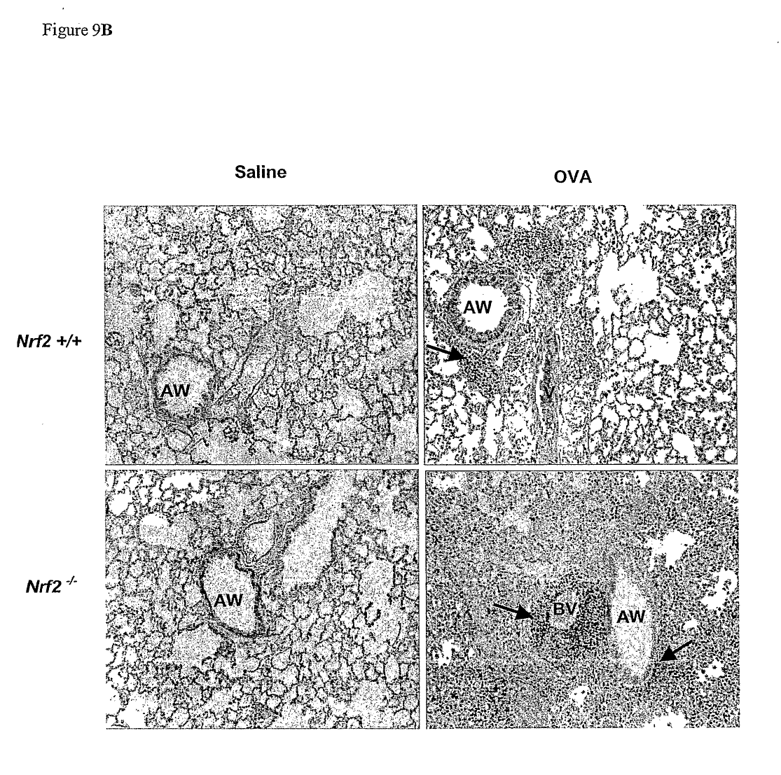

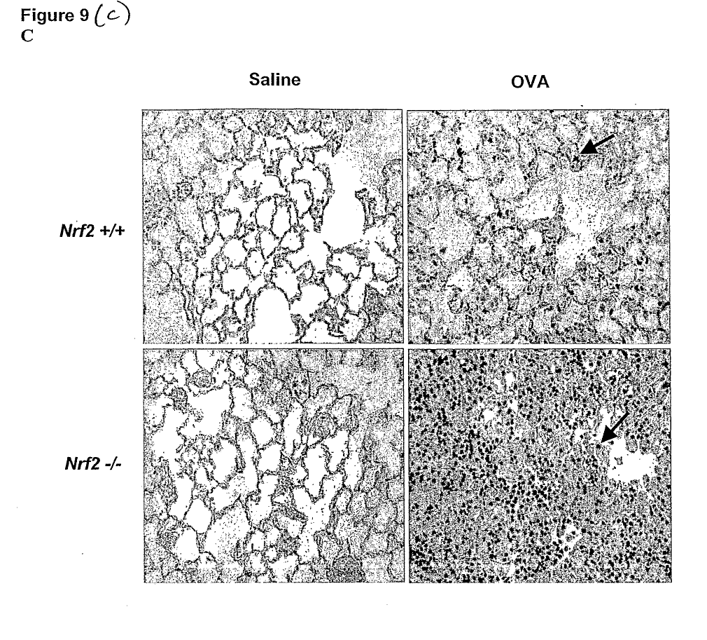

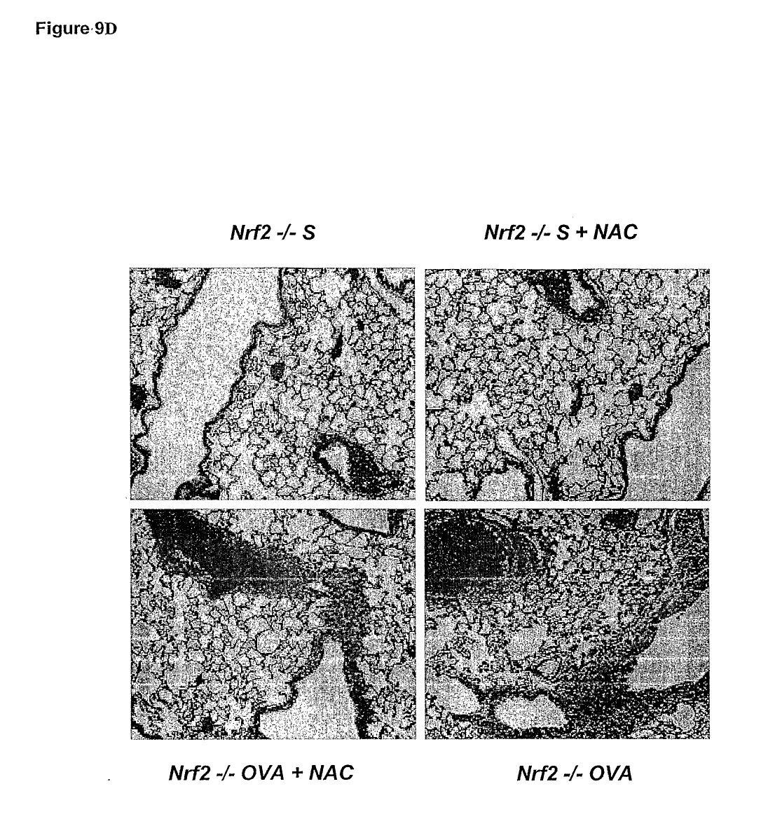

There was a marked extravasation of inflammatory cells into the lungs of Nrf2 OVA mice (3^{rd} challenge) relative to the mild cellular infiltration in the lungs of Nrf2^{+/+} OVA mice, as determined by staining of the lung sections with hematoxylin and eosin (H&E). A higher number of inflammatory cells was observed in the perivascular, peribronchial and parenchymal tissues of the Nrf2^{-/-} OVA mice as compared to a few inflammatory cell infiltrates observed in the Nrf2^{+/+} OVA mice (FIG. 9 A). Immunohistochemical staining with anti-major basophilic protein (anti-MBP) antibody showed numerous eosinophils around the blood vessels and airways (FIG. 9 B) and in the parenchymal tissues (FIG. 9 C) of Nrf2^{-/-} OVA mice compared to the Nrf2^{+/+} OVA mice. Lung tissues from the saline and OVA challenged (3^{rd} challenge) Nrf2^{+/+} and Nrf2^{-/-} mice (n=6) were stained with H&E and examined by light microscopy (20×). OVA challenge caused a marked infiltration of inflammatory cells into the lungs of Nrf2^{-/-} than Nrf2+/+ mice (FIG. 9A). Immunohistochemical staining showed the presence of numerous eosinophils around the blood vessels (BV) and airways (AW) (FIG. 9B), and in the parenchyma (FIG. 9C) of OVA challenged (3^{rd} challenge) Nrf2⁴ mice as compared to Nrf2+/+ mice. These histological data are consistent with the differential cell counts in the BAL fluid obtained from the OVA challenged Nrf2^{+/+} and Nrf2^{-/-} mice. These results demonstrate increased infiltration of inflammatory cells into lungs of OVA challenged Nrf2−/− mice.

In order to determine if reducing oxidative burden would attenuate airway inflammation, mice were treated for 7 days with N-acetyl L-cysteine (NAC) before the 1^{st} OVA challenge. Histological analysis showed a widespread peribronchial and perivascular inflammatory infiltrates in the OVA challenged (1^{st} challenge) Nrf2^{-/-} mice when compared with the saline challenged control mice. NAC-pretreated mice showed a marked reduction in the infiltration of inflammatory cells in the peribronchiolar and perivascular region (FIG. 9 D). Concomitant with histological assessment, airway inflammation was evaluated in the BAL fluid. Antigen-challenged Nrf2^{-/-} mice showed a marked increase in the total number of inflammatory cells (21×10⁴ cells/ml BAL fluid versus 3.2×10⁴ cells/ml BAL fluid in saline group) in the BAL fluid 24 h post OVA challenge (FIG. 8 F). Among the inflammatory cell population, eosinophils were the predominant cells in the BAL fluid (14.38×10⁴ million cells/ml BAL fluid) and were significantly diminished (7.8×10⁴ million cells/ml BAL fluid) by treatment with NAC (FIG. 8 G) in the OVA challenged Nrf2-deficient mice. NAC treatment did not have any significant inhibitory effect on other cell types such as macrophages, neutrophils, lymphocytes and epithelial cells 24 h post 1^{st} OVA challenge. The total and differential cell counts observed in saline-challenged mice treated with NAC did not differ from counts obtained in saline-challenged untreated mice.

### Example 9

### Nrf2^{-/-} OVA Mice Had Increased Level of Oxidative Stress Markers, Eotaxin and Enhanced Activation of Nf-κb

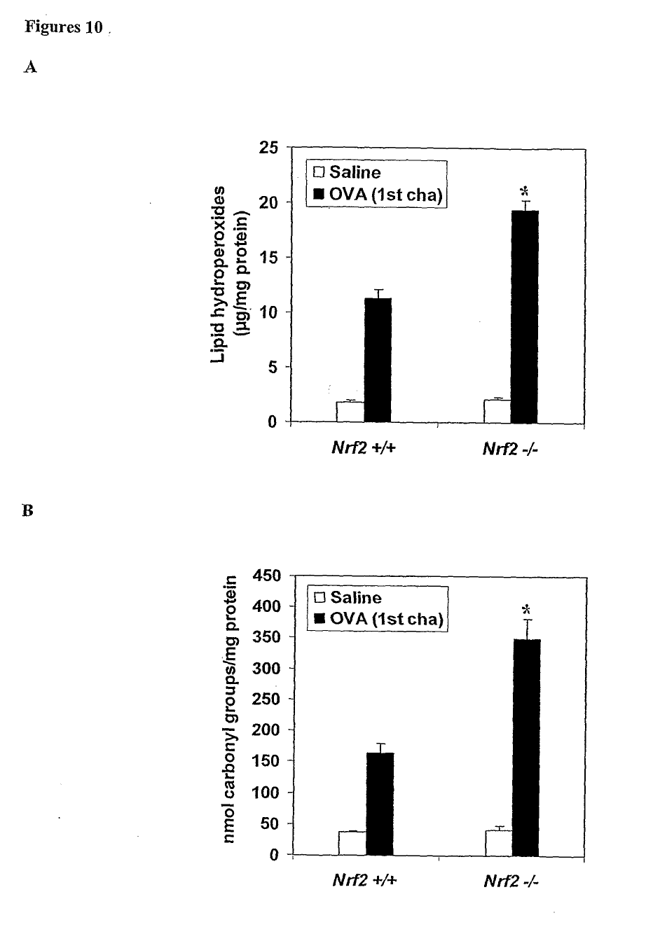

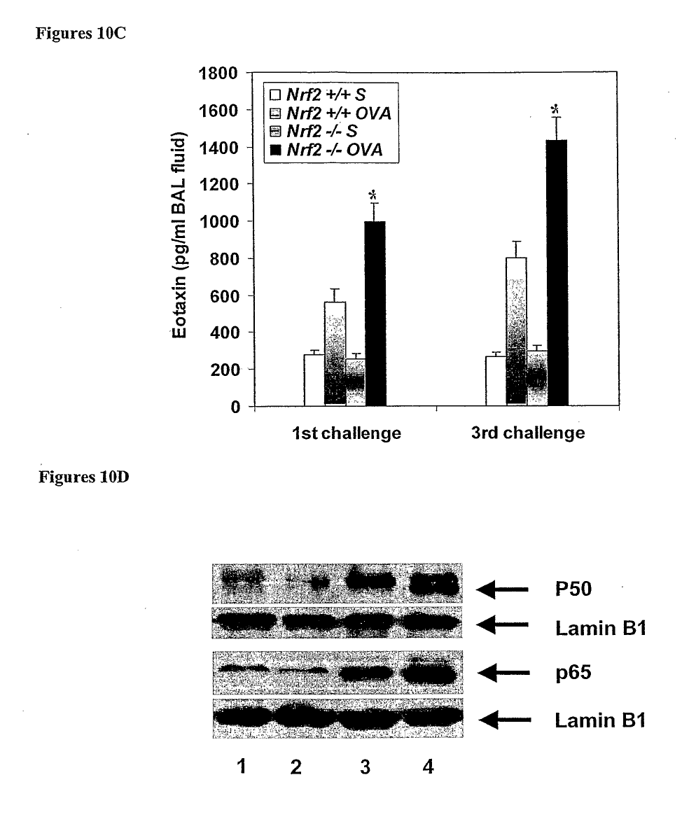

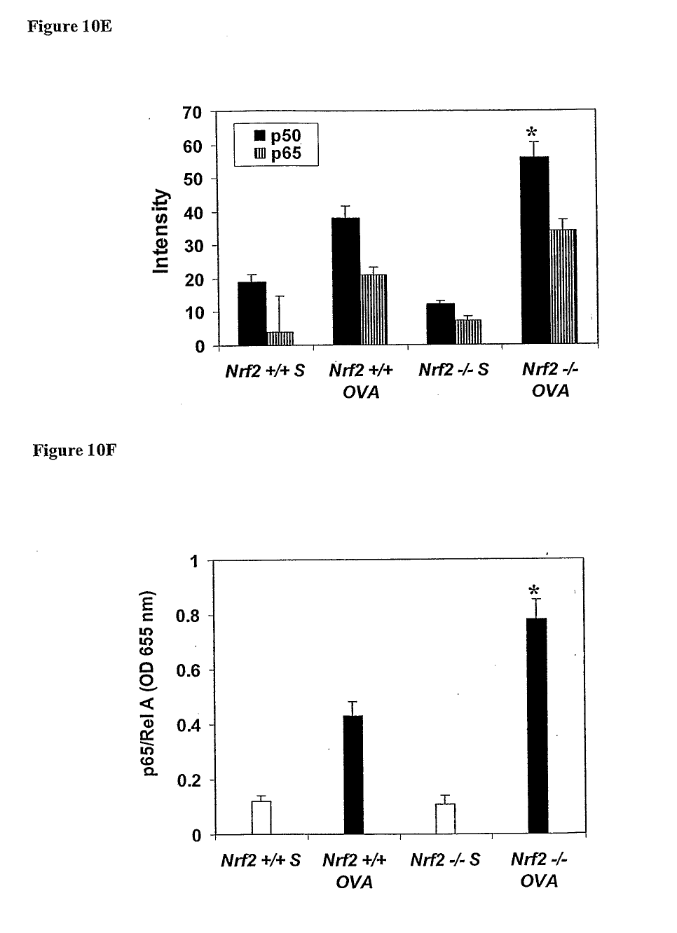

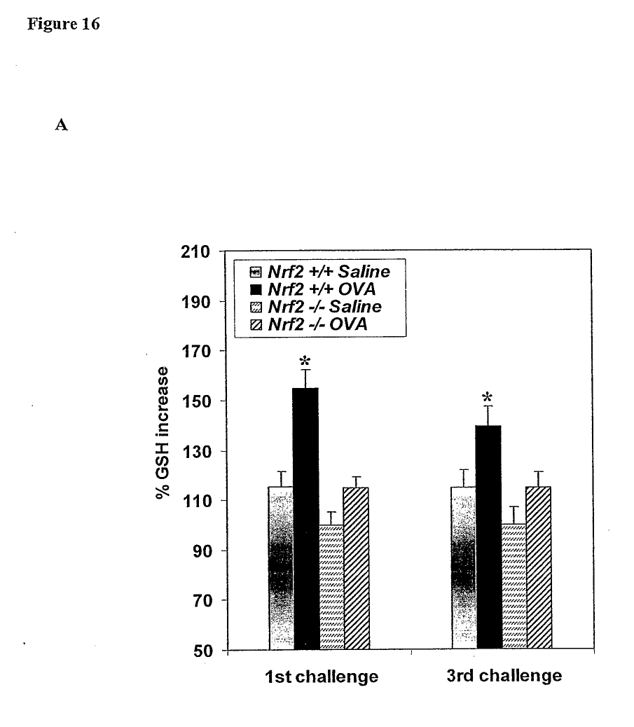

Levels of lipid hydroperoxides and protein carbonyls in the lungs as markers of oxidative stress were measured. When compared to OVA challenged Nrf2 wild-type mice, there was a significantly increased amount of lipid hydroperoxides (11.3 μg/mg protein vs. 19.4 μg/mg protein, FIG. 10 A) and protein carbonyls (165 nmol/mg protein vs 349 nmol/mg protein, FIG. 10 B) in the lungs of Nrf2^{-/-} OVA mice, suggesting the occurrence of excessive oxidative stress in response to allergen challenge. There was a significant increase in GSH level and GSH/GSSG ratio in the lungs of OVA challenged (1^{st} and 3^{rd} challenge) Nrf2^{+/+} mice when compared to the lungs of Nrf2^{-/-} OVA mice (FIGS. 16 A & B).

The level of the eosinophil chemottractant, eotaxin, in the BAL fluid of 1^{st} and 3^{rd} OVA challenged Nrf2-deficient mice was significantly higher when compared to its wild-type counterpart (FIG. 10 C). A significant increase in the level of eotaxin was observed in the BAL fluid of 3^{rd} OVA challenged animals which was concomitant with the increased infiltration of eosinophils in the lungs (FIGS. 9 B and C).

NF-κB has been reported to be activated by oxidative stress and also regulate eotaxin production. Next, the activation of NF-κB in the lungs of Nrf2^{+/+} and Nrf2^{-/-} mice was determined by Western blot analysis with anti-NF-κB p65 and anti-NF-κB p50 antibodies. Immunoblot analysis showed significantly higher levels of both p65 and p50 subunits of NF-κB in the lung nuclear extracts of Nrf^{-/-} OVA mice as compared to the lung nuclear extracts of Nrf2^{+/+} OVA mice (FIGS. 10 D and E). A DNA binding activity assay performed with the Mercury TransFactor ELISA kit showed the increased binding of p65/Rel A subunit to NF-κB from the lung nuclear extracts of Nrf2^{-/-} OVA mice to as compared to its wild-type counterpart (FIG. 10 F). These results demonstrate an increase in oxidative stress markers and activation of NF-κB in the lungs of Nrf2^{-/-} OVA mice.

### Example 10

### Nrf2^{-/-} OVA Mice Had Increased Mucus Cell Hyperplasia

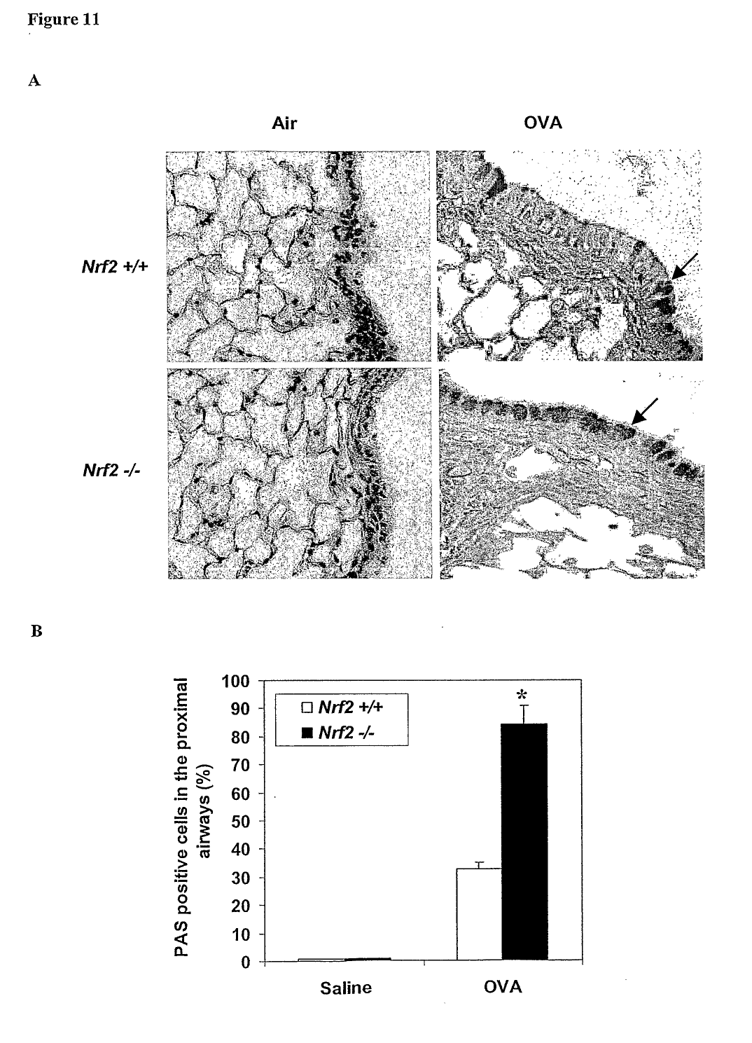

Periodic acid-Schiff's (PAS) staining of lung sections showed a marked increase in the mucus producing granular goblet cells in the proximal airways of Nrf2^{-/-} OVA mice relative to a fewer number of purple staining goblet cells in the Nrf2^{+/+} OVA mice after the 3^{rd} challenge (FIG. 11 A). There were no or few PAS positive cells in the proximal airways of saline challenged mice and distal airways of both Nrf2^{+/+} OVA and Nrf2 OVA mice. The percentage of airway epithelial cells staining for mucus glycoproteins by PAS was significantly higher in the proximal airways of Nrf2^{-/-} OVA mice than the Nrf2^{+/+} OVA mice, and the respective saline challenged mice (FIG. 11 B). This data demonstrates that Nrf2^{-/-} deficient mice show increased mucus cell hyperplasia in response to allergen challenge.

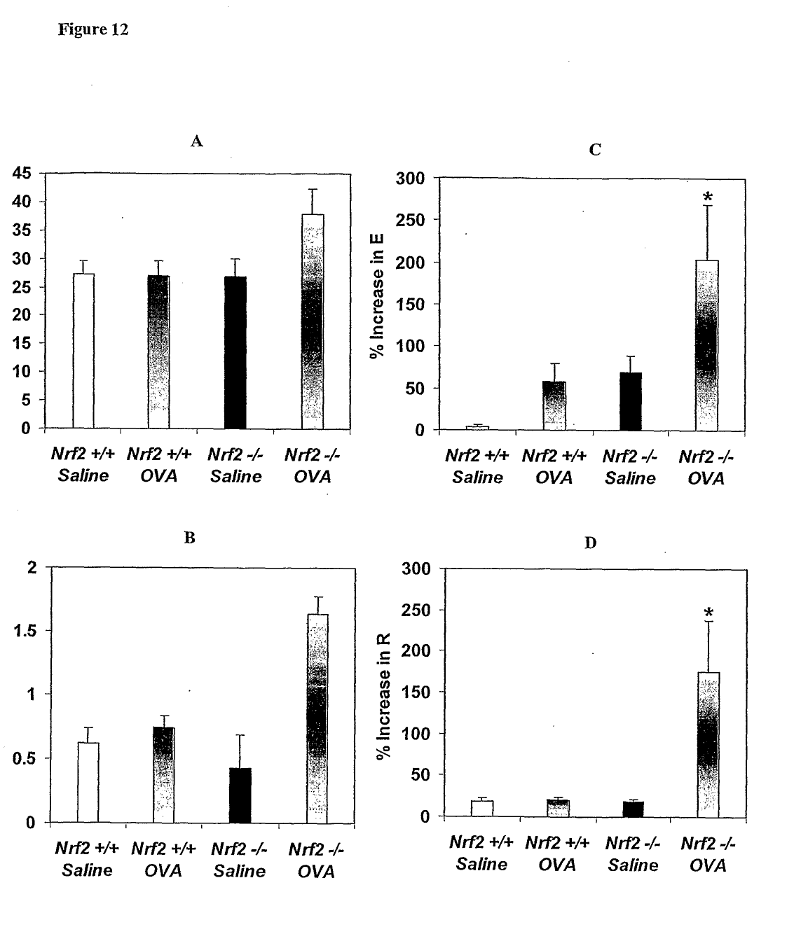

After systemic sensitization and challenges to OVA, airway responsiveness to acetylcholine aerosol was measured. In the absence of acetylcholine challenge, no substantial differences in baseline elastance (FIG. 12 A) and resistance (FIG. 12 B) were observed in both saline and OVA challenged Nrf2^{-/-} and wild type mice. However, 96 h post-3^{rd} OVA challenge, the Nrf2^{-/-} mice showed significant increase in baseline elastance (E)(FIG. 12 C) and resistance (R)(FIG. 12 D) to acetylcholine than the wild-type counterpart. These experiments show that Nrf2^{-/-} mice show increased airway responsiveness to acetylcholine challenge.

### Example 11

### Cytokine Levels in BAL Fluid

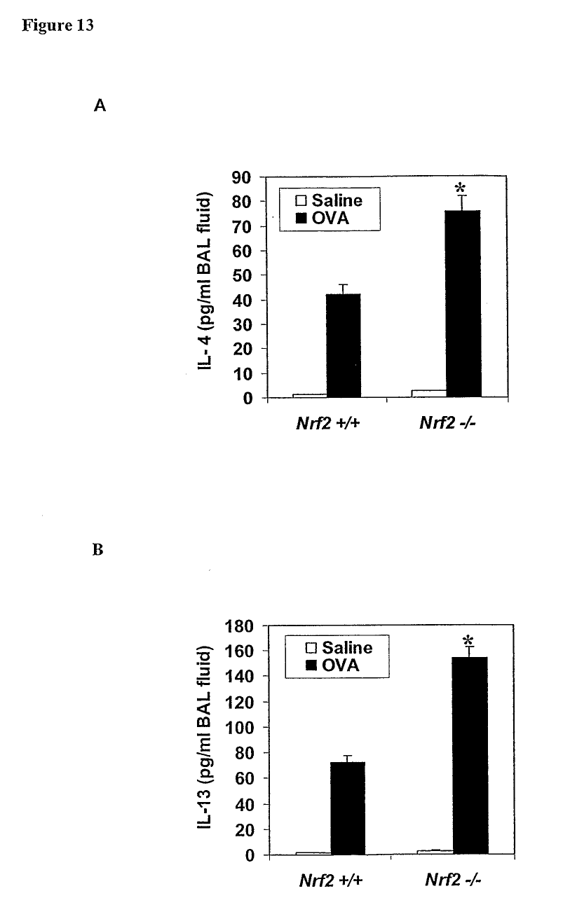

Analysis of BAL fluid by ELISA showed a significant increase in the levels of IL-4 (42 vs 76) and IL-13 (72 vs. 154) in the Nrf2^{-/-} OVA relative to the Nrf2^{+/+} OVA mice. The levels of these cytokines were very low in the BAL fluid of saline treated control mice of both genotypes (FIGS. 13 A and B). Thus, this data shows a difference in the Th2 cytokine levels in the BAL fluid of Nrf2^{+/+} and Nrf2^{-/-} mice challenged with OVA.

In order to determine if enhanced Th2 secretion in OVA challenged mice was reflected at the level of systemic sensitization, splenocytes were isolated from mice 48 h after the 2^{nd} challenge and cytokine secretion was examined in vitro following culture with OVA, or antibodies directed against CD3 and CD28. Table 3 shows the results from these experiments.

TABLE 3
Inflammatory cytokine response of the splenocytes from the OVA challenged
Nrf2^{+/+} and Nrf2^{-/-} mice. Stimulation of splenocytes from Nrf2^{-/-} OVA
mice with anti-CD3 plus anti-CD28 antibodies showed a significantly increased
secretion of IL-4 and IL-13 than the ex vivo stimulated splenocytes from Nrf2^{+/+}
OVA mice. Recall production of IL-4 was generally low in these mice (n = 3).

| | Experiments | | | | | |
|---|---|---|---|---|---|---|
| | Experiment No. 1 | | Experiment No. 2 | | Experiment No. 3 | |
| Genotype | Nrf2^{+/+} | Nrf2^{-/-} | Nrf2^{+/+} | Nrf2^{-/-} | Nrf2^{+/+} | Nrf2^{-/-} |
| IL-4 (pg/ml) | | | | | | |
| None | ND | ND | 2.7 | 1.4 | ND | ND |
| Ova | 2.0 | 2.0 | 2.9 | 2.1 | ND | ND |
| α-CD3/α-CD28 | 7.4 | 25.4 | 32.5 | 82.4 | 3.9 | 23.7 |
| IL-13 (pg/ml) | | | | | | |
| None | 11.1 | 13.2 | 13.6 | 20.0 | 25.2 | 17.0 |
| Ova | 14.6 | 85.0 | 14.9 | 35.9 | 13.4 | 14.4 |
| α-CD3/α-CD28 | 67.2 | 312.3 | 91.0 | 437.4 | 38.9 | 74.0 |

The data presented in Table 3 show that the production of IL-4 and IL-13 were consistently higher using splenocytes from Nrf2^{-/-} mice vs. wild-type mice when stimulated ex vivo. Production of IL-4 was generally low in these mice, consistent with prior experimentation with this strain. Enhanced Th2 cytokine production in these experiments may be a result of direct repressive effect of Nrf2 on Th2 cytokine gene expression, or alternatively a result of an indirect effect via regulation of the oxidant/antioxidant balance. To distinguish between these possibilities, spleen CD4⁺ cells from unchallenged wild-type and Nrf2−/− mice were isolated, and cytokine production was examined ex vivo. No significant differences in IL-4 or IL-13 secretion were observed in these experiments, as shown in Table 4 below.

TABLE 4
Inflammatory cytokine response of the CD4⁺ T cells
isolated from the spleen of control Nrf2^{+/+} and
Nrf2^{-/-} mice. No significant differences in IL-4 or
IL-13 secretion were observed in splenocytes from the room air
exposed Nrf2^{+/+} and Nrf2^{-/-} mice. Data are in pg/ml/million
cells, and represent mean ± SEM of 3 experiments.

| | Nrf2^{+/+} | Nrf2^{-/-} |
|---|---|---|
| IL-4 (pg/ml) | | |
| Anti-CD3 + anti-CD28 | 64 ± 4.7 | 52.5 ± 7 |
| A23187 + PMA | 76.7 ± 37.8 | 90.3 ± 17.5 |
| IL-13 (pg/ml) | | |
| Anti-CD3 + anti-CD28 | 4.7 ± 1.8 | 3.4 ± 0.9 |
| A23187 + PMA | 4.6 ± 1.2 | 3.9 ± 0.6 |

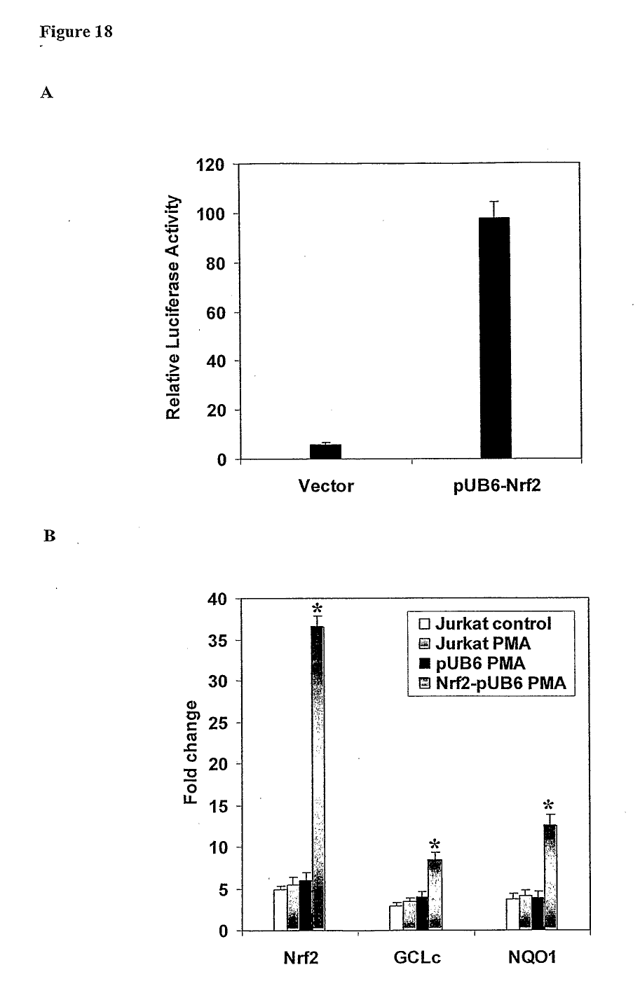

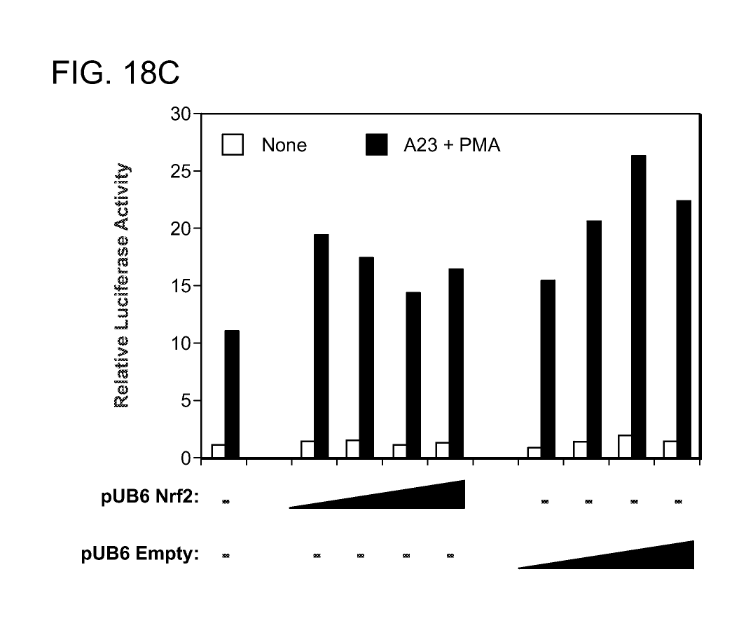

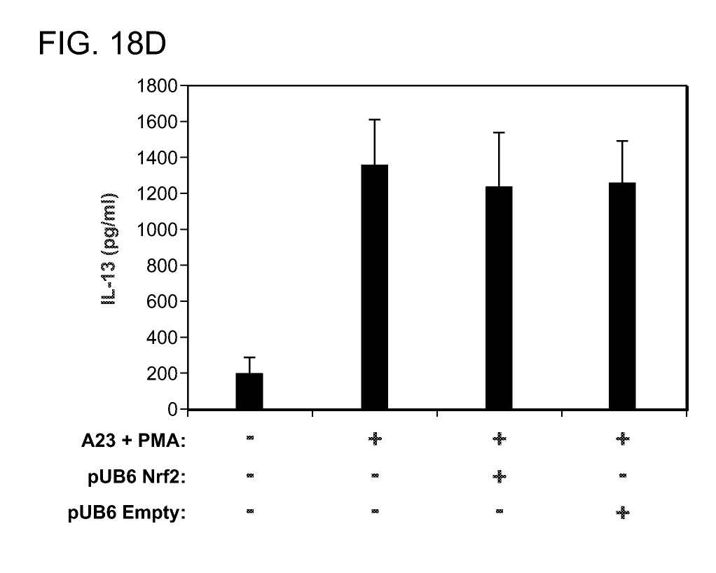

Next, the ability of Nrf2 to directly regulate IL-4 or IL-13 gene expression or promoter activity in transient transfection assays was examined. Although overexpression of Nrf2 substantially increased the expression of its known target genes glutathione cysteine ligase catalytic subunit (GCLc) and NADPH:quinone oxidoreductase (NQO1), there was no effect on IL-13 gene expression (FIG. 18). In parallel experiments, overexpressing Nrf2 did not affect transcription driven by the IL-4 or IL-3 promoters (FIGS. 18 A-D). Thus, these results demonstrate that Nrf2-deficiency indirectly enhanced Th2 cytokine production via regulation of the oxidant/antioxidant balance.

### Example 12

### Activation of Nrf2 in the Lungs of Nrf2^{+/+} Mice

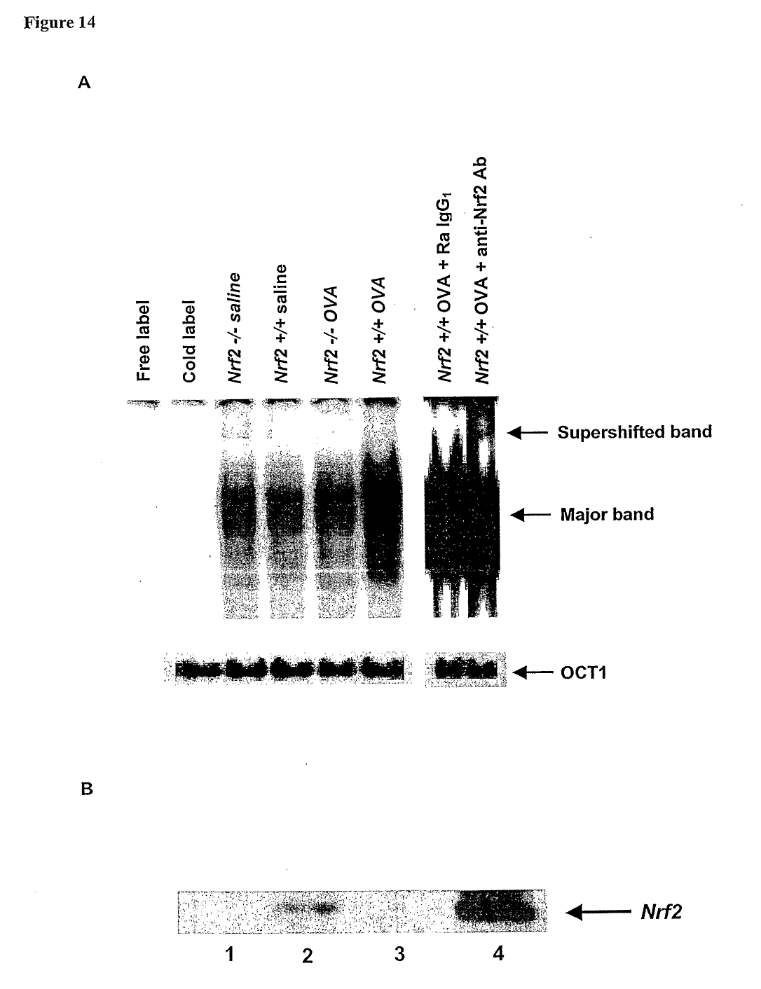

Electrophoretic mobility shift assay (EMSA) was used to determine the activation and DNA binding activity of Nrf2 in the lungs in response to allergen challenge (FIG. 14 A). EMSA analysis showed increased binding of nuclear proteins to ARE isolated from the lungs of OVA challenged Nrf2^{+/+} mice to ARE consensus sequence relative to the OVA challenged Nrf2^{-/-} mice, or the saline challenged control mice. Supershift analysis with anti-Nrf2 antibody also showed the binding of Nrf2 to the ARE consensus sequence, suggesting that OVA challenge leads to the activation of Nrf2 in the lungs of Nrf2^{+/+} mice.

Immunoblot analysis (FIG. 14 B) showed increased level of Nrf2 in the lung nuclear extracts of Nrf2^{+/+} OVA mice as compared to its saline challenged counterpart, suggesting an accumulation of Nrf2 in the lungs of wild-type mice in response to allergen challenge. These data show the activation of Nrf2 in the lungs of OVA challenged Nrf2^{+/+} mice.

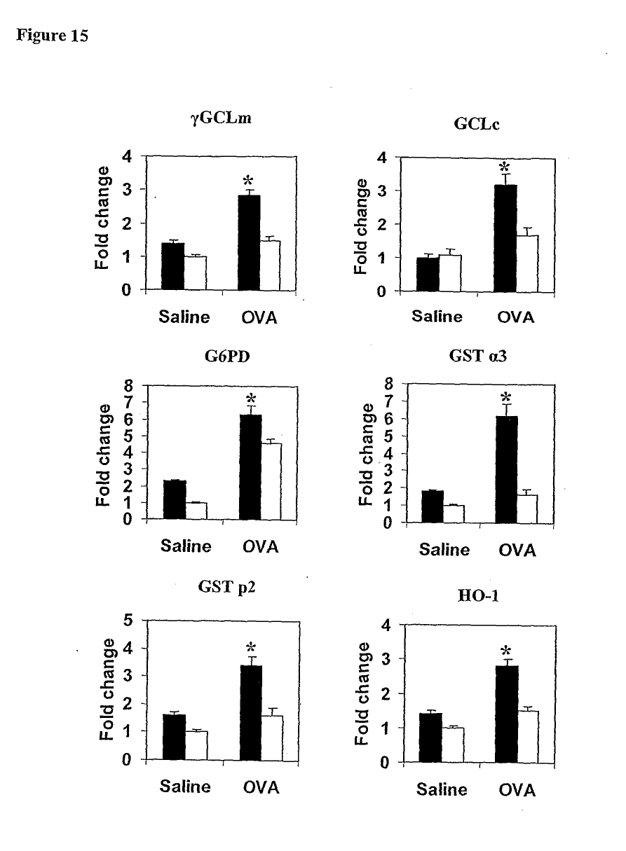

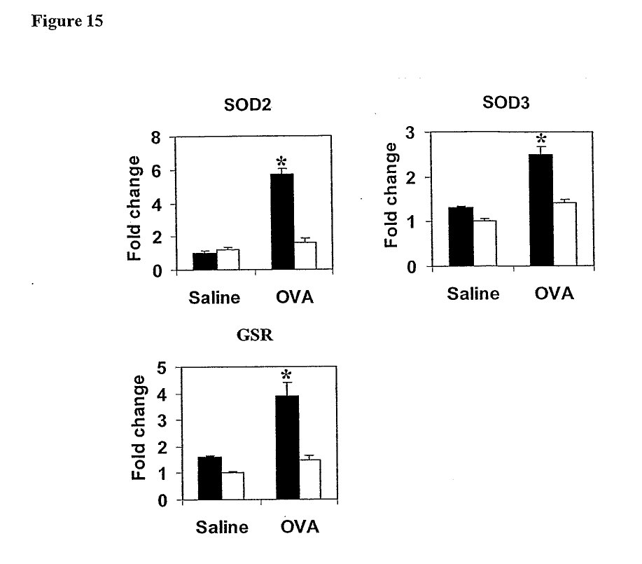

An increase in nuclear Nrf2 is needed for the activation of ARE and the transcriptional induction of various antioxidant genes. There was a substantial and coordinated elevation in transcript levels of several antioxidant genes in the lungs of Nrf2^{+/+} OVA mice when compared to the OVA challenged Nrf2-disrupted mice. Real time-PCR (RT-PCR) analysis was used to determine the fold changes in mRNA of the following antioxidant genes in the lungs of Nrf2^{+/+} OVA (24 h post-1^{st} challenge) and Nrf2^{-/-} OVA mice, respectively: gamma GCL modifier subunit (γGCLm) (2.9 vs. 1.6), GCLc (3.2 vs 1.7), glucose 6 phosphate dehydrogenase (G6PD) (6.3 vs. 4.6), GST α3 (6.2 vs. 1.7), GST p2 (3.4 vs. 1.6), HO-1 (2.8 vs. 1.5), SOD2 (5.7 vs 1.6), SOD3 (2.5 vs. 1.5) and glutathione S-reductase (GSR) (3.9 vs. 1.5) (FIG. 15). The magnitude of the induction of these antioxidant genes was significantly higher in Nrf2 wild-type mice as compared to Nrf2-disrupted mice, thus showing their association with the activation of Nrf2 in response to allergen induced lung inflammation.

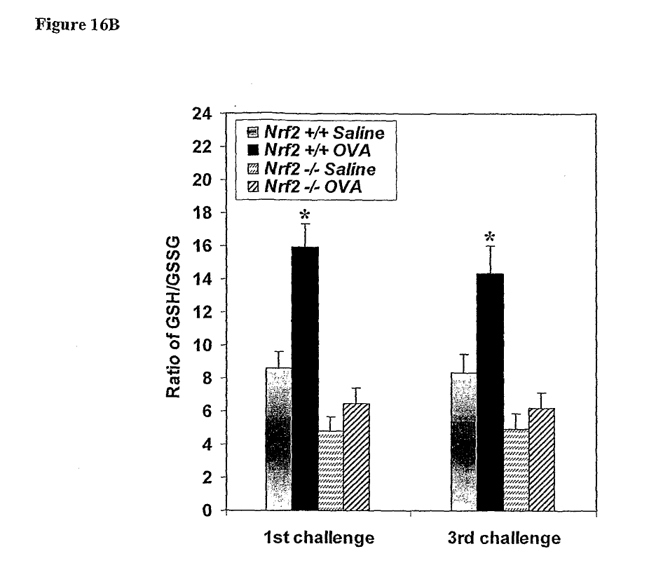

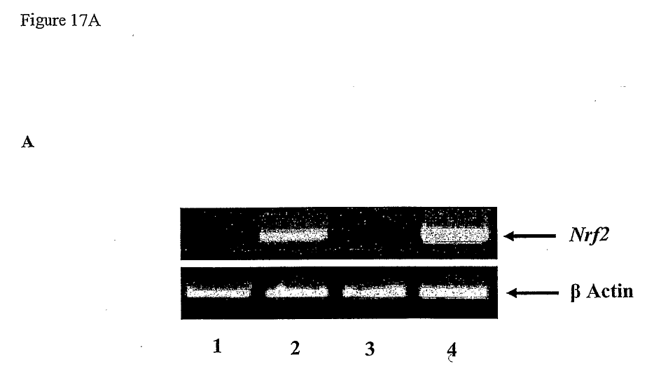

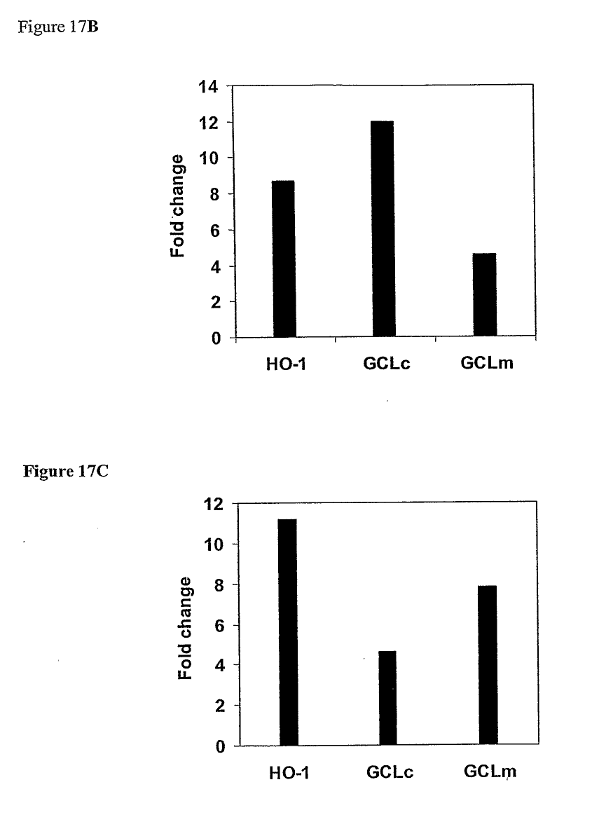

FIGS. 16 A & B shows the % GSH increase and GSH/GSSG ratios in the lungs of saline and OVA challenged Nrf2^{+/+} and Nrf2^{-/-} mice. FIGS. 17 A-C shows the expression of Nrf2 and Nrf2 dependent antioxidant genes (HO-1, GCLc and GCLm) in the lung CD4⁺ T cells and macrophages isolated from the OVA challenged Nrf2^{+/+} and Nrf2^{-/-} mice.

FIG. 18 shows the Nrf2 overexpression in mouse Hepa cells (A), overexpression of Nrf2 in Jurkat cell line and the analysis of Nrf2 dependent antioxidant genes (B), effect of Nrf2 overexpression on IL-13 promoter activity (C) and IL-13 protein level (D) in Jurkat cell line.

Additional RT-PCR analysis showed the expression of Nrf2 in CD4⁺ T cells and macrophages isolated from the lungs of Nrf2^{+/+} OVA mice (FIG. 17 A). Quantitative real time RT-PCR revealed the increased expression of the following Nrf2-regulated antioxidant genes: HO-1 (CD4⁺ T cells, 2.5 fold; macrophages, 11.2 fold), GCLc (CD4⁺ T cells, 2.5-fold; macrophages 4.6 fold), and GCLm (CD4⁺ T cells, 2.5-fold; macrophages, 7.8 fold) in the CD4⁺ T cells and macrophages isolated from the lungs of Nrf2^{+/+} OVA mice when compared to its knock out counterpart (FIG. 17 B). Taken together, the RT-PCR analysis demonstrated increased levels of selected antioxidant genes in the lungs of OVA challenged Nrf2^{+/+} and Nrf2^{-/-} mice.

### Example 13

### Disruption of Nrf2 Caused Increased Septic Shock Lethality

Host genetic factors that regulate innate immunity determine the susceptibility to sepsis. As reported below, disruption of nuclear factor-erythroid 2-p45-related factor 2 (nrf2) dramatically increased the mortality of mice to endotoxin and cecal ligation and puncture induced septic shock. Thus, nrf2 is a novel modifier gene of sepsis that determines survival by mounting an appropriate innate immune response.

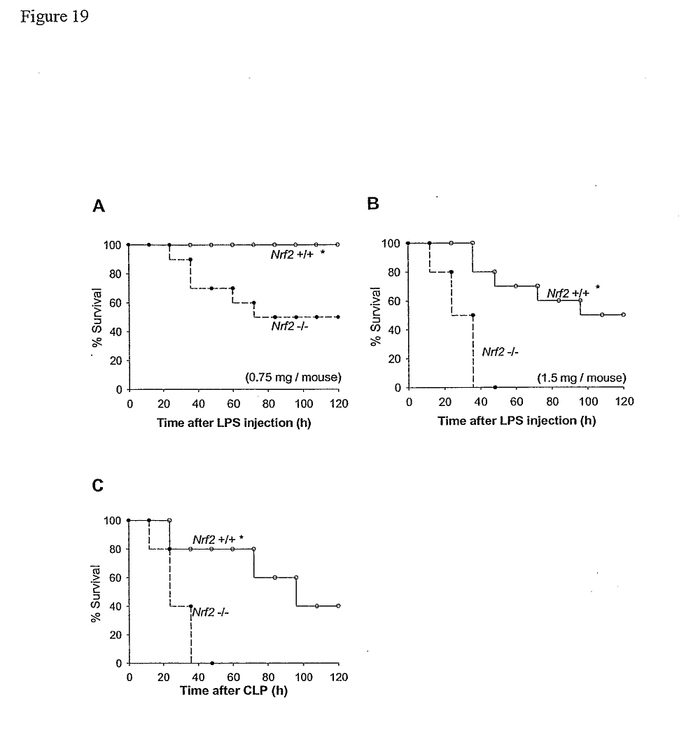

The role of Nrf2 on the survival of wild-type (nrf2+/+) and nrf2-deficient (nrf2−/−) mice during an endotoxic shock was examined. Nrf2+/+ and nrf2−/− mice were treated intraperitoneally with a lethal dose of LPS (0.75 and 1.5 mg per mouse) and survival was monitored for 5 days. The lower dose resulted in the death of 50% of the nrf2−/− mice but no death of the nrf2+/+ mice (FIG. 19 A). At the higher dose, 100% of the nrf2−/− mice died within 48 h, whereas only 50% of the nrf2+/+ mice died by day 5 (FIG. 19 B). Next, the role of Nrf2 on survival in a clinically relevant model of septic shock induced by cecal ligation and puncture (CLP) was examined. By 48 h after CLP, all nrf2−/− mice died, while only 20% of wild-type littermates died. After 5 days, 40% of wild-type mice survived (FIG. 19 C). No death was observed in sham operated mice of both genotypes. This data indicated that Nrf−/− mice were more sensitive to LPS-induced septic shock.

### Example 14

### LPS Elicited Greater Pulmonary Inflammation in Nrf2-Deficient Mice

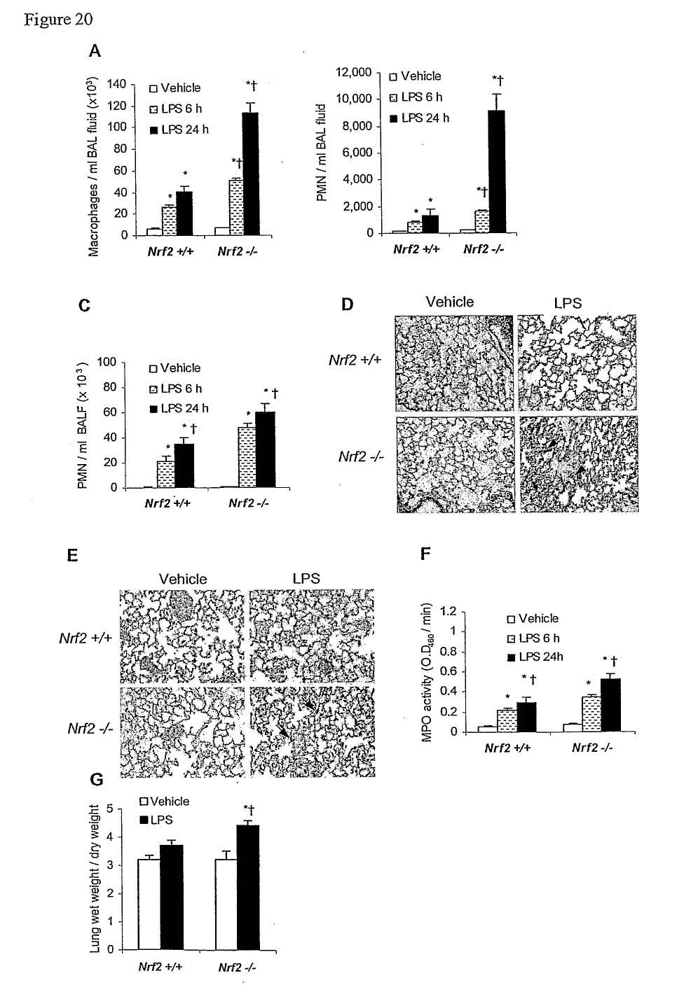

Because Nrf2 was found to be necessary for survival during lethal septic shock, the role of this transcription factor in regulating non-lethal inflammatory stimulus was investigated. Lungs were examined after systemic [intraperitoneal (ip) injection of 60 μg per mouse] or local (intratracheal instillation of 10 μg per mouse) administration of LPS. For both modes of LPS administration, the inflammatory response was greater in the lungs of nrf2−/− mice than in their wild-type littermates. The influx of inflammatory cells (neutrophils and macrophages) was greater in the lungs of nrf2−/− mice at both 6 and 24 h after LPS challenge by either route. After ip administration of LPS, macrophages were the predominant cell type in bronchoalveolar lavage (BAL) fluid, although both macrophages and neutrophils showed temporal increase in numbers (FIGS. 20 A & B). In contrast, intratracheal instillation attracted predominantly neutrophils, constituting as much as 80% of the total inflammatory cell population, in BAL fluid (FIG. 20 C). Consistent with the BAL fluid analysis, histopathology showed a greater recruitment of inflammatory cells in perivascular, peribronchial, and alveolar spaces of nrf2−/− mice 24 h after LPS treatment (FIG. 20 D). Immunohistochemical examination of LPS-instilled lungs with anti-neutrophil antibody also confirmed a greater number of neutrophils in the lungs of nrf2−/− mice (FIG. 20 E), which was further evident from myeloperoxidase activity in these lungs (FIG. 20 F). As a marker of lung injury, pulmonary edema was observed to be markedly higher in nrf2−/− mice 24 h after LPS instillation (FIG. 20 G). A similar pattern of lung pathological injury was induced by systemic delivery of LPS. Taken together, these results show that disruption of the nrf2 gene augments the innate immune response to bacterial endotoxin.

### Example 15

### LPS and CLP Induced Greater Secretion of TNF-α in nrf2-Deficient Mice

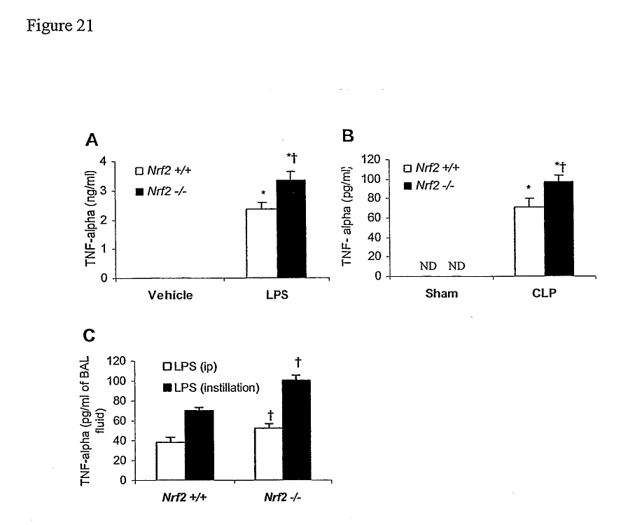

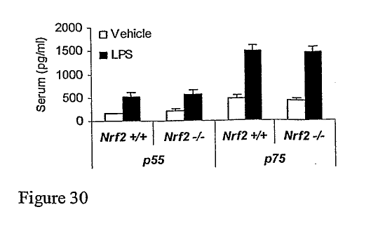

Because TNF-α is one of the early proinflammatory cytokines that is elevated during LPS and CLP-induced inflammation, serum concentrations of TNF-α were measured by ELISA. After 1.5 h of LPS challenge (1.5 mg per mouse), serum TNF-α was significantly higher in nrf2−/− mice compared to nrf2+/+ (FIG. 21 A). Similarly, after 6 h of CLP, serum levels of TNF-α was greater in nrf2−/− compared to nrf2+/+ mice (FIG. 21 B). Furthermore, TNF-α concentrations in BAL fluid was also greater 2 h after non-lethal LPS challenge (ip and intratracheal instillation) in nrf2−/− mice as compared to wild-type mice (FIG. 21 C). The concentrations of TNF receptors, TNFRI (p55) and TNFRII (p75) in nrf2+/+ and nrf2−/− mice after a lethal dose of LPS was measured. While there was no difference in the constitutive serum levels of p55 and p75, after 6 h of LPS treatment, the serum concentrations of both receptors were increased significantly; however there were no significant differences in the TNF receptors between the nrf2−/− and nrf2+/+ mice (FIG. 30) after LPS challenge.

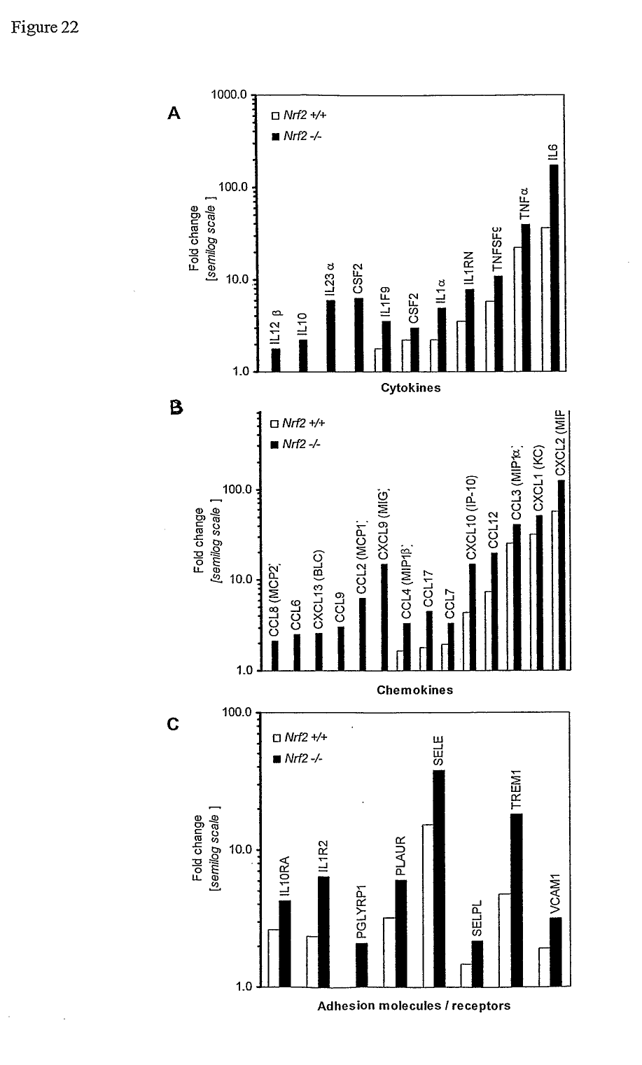

Temporal global changes in gene expression reflect the impact of Nrf2 on the innate immune response. Moderate increase in TNF-α production alone cannot explain the markedly higher CLP and LPS induced mortality as well as LPS-induced lung inflammation in nrf2−/− mice (Eskandari M K et al. J Immunol 148:2724-2730.1993). To systematically understand the role of Nrf2 during LPS induced inflammation, the global gene expression profiles were examined in lungs of nrf2−/− and nrf2+/+ mice over time, in response to a non-lethal LPS stimulus. After ip injection of LPS, microarray analyses of lungs were performed at 30 min, 1 h, 6 h, 12 h, and 24 h. Nrf2 deficiency resulted in the enhanced expression of several clusters of genes associated with the innate immune response, even as early as 30 min (FIGS. 22 A-C). The genes expressed included specific cytokines, chemokines, and cell surface adhesion molecules and receptors, among others. Differences between genotypes in expression of most of the proinflammatory genes in the lungs of mice were significant at the early time points (30 min and 1 h) following LPS challenge. At later time points, with few exceptions there was no significant difference in expression of proinflammatory genes between the genotypes. Henceforth, unless otherwise stated, a more detailed presentation of the gene expression profile obtained at 30 min is provided while the remaining data for the time-course is presented as supplemental data. The microarray results indicate that Nrf2 functionality is indispensable for controlling the early surge of a large number of proinflammatory genes associated with innate immune response. Presented as follows are results from the microarray analysis.

Cytokines and Chemokines.

At 30 min after LPS challenge, gene expression of cytokines such as TNF-α, TNFSF9, IL-1α, IL-6, IL1F9, IL-10, IL-12β, IL-23p19, CSF1 and CSF2 was significantly higher in lungs of nrf2−/− compared to nrf2+/+ mice. Among all cytokines, the expression of IL-6 was highest. Members of C—C family [CCL12 (MCPS), CCL17 (TARC), CCL2 (MCP1), CCL3 (MIP1α), CCL4 (MIP1β), CCL6 and CCL8 (MCP2)] and C—X—C chemokines [MIP2, MIG, KC, ITAC, IP-10 and CXCL13] were greatly upregulated in LPS challenged nrf2−/− lungs relative to nrf2+/+ [(FIG. 22 and Table 4a).

TABLE 4a
Differential expression of cytokine and chemokine related genes in the lungs of
nrf2-deficient and wild-type mice following treatment with LPS.

| | | 30 min | | 1 h | | 6 h | | 12 h | | 24 h | |
|---|---|---|---|---|---|---|---|---|---|---|---|
| | | (LPS/Vehicle) | | (LPS/Vehicle) | | (LPS/Vehicle) | | (LPS/Vehicle) | | (LPS/Vehicle) | |
| Gene title | Gene symbol | Nrf2−/− | Nrf2+/+ | Nrf2−/− | Nrf2+/+ | Nrf2−/− | Nrf2+/+ | Nrf2−/− | Nrf2+/+ | Nrf2−/− | Nrf2+/+ |
| Chemokine (C-C motif) | CCL12 | 19.7 ± 0.6 | 7.5 ± 0.4 | 27.7 ± 0.6 | 12.5 ± 0.4 | 19.3 ± 0.6 | 8.6 ± 0.4 | 19.6 ± 0.7 | 15.0 ± 0.4 | 29.2 ± 0.7 | 14.9 ± 0.4 |
| ligand 12 (Monocyte | (MCP5) | | | | | | | | | | |
| chemotactic protein 5) | | | | | | | | | | | |
| Chemokine (C-C motif) | CCL17 | 4.5 ± 0.4 | 1.8 ± 0.4 | 6.1 ± 0.4 | 4.2 ± 0.4 | 9.1 ± 0.4 | 7.0 ± 0.4 | 7.1 ± 0.4 | 7.1 ± 0.4 | — | — |
| ligand 17 (Thymus- and | (TARC) | | | | | | | | | | |
| activation-regulated | | | | | | | | | | | |
| chemokine) | | | | | | | | | | | |
| Chemokine (C-C motif) | CCL2 | 6.3 ± 0.5 | — | 24.8 ± 0.4 | 20.5 ± 0.6 | 20.4 ± 0.5 | 11.9 ± 0.6 | 6.0 ± 0.6 | 8.8 ± 0.6 | 4.7 ± 0.6 | 5.7 ± 0.5 |
| ligand 2 (Monocyte | (MCP1) | | | | | | | | | | |
| chemoattractant protein-1) | | | | | | | | | | | |
| Chemokine (C-C motif) | CCL20 | — | — | 21.4 ± 0.5 | 32.0 ± 0.7 | — | — | — | — | — | — |
| ligand 20 (Macrophage | (MIP3α) | | | | | | | | | | |
| inflammatory protein 3 alpha) | | | | | | | | | | | |
| Chemokine (C-C motif) | CCL3 | 40.5 ± 0.9 | 25.3 ± 0.5 | 321.8 ± 0.8 | 501.5 ± 0.5 | 120.3 ± 0.8 | 170.1 ± 0.4 | 39.1 ± 0.8 | 73.5 ± 0.5 | — | — |
| ligand 3 (Macrophage | (MIP1α) | | | | | | | | | | |
| inflammatory protein 1-alpha) | | | | | | | | | | | |
| Chemokine (C-C motif) | CCL4 | 3.3 ± 0.4 | 1.7 ± 0.4 | 12.8 ± 0.4 | 11.4 ± 0.5 | 8.1 ± 0.5 | 8.2 ± 0.4 | 1.9 ± 0.4 | 2.3 ± 0.4 | — | 1.6 ± 0.4 |
| ligand 4 (Macrophage | (MIP1β) | | | | | | | | | | |
| inflammatory protein 1-beta) | | | | | | | | | | | |
| Chemokine (C-C motif) | CCL6 | 2.5 ± 0.4 | — | 1.4 ± 0.4 | 1.7 ± 0.4 | 1.6 ± 0.5 | 1.7 ± 0.4 | — | — | — | — |
| ligand 6 | | | | | | | | | | | |
| Chemokine (C-C motif) | CCL8 | 2.1 ± 0.5 | — | — | — | — | — | 1.6 ± 0.4 | — | — | — |
| ligand 8 (Monocyte | (MCP2) | | | | | | | | | | |
| chemoattractant protein 2) | | | | | | | | | | | |
| Chemokine (C-C motif) | CCR7 | — | — | — | — | 3.5 ± 0.4 | 2.4 ± 0.5 | 3.1 ± 0.4 | 2.3 ± 0.5 | 1.5 ± 0.4 | — |
| receptor 7 | | | | | | | | | | | |
| Chemokine (C-C motif) | CCRL2 | 5.3 ± 0.4 | 3.3 ± 0.4 | 8.7 ± 0.4 | 11.6 ± 0.4 | 3.9 ± 0.4 | 3.7 ± 0.4 | 1.7 ± 0.4 | 1.8 ± 0.4 | — | — |
| receptor-like 2 | | | | | | | | | | | |
| Chemokine (C-X3-C | CX3CL1 | — | — | 2.8 ± 0.4 | 5.0 ± 0.7 | — | — | — | — | — | — |
| motif) ligand 1 | | | | | | | | | | | |
| Chemokine (C—X—C | CXCL1 | 16.0 ± 0.4 | 6.8 ± 0.5 | 34.1 ± 0.4 | 26.0 ± 0.4 | 12.9 ± 0.5 | 9.7 ± 0.4 | 5.3 ± 0.4 | 5.7 ± 0.4 | 1.7 ± 0.5 | 2.0 ± 0.4 |
| motif) ligand 1 | (KC) | | | | | | | | | | |
| (Platelet-derived growth | | | | | | | | | | | |
| factor-inducible protein) | | | | | | | | | | | |
| Chemokine (C—X—C | CXCL10 | 14.7 ± .6 | 4.3 ± 0.5 | 40.5 ± 0.5 | 25.8 ± 0.4 | 187.4 ± 0.6 | 112.2 ± 0.4 | 40.2 ± 0.6 | 34.3 ± 0.4 | 5.0 ± 0.7 | 5.6 ± 0.4 |
| motif) ligand 10 | (IP-10) | | | | | | | | | | |
| (Gamma-IP10) | | | | | | | | | | | |
| Chemokine (C—X—C | CXCL11 | — | — | 3.9 ± 0.5 | — | 177.3 ± 0.5 | 198.1 ± 0.8 | 24.8 ± 0.5 | 41.6 ± 0.9 | — | — |
| motif) ligand | (ITAC) | | | | | | | | | | |
| 11(Interferon-inducible | | | | | | | | | | | |
| T-cell alpha | | | | | | | | | | | |
| chemoattractant) | | | | | | | | | | | |
| Chemokine (C—X—C | CXCL13 | 2.6 ± 0.5 | — | — | 1.9 ± 0.5 | 8.6 ± 0.5 | 4.9 ± 0.4 | 9.2 ± 0.4 | 8.0 ± 0.5 | 10.6 ± 0.4 | 8.3 ± 0.4 |
| motif) ligand 13 (B | (BLC) | | | | | | | | | | |
| lymphocyte | | | | | | | | | | | |
| chemoattractant) | | | | | | | | | | | |
| Chemokine (C—X—C | CXCL14 | — | — | — | — | 1.5 ± 0.4 | — | 2.3 ± 0.5 | — | — | — |
| motif) ligand 14 | | | | | | | | | | | |
| Chemokine (C—X—C | CXCL2 | 123.6 ± 0.4 | 56.9 ± 0.4 | 250.7 ± 0.4 | 215.3 ± 0.4 | 76.6 ± 0.5 | 66.7 ± 0.4 | 35.8 ± 0.5 | 28.2 ± 0.5 | 3.9 ± 0.4 | 5.1 ± 0.4 |
| motif) ligand 2 | (MIP2) | | | | | | | | | | |
| (Macrophage | | | | | | | | | | | |
| inflammatory protein 2) | | | | | | | | | | | |
| Chemokine (C—X—C | CXCL5 | — | — | — | 3.2 ± 0.7 | 4.1 ± 0.4 | 2.4 ± 0.5 | — | — | — | — |
| motif) ligand 5 | (LIX) | | | | | | | | | | |
| (lipopoly-saccharide | | | | | | | | | | | |
| induced C—X—C | | | | | | | | | | | |
| chemokine) | | | | | | | | | | | |
| Chemokine (C—X—C motif) | CXCL9 | 14.7 ± 0.5 | — | 11.7 ± 0.5 | — | 820.3 ± 0.5 | 576.0 ± 0.5 | 837.5 ± 0.5 | 739.3 ± 0.6 | 116.2 ± 0.7 | 68.6 ± 0.7 |
| ligand 9 (Gamma interferon | (MIG) | | | | | | | | | | |
| induced monokine) | | | | | | | | | | | |
| Colony stimulating factor | CSF1 | 3.0 ± 0.4 | 2.2 ± 0.4 | 8.2 ± 0.4 | 7.0 ± 0.4 | 4.9 ± 0.4 | 4.9 ± 0.4 | 3.4 ± 0.4 | 3.9 ± 0.4 | 1.7 ± 0.4 | 2.0 ± 0.4 |
| 1 (macrophage) | | | | | | | | | | | |
| Colony stimulating factor | CSF2 | 6.3 ± 0.8 | — | 70.5 ± 1.0 | 49.9 ± 0.5 | 65.8 ± 0.9 | 106.9 ± 0.4 | 12.5 ± 1.0 | 24.3 ± 0.5 | — | — |
| 2 (granulocyte- | | | | | | | | | | | |
| macrophage) | | | | | | | | | | | |
| Colony stimulating factor | CSF3 | — | — | 40.2 ± 0.5 | 27.5 ± 0.5 | 39.9 ± 0.6 | 20.1 ± 0.5 | 13.2 ± 0.6 | 10.8 ± 0.5 | — | — |
| 3 (granulocyte) | | | | | | | | | | | |
| Interferon gamma | IFNG | — | — | — | — | 7.5 ± 0.8 | 5.3 ± 0.9 | — | — | — | — |
| Interleukin 1 alpha | IL1α | 4.9 ± 0.6 | 2.2 ± 0.4 | 11.2 ± 0.6 | 6.2 ± 0.5 | — | — | — | — | — | — |
| Interleukin 1 beta | IL1β | 21.0 ± 0.4 | 17.6 ± 0.4 | 27.7 ± 0.4 | 40.8 ± 0.5 | 13.8 ± 0.4 | 14.3 ± 0.4 | 10.6 ± 0.4 | 11.8 ± 0.4 | 4.9 ± 0.4 | 6.7 ± 0.4 |
| Interleukin 1 family, | IL1F9 | 3.6 ± 0.6 | 1.8 ± 0.4 | 25.6 ± 0.4 | 19.0 ± 0.5 | 3.8 ± 0.4 | 3.7 ± 0.5 | 6.1 ± 0.4 | 5.9 ± 0.5 | 1.8 ± 0.4 | 2.1 ± 0.5 |
| member 9 | | | | | | | | | | | |
| Interleukin 1 receptor | IL1RN | 9.8 ± 0.6 | 5.0 ± 0.5 | 34.1 ± 0.4 | 36.3 ± 0.4 | 42.8 ± 0.4 | 38.9 ± 0.4 | 22.6 ± 0.4 | 23.3 ± 0.4 | 5.4 ± 0.5 | 6.2 ± 0.4 |
| antagonist | | | | | | | | | | | |
| Interleukin 10 | IL10 | 2.2 ± 0.4 | — | 2.2 ± 0.5 | 1.8 ± 0.4 | 2.7 ± 0.4 | 2.0 ± 0.4 | 4.3 ± 0.6 | 2.6 ± 0.4 | — | — |
| Interleukin 12b | IL12β | 1.8 ± 0.4 | — | 4.4 ± 0.4 | 3.1 ± 0.4 | — | — | — | — | — | — |
| Interleukin 15 receptor, | IL15Rα | — | — | — | — | 4.3 ± 0.4 | — | 2.5 ± 0.5 | 1.9 ± 0.4 | — | — |
| alpha chain | | | | | | | | | | | |
| Interleukin 22 | IL22 | — | — | — | — | 3.4 ± 0.8 | — | — | — | — | — |
| Interleukin 23, alpha | IL23p19 | 6.0 ± 0.5 | — | 8.1 ± 0.5 | 14.5 ± 0.5 | — | — | — | — | — | — |
| subunit p19 | | | | | | | | | | | |
| Interleukin 6 | IL6 | 171.3 ± 0.7 | 36.3 ± 0.9 | 362.0 ± 0.7 | 176.1 ± 0.9 | 97.7 ± 0.8 | 38.6 ± 0.9 | 25.5 ± 0.8 | 14.5 ± 0.9 | 5.2 ± 0.7 | 5.2 ± 0.8 |
| Suppressor of cytokine | SOCS1 | — | — | 1.9 ± 0.5 | — | 7.9 ± 0.6 | 7.9 ± 0.6 | 3.1 ± 0.6 | 2.2 ± 0.5 | — | — |
| signaling 1 | | | | | | | | | | | |
| Suppressor of cytokine | SOCS3 | 3.5 ± 0.4 | 2.5 ± 0.4 | 8.7 ± 0.4 | 7.0 ± 0.4 | 6.5 ± 0.4 | 5.3 ± 0.4 | 3.4 ± 0.4 | 3.1 ± 0.4 | 1.8 ± 0.4 | 2.0 ± 0.4 |
| signaling 3 | | | | | | | | | | | |
| Tumor necrosis factor | TNF | 39.4 ± 0.4 | 21.9 ± 0.5 | 24.3 ± 0.6 | 28.6 ± 0.4 | 29.4 ± 0.4 | 23.9 ± 0.4 | 18.3 ± 0.4 | 19.6 ± 0.4 | 7.8 ± 0.5 | — |
| Tumor necrosis factor | TNFSF14 | — | — | — | — | 3.4 ± 0.6 | — | — | — | — | — |
| (ligand) superfamily, | | | | | | | | | | | |
| member 14 | | | | | | | | | | | |
| Tumor necrosis factor | TNFSF9 | 10.8 ± 0.4 | 5.8 ± 0.5 | 16.1 ± 0.4 | 14.4 ± 0.4 | 2.4 ± 0.4 | — | — | — | — | — |
| (ligand) superfamily, | | | | | | | | | | | |
| member 9 | | | | | | | | | | | |

Values are mean fold change ± SE;
—, No change or less than 1.5 fold.

Cell Surface Adhesion Molecules and Receptors.

Disruption of nrf2 had no effect on the expression of the LPS signaling receptor, TLR4 after LPS challenge. CD14 transcript was markedly higher in nrf2−/− lungs. Expression of several adhesion molecules such as PGLYRP1, TREM-1, SELE, SELP, VCAM1, and members of the C-type lectin family (CLEC4D, CLEC4E) were highly upregulated in nrf2−/− lungs (Table 5). C5R1, which mediates C5A response and augments sepsis, was upregulated to a greater extent in nrf2−/− mice, as shown in Table 5. Among the cell surface adhesion molecules, TREM1 and CD14 were highly upregulated in nrf2−/− lungs.

TABLE 5
Differential expression of transcripts for cell surface adhesion molecules and receptors associated with
inflammation in the lungs of nrf2-deficient and wild-type mice following treatment with LPS.

| | | 30 min | | 1 h | | 6 h | |
|---|---|---|---|---|---|---|---|
| | Gene | (LPS/Vehicle) | | (LPS/Vehicle) | | (LPS/Vehicle) | |
| Gene title | symbol | Nrf2−/− | Nrf2+/+ | Nrf2−/− | Nrf2+/+ | Nrf2−/− | Nrf2+/+ |
| CD14 antigen | CD14 | 9.6 ± 0.4 | 3.7 ± 0.5 | 20.3 ± 0.4 | 14.6 ± 0.4 | 10.9 ± 0.4 | 7.7 ± 0.4 |
| C-type lectin domain | CLEC4D | 8.9 ± 0.5 | 3.6 ± 0.5 | 33.6 ± 0.4 | 28.2 ± 0.4 | 6.6 ± 0.4 | 5.9 ± 0.4 |
| family 4, member d | | | | | | | |
| C-type lectin domain | CLEC4E | 34.8 ± 0.5 | 15.9 ± 0.5 | 111.4 ± 0.4 | 93.1 ± 0.5 | 11.2 ± 0.4 | 9.3 ± 0.5 |
| family 4, member e | | | | | | | |
| Complement component | C5R1 | 3.4 ± 0.5 | — | 7.8 ± 0.4 | 9.1 ± 0.4 | 5.4 ± 0.4 | 4.1 ± 0.4 |
| 5, receptor 1 | | | | | | | |
| Peptidoglycan | PGLYRP1 | 2.1 ± 0.4 | — | 7.9 ± 0.4 | 4.0 ± 0.5 | 4.8 ± 0.4 | 2.4 ± 0.5 |
| recognition protein 1 | | | | | | | |
| Selectin, endothelial cell | SELE | 37.8 ± 0.5 | 15.2 ± 0.5 | 69.6 ± 0.5 | 67.2 ± 0.5 | 4.7 ± 0.5 | 5.4 ± 0.5 |
| Selectin, platelet | SELP | — | — | 44.6 ± 0.7 | 17.4 ± 0.5 | 49.5 ± 0.7 | 26.2 ± 0.4 |
| Toll-like receptor 2 | TLR2 | 4.2 ± 0.5 | 2.4 ± 0.4 | 11.6 ± 0.4 | 12.3 ± 0.4 | 7.0 ± 0.4 | 6.0 ± 0.4 |
| Triggering receptor | TREM1 | 18.0 ± 0.6 | 4.7 ± 0.7 | 151.2 ± 0.4 | 121.9 ± 0.7 | 51.3 ± 0.4 | 45.6 ± 0.6 |
| expressed on myeloid | | | | | | | |
| cells 1 | | | | | | | |
| Triggering receptor | TREM3 | 3.9 ± 0.7 | — | 44.3 ± 0.6 | 52.7 ± 0.8 | 17.4 ± 0.7 | 27.1 ± 0.8 |
| expressed on myeloid | | | | | | | |
| cells 3 | | | | | | | |
| Urokinase plasminogen | PLAUR | 6.1 ± 0.4 | 3.2 ± 0.4 | 7.2 ± 0.4 | 6.0 ± 0.4 | 4.8 ± 0.4 | 4.3 ± 0.4 |
| activator receptor | | | | | | | |
| Vascular cell adhesion | VCAM1 | 3.0 ± 0.4 | 1.9 ± 0.4 | 5.0 ± 0.4 | 4.9 ± 0.4 | 3.8 ± 0.4 | 3.2 ± 0.4 |
| molecule 1 | | | | | | | |

| | | 12 h | | 24 h | |
|---|---|---|---|---|---|
| | Gene | (LPS/Vehicle) | | (LPS/Vehicle) | |
| Gene title | symbol | Nrf2−/− | Nrf2+/+ | Nrf2−/− | Nrf2+/+ |
| CD14 antigen | CD14 | 8.6 ± 0.4 | 5.9 ± 0.4 | 3.4 ± 0.4 | 3.4 ± 0.4 |
| C-type lectin domain | CLEC4D | 7 ± 0.4 | 5.7 ± 0.4 | 2.9 ± 0.4 | 3.5 ± 0.4 |
| family 4, member d | | | | | |
| C-type lectin domain | CLEC4E | 13.9 ± 0.4 | 11.2 ± 0.5 | 6.2 ± 0.4 | 8.5 ± 0.5 |
| family 4, member e | | | | | |
| Complement component | C5R1 | 5.4 ± 0.4 | 4.8 ± 0.4 | 3.2 ± 0.4 | 2.8 ± 0.4 |
| 5, receptor 1 | | | | | |
| Peptidoglycan | PGLYRP1 | 6.6 ± 0.4 | 3.9 ± 0.5 | 4.2 ± 0.4 | 2.5 ± 0.5 |
| recognition protein 1 | | | | | |
| Selectin, endothelial cell | SELE | 3.8 ± 0.6 | 6.2 ± 0.5 | — | — |
| Selectin, platelet | SELP | 15.1 ± 0.9 | 10.6 ± 0.4 | — | 3.2 ± 0.5 |
| Toll-like receptor 2 | TLR2 | 3.3 ± 0.5 | 3.6 ± 0.4 | 2.0 ± 0.4 | 1.9 ± 0.4 |
| Triggering receptor | TREM1 | 42.5 ± 0.4 | 19.7 ± 0.6 | 8.5 ± 0.5 | 2.9 ± 0.7 |
| expressed on myeloid | | | | | |
| cells 1 | | | | | |
| Triggering receptor | TREM3 | 13.1 ± 0.7 | 17.9 ± 0.8 | 13.3 ± 0.6 | 17.8 ± 0.8 |
| expressed on myeloid | | | | | |
| cells 3 | | | | | |
| Urokinase plasminogen | PLAUR | 3.1 ± 0.4 | 2.7 ± 0.4 | 1.8 ± 0.4 | 1.6 ± 0.4 |
| activator receptor | | | | | |
| Vascular cell adhesion | VCAM1 | 1.5 ± 0.4 | 1.9 ± 0.4 | — | — |
| molecule 1 | | | | | |

Regulators of Cytokine Signaling and Transcription.

Transcripts of SOCS3, which are involved in down-regulating cytokine signaling, were induced to a greater extent in nrf2−/− lungs at early time points (Table 6). Transcription factors belonging to the NF-κB family (C-RELC, RELB, NFKBIZ, NFKB2, NFKBIE), the interferon family (IRF5, IRF1, IFI202B, IFI204, IRF1), the early growth response family (EGR2, EGR3) and STAT4 that collectively regulate different inflammatory cascade pathways were expressed to higher levels in nrf2−/− lungs when compared to wild-type mice (Table 6).

TABLE 6
Differential expression of genes associated with transcriptional regulation of
inflammatory molecules in the lungs of nrf2-deficient and wild-type mice following
treatment with LPS.

| | | 30 min | | 1 h | | 6 h | |
|---|---|---|---|---|---|---|---|
| | Gene | (LPS/Vehicle) | | (LPS/Vehicle) | | (LPS/Vehicle) | |
| Gene title | symbol | Nrf2−/− | Nrf2+/+ | Nrf2−/− | Nrf2+/+ | Nrf2−/− | Nrf2+/+ |
| Stat | | | | | | | |
| Signal transducer and | STAT4 | 6.8 ± 1.0 | — | 5.1 ± 0.9 | — | — | — |
| activator of transcription 4 | | | | | | | |
| NF-κB related | | | | | | | |
| Ankyrin repeat domain | ANKRD22 | — | — | 34.1 ± 0.7 | 11.6 ± 0.4 | — | — |
| 22 | | | | | | | |
| Avian reticulo- | RELB | 2.5 ± 0.4 | 1.5 ± 0.4 | 6.6 ± 0.4 | 4.3 ± 0.4 | 4.3 ± 0.4 | 3.2 ± 0.4 |
| endotheliosis viral (v- | | | | | | | |
| rel) oncogene related B | | | | | | | |
| Reticuloendotheliosis | C-REL | 3.5 ± 0.4 | 2.2 ± 0.4 | 7.3 ± 0.4 | 7.1 ± 0.4 | — | — |
| oncogene | | | | | | | |
| B-cell | BCL3 | 3.0 ± 0.4 | 1.8 ± 0.4 | 8.5 ± 0.4 | 6.5 ± 0.4 | 9.1 ± 0.4 | 8.4 ± 0.4 |
| leukemia/lymphoma 3 | | | | | | | |
| CAMP responsive element | CREB5 | 2.5 ± 0.4 | — | — | — | — | — |
| binding protein 5 | | | | | | | |
| CCAAT/enhancer | CEBPB | 4.9 ± 0.4 | 3.1 ± 0.4 | 6.4 ± 0.4 | 5.8 ± 0.4 | 5.6 ± 0.4 | 4.6 ± 0.4 |
| binding protein | | | | | | | |
| (C/EBP), beta | | | | | | | |
| Inhibitor of kappa b | IKBKE | — | — | 11.0 ± 0.5 | 4.5 ± 0.6 | 17.1 ± 0.5 | 11.0 ± 0.4 |
| kinase epsilon | | | | | | | |
| Interleukin-1 receptor- | IRAK3 | — | — | 7.2 ± 0.4 | 4.0 ± 0.4 | 8.3 ± 0.4 | 5.9 ± 0.4 |
| associated kinase 3 | | | | | | | |
| Max dimerization | MAD | 5.5 ± 0.6 | 3.5 ± 0.4 | 17.3 ± 0.4 | 18.6 ± 0.4 | 13.1 ± 0.4 | 12.9 ± 0.4 |
| protein | | | | | | | |
| Nuclear factor of kappa | NFKBIZ | 20.5 ± 0.4 | 16.7 ± 0.4 | 22.5 ± 0.4 | 32.7 ± 0.3 | 6.0 ± 0.4 | 7.7 ± 0.4 |
| light polypeptide gene | | | | | | | |
| enhancer in B-cells | | | | | | | |
| inhibitor, zeta | | | | | | | |
| Nuclear factor of kappa | NFKB2 | 2.5 ± 0.4 | 2.2 ± 0.4 | 7.7 ± 0.4 | 4.9 ± 0.4 | 3.5 ± 0.4 | 2.8 ± 0.4 |
| light polypeptide gene | | | | | | | |
| enhancer in B-cells 2, | | | | | | | |
| p49/p100 | | | | | | | |
| Nuclear factor of kappa | NFKBIE | 3.2 ± 0.4 | 1.8 ± 0.4 | 5.9 ± 0.4 | 5.7 ± 0.4 | 3.7 ± 0.4 | 3.2 ± 0.4 |
| light polypeptide gene | | | | | | | |
| enhancer in B-cells | | | | | | | |
| inhibitor, epsilon | | | | | | | |
| TRAF family member- | TANK | 2.6 ± 0.4 | 1.9 ± 0.4 | 4.3 ± 0.4 | 5.7 ± 0.4 | — | — |
| associated NF-kappa B | | | | | | | |
| activator | | | | | | | |
| Interferon related | | | | | | | |
| Interferon activated gene | IFI202B | 2.5 ± 0.4 | — | 3.5 ± 0.5 | 1.9 ± 0.5 | 39.4 ± 0.4 | 21.0 ± 0.4 |
| 202B | | | | | | | |
| Interferon activated gene | IFI204 | 4.3 ± 0.4 | — | 4.8 ± 0.7 | 1.9 ± 0.5 | 31.8 ± 0.4 | 29.9 ± 0.4 |
| 204 | | | | | | | |
| Interferon regulatory | IRF1 | 5.7 ± 0.4 | 4.2 ± 0.4 | 4.5 ± 0.4 | 3.7 ± 0.4 | 4.9 ± 0.4 | 4.5 ± 0.4 |
| factor 1 | | | | | | | |
| Interferon regulatory | IRF5 | 1.7 ± 0.4 | — | 2.4 ± 0.4 | 1.7 ± 0.4 | 3.8 ± 0.4 | 3.1 ± 0.4 |
| factor 5 | | | | | | | |
| Interferon regulatory | IRF7 | — | — | 1.9 ± 0.4 | — | 22.6 ± 0.4 | 15.6 ± 0.4 |
| factor 7 | | | | | | | |
| Interferon-induced | IFI44 | — | — | — | — | 17.9 ± 0.4 | 10.6 ± 0.4 |
| protein 44 | | | | | | | |
| Interferon-induced | IFIT2 | — | — | — | — | 39.9 ± 0.4 | 23.1 ± 0.4 |
| protein with tetra- | | | | | | | |
| tricopeptide repeats 2 | | | | | | | |
| (ISG54) | | | | | | | |
| Interferon-induced | IFIT3 | — | — | — | — | 18.4 ± 0.4 | 9.9 ± 0.4 |
| protein with tetra- | | | | | | | |
| tricopeptide repeats 3 | | | | | | | |
| (GARG-49) | | | | | | | |
| Myxovirus (influenza | Mx1 | — | — | — | 2.1 ± 0.5 | 49.9 ± 0.4 | 23.8 ± 0.4 |
| virus) resistance 1 | | | | | | | |
| Stat | | | | | | | |
| Signal transducer and | STAT4 | 6.8 ± 1.0 | — | 5.1 ± 0.9 | — | — | — |
| activator of transcription 4 | | | | | | | |
| Other transcription | | | | | | | |
| factors | | | | | | | |
| Early growth response 2 | EGR2 | 8.5 ± 0.4 | 6.5 ± 0.4 | 6.1 ± 0.4 | 5.6 ± 0.4 | — | — |
| Early growth response 3 | EGR3 | 84.4 ± 0.4 | 71.0 ± 0.4 | 44 ± 0.4 | 67.6 ± 0.4 | — | — |
| Spi-C transcription | SPIC | — | — | — | — | 31.8 ± 1.0 | 19.2 ± 0.6 |
| factor (Spi-1/PU.1 | | | | | | | |
| related) | | | | | | | |
| TGFB-induced factor 2 | TGIF2 | 8.1 ± 0.4 | 4.1 ± 0.8 | 7.0 ± 0.5 | 10.9 ± 0.5 | — | — |
| Transcription factor E3 | TCFE3 | 1.4 ± 0.4 | — | 2.1 ± 0.3 | — | — | — |
| Transforming growth | TGFBI | 1.5 ± 0.4 | — | 1.5 ± 0.4 | 1.5 ± 0.4 | 2.1 ± 0.4 | 2.4 ± 0.4 |
| factor, beta induced | | | | | | | |
| V-maf musculo- | MAFF | 5.5 ± 0.4 | 3.5 ± 0.4 | 8.5 ± 0.4 | 7.0 ± 0.4 | 6.1 ± 0.4 | 5.4 ± 0.4 |
| aponeurotic fibro- | | | | | | | |
| sarcoma oncogene | | | | | | | |
| family, protein F (avian) | | | | | | | |

| | | 12 h | | 24 h | |
|---|---|---|---|---|---|
| | Gene | (LPS/Vehicle) | | (LPS/Vehicle) | |
| Gene title | symbol | Nrf2−/− | Nrf2+/+ | Nrf2−/− | Nrf2+/+ |
| Stat | | | | | |
| Signal transducer and | STAT4 | — | — | — | — |
| activator of transcription 4 | | | | | |
| NF-κB related | | | | | |
| Ankyrin repeat domain | ANKRD22 | — | — | — | — |
| 22 | | | | | |
| Avian reticulo- | RELB | 2.9 ± 0.4 | 2.6 ± 0.4 | 2.0 ± 0.4 | 1.8 ± 0.4 |
| endotheliosis viral (v- | | | | | |
| rel) oncogene related B | | | | | |
| Reticuloendotheliosis | C-REL | — | — | — | — |
| oncogene | | | | | |
| B-cell | BCL3 | 3.5 ± 0.4 | 3.4 ± 0.4 | 1.6 ± 0.5 | 2.0 ± 0.4 |
| leukemia/lymphoma 3 | | | | | |
| CAMP responsive element | CREB5 | — | — | — | — |
| binding protein 5 | | | | | |
| CCAAT/enhancer | CEBPB | 4.4 ± 0.4 | 3.4 ± 0.4 | 2.4 ± 0.4 | 2.2 ± 0.4 |
| binding protein | | | | | |
| (C/EBP), beta | | | | | |
| Inhibitor of kappa b | IKBKE | 21.9 ± 0.5 | 17.1 ± 0.4 | 6.9 ± 0.5 | 6.8 ± 0.4 |
| kinase epsilon | | | | | |
| Interleukin-1 receptor- | IRAK3 | 6.9 ± 0.4 | 6.0 ± 0.4 | 3.6 ± 0.4 | 3.6 ± 0.4 |
| associated kinase 3 | | | | | |
| Max dimerization | MAD | 7.2 ± 0.4 | 6.7 ± 0.5 | 1.8 ± 0.4 | 2.3 ± 0.4 |
| protein | | | | | |
| Nuclear factor of kappa | NFKBIZ | 4.2 ± 0.4 | 5.2 ± 0.4 | 1.9 ± 0.4 | 2.3 ± 0.4 |
| light polypeptide gene | | | | | |
| enhancer in B-cells | | | | | |
| inhibitor, zeta | | | | | |
| Nuclear factor of kappa | NFKB2 | 2.5 ± 0.4 | 2.3 ± 0.4 | 1.7 ± 0.4 | 1.8 ± 0.4 |
| light polypeptide gene | | | | | |
| enhancer in B-cells 2, | | | | | |
| p49/p100 | | | | | |
| Nuclear factor of kappa | NFKBIE | 2.8 ± 0.4 | 2.5 ± 0.4 | 1.7 ± 0.4 | 1.8 ± 0.4 |
| light polypeptide gene | | | | | |
| enhancer in B-cells | | | | | |
| inhibitor, epsilon | | | | | |
| TRAF family member- | TANK | — | — | — | — |
| associated NF-kappa B | | | | | |
| activator | | | | | |
| Interferon related | | | | | |
| Interferon activated gene | IFI202B | 14.9 ± 0.4 | 8.7 ± 0.4 | 6.5 ± 0.4 | 4.8 ± 0.4 |
| 202B | | | | | |
| Interferon activated gene | IFI204 | 12 ± 0.5 | 9.4 ± 0.4 | 7.1 ± 0.5 | 3.7 ± 0.4 |
| 204 | | | | | |
| Interferon regulatory | IRF1 | 2.5 ± 0.4 | 2.4 ± 0.4 | — | — |
| factor 1 | | | | | |
| Interferon regulatory | IRF5 | 2.5 ± 0.4 | 2.2 ± 0.4 | 2.2 ± 0.4 | 2.1 ± 0.4 |
| factor 5 | | | | | |
| Interferon regulatory | IRF7 | 16.3 ± 0.4 | 13.1 ± 0.4 | 7.7 ± 0.5 | 6.0 ± 0.4 |
| factor 7 | | | | | |
| Interferon-induced | IFI44 | 6.6 ± 0.4 | 5.5 ± 0.4 | 3.1 ± 0.4 | 1.8 ± 0.4 |
| protein 44 | | | | | |
| Interferon-induced | IFIT2 | 11.8 ± 0.6 | 8.2 ± 0.5 | 2.5 ± 0.5 | 2.1 ± 0.4 |
| protein with tetra- | | | | | |
| tricopeptide repeats 2 | | | | | |
| (ISG54) | | | | | |
| Interferon-induced | IFIT3 | 6.3 ± 0.4 | 5.8 ± 0.4 | 2.9 ± 0.5 | 2.4 ± 0.4 |
| protein with tetra- | | | | | |
| tricopeptide repeats 3 | | | | | |
| (GARG-49) | | | | | |
| Myxovirus (influenza | Mx1 | 6.9 ± 0.7 | 4.7 ± 0.4 | 2.1 ± 0.4 | 1.9 ± 0.5 |
| virus) resistance 1 | | | | | |
| Stat | | | | | |
| Signal transducer and | STAT4 | — | — | — | — |
| activator of transcription 4 | | | | | |
| Other transcription | | | | | |
| factors | | | | | |
| Early growth response 2 | EGR2 | — | — | — | — |
| Early growth response 3 | EGR3 | — | — | — | — |
| Spi-C transcription | SPIC | 20.0 ± 0.8 | 21.4 ± 0.5 | 35.0 ± 0.8 | 35.0 ± 0.5 |
| factor (Spi-1/PU.1 | | | | | |
| related) | | | | | |
| TGFB-induced factor 2 | TGIF2 | — | — | — | — |
| Transcription factor E3 | TCFE3 | — | — | — | — |
| Transforming growth | TGFBI | 2.8 ± 0.4 | 2.5 ± 0.4 | 3.1 ± 0.4 | 3.3 ± 0.4 |
| factor, beta induced | | | | | |
| V-maf musculo- | MAFF | 5.1 ± 0.4 | 4.0 ± 0.4 | — | — |
| aponeurotic fibro- | | | | | |
| sarcoma oncogene | | | | | |
| family, protein F (avian) | | | | | |

### Immunoglobulin and MHC.

Transcripts of many members of the immunoglobulin (IGHG, IGH-VJ558, IGH-4, IGH-6, IGJ, IGK-V21, IGk-V32, IGK-V8, IGL-V1, IGSF6, IGM) as well as MHC class II family (H2-AA, H2-AB1, H2-EA, H2-DMA, H2-DMB1, H2-DMB2) were selectively upregulated in the lungs of nrf2−/− mice at 30 min (Table 7) indicating severe immune dysfunction.

TABLE 7
Differential expression of members of immunoglobulin and MHC class II family in
the lungs of nrf2-deficient and wild-type mice 30 min after LPS challenge.
Values are mean fold change ± SE; —, No change or less than 1.5 fold.

| | | Nrf2−/−, | Nrf2+/+, |
|---|---|---|---|
| | | LPS/ | LPS/ |
| Gene name | Gene symbol | Vehicle | Vehicle |
| Histocompatibility 2, class II antigen A, | H2-Aα | 1.6 ± 0.4 | — |
| alpha | | | |
| Histocompatibility 2, class II antigen A, | H2-Aβ1 | 2.0 ± 0.4 | — |
| beta 1 | | | |
| Histocompatibility 2, class II antigen E | H2-Eα | 5.1 ± 0.7 | — |
| alpha | | | |
| Histocompatibility 2, class II, locus dma | H2-DMA | 2.3 ± 0.4 | — |
| Histocompatibility 2, class II, locus Mb1 | H2-DMB1 | 2.3 ± 0.4 | — |
| Histocompatibility 2, class II, locus Mb2 | H2-DMB2 | 1.6 ± 0.4 | — |
| Immunoglobulin heavy chain (gamma | IGHγ | 12.9 ± 0.7 | — |
| polypeptide) | | | |
| Immunoglobulin heavy chain (J558 | IGH-VJ558 | 4.7 ± 0.4 | — |
| family) | | | |
| Immunoglobulin heavy chain 4 (serum | IGH-4 | 38.9 ± 1.0 | — |
| igg1) | | | |
| Immunoglobulin heavy chain 6 (heavy | IGH-6 | 29.7 ± 0.8 | 2.1 ± 0.4 |
| chain of igm) | | | |
| Immunoglobulin joining chain | IGJ | 7.5 ± 0.5 | — |
| Immunoglobulin kappa chain variable 21 | IGK-V21 | 9.9 ± 0.6 | — |
| (V21) | | | |
| Immunoglobulin kappa chain variable 32 | IGK-V32 | 13.9 ± 0.9 | — |
| (V32) | | | |
| Immunoglobulin kappa chain variable 8 | IGK-V8 | 4.1 ± 0.4 | — |
| (V8) | | | |
| Immunoglobulin lambda chain, variable 1 | IGL-V1 | 3.7 ± 0.7 | — |
| Immunoglobulin superfamily, member 6 | IGSF6 | 10.3 ± 0.5 | 4.3 ± 0.5 |
| Ig kappa chain | IGM | 6.7 ± 0.5 | — |

### Acute Phase Proteins, Heat Shock Proteins and Other Inflammation-Modulating Molecules and Enzymes.

Many genes that encode for acute phase proteins belonging to the family of proteinase inhibitors (SERPINA3M, SERPINB2, and SERPINE1), serum amyloid (SAA2, SAA3), and orsomucoid (ORM1, ORM2) and HSP1A were markedly increased in nrf2 lungs (Table 8).

TABLE 8
Differential expression of genes encoding acute phase proteins in the lungs of nrf2-
deficient and wild-type mice following treatment with LPS. Values are mean fold change ±
SE; —, No change or less than 1.5 fold

| | | 30 min | | 1 h | | 6 h | |
|---|---|---|---|---|---|---|---|
| | Gene | (LPS/Vehicle) | | (LPS/Vehicle) | | (LPS/Vehicle) | |
| Gene title | symbol | Nrf2−/− | Nrf2+/+ | Nrf2−/− | Nrf2+/+ | Nrf2−/− | Nrf2+/+ |
| Heat shock protein 1A | HSPA1A | 30.1 ± 0.4 | 23.3 ± 0.5 | 2.8 ± 0.5 | 1.5 ± 0.4 | — | — |
| Heat shock protein 8 | HSPA8 | 2.1 ± 0.4 | 4.3 ± 0.5 | 1.5 ± 0.4 | — | — | — |
| Metallothionein 2 | MT2 | 1.8 ± 0.5 | — | 5.6 ± 0.5 | 3.6 ± 0.4 | 8.5 ± 0.5 | 6.2 ± 0.4 |
| Orosomucoid 1 | ORM1 | — | — | 1.6 ± 0.5 | — | 22.9 ± 0.4 | 14.8 ± 0.7 |
| Orosomucoid 2 | ORM2 | — | — | — | — | 6.0 ± 0.4 | 3.8 ± 0.6 |
| Serine (or cysteine) | SERPINA1A | — | — | — | — | — | — |
| proteinase inhibitor, | | | | | | | |
| clade A, member 1a | | | | | | | |
| Serine (or cysteine) | SERPINA3C | — | — | 1.8 ± 0.5 | — | 6.7 ± 0.4 | 8.2 ± 0.5 |
| proteinase inhibitor, | | | | | | | |
| clade A, member 3C | | | | | | | |
| Serine (or cysteine) | SERPINA3G | 1.9 ± 0.5 | — | 3.2 ± 0.5 | 1.5 ± 0.4 | 14.7 ± 0.4 | 9.4 ± 0.4 |
| proteinase inhibitor, | | | | | | | |
| clade A, member 3G | | | | | | | |
| Serine (or cysteine) | SERPINA3M | — | — | — | — | 8.0 ± 0.4 | 5.7 ± 0.4 |
| proteinase inhibitor, | | | | | | | |
| clade A, member 3M | | | | | | | |
| Serine (or cysteine) | SERPINA3N | — | — | 4.2 ± 0.6 | 3.7 ± 0.6 | 11.2 ± 0.5 | 31.3 ± 0.4 |
| proteinase inhibitor, | | | | | | | |
| clade A, member 3N | | | | | | | |
| Serine (or cysteine) | SERPINB2 | 14.3 ± 0.6 | — | 18.5 ± 0.5 | 10.1 ± 0.6 | 5.0 ± 0.6 | 2.1 ± 0.5 |
| proteinase inhibitor, | | | | | | | |
| clade B, member 2 | | | | | | | |
| Serine (or cysteine) | SERPINE1 | 10.9 ± 0.4 | 8.1 ± 0.4 | 32.4 ± 0.4 | 24.3 ± 0.4 | 23.8 ± 0.4 | 23.8 ± 0.4 |
| proteinase inhibitor, | | | | | | | |
| clade E, member 1 | | | | | | | |
| Serum amyloid A 1 | SAA1 | — | — | 3.1 ± 0.5 | — | 93.1 ± 0.4 | 95.7 ± 0.5 |
| Serum amyloid A 2 | SAA2 | — | — | — | — | 28.1 ± 0.4 | 19.8 ± 0.4 |
| Serum amyloid A 3 | SAA3 | 3.0 ± 0.5 | — | 18.0 ± 0.4 | 4.0 ± 0.9 | 85.6 ± 0.4 | 25.5 ± 0.8 |

| | | 12 h | | 24 h | |
|---|---|---|---|---|---|
| | Gene | (LPS/Vehicle) | | (LPS/Vehicle) | |
| Gene title | symbol | Nrf2−/− | Nrf2+/+ | Nrf2−/− | Nrf2+/+ |
| Heat shock protein 1A | HSPA1A | — | — | — | 1.7 ± 0.4 |
| Heat shock protein 8 | HSPA8 | — | — | 1.7 ± 0.4 | 2.4 ± 0.4 |
| Metallothionein 2 | MT2 | 7.5 ± 0.5 | 5.2 ± 0.4 | 2.0 ± 0.6 | 1.6 ± 0.4 |
| Orosomucoid 1 | ORM1 | 21.1 ± 0.5 | 12.0 ± 0.7 | 3.1 ± 0.6 | 5.1 ± 0.7 |
| Orosomucoid 2 | ORM2 | 7.2 ± 0.5 | 3.8 ± 0.5 | 3.5 ± 0.5 | 3.3 ± 0.5 |
| Serine (or cysteine) | SERPINA1A | — | 43.1 ± 0.5 | — | — |
| proteinase inhibitor, | | | | | |
| clade A, member 1a | | | | | |
| Serine (or cysteine) | SERPINA3C | 3.6 ± 0.7 | 3.3 ± 0.5 | — | 1.6 ± 0.4 |
| proteinase inhibitor, | | | | | |
| clade A, member 3C | | | | | |
| Serine (or cysteine) | SERPINA3G | 10.1 ± 0.4 | 7.0 ± 0.4 | 2.6 ± 0.5 | — |
| proteinase inhibitor, | | | | | |
| clade A, member 3G | | | | | |
| Serine (or cysteine) | SERPINA3M | 10.9 ± 0.5 | 3.5 ± 0.4 | 3.2 ± 0.5 | 2.0 ± 0.4 |
| proteinase inhibitor, | | | | | |
| clade A, member 3M | | | | | |
| Serine (or cysteine) | SERPINA3N | 12.5 ± 0.5 | 30.7 ± 0.4 | 6.7 ± 0.5 | 16.3 ± 0.4 |
| proteinase inhibitor, | | | | | |
| clade A, member 3N | | | | | |
| Serine (or cysteine) | SERPINB2 | 3.9 ± 0.7 | — | 2.9 ± 0.6 | — |
| proteinase inhibitor, | | | | | |
| clade B, member 2 | | | | | |
| Serine (or cysteine) | SERPINE1 | 9.3 ± 0.5 | 15.7 ± 0.4 | 2.3 ± 0.5 | 3.8 ± 0.5 |
| proteinase inhibitor, | | | | | |
| clade E, member 1 | | | | | |
| Serum amyloid A 1 | SAA1 | 66.3 ± 0.4 | 76.6 ± 0.5 | 23.4 ± 0.4 | 32.7 ± 0.5 |
| Serum amyloid A 2 | SAA2 | 16.2 ± 0.4 | 12.5 ± 0.4 | 5.1 ± 0.5 | — |
| Serum amyloid A 3 | SAA3 | 90.5 ± 0.5 | 24.9 ± 0.8 | 61.0 ± 0.4 | 22 ± 0.8 |

Expression levels of ARG2 [an endogenous inhibitor of iNOS that regulates arginine metabolism (Mori M et al J Nutr 134:2820S-2825S; discussion 2853S. 1994)], INDO [which exerts immunosuppressive effects through induction of apoptosis in T cells by regulating tryptophan metabolism (Terness P. J Exp Med 196:447-457. 2002], PLEK [which regulates phagocytosis activity by macrophages (Brumell J H et al. J Immunol 163:3388-3395. 1999)], and PFC [which is a regulator of alternative complement system were all higher in nrf2−/− lungs at 30 min (Table 9).

TABLE 9
Differential expression of selected genes that modulate inflammation in the lungs of
nrf2-deficient and wild-type mice following treatment with LPS.
Values are mean fold change ± SE; —, No change or less than 1.5 fold.

| | | 30 min | | 1 h | | 6 h | |
|---|---|---|---|---|---|---|---|
| | Gene | (LPS/Vehicle) | | (LPS/Vehicle) | | (LPS/Vehicle) | |
| Gene title | symbol | Nrf2−/− | Nrf2+/+ | Nrf2−/− | Nrf2+/+ | Nrf2−/− | Nrf2+/+ |
| Arginase II | ARG2 | 4.1 ± 0.4 | 1.8 ± 0.4 | 7.0 ± 0.4 | 7.5 ± 0.4 | 7.0 ± 0.4 | 5.2 ± 0.4 |
| Immune-responsive | IRG1 | 286.0 ± 0.6 | 29.0 ± 0.8 | 1858.0 ± 0.4 | 1082.0 ± 0.4 | 552.0 ± 0.4 | 304.0 ± 0.5 |
| gene 1 | | | | | | | |
| Indoleamine-pyrrole 2,3 | INDO | 2.2 ± 0.5 | — | — | — | 25.6 ± 0.6 | 19.8 ± 0.5 |
| dioxygenase | | | | | | | |
| Neutrophil cytosolic | NCF1 | 4.9 ± 0.5 | 2.0 ± 0.4 | 16.3 ± 0.4 | 13.5 ± 0.4 | 5.8 ± 0.4 | 4.3 ± 0.4 |
| factor 1 | | | | | | | |
| Neutrophil cytosolic | NCF4 | 2.7 ± 0.4 | — | 5.7 ± 0.4 | 4.7 ± 0.4 | 5 ± 0.3 | 4.1 ± 0.4 |
| factor 4 | | | | | | | |
| Nitric oxide synthase 2, | NOS2 | — | — | — | — | 14.7 ± 0.5 | 7.9 ± 0.6 |
| inducible, macrophage | | | | | | | |
| Pleckstrin | PLEK | 4.3 ± 0.4 | 2.5 ± 0.4 | 9.6 ± 0.4 | 10.3 ± 0.4 | 3.3 ± 0.4 | 3.1 ± 0.4 |
| Properdin factor, | PFC | 2.6 ± 0.5 | — | 2.6 ± 0.5 | 2.4 ± 0.4 | 3.0 ± 0.5 | |
| complement | | | | | | | |

| | | 12 h | | 24 h | |
|---|---|---|---|---|---|
| | Gene | (LPS/Vehicle) | | (LPS/Vehicle) | |
| Gene title | symbol | Nrf2−/− | Nrf2+/+ | Nrf2−/− | Nrf2+/+ |
| Arginase II | ARG2 | 4.6 ± 0.4 | 2.9 ± 0.4 | 1.8 ± 0.4 | 1.5 ± 0.4 |
| Immune-responsive | IRG1 | 313.0 ± 0.4 | 183.5 ± 0.7 | 53.0 ± 0.4 | 64.1 ± 0.5 |
| gene 1 | | | | | |
| Indoleamine-pyrrole 2,3 | INDO | 9.3 ± 0.5 | 8.5 ± 0.6 | — | — |
| dioxygenase | | | | | |
| Neutrophil cytosolic | NCF1 | 6.6 ± 0.4 | 4.7 ± 0.4 | 2.8 ± 0.4 | 2.4 ± 0.4 |
| factor 1 | | | | | |
| Neutrophil cytosolic | NCF4 | 6.2 ± 0.3 | 4.8 ± 0.4 | 4.0 ± 0.4 | 3.9 ± 0.4 |
| factor 4 | | | | | |
| Nitric oxide synthase 2, | NOS2 | — | — | — | — |
| inducible, macrophage | | | | | |
| Pleckstrin | PLEK | 2.2 ± 0.4 | 2.4 ± 0.4 | 2.0 ± 0.4 | 2.1 ± 0.4 |
| Properdin factor, | PFC | 3.6 ± 0.5 | 2.5 ± 0.4 | 5.5 ± 0.5 | 3.8 ± 0.4 |
| complement | | | | | |

### ROS/RNS Generators:

The expression of NCF1 (p47phox) and NCF4 (p40phox), which are members of the NADPH oxidase family involved in generation of reactive oxygen species during phagocytic activity by neutrophils and macrophages, were significantly higher in nrf2−/− lungs at early stages (until 1 h; Table 9, above). Expression of NOS2 (iNOS), which is involved in nitric oxide generation, was induced at the 6 h time point and was greater in the lungs of nrf2−/− mice (Table 9, above).

### Antioxidants.

Nrf2 is a key transcription factor for regulating the expression of antioxidative genes. Differential gene expression profiling of vehicle-treated nrf2+/+ and nrf2−/− lungs showed constitutively elevated expression of antioxidative genes such as glutathione peroxidase 2 (GPX2), glutamate cysteine ligase catalytic subunit (GCLC), thioredoxin reductase 1, and members of the glutathione S-transferase family in wild-type mice (Table 10).

TABLE 10
Antioxidative genes that are constitutively elevated in the lungs of wild-type
compared to nrf2-deficient mice.

| | Vehicle, | | | | | |
|---|---|---|---|---|---|---|
| | Nrf2+/+ // | LPS, Nrf2+/+ // Nrf2−/− | | | | |
| Gene name (Gene symbol) | Nrf2−/− | 30 min | 1 h | 6 h | 12 h | 24 h |
| Glutamate-cysteine ligase, | 2.1 ± 0.4 | — | 1.9 ± 0.4 | 1.7 ± 0.5 | 1.6 ± 0.4 | 2.1 ± 0.4 |
| catalytic subunit (GCLC) | | | | | | |
| Glutathione peroxidase 2 | 5.3 ± 0.5 | 4.8 ± 0.5 | 4.4 ± 0.5 | 3.4 ± 0.6 | 2.3 ± 0.5 | 4.0 ± 0.7 |
| (GPX2) | | | | | | |
| Glutathione S-transferase, | 2.6 ± 0.4 | 3.3 ± 0.4 | 2.5 ± 0.4 | 2.7 ± 0.5 | 4.0 ± 0.5 | 2.4 ± 0.4 |
| alpha 3 (GSTA3) | | | | | | |
| Glutathione S-transferase, | 1.7 ± 0.4 | — | 1.5 ± 0.4 | — | — | — |
| alpha 4 (GSTA4) | | | | | | |
| Glutathione S-transferase, mu | 2.4 ± 0.4 | 2.6 ± 0.4 | 2.4 ± 0.3 | 1.9 ± 0.4 | 1.7 ± 0.4 | 1.5 ± 0.4 |
| 1 (GSTM1) | | | | | | |
| Glutathione S-transferase, mu | 1.6 ± 0.4 | 1.9 ± 0.3 | 1.6 ± 0.3 | — | 1.5 ± 0.4 | — |
| 2 (GSTM2) | | | | | | |
| Malic enzyme, supernatant | 1.9 ± 0.8 | 1.9 ± 0.3 | 1.8 ± 0.4 | 1.5 ± 0.4 | 1.5 ± 0.4 | 1.6 ± 0.4 |
| (MOD1) | | | | | | |
| Catalase (CAT) | — | — | — | — | — | 3.3 ± 0.5 |
| Thioredoxin reductase 1 | 1.8 ± 0.4 | — | — | — | — | — |
| (TXNRD1) | | | | | | |

Values are mean fold change ± SE;
—, No change or less than 1.5 fold.

Although expression of these genes were not altered significantly in wild-type mice after LPS challenge, at all time points, transcript levels of these antioxidative genes were higher in the lungs of wild-type mice compared to nrf2−/− mice.

Genes that were selected for validation included chemokines (MCPS, MCP1, MIP2), cytokines (IL-6, IL-1α, TNF-α, CSF2), LPS membrane receptor (CD14), immunoglobulins (IGH-4, IHSF6), an MHC class II member (H2-EA), and the transcription factor STAT4. Expression values of these gene's obtained from real time PCR were consistent with the microarray values in terms of magnitude and pattern across all the time points (Table 11).

TABLE 11
Validation by real time-PCR of selected LPS inducible genes identified by microarray analysis in the lungs of mice of both
genotypes challenged with LPS. Values are the ratio of mean fold change of LPS treatment to vehicle control (n = 3).

| | Nrf2−/−, LPS/Vehicle | | | | | | | | | |
|---|---|---|---|---|---|---|---|---|---|---|
| | 30 min | | 1 h | | 6 h | | 12 h | | 24 h | |
| | Real Time | Micro- | Real Time | Micro- | Real Time | Micro- | Real Time | Micro- | Real Time | Micro- |
| Gene symbol | PCR | array | PCR | array | PCR | array | PCR | array | PCR | array |
| CCL12/MCP5 | 18.1 | 19.7 | 27.8 | 27.7 | 15.3 | 19.3 | 18.3 | 19.6 | 25.3 | 29.2 |
| CCL2/MCP1 | 7.2 | 6.3 | 23.1 | 24.8 | 15.6 | 20.4 | 7.6 | 6.0 | 1.3 | 4.7 |
| CD14 | 8.1 | 9.6 | 22.3 | 20.3 | 12.3 | 10.9 | 7.5 | 8.6 | 2.6 | 3.4 |
| CSF2 | 7.2 | 6.3 | 58.6 | 70.5 | 44.6 | 65.8 | 12.8 | 12.5 | 1.4 | 1.3 |
| CXCL2/MIP2 | 75.2 | 123.6 | 210.2 | 250.0 | 48.3 | 76.6 | 25.6 | 35.8 | 4.1 | 3.9 |
| H2-Eα | 3.9 | 5.1 | 1.2 | — | 0.7 | — | 0.5 | — | 0.4 | — |
| IGH-4 | 12.9 | 38.9 | 0.5 | — | 0.3 | — | 0.2 | — | 0.4 | — |
| IHSF6 | 10.5 | 10.3 | 15.2 | | 3.2 | | 4.1 | | 3.2 | |
| IL-1α | 5.1 | 4.9 | 9.8 | 11.2 | 1.6 | — | 1.6 | — | 1.3 | — |
| IL-6 | 99.1 | 171.3 | 202.1 | 362.0 | 70.0 | 97.7 | 7.94 | 25.5 | 2.28 | 5.2 |
| IRG1 | 486.3 | 286.0 | 2548.4 | 1858.4 | 370.5 | 552.0 | 208.9 | 313.0 | 63.2 | 53.0 |
| STAT4 | 3.6 | 6.8 | 3.0 | 5.1 | 0.8 | — | 0.7 | — | 0.8 | — |
| TNFα | 35.2 | 39.4 | 21.1 | 24.3 | 25.3 | 29.6 | 16.5 | 18.3 | 6.4 | 7.8 |
| | Nrf2+/+, LPS/Vehicle | | | | | | | | | |
| | 30 min | | 1 h | | 6 h | | 12 h | | 24 h | |
| | Real Time | Micro- | Real Time | Micro- | Real Time | Micro- | Real Time | Micro- | Real Time | Micro- |
| Gene symbol | PCR | array | PCR | array | PCR | array | PCR | array | PCR | array |
| CCL12/MCP5 | 6.5 | 7.5 | 11.3 | 12.5 | 7.2 | 8.6 | 6.5 | 15.0 | 15.2 | 14.9 |
| CCL2/MCP1 | 1.8 | 1.8 | 4.8 | 4.2 | 6.3 | 7.0 | 6.8 | 7.1 | 1.3 | 1.2 |
| CD14 | 4.3 | 3.7 | 11.3 | 14.6 | 7.8 | 7.7 | 4.5 | 5.9 | 2.4 | 3.4 |
| CSF2 | 1.3 | 1.2 | 38.2 | | 75.8 | 49.9 | 20.4 | 106.9 | 1.5 | 24.3 |
| CXCL2/MIP2 | 32.1 | 56.9 | 175.2 | 215.3 | 36.2 | 66.7 | 26.3 | 28.2 | 4.7 | 5.1 |
| H2-Eα | 3.0 | — | 1.0 | — | 0.5 | — | 0.7 | — | 0.4 | — |
| IGH-4 | 3.0 | — | 0.9 | — | 0.6 | — | 0.7 | — | 1.1 | — |
| IHSF6 | 3.1 | | 10.6 | | 2.8 | | 4.0 | | 2.6 | |
| IL-1α | 1.3 | — | 1.9 | — | 1.32 | — | 0.8 | — | 0.9 | — |
| IL-6 | 30.2 | 36.3 | 140.6 | 176.1 | 20.9 | 38.6 | 20.5 | 14.5 | 2.9 | 5.2 |
| IRG1 | 64.6 | 29 | 2100.0 | 1082.0 | 332.4 | 304 | 170.8 | 183.5 | 73.3 | 64.1 |
| STAT4 | 1.5 | — | 1.8 | — | 0.7 | — | 0.6 | — | 0.7 | — |
| TNFα | 21.3 | 21.9 | 19.5 | 28.6 | 23.1 | 23.9 | 17.2 | 19.6 | 1.3 | — |

### Example 16

### TNF-α Stimulus Induces a Greater Pulmonary Inflammatory Response in Nrf2-Deficient Mice

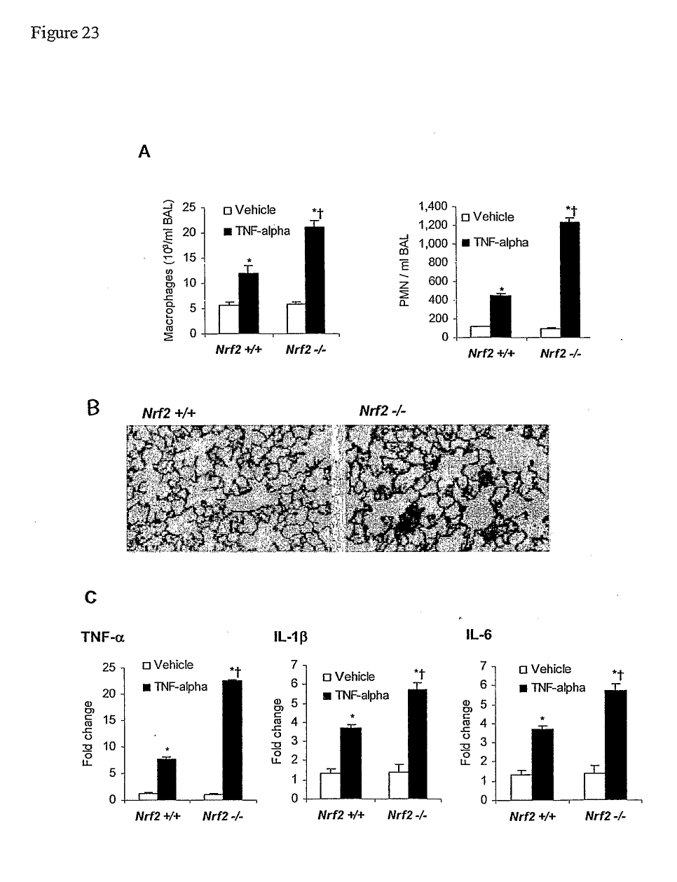

Microarray and BAL fluid analysis showed greater expression of TNF-α in the lungs of nrf2−/− mice compared to nrf2+/+ mice in response to LPS. To characterize the effect of TNF-α mediated inflammation, mice of both genotypes were administered with TNF-α (ip). Following TNF-α treatment, lungs of nrf2−/− mice showed increased infiltration of inflammatory cells as measured by BAL analysis and histopathology (FIGS. 23 A and B) when compared to wild-type litter mates. Real time PCR analysis of selected genes (TNF-α, IL-1β, and IL-6) in the lungs of mice 30 min after TNF-α treatment revealed greater expression in nrf2−/− mice compared to nrf2+/+(FIG. 23 C).

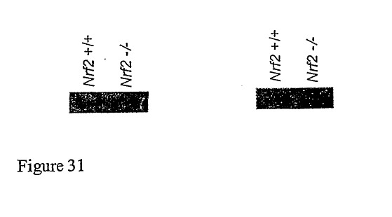

Further, FIG. 31 shows the result of Western blot analysis to examine the levels of TLR4 and CD14 from whole cell extracts obtained from peritoneal macrophages of nrf2−/− and nrf2+/+ mice. Constitutive protein levels of TLR4 are shown in the left panel, and protein levels of CD14 are shown in the right panel. Nrf2−/− mice show increased levels of TLR4 and CD14.

Taken together, similar to the response to LPS, treatment with TNF-α also induced greater inflammation in nrf2−/− lungs.

### Example 17

### NF-κB Activity is Greater in Lungs of LPS Treated Nrf2-Deficient Mice

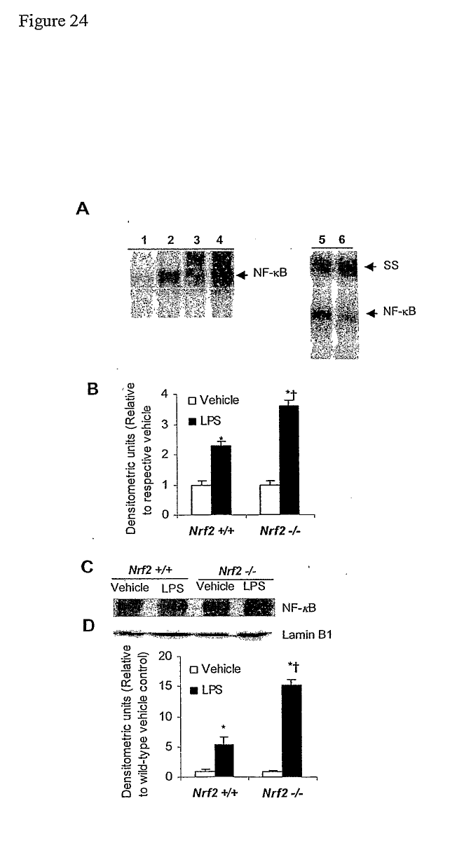

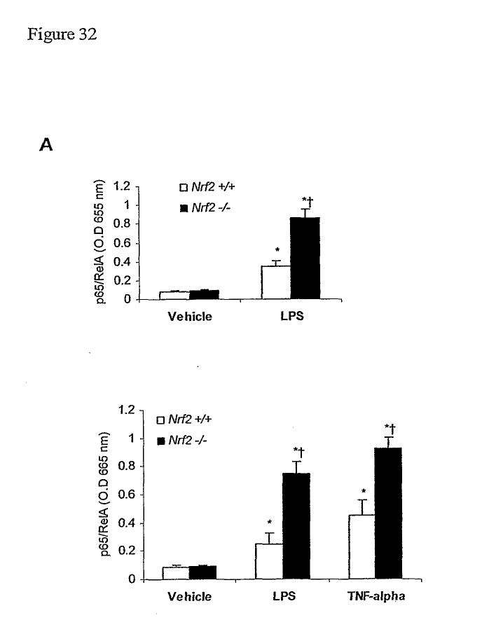

Because the lungs of nrf2−/− mice showed greater infiltration of inflammatory cells and higher expression of largely inflammation-associated genes, NF-κB activity, which regulates the expression of several genes that are essential for initiating and promoting inflammation, was assessed. At 30 min after LPS instillation, NF-κB-DNA binding activity was significantly higher in nuclear extracts from lungs of nrf2−/− mice than their wild-type counterparts suggesting an inhibitory role of nrf2 on NF-κB activation (FIGS. 24 A and B). Western blot analysis confirmed a greater increase in nuclear levels of p65, an NF-κB subunit, in the LPS-treated lungs of nrf2−/− mice than in nrf2+/+ mice (FIGS. 24C and D). Similarly, nuclear extracts from the lungs of nrf2−/− mice showed increased binding of p65/RelA subunits to NF-κB binding sequence as measured by ELISA using Mercury TransFactor ELISA kit (FIG. 32 B). A similar trend towards increased NF-κB activation in nrf2−/− mice was observed at 30 min and 1 h following ip injection of LPS at a non-lethal dose.

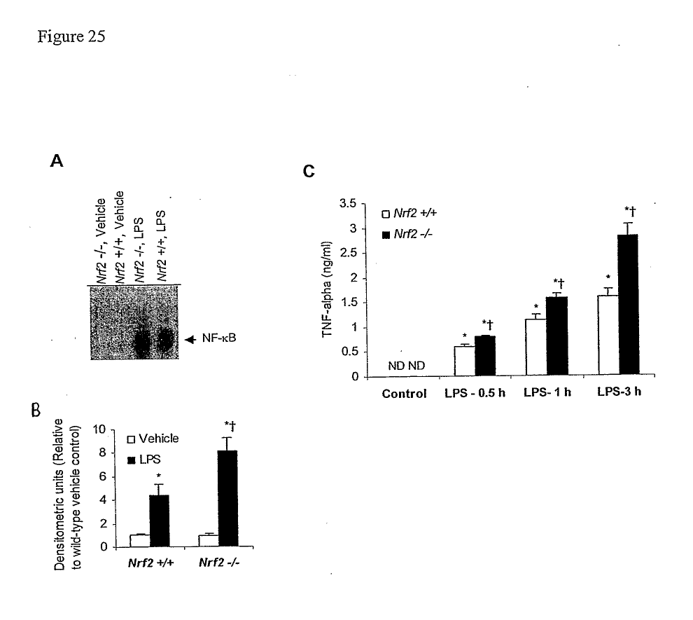

Macrophages play a central role in immune dysfunction during endotoxic shock. To examine the effect of nrf2 deficiency on NF-κB activation in macrophages, resident peritoneal macrophages were stimulated with LPS. After 20 min, the DNA binding activity of NF-κB was substantially higher in nrf2−/− macrophages than in the wild-type counterparts as determined by EMSA (FIGS. 25 A and B). The greater increase in NF-κB activity in nrf2−/− macrophages correlated well with the increase in TNF-α levels measured 0.5 h, 1 h and 3 h after LPS treatment (FIG. 25 C). This data shown that LPS induces greater NF-κB activity and TNF-α secretion in peritoneal macrophages from nrf2-deficient mice.

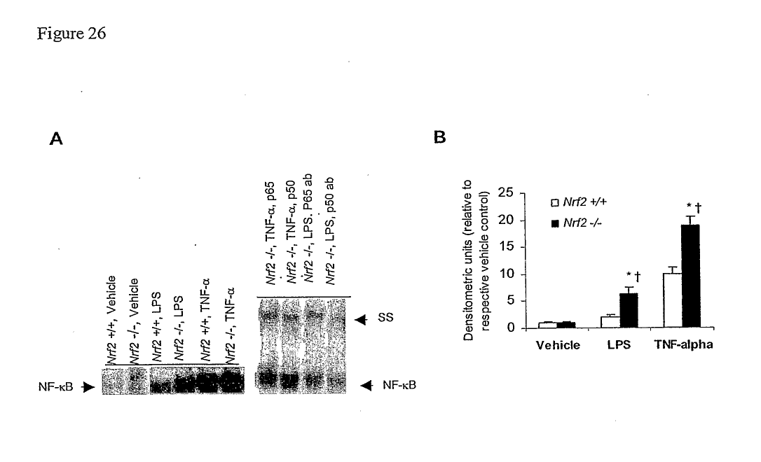

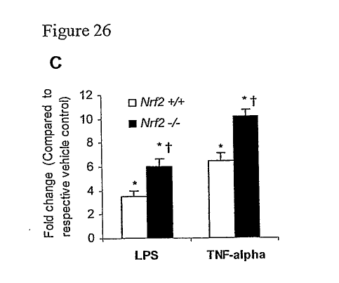

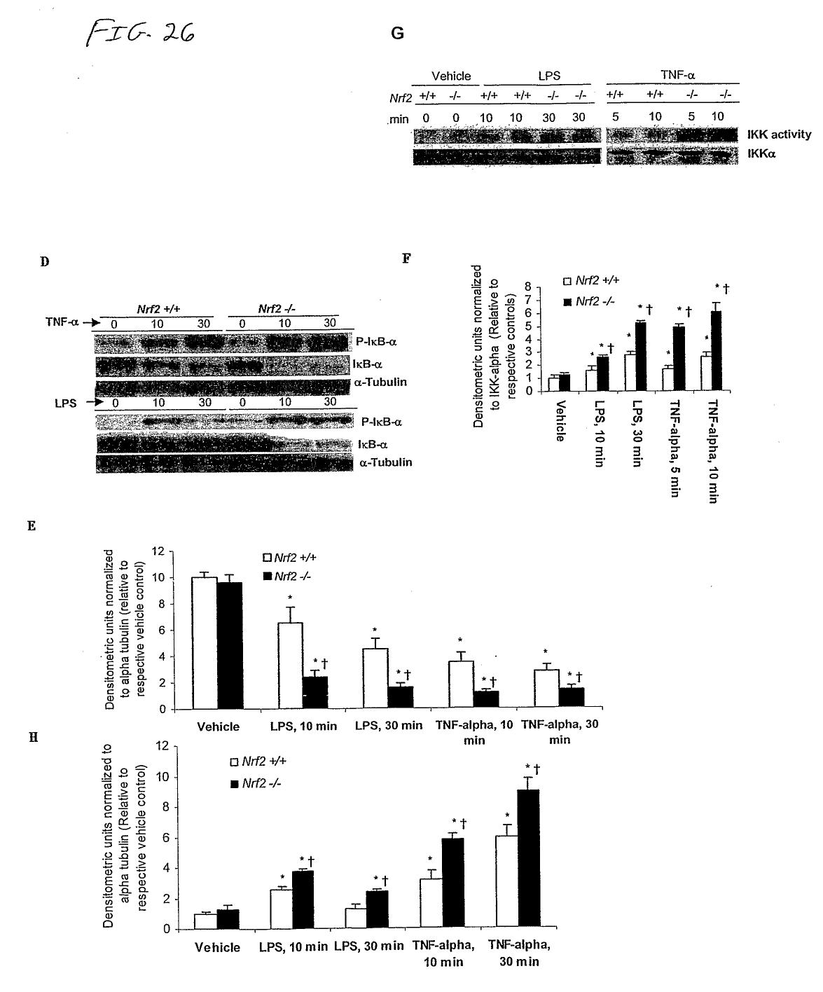

To further probe the role of Nrf2 in regulating NF-κB, mouse embryonic fibroblasts (MEFs) derived from nrf2−/− and nrf2+/+ mice were exposed to LPS or TNF-α. Both LPS and TNF-α stimulation resulted in enhanced activation of NF-κB in nrf2−/− MEFs compared to nrf2+/+ cells as measured by EMSA (FIG. 26 A). There were 3- and 5-fold increases in NF-κB activation in nrf2−/− MEFs relative to wild-type in response to LPS or TNF-α stimulation, respectively (FIG. 26 B). The specificity of NF-κB binding was assessed by adding an excess of cold mutant NF-κB oligo to the binding reactions. Supershift analysis of nuclear extracts from LPS and TNF-α treated nrf2−/− MEFs with p65 and p50 antibody demonstrated heterodimers of p50 and p65. Nuclear extracts from the nrf2−/− MEFs cells treated with LPS or TNF-α also demonstrated increased binding of p65/RelA subunits to NF-κB binding sequence as determined by ELISA based method of detecting NF-κB-DNA binding activity using Mercury TransFactor ELISA kit (FIG. 32 B). NF-κB mediated luciferase reporter activity was also greater in nrf2−/− MEFs than the nrf2+/+ MEFs in response to LPS or TNF-α (FIG. 26 C). In general, the nrf2−/− MEFs showed greater NF-κB activation in response to TNF-α compared to LPS stimulation. Thus, the data shown increased NF-κB activation by LPS or TNF-α in nrf2-deficient mouse embryonic fibroblasts.

### Example 18

### Nrf2 Regulates NF-κB Activation by Modulating IκB-α Degradation

To understand the mechanism of augmented NF-κB activation in nrf2−/− MEFs, and phosphorylated IκB-α (P-IκB-α) was measured in the whole cell extracts of nrf2−/− and nrf2+/+ MEFs after treatment with LPS or TNF-α. In response to LPS or TNF-α, IκB-α degradation was significantly higher in nrf2−/− MEFs compared to wild-type cells (FIGS. 26 D & E). TNF-α stimulus induced greater phosphorylation of IκB-α while LPS induced moderate but statistically significant increase in phosphorylation of IκB-α in nrf2−/− MEFs compared to nrf2+/+ MEFs (FIGS. 26 D & F). Furthermore, activity of IKK kinase, which regulates phosphorylation of IκB-α was also greater in nrf2−/− MEFs in response to LPS or TNF-α (FIGS. 26G and H)

### Example 19

### Nrf2 Affects Both MyD88-Dependent and MyD88-Independent Signaling

Microarray gene expression analysis after LPS challenge revealed that, in addition to NF-κB regulated genes; several IRF3 regulated genes (such as IP-10, MIG, ITAC, ISG54; Table 12 were expressed to a greater magnitude in the lungs of nrf2−/− mice.

TABLE 12
Differential expression of IRF3 regulated genes in lungs of nrf2-deficient and wild-
type mice after LPS stimulus

| | | 30 min | | 1 h | | 6 h | |
|---|---|---|---|---|---|---|---|
| | Gene | (LPS/Vehicle) | | (LPS/Vehicle) | | (LPS/Vehicle) | |
| Gene title | symbol | Nrf2−/− | Nrf2+/+ | Nrf2−/− | Nrf2+/+ | Nrf2−/− | Nrf2+/+ |
| Chemokine (C—X—C | CXCL10 | 14.7 ± .6 | 4.3 ± 0.5 | 40.5 ± 0.5 | 25.8 ± 0.4 | 187.4 ± 0.6 | 112.2 ± 0.4 |
| motif) ligand 10 | (IP-10) | | | | | | |
| (Gamma-IP10) | | | | | | | |
| Chemokine (C—X—C | CXCL11 | — | — | 3.9 ± 0.5 | — | 177.3 ± 0.5 | 198.1 ± 0.8 |
| motif) ligand | (ITAC) | | | | | | |
| 11(Interferon-inducible | | | | | | | |
| T-cell alpha | | | | | | | |
| chemoattractant) | | | | | | | |
| Chemokine (C—X—C | CXCL9 | 14.7 ± 0.5 | — | 11.7 ± 0.5 | — | 820.3 ± 0.5 | 576.0 ± 0.5 |
| motif) ligand 9 (Gamma | (MIG) | | | | | | |
| inter-feron induced | | | | | | | |
| monokine) | | | | | | | |
| Epstein-Barr virus | Ebi3 | — | — | 9.6 ± 0.4 | 12.2 ± 0.4 | 8.8 ± 0.4 | 6.2 ± 0.4 |
| induced gene 3 | | | | | | | |
| Immune-responsive | IRG1 | 286.0 ± 0.6 | 1858 ± 0.4 | 552 ± 0.4 | 313 ± 0.4 | 53 ± 0.4 | 29 ± 0.8 |
| gene 1 | | | | | | | |
| Interferon activated | IFI202B | 2.5 ± 0.4 | — | 3.5 ± 0.5 | 1.9 ± 0.5 | 39.4 ± 0.4 | 21.0 ± 0.4 |
| gene 202B | | | | | | | |
| Interferon activated | IFI204 | 4.3 ± 0.4 | — | 4.8 ± 0.7 | 1.9 ± 0.5 | 31.8 ± 0.4 | 29.9 ± 0.4 |
| gene 204 | | | | | | | |
| Interferon regulatory | IRF1 | 5.7 ± 0.4 | 4.2 ± 0.4 | 4.5 ± 0.4 | 3.7 ± 0.4 | 4.9 ± 0.4 | 4.5 ± 0.4 |
| factor 1 | | | | | | | |
| Interferon regulatory | IRF5 | 1.7 ± 0.4 | — | 2.4 ± 0.4 | 1.7 ± 0.4 | 3.8 ± 0.4 | 3.1 ± 0.4 |
| factor 5 | | | | | | | |
| Interferon regulatory | IRF7 | — | — | 1.9 ± 0.4 | — | 22.6 ± 0.4 | 15.6 ± 0.4 |
| factor 7 | | | | | | | |
| Interferon-induced | IFI44 | — | — | — | — | 17.9 ± 0.4 | 10.6 ± 0.4 |
| protein 44 | | | | | | | |
| Interferon-induced | IFIT2 | — | — | — | — | 39.9 ± 0.4 | 23.1 ± 0.4 |
| protein with tetra- | | | | | | | |
| tricopeptide repeats 2 | | | | | | | |
| (ISG54) | | | | | | | |
| Interferon-induced | IFIT3 | — | — | — | — | 18.4 ± 0.4 | 9.9 ± 0.4 |
| protein with tetra- | | | | | | | |
| tricopeptide repeats 3 | | | | | | | |
| (GARG-49) | | | | | | | |
| Myxovirus (influenza | Mx1 | — | — | — | 2.1 ± 0.5 | 49.9 ± 0.4 | 23.8 ± 0.4 |
| virus) resistance 1 | | | | | | | |

| | | 12 h | | 24 h | |
|---|---|---|---|---|---|
| | Gene | (LPS/Vehicle) | | LPS/Vehicle | |
| Gene title | symbol | Nrf2−/− | Nrf2+/+ | Nrf2−/− | Nrf2+/+ |
| Chemokine (C—X—C | CXCL10 | 40.2 ± 0.6 | 34.3 ± 0.4 | 5.0 ± 0.7 | 5.6 ± 0.4 |
| motif) ligand 10 | (IP-10) | | | | |
| (Gamma-IP10) | | | | | |
| Chemokine (C—X—C | CXCL11 | 24.8 ± 0.5 | 41.6 ± 0.9 | — | — |
| motif) ligand | (ITAC) | | | | |
| 11(Interferon-inducible | | | | | |
| T-cell alpha | | | | | |
| chemoattractant) | | | | | |
| Chemokine (C—X—C | CXCL9 | 837.5 ± 0.5 | 739.3 ± 0.6 | 116.2 ± 0.7 | 68.6 ± 0.7 |
| motif) ligand 9 (Gamma | (MIG) | | | | |
| inter-feron induced | | | | | |
| monokine) | | | | | |
| Epstein-Barr virus | Ebi3 | 8.2 ± 0.4 | 6.7 ± 0.4 | 4.2 ± 0.5 | 4.0 ± 0.4 |
| induced gene 3 | | | | | |
| Immune-responsive | IRG1 | 1082 ± 0.4 | 304 ± 0.5 | 183.5 ± 0.7 | 64.1 ± 0.5 |
| gene 1 | | | | | |
| Interferon activated | IFI202B | 14.9 ± 0.4 | 8.7 ± 0.4 | 6.5 ± 0.4 | 4.8 ± 0.4 |
| gene 202B | | | | | |
| Interferon activated | IFI204 | 12 ± 0.5 | 9.4 ± 0.4 | 7.1 ± 0.5 | 3.7 ± 0.4 |
| gene 204 | | | | | |
| Interferon regulatory | IRF1 | 2.5 ± 0.4 | 2.4 ± 0.4 | — | — |
| factor 1 | | | | | |
| Interferon regulatory | IRF5 | 2.5 ± 0.4 | 2.2 ± 0.4 | 2.2 ± 0.4 | 2.1 ± 0.4 |
| factor 5 | | | | | |
| Interferon regulatory | IRF7 | 16.3 ± 0.4 | 13.1 ± 0.4 | 7.7 ± 0.5 | 6.0 ± 0.4 |
| factor 7 | | | | | |
| Interferon-induced | IFI44 | 6.6 ± 0.4 | 5.5 ± 0.4 | 3.1 ± 0.4 | 1.8 ± 0.4 |
| protein 44 | | | | | |
| Interferon-induced | IFIT2 | 11.8 ± 0.6 | 8.2 ± 0.5 | 2.5 ± 0.5 | 2.1 ± 0.4 |
| protein with tetra- | | | | | |
| tricopeptide repeats 2 | | | | | |
| (ISG54) | | | | | |
| Interferon-induced | IFIT3 | 6.3 ± 0.4 | 5.8 ± 0.4 | 2.9 ± 0.5 | 2.4 ± 0.4 |
| protein with tetra- | | | | | |
| tricopeptide repeats 3 | | | | | |
| (GARG-49) | | | | | |
| Myxovirus (influenza | Mx1 | 6.9 ± 0.7 | 4.7 ± 0.4 | 2.1 ± 0.4 | 1.9 ± 0.5 |
| virus) resistance 1 | | | | | |

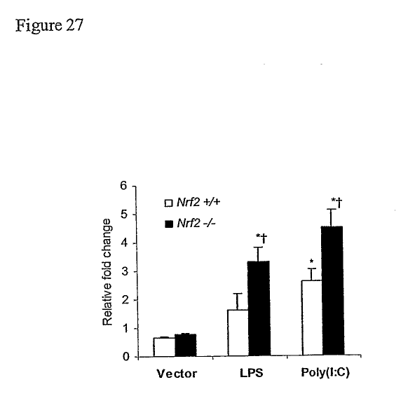

PS via TLR4 can activate Myd88-dependent signaling leading to NF-κB activation as well as Myd88-independent signaling (TRIF/IRF3) resulting in IRF3 activation (Doyle S et al. Immunity 17:251-263.2002). As shown in FIG. 26 C, Nrf2 deficiency upregulates NF-κB mediated luciferase activity in MEFs in response to LPS, thus suggesting effect on MyD88-dependent signaling. In order to understand the influence of Nrf2 deficiency on MyD88-independent signaling, MEFs of both genotypes were transfected with a luciferase reporter vector containing interferon stimulated response element (ISRE) and treated with LPS or poly (I:C). LPS elicited greater IRF3-mediated luciferase reporter activity in nrf2−/− MEFs compared to nrf2+/+ MEFs (FIG. 27). Similarly, in response to poly(I:C), which acts specifically via MyD88-independent signaling (Yamamoto M et al. Science 301:640-643.2003), IRF3 mediated reporter activity was significantly higher in nrf2−/− MEFs (FIG. 27).

### Example 20

### Glutathione Levels are Lower in Lungs and Mouse Embryonic Fibroblasts of nrf2-Deficient Mice

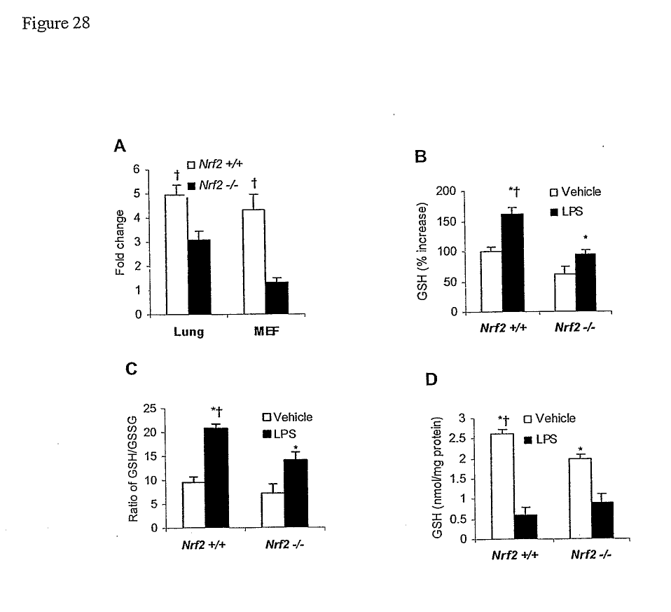

Nrf2 is a regulator of a battery of cellular antioxidants, including glutathione-synthesizing enzyme, glutamate cysteine ligase. Constitutive expression of glutamate cysteine ligase catalytic subunit (GCLC) was significantly lower in the lungs as well as MEFs of nrf2−/− mice compared to nrf2+/+ mice (FIG. 28 A). This difference in expression is reflected in significantly lower endogenous levels of GSH in the lungs and MEFs of nrf2−/− mice than in nrf2+/+ mice (FIGS. 28 B & C). In response to LPS stimulus, there was a significant decrease in the levels of GSH in MEFs of both genotypes at 1 h (FIG. 28 C). By contrast, after 24 h of LPS treatment a greater increase in GSH was observed in the lungs of nrf2+/+ mice compared to nrf2−/− (FIG. 28 B). The ratio of GSH to oxidized glutathione (GSSG) after LPS challenge was significantly higher in the lungs of wild-type mice, implying greater amounts of GSSG in nrf2−/− lungs and thus a difference in redox status between the two genotypes (FIG. 28 D).

### Example 21

### N-Acetyl Cysteine (NAC) and GSH-Monoethyl Ester Decrease LPS and TNF-α Induced NF-κB Activation in Nrf2-Deficient MEFs

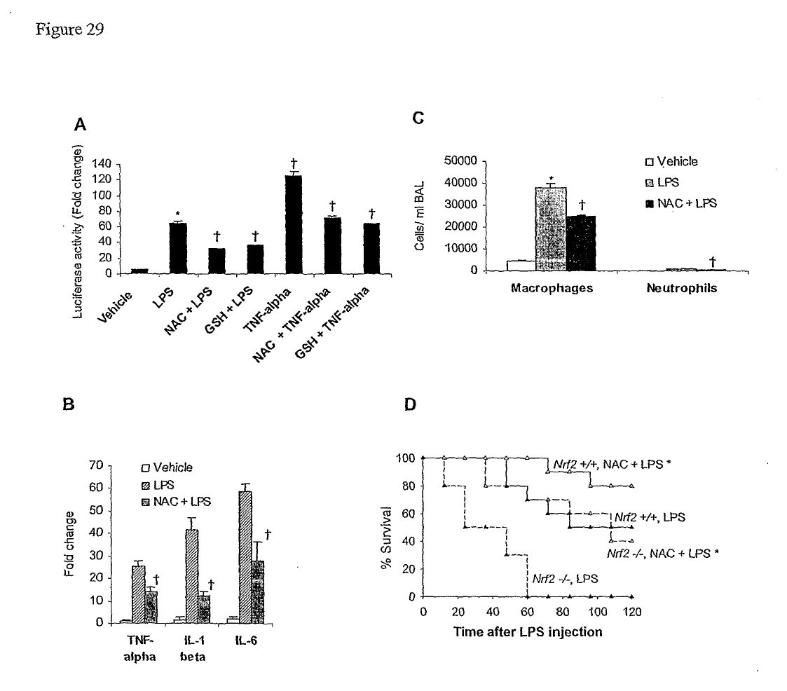

To investigate whether replenishing antioxidants could suppress the enhanced NF-κB activation observed in nrf2−/− cells, MEFs transfected with NF-κB-luc reporter vector were pretreated with NAC or GSH-monoethyl ester for 1 h and then challenged with LPS or TNF-α. Pretreatment with NAC or GSH-monoethyl ester, significantly attenuated NF-κB mediated reporter activity in nrf2−/− cells elicited in response to LPS or TNF-α (FIG. 29 A).

Since LPS challenge enhanced the expression of several NF-κB regulated proinflammatory genes in lungs of nrf2−/− mice compared to wild-type litter mates, administration of an exogenous antioxidant could attenuate this augmented proinflammatory cascade was examined. Mice were pretreated with NAC (500 mg/kg body weight) and then challenged with non-lethal dose of LPS. After 30 min of LPS challenge, selected proinflammatory genes were measured by real time PCR analysis. Transcript levels of TNF-α IL-1β and IL-6 were significantly reduced in the lungs of nrf2−/− mice by pretreatment with NAC (FIG. 29 B). Influx of inflammatory cells was also significantly reduced by pretreatment of nrf2−/− mice with NAC (FIG. 29 C). Next, exogenous NAC supplementation was examined as providing protection against LPS induced septic shock in nrf2−/− mice. Mice of both genotypes were pretreated with NAC (500 mg/kg body weight) for 4 days prior to LPS challenge (1.5 mg per mouse). All nrf2−/− mice pretreated with saline died within 56 h while 40% of mice pretreated with NAC survived (FIG. 29 D). Pretreatment of wild-type mice with NAC provided modest protection. These results suggest that exogenous antioxidants such as NAC can partially ameliorate the phenotype of nrf2−/− mice.

### Example 22

### Comparison of Rigid and Flexible Probe: Effects on Stroke, Subarachnoid Hemorrhage and Mortality

Intraluminal occlusion of the middle cerebral artery in rodents is widely used for investigating cerebral ischemia and reperfusion injury. Recently, many studies have been published that have used different types of filaments to induce transient or permanent occlusion of the middle cerebral artery (MCA) in rodents (Bonventre J V et al. Nature; 390:622-625. 1997; Sharp Fr et al. J Cereb Blood Flow Metab 20:1011-1032.2000; Chen J F et al. J Neurosci:19: 9192-9200. 1999; Pan Y et al. Brain Res. 1043:195-204.2-5. 2005). Filaments or sutures can vary in size from 4-0 to 8-0, and have produced promising effects in MCA occlusion (MCAO) studies (Pan Y et al. Brain Res. 1043:195-204.2005; Shah Z A et al. Pharmacol Toxicol. 90:254-259.2005; Namiranian K et al. Curr Neurovasc Res. 22:23-27.2005).

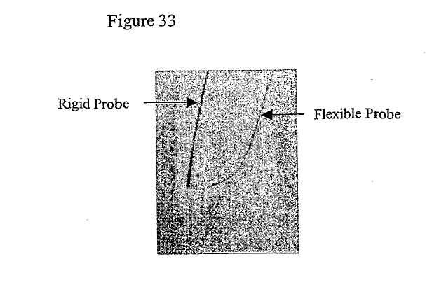

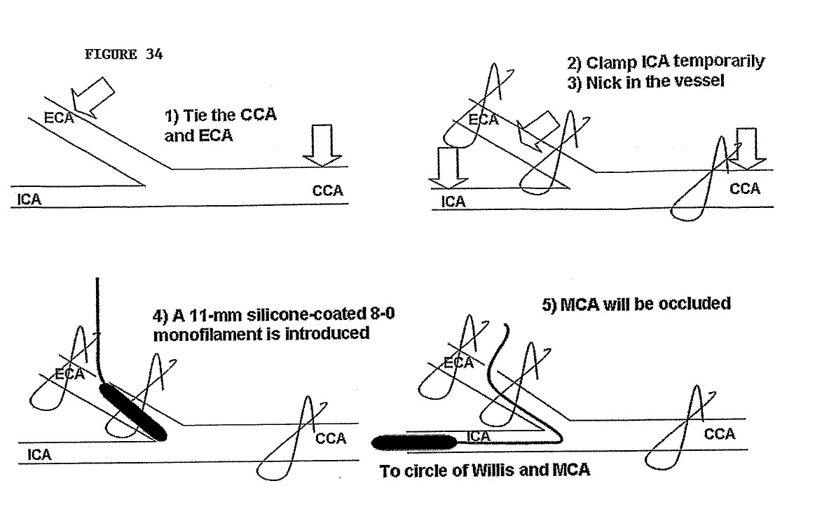

FIG. 33 shows the rigid and flexible probes. The probe on the left is a 6-0 monofilament that was preheated and coated with methyl methacrylate glue. This is the rigid probe. The probe on the right is an 8-0 monofilament coated with silicone. This is the flexible probe. FIG. 34 is a schematic diagram showing the technique of middle cerebral artery occlusion with 8-0 monofilament coated with silicone (flexible probe).

Here, the percentage of successful strokes observed in WT mice was 46.6% with rigid probe and 73.5% with flexible probe (P<0.05). In addition, subarachnoid hemorrhage occurred much less frequently (3.7%) with flexible probes than with rigid probes (26.6%) in WT mice (P<0.01; Table 13).

TABLE 13
Evaluation of nonparametric parameters

| | | | | | | | Failed | |
|---|---|---|---|---|---|---|---|---|
| | | | | | | Failure to | Surgery | |
| | | Number of | | Subarachnoid | | induce | for other | Mortality |
| Mouse | Probe | successful | | Hemorrhage | | lesion [n, | reasons | Rate [n, |
| Strain (n) | used | strokes (%) | | [n, (%)] | | (%)] | [n, (%)] | (%)] |
| WT (45) | Rigid | 21 | (46.6%) | 12 | (26.6%) | 4 (8.8%) | 3 (6.6%) | 5 (11.1%) |
| WT (53) | Flexible | 39 | (73.5%)* | 2 | (3.7%)* | 5 (9.4%) | 4 (7.5%) | 3 (5.6%)* |
| WT (10) | Rigid | 8 | (80%) | 1 | (10%) | 0 (0%) | 0 (0%) | 1 (10%) |
| HO-1^{-/-} | Rigid | 6 | (60%) | 2 | (20%) | 0 (0%) | 0 (0%) | 2 (20%) |
| (10) | | | | | | | | |
| WT (7) | Flexible | 7 | (100%)* | 0 | (0%)* | 0 (0%) | 0 (0%) | 0 (0%)* |
| HO-1^{-/-} | Flexible | 7 | (100%)* | 0 | (0%)* | 0 (0%) | 0 (0%) | 0 (0%)* |
| (7) | | | | | | | | |

Rigid probe: 6-0 filament coated with methyl methacrylate.
Flexible probe: 8-0 monofilament coated with silicone.

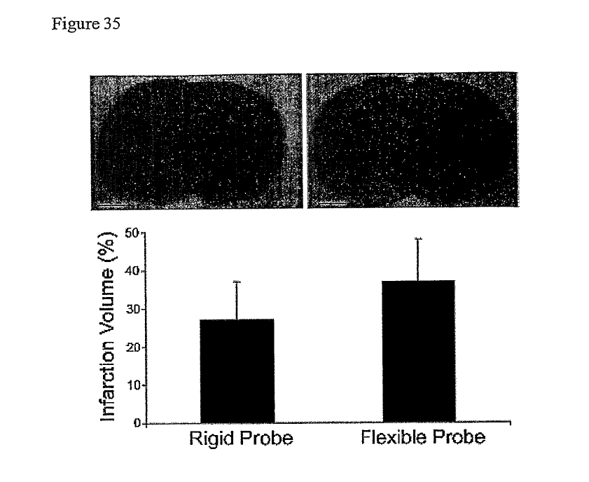

Table 13 illustrated that the incidence of subarachnoid hemorrhage was significantly lower with flexible probes than with the rigid probes (P<0.01). Further, the success rate was higher with the flexible probes (P<0.05). Subarachnoid hemorrhage was considerably less in WT (10%) than in HO-1^{-/-} mice (20%) when rigid probes were used. No mortality occurred after middle cerebral artery occlusion in mice that received the flexible probe. *P<0.05 versus use of rigid probe. Further, mortality was significantly lower (P<0.05) with the flexible probe (5.6%) than with the rigid probe (11.1%). However, the type of probe used did not affect the infarction volume in WT mice, as no significant differences were observed in cerebral infarction volume between rigid probe (27.0±3.3) and flexible probe (37.0±3.6) (FIG. 35).

### Example 23

### Comparison of Rigid and Flexible Probe-Effect on Cerebral Infarction Volume

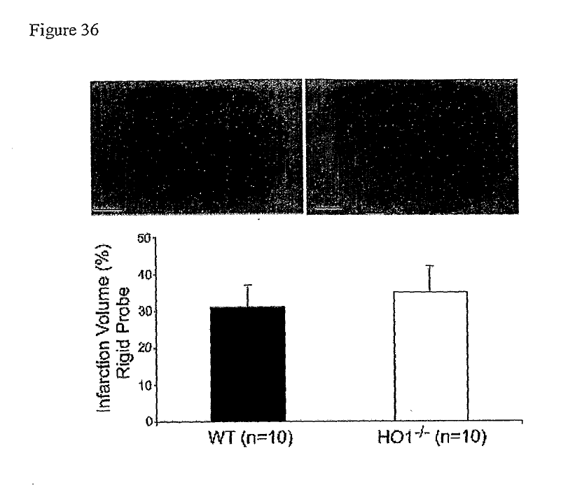

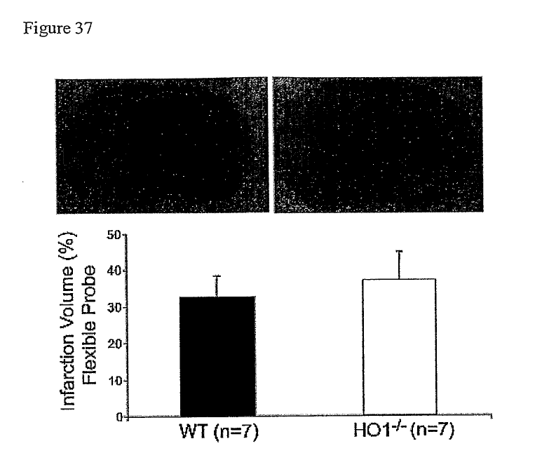

A comparison of the effect of rigid and flexible probes on cerebral infarction volume was carried out. No significant difference in cerebral infarction volume was observed between HO-1^{-/-} and WT mice with either the rigid or flexible probe. The percentage-corrected infarction with the rigid probe represented 31.0±2.0% of the hemisphere in WT mice (n=10) and 35.0±2.3% of the hemisphere in HO-1^{-/-} mice (n=10) (FIG. 36). The percentage corrected infarction with the flexible probe represented 32.7±5.6% of the hemisphere in WT mice (n=7) and 37.1±7.8% of the hemisphere in Ho-1−/− mice (n=7), as shown in FIG. 37.

Two of the ten (20.0%) HO-1^{-/-} mice that received the rigid probe died, whereas only one of the ten (10.0%) WT mice died. Of 20 surgeries that used the rigid probe, two cases of subarachnoid hemorrhage in HO-1^{-/-}, and only one case in WT mice was observed. However, the percentage of successful strokes was significantly higher in WT mice (80.0%) than in HO-1^{-/-} mice (60.0%, P<0.05; Table 13, above). Of the 14 surgeries in WT and HO-1^{-/-} mice that made use of the flexible probe, all were successful. None of these mice suffered a subarachnoid hemorrhage, and there were no mortalities as shown above in Table 13. Finally, the neurological scores obtained after 24 h of reperfusion were not significantly different between the two stroke methods or between the WT and HO-1^{-/-} mice.

Taken together, the data presented herein demonstrated that the flexible filament substantially increases the rate of successful strokes and survival. Thus, this novel model may provide an easier and more reproducible alternative for inducing stroke in mice than previously used models.

### Example 24

### MCA Occlusion and Reperfusion

Nuclear factor erythroid 2-related factor 2 (Nrf2), a basic leucine zipper transcriptional factor, coordinately upregulates antioxidant-responsive element-mediated gene expression. Recent work has indicated a unique role for Nrf2 in various physiological stress conditions, but its contribution to ischemic-reperfusion injury has not been ascertained.

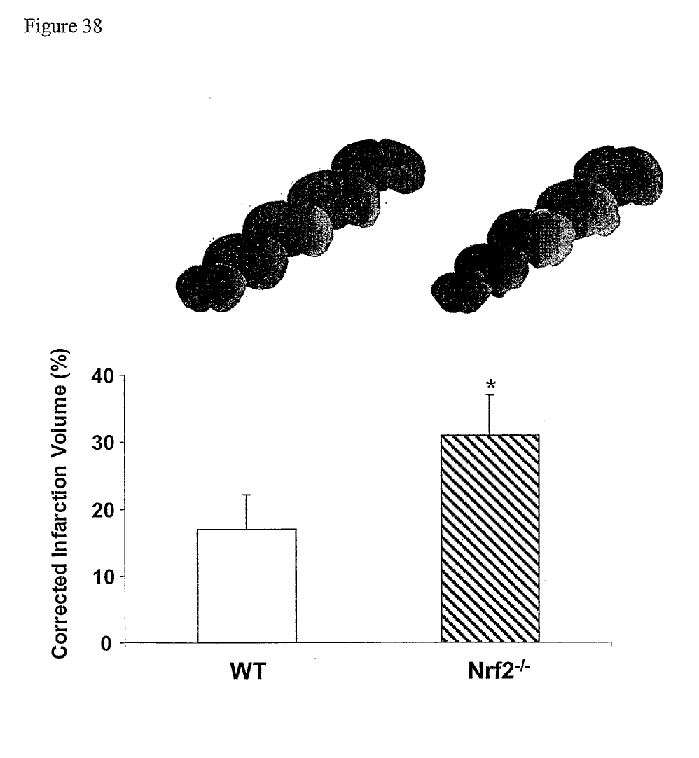

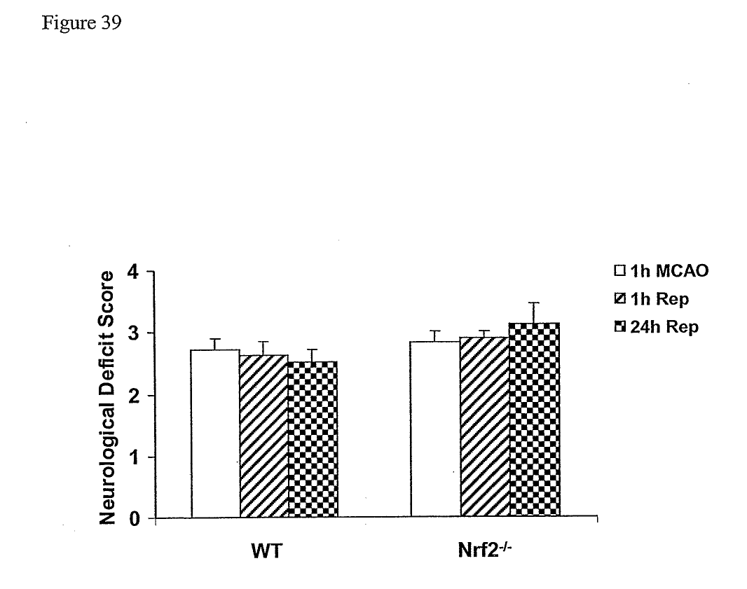

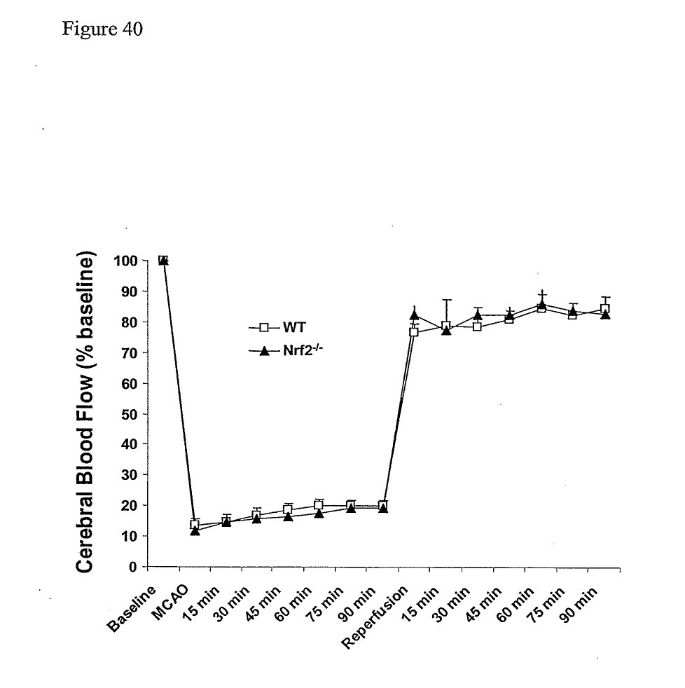

Here, 2,3,5-triphenyltetrazolium chloride (TTC) staining revealed that the percentage corrected ischemic region of the Nrf2^{-/-} mice (30.8±6.1%) was significantly larger than that of the WT mice (17.0±5.1%; P<0.01) (FIG. 38). Additionally, neurological deficit was significantly greater in the Nrf2^{-/-} mice (3.1±0.3) than in the WT mice (2.5±0.2) 24 hours after ischemia, P<0.04 (FIG. 39). In a second cohort of mice, no significant differences in cerebral blood flow (CBF) were observed in the WT and Nrf2^{-/-} mice at any time point during MCA occlusion (MCAO) or reperfusion. Relative cerebral blood flow in the MCA territory was reduced to the same level during occlusion in WT and Nrf2^{-/-} mice (13.5±2.0% and 11.9±1.8% of baseline, respectively; FIG. 40). Finally, blood drawn 30 minutes before MCAO, 1 hour after MCAO, and 1 hour after reperfusion revealed that blood gases were within the physiological range before and during surgery and were not different between the groups (Table 14). Together, this data shows that the the corrected ischemic region of the Nrf2^{-/-} mice was significantly larger than that of the WT mice, and further, neurological deficit was greater in the Nrf2^{-/-} mice than in the WT mice.

TABLE 14
Blood gas measurements before, during and after middle cerebral artery occlusion.

| | WT | | | Nrf2^{-/-} | | |
|---|---|---|---|---|---|---|
| | 1 h before | 1 h after | 1 h after | 1 h before | 1 h after | 1 h after |
| Parameter | MCAO | MCAO | Reperfusion | MCAO | MCAO | Reperfusion |
| pH | 7.39 ± 0.01 | 7.39 ± 0.02 | 7.40 ± 0.04 | 7.40 ± 0.02 | 7.30 ± 0.04 | 7.40 ± 0.03 |
| PaCO₂ | 44.0 ± 1.7 | 44.2 ± 1.9 | 44.2 ± 1.9 | 46.0 ± 2.3 | 45.2 ± 2.6 | 45.2 ± 2.0 |
| PaO₂ | 122 ± 6 | 127 ± 5 | 128 ± 6 | 128 ± 4 | 128 ± 4 | 128 ± 6 |

Data are given as mean ± SE.

### Example 25

### t-BuOOH, Glutamate, and NMDA-Mediated Effects on Nrf2

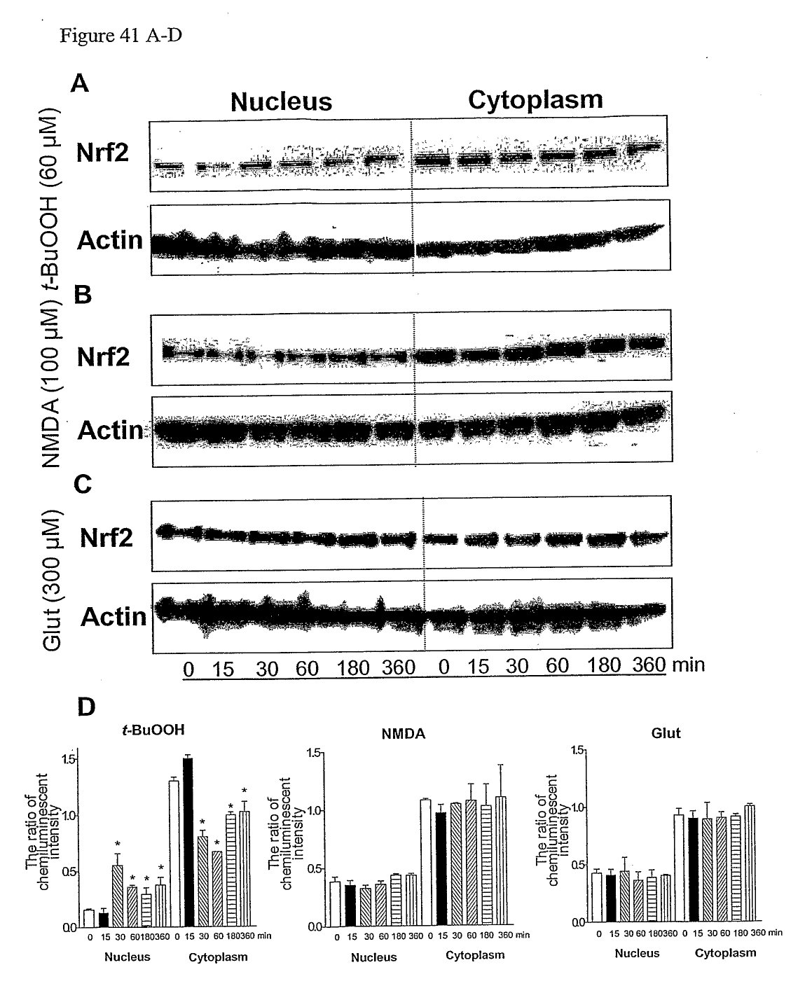

Mouse cultured cortical neurons were exposed to test-butyl hydroperoxide t-BuOOH, glutamate, or NMDA to determine the effects of these compounds on Nrf2 location in the nuclear and cytosolic fractions. t-BuOOH induced time-dependent changes in Nrf2 presence in the nuclear fraction. Protein expression was elevated at 30 min, and continued to increase through the full time course of the experiment, 360 minutes (FIG. 41 A). In the cytosolic fraction, Nrf2 remained at baseline levels for 15 minutes, and then decreased to below the basal level after 30 minutes. In contrast, glutamate and NMDA had no effect on Nrf2 expression in either the nuclear or cytosolic fractions (FIGS. 41 B and 41 C). The expression levels of actin were unaffected by any of the treatments shown in A-C. FIG. 41 D shows the ratio of chemiluminescence emitted from the Nrf2 to that for the actin of each sample.

### Example 26

### Effect of the Nrf2 Inducer Tert-Butylhydroquinone (t-BHQ) on Cell Death Induced by t-BuOOH, NMDA, and Glutamate

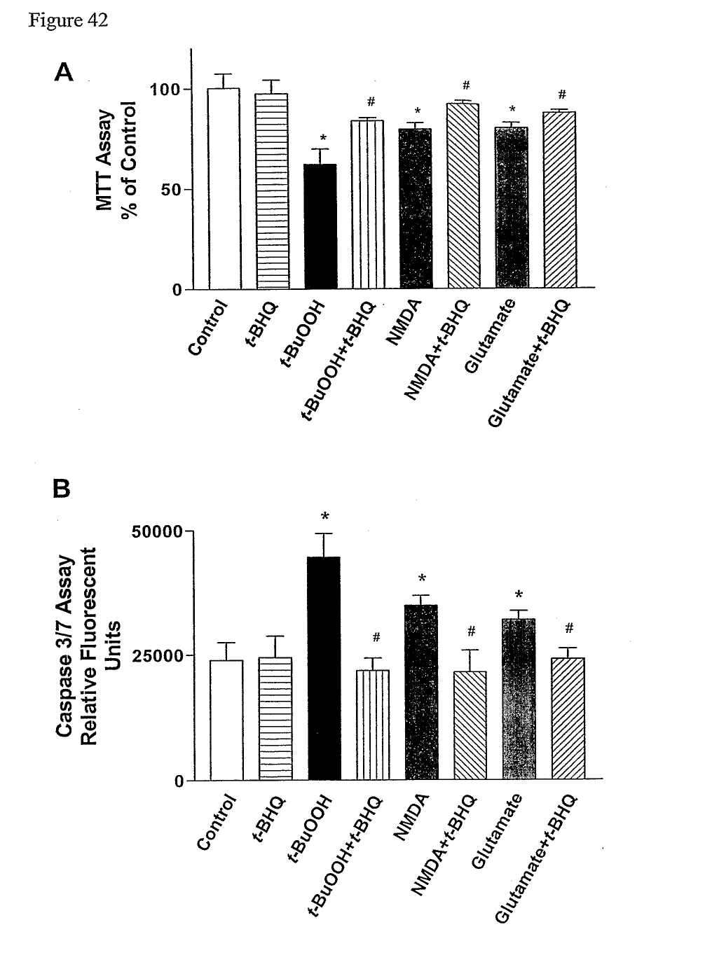

Application of t-BuOOH (60 μM), NMDA (100 μM), and glutamate (300 μM) each significantly decreased the number of viable neurons after 24 hours, compared to the number of untreated control neurons (FIG. 42 A). This decrease was abolished by 20 μM t-BHQ (tert-butylhydroquinone). Furthermore, t-BHQ alone had no effect on neuronal viability.

To substantiate the protection observed by t-BHQ treatment, the activity of caspase-3 was examined. Caspase-3 has been described as a terminal effector of the apoptotic-like cell death pathway. t-BuOOH, NMDA and glutamate each induced an increase in caspase-3 activity (FIG. 42 B). t-BHQ had no effect on basal levels of caspase-3 activity, but was able to prevent the increase evoked by all three stressors (FIG. 42 B).

Taken together, the above data suggests that 1) Nrf2 translocation mediated by oxidative stress-induced injury is protective in cultured neurons, and 2) nuclear Nrf2 increases in response to t-BuOOH-mediated oxidative stress, but not in response to NMDA/glutamate-mediated excitotoxicity.

### Example 27

### EGb 761 Improves Neurological Score

In the central nervous system, Ginkgo biloba extract (EGb 761) has been reported to protect neurons exposed to oxidative stress. Although it is thought that EGb 761 has antioxidative properties, the mechanisms involved in the pharmacologic activity are unclear.

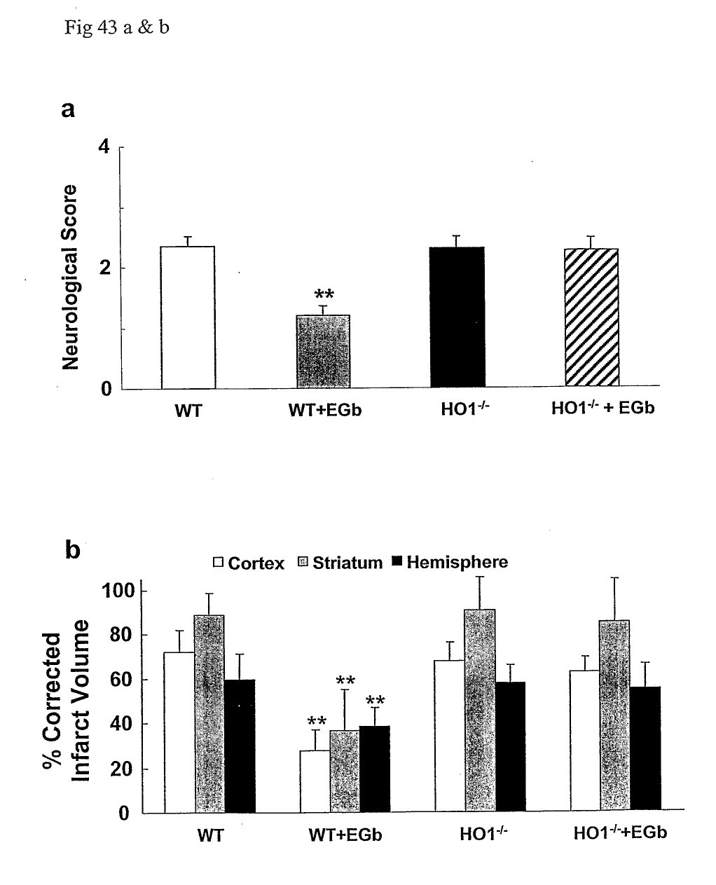

Twenty-four hours after MCAO and reperfusion, WT mice that had been pretreated for 7 d with EGb 761 had significantly less neurological dysfunction (P<0.01) as compared to those that had received vehicle (FIG. 43 a). There was no significant difference in neurological function between HO-1^{-/-} mice that received EGb 761 and those that did not receive EGb 761. Further, there was no difference between vehicle-treated WT and HO-1^{-/-} mice (FIG. 43 a).

### Example 28

### EGb 761 Reduces Infarct Size and Improves CBF

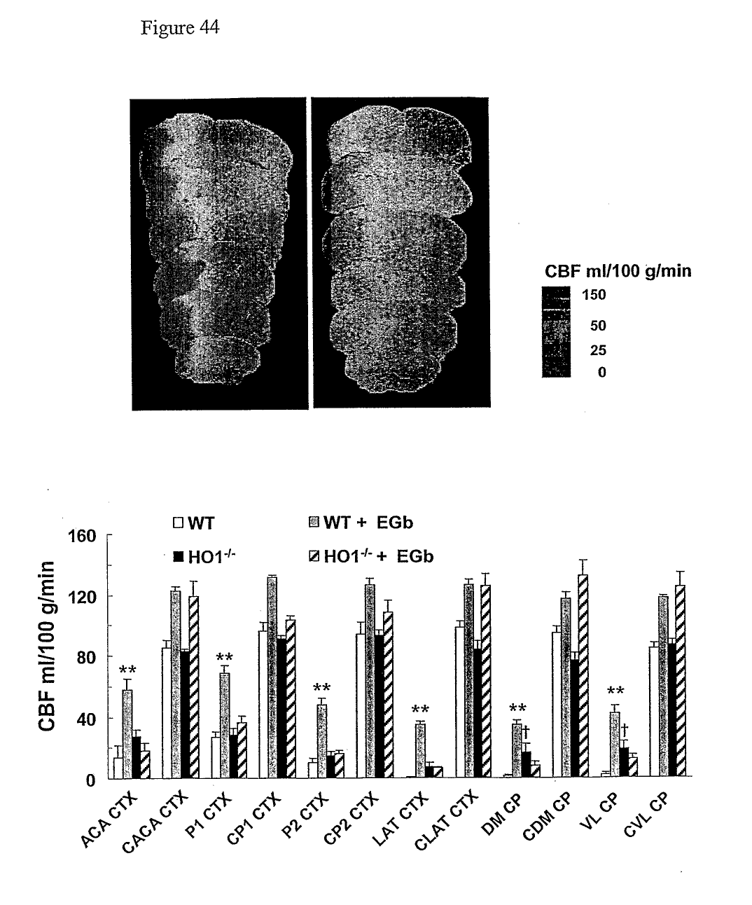

2,3,5-triphenyltetrazolium chloride (TTC) staining revealed that WT mice pretreated for 7 d with EGb 761 had significantly smaller corrected infarct volumes 24 h after MCAO and reperfusion than vehicle-treated mice (P<0.01; FIG. 43 b). EGb 761 treatment did not affect the infarct size of HO-1^{-/-} mice, and there was no significant difference in infarct size between vehicle-treated WT and HO-1^{-/-} mice, as reported in FIG. 43b. To determine the role of EGb 761 in regulation of CBF, CBF was calculated with quantitative [14C]-IAP autoradiography. Potential differences in vascular responsiveness between WT mice treated with vehicle, and those treated with EGb 761 were examined by quantifying absolute regional CBF in the anterior cerebral artery cortex, parietal cortex, lateral cortex, and ventrolateral and dorsomedial caudate putamen of the ipsilateral and contralateral hemispheres (FIG. 44, top panel). After 60 min of MCAO, the ipsilateral CBF (ml/100 g/min) was significantly higher in the EGb 761-treated WT mice than in the vehicle-treated WT mice in all regions measured (FIG. 44, bottom panel; P<0.01).

### Example 29

### EGb 761, but not Bilobalide or Ginkgolides, Induces HO-1

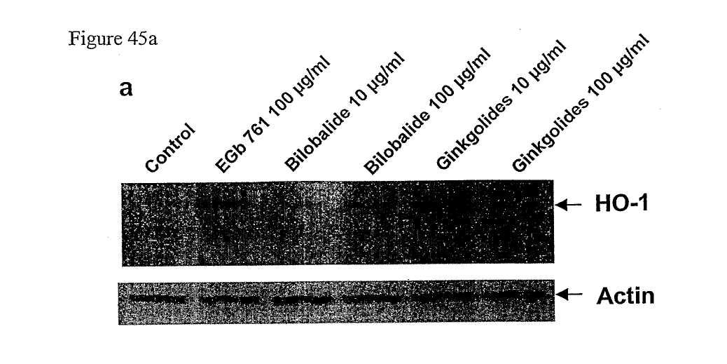

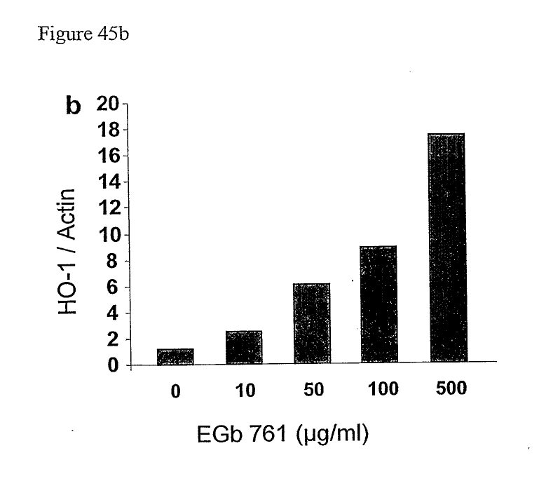

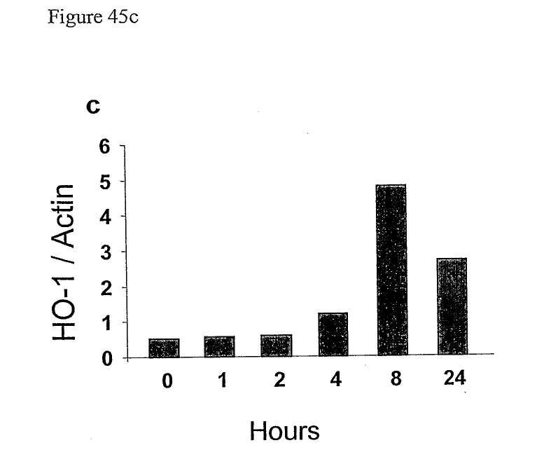

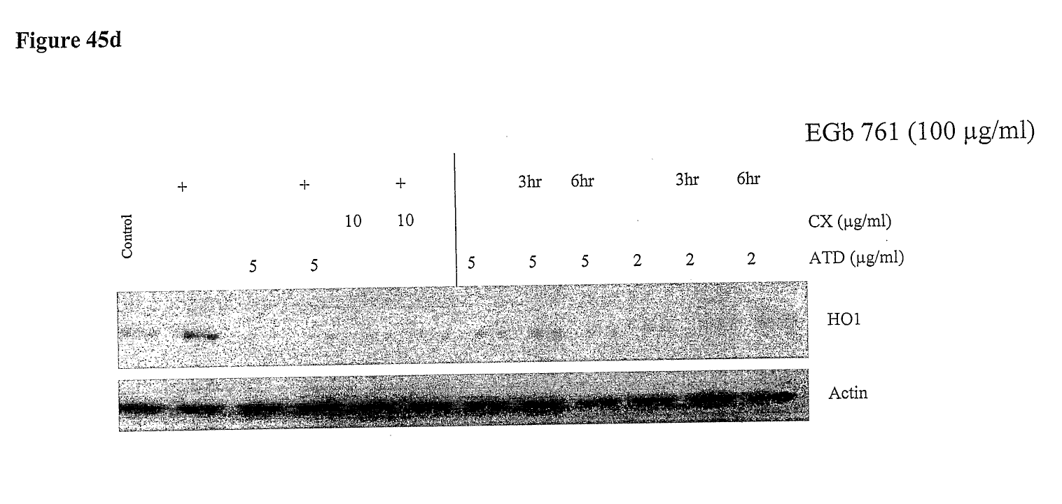

HO-1 protein expression increased in mouse cortical neurons treated for 8 h with EGb 761 (100 μg/ml), but not in those treated with bilobalide (10 and 100 μg/ml) or ginkgolides (10 and 100 μg/ml; FIG. 45a). FIG. 45a shows the results of a Western blot analysis to examine the levels of HO-1. When the cultured neurons were treated for 8 h with various concentrations (0, 10, 50, 100, and 500 μg/ml) of EGb 761, HO-1 induction was evident at a concentration as low as 10 μg/ml and increased in a dose-dependent manner (FIG. 45b). To define the time course of effect of EGb 761 on HO-1 protein expression, cultured neurons were treated with 100 μg/ml EGb 761 for different periods of time (0, 1, 2, 4, 8, and 24 h). The data indicate that EGb 761 can induce HO-1 protein expression after 4 h of treatment and that maximum induction occurs at approximately 8 h (FIG. 45c). Both the protein synthesis inhibitor cycloheximide (CHX), and the mRNA synthesis inhibitor actinomicin (ATD) were able to completely block the HO-1 induction by EGb 761 (FIG. 45d).

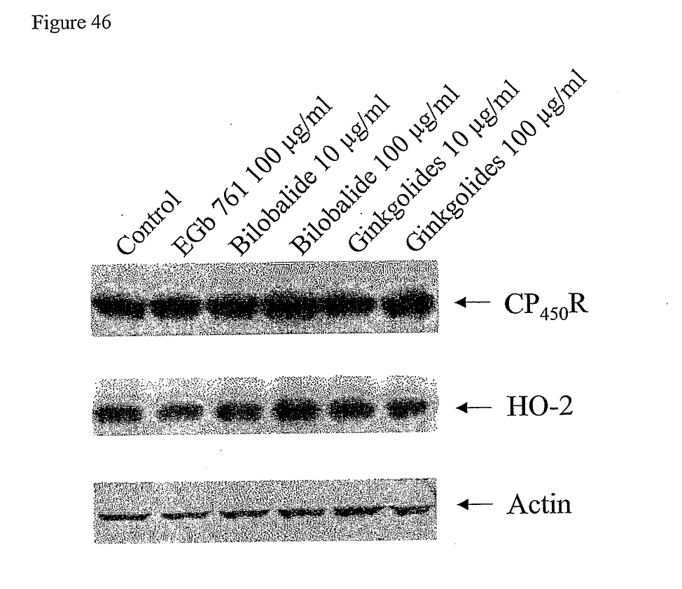

Using primary mouse cortical neuronal cultures, the effect of Ginkgo biloba extracts on the HO-2 protein expression level was examined. Neither the whole Ginkgo biloba extract (EGb 761), nor its chemical components (bilobalide and ginkgolides) affected HO-2 expression level in cultured neurons, as shown in the Western blot analysis of FIG. 46. Further, the ability of Ginkgo biloba extracts to affect the expression of NADPH-cytochrome P₄₅₀ reductase (CP₄₅₀R), which acts as an electron donor to the HO system enzyme activity, was examined. None of the Ginkgo biloba extracts affected CP₄₅₀R protein expression in cultured neurons (FIG. 46). Together, these results demonstrate that EGb 761, but not bilobalide or ginkgolides, induces HO-1 and that Ginkgo biloba extracts do not affect the expression level of HO-2 or NADPH-cytochrome P₄₅₀ reductase.

### Example 30

### EGb 761 can Act on HO-1 Promoter

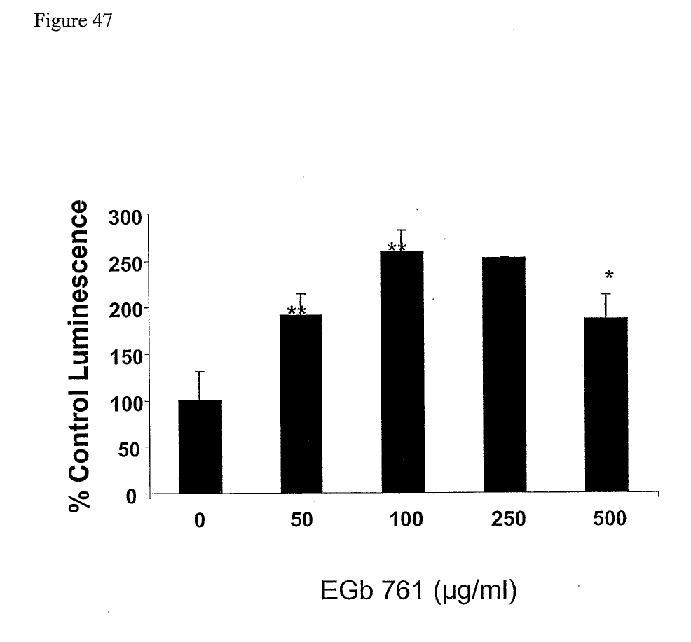

Hepa pARE-luc cells use the firefly luciferase gene as a reporter under the control of three copies of an antioxidant/electrophilic response element (ARE) with a minimal promoter from the mouse HO-1 gene. Here, Hepa pARE-luc cells were treated with various concentrations (0, 50, 100, 250, and 500 μg/ml) of EGb 761 for 18 h. The graph of FIG. 47 shows that EGb 761 stimulated the minimal HO-1 promoter in a dose-dependent manner to increase the transcription of HO-1. Results are reported as % control of luminescence. The effect of EGb 761 peaked at 100 μg/ml treatment and fell off slightly at 500 μg/ml. Thus, this data shows a dose response effect of EGb 761 on the minimal HO-1 promoter.

### Example 31

### EGb 761 Offers In Vitro Neuroprotection that can be Blocked by Tin Protoporphyrin IX (SnPPIX)

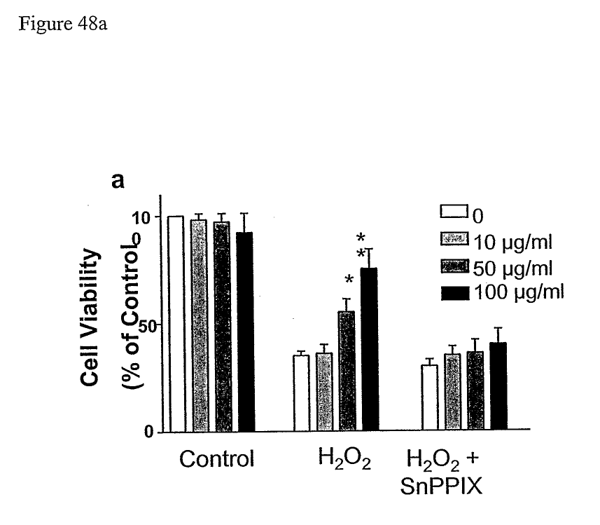

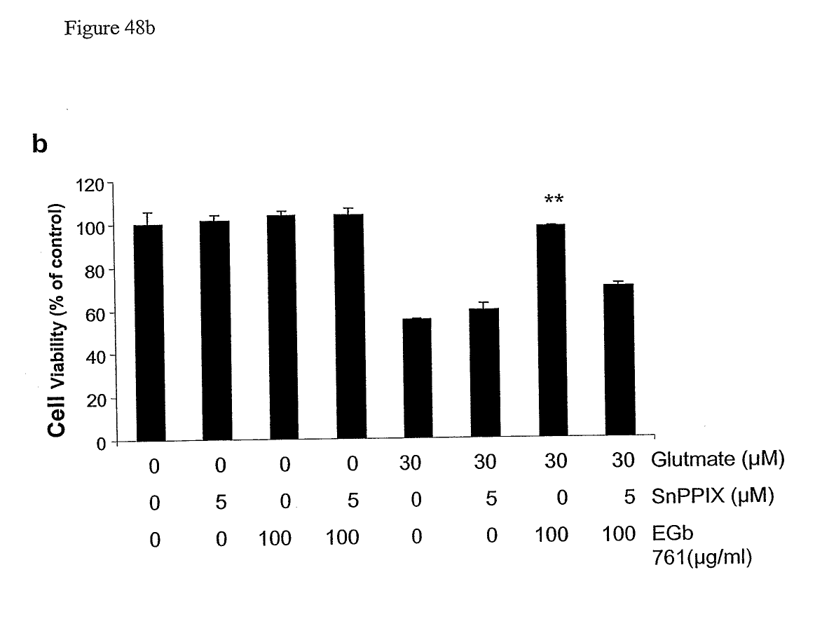

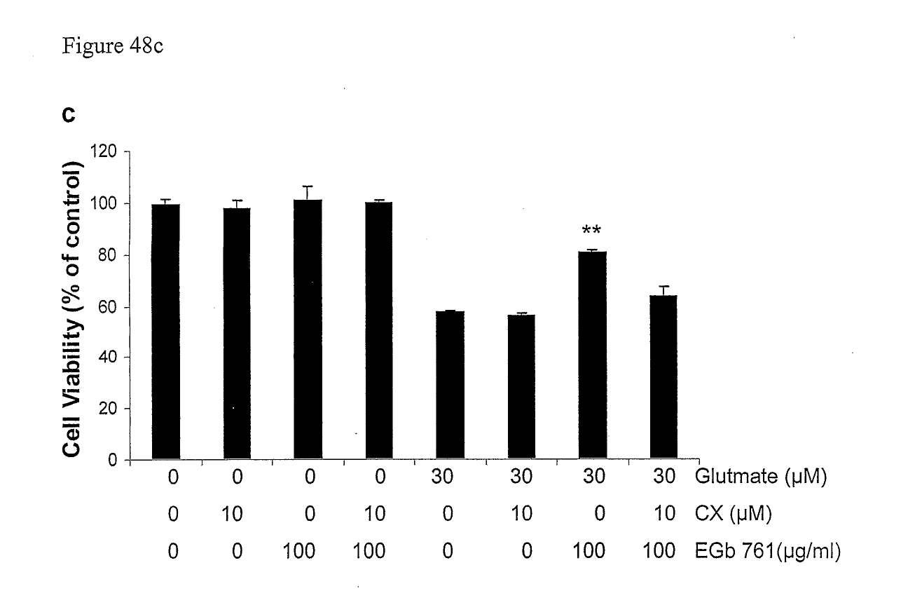

Treatment with EGb 761 at 10, 50 and 100 μg/ml protected mouse cortical neuronal cells against H₂O₂-induced oxidative stress, as shown in the graph of FIG. 48a. Here, the HO inhibitor SnPPIX was also used. Treatment with SnPPIX (5 μM) blocked the protective effect of EGb 761 (FIG. 48a). Further, 100 μg/ml EGb 761 protected mouse cortical neuronal cells against the excitotoxicity induced by glutamate, as shown in FIG. 48b and c. The graphs of FIGS. 6 b and 6c report cell viability (% of control) of neuronal cells treated with various combinations of glutamate, SnPPIS and Egb 761. Both SnPPIX (5 μM) and the protein synthesis inhibitor CHX (10 μM) prevented the protective effect of Egb 761(FIG. 48b and c). Together, this data demonstrates that EGb 761 is neuroprotective against H₂O₂— and glutamate-induced toxicity.

### Example 32

### Effect of EC Pre-Treatment Using HO1 WT Mice on Various Parameters

Numerous epidemiological studies have revealed a strong inverse correlation between ischemic heart disease and consumption of wine, other alcoholic beverages, and fruits and vegetables containing flavonoids and other polyphenols. Cocoa (Theobroma cacao) is a flavonoid-rich food that has the potential to improve an individual's oxidant defense systems and activate other protective cellular pathways.

Infarct Volume

To assess the protective effect of EC (epicatechin) in pre-treatment, 4 different doses of EC were selected on the basis of previous toxicological studies (Galati, et al. Free Radic Biol Med. 40: 570-580. 2006.). 4 doses of EC at: 2.5 mg/kg, 5 mg/kg, 15 mg/kg, and 30 mg/kg were used for experimentation. Polyphenols induce phase II enzymes to enhance the antioxidant defense system, thus HO1, a potential phase II enzyme, was targeted to evaluate its role in mediating the protection of EC. First, HO1 wildtype mice (HO1WT) were selected based on the knowledge that these mice have HO1 present, and thus can be tested for gene up-regulation based on the dietary intervention of EC.

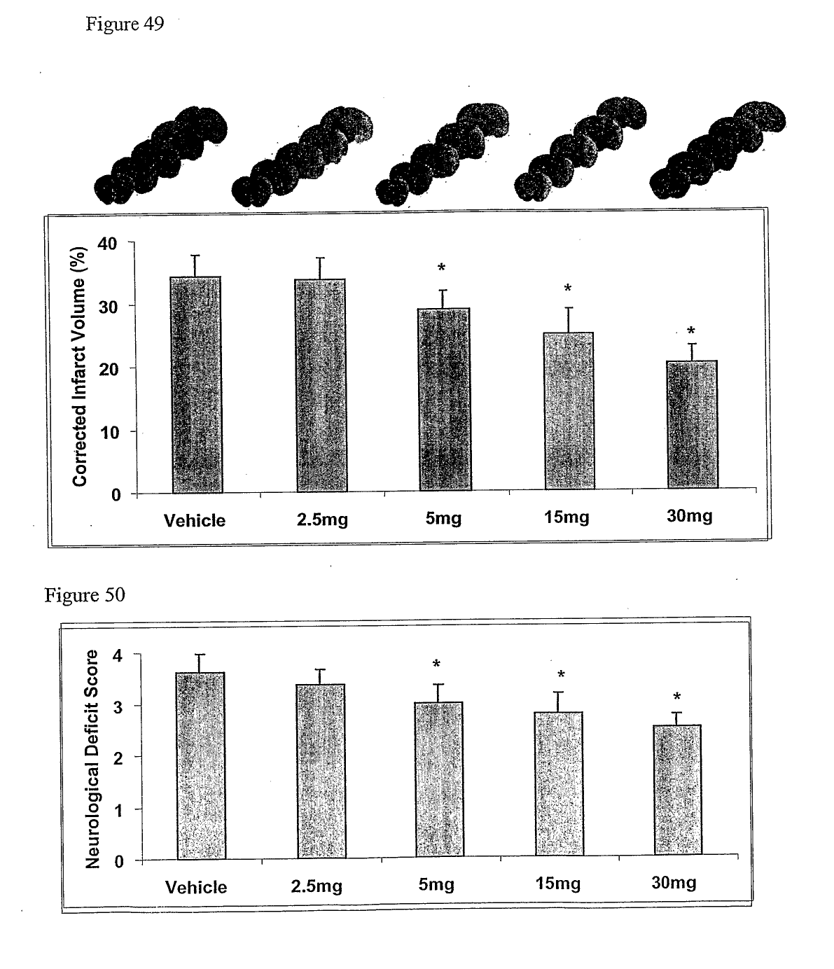

Male mice, weighing 20-25 g were divided in to 5 groups of 8-12 mice in each group. The mice were orally administered a single dose of EC or normal saline through oral gavage, 90 minutes before MCAO. Mice underwent microsurgery and MCA was occluded for 90 min, and then survived for 24 h. After evaluation of neurological deficit scores (NDS), mice were sacrificed and TTC was performed on brain sections. EC dose-dependently protected MCAO induced brain injury and infarct volumes as shown in FIG. 49. Infarct volumes were observed to be significantly smaller at doses of 30 mg/kg (20.1±2.7%; p<0.007); 15 mg/kg (24.9±3.8%; p<0.01); 5 mg/kg (28.8±2.9%; p<0.04), as compared to the vehicle group (34.2±3.4%). However, there were no significant differences observed in infarct volumes at 2.5 mg (33.8±3.3%).

### Neurological Deficit Scores (NDC)

EC was found to have protective effects in mice as shown by the significant differences in Neurological deficit scores (NDC) (FIG. 50). EC significantly and dose-dependently restored neurological deficits found in the mice at 30 mg/kg (2.5±0.25; p<0.01); 15 mg/kg (2.7±0.39; p<0.01) and 5 mg/kg (3±0.35; p<0.03) as compared to the vehicle treatment. However, no differences were observed in 2.5 mg/kg (3.3±0.29) treatment group animals, as shown in FIG. 50.

Physiological Parameters

There were no differences observed in physiological parameters (pH, PaCo2, Pao2) in the different drug concentrations and vehicle treatments, as shown in Table 15 below.

TABLE 15
Physiological parameters of the mice treated with vehicle and EC

| | 1 hr | | |
|---|---|---|---|
| Parameters | before MCAO | 1 hr after MCAO | 1 hr after reperfusion |
| | Vehicle | | |
| pH | 7.382 ± 0.05 | 7.386 ± 0.05 | 7.400 ± 0.03 |
| PaCO₂ | 44.4 ± 1.9 | 45.8 ± 1.4 | 42.0 ± 1.1 |
| PaO₂ | 138.8 ± 5.3 | 129.2 ± 6.4 | 132.0 ± 4.2 |
| | 2.5 mg | | |
| pH | 7.30 ± 0.03 | 7.37 ± 0.01 | 7.38 ± 0.03 |
| PaCO₂ | 43.0 ± 1.8 | 43.2 ± 1.8 | 44.4 ± 1.7 |
| PaO₂ | 132.8 ± 4.6 | 129.2 ± 2.6 | 131.6 ± 6.9 |
| | 5 mg | | |
| pH | 7.39 ± 0.03 | 7.4 ± 0.03 | 7.360 ± 0.03 |
| PaCO₂ | 49.2 ± 3.3 | 45.2 ± 1.2 | 44.2 ± 2.2 |
| PaO₂ | 141.4 ± 7.4 | 129.2 ± 5.1 | 139.0 ± 9.7 |
| | 15 mg | | |
| pH | 7.38 ± 0.05 | 7.35 ± 0.03 | 7.4 ± 0.04 |
| PaCO₂ | 48.8 ± 1.2 | 45.8 ± 1.3 | 47.6 ± 3.7 |
| PaO₂ | 138.8 ± 7.5 | 127.6 ± 5.2 | 148.0 ± 8.0 |
| | 30 mg | | |
| pH | 7.40 ± 0.05 | 7.38 ± 0.15 | 7.40 ± 0.03 |
| PaCO₂ | 44.8 ± 1.8 | 46.8 ± 2.7 | 44.4 ± 1.6 |
| PaO₂ | 130.0 ± 6.5 | 139.0 ± 4.4 | 131.6 ± 6.9 |

Cerebral Blood Flow:

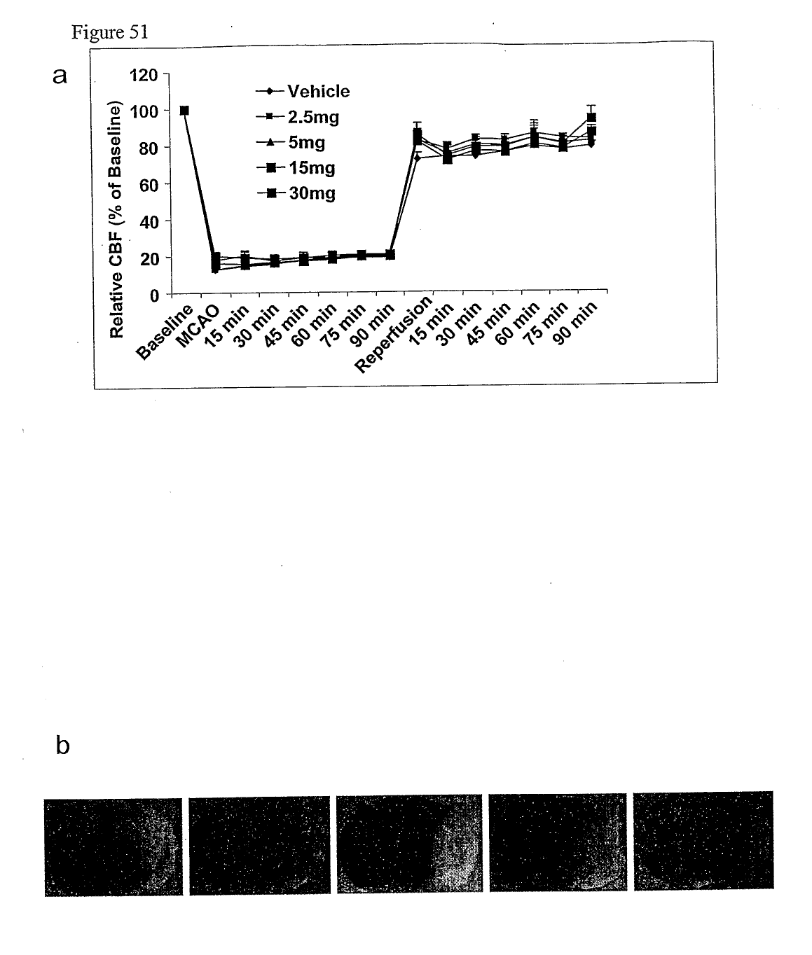

FIG. 51, a and b shows that there were no significant differences observed between 4 different treatments in cerebral blood flow as monitored by Laser Doppler. In a cohort of pre-treatment experiments, male HO1WT mice weighing 20-25 g were distributed in 5 groups (n=5) and CBF was monitored. Here, 90 minutes after the vehicle and drug (2.5, 5, 15, 30 mg) administration, relative CBF was measured from 30 minutes before occlusion through 1 h of reperfusion. There were no significant differences observed between vehicle and 4 different drug treatments (2.5, 5, 15, 30 mg) in cerebral blood flow as monitored by Laser Doppler (FIG. 51).

### Example 33

### EC Post-Treatment (3.5 and 6 h after MCAO) and 72 h Survival Using HO1WT Mice

After observing dose dependent protective effects of EC in pre-treatment paradigms, experimentation shifted to the post-treatment therapeutic potential time window. Here again, HO1WT mice were used for post-treatment experiments, based on the premise that HO1 would serve as the target molecule, and also due to the observed survival rates and resistance to MCAO shown previously with these mice (Shah et al 2006). Further, when these mice were used in the silicone filament model, less mortality in pretreatment paradigms was observed, and therefore HO1WT was an ideal model to test a number of post treatment therapeutic windows. The selection of 2 drug doses for post-treatment parameters was based on previous toxicological studies. Higher doses (>150 mg) of polyphenols has resulted in mortality of mice. Therefore, a safe and effective dose of EC was determined. Another concern in post-treatment experiments is mortality. Previously, high mortalities and subarachnoid hemorrhages were observed in preheated glue coated suture models. Thus, HO1WT mice were used, and MCA was occluded with a silicone-coated filament (180-200micrometer). The highest therapeutic dose (30 mg/kg) with maximum protection and the fewest deleterious side effects was used.

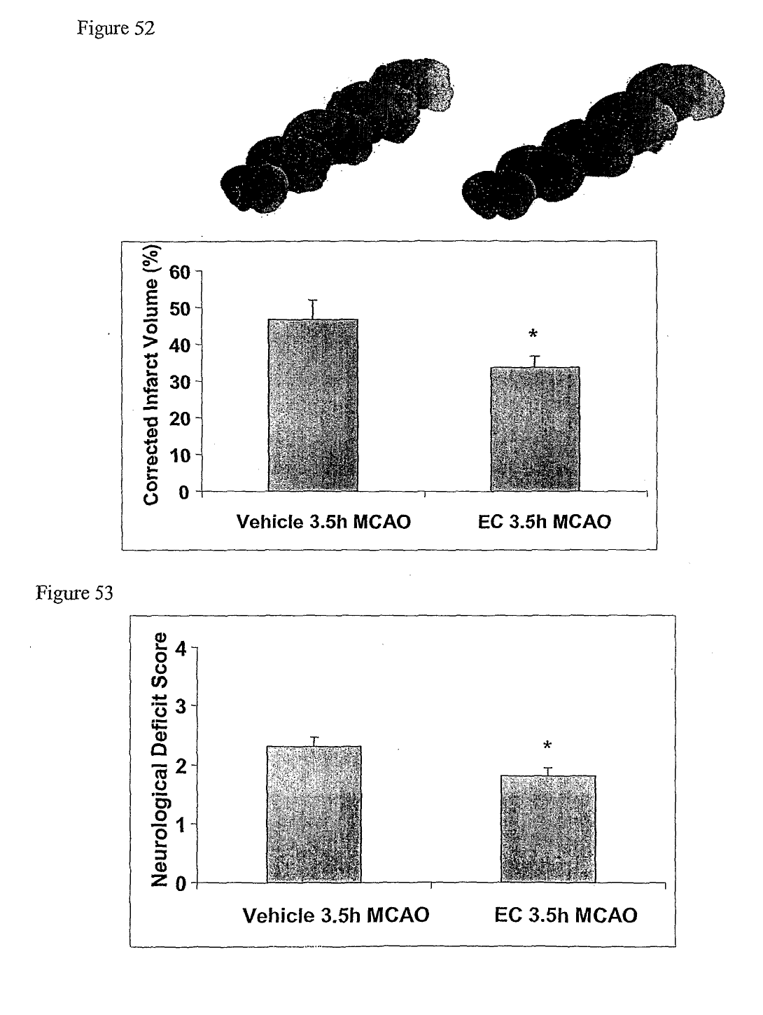

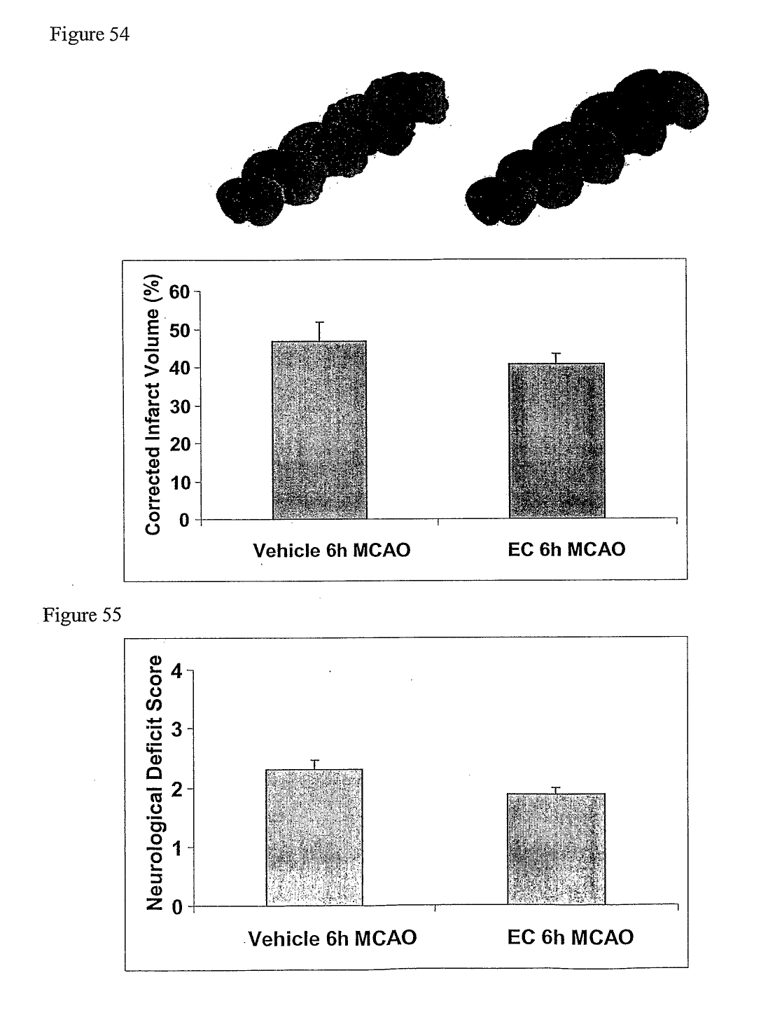

Previous toxicological studies on EC have shown it least toxic when compared to other phenols, and even safe up to 150 mg (Galati et al Free Radic Biol Med. 40: 57-580, 2006). In a separate cohort of experiments, HO1WT mice were distributed into 4 groups of 12 mice each. Mice were subjected to MCAO (90 min), and after 2 and 4.5 h of reperfusion a single dose of 30 mg/kg EC or vehicle was administered. Mice were allowed to survive for 72 h. Mice from all the groups were monitored regularly for weight loss. 1 ml of 5% dextrose was injected (i.p) at 24 and 48 h to counteract the dehydration that may lead to higher mortality rates in post-treatment paradigms. 5% dextrose has been observed to have no significant protective effects if given alone, as compared with normal saline and distilled water. 5% dextrose increased survival rates in MCAO treated mice. NDS were also observed on daily basis, and after 72 h mice were sacrificed and brains harvested for TTC staining, followed by analysis of infarction volume. All the mice survived and no mortality was observed in both EC treated mice groups, while in vehicle treatment groups, 2-3 mice each died after 48 h. Upon opening the skulls of the dead mice, it was observed that the cause of death was excessive edema. There was no surgical cause of death. Significant (p<0.03) protection in infarction volumes was observed in the EC post-treatment (33.5±3.2%) group, as compared to the vehicle (46.6±5.3%) treated group (FIG. 52). Similarly, there was a significant (p<0.01) difference observed in the NDS between EC (1.8±0.1) and vehicle (2.3±0.1) treated groups (FIG. 53). In the 6 h post-treatment group, EC showed a protective trend of neuroprotection, but was not found statistically significant (40.5±2.7) as compared to the control (46.6±5.3) group (FIG. 54). NDS were also not significantly different between the EC 6 h post-treatment (1.8±0.1) as compared to the vehicle control (2.3±0.16) groups (FIG. 55).

### Example 34

### EC Pre-Treatment in Ho1^{-/-} Mice

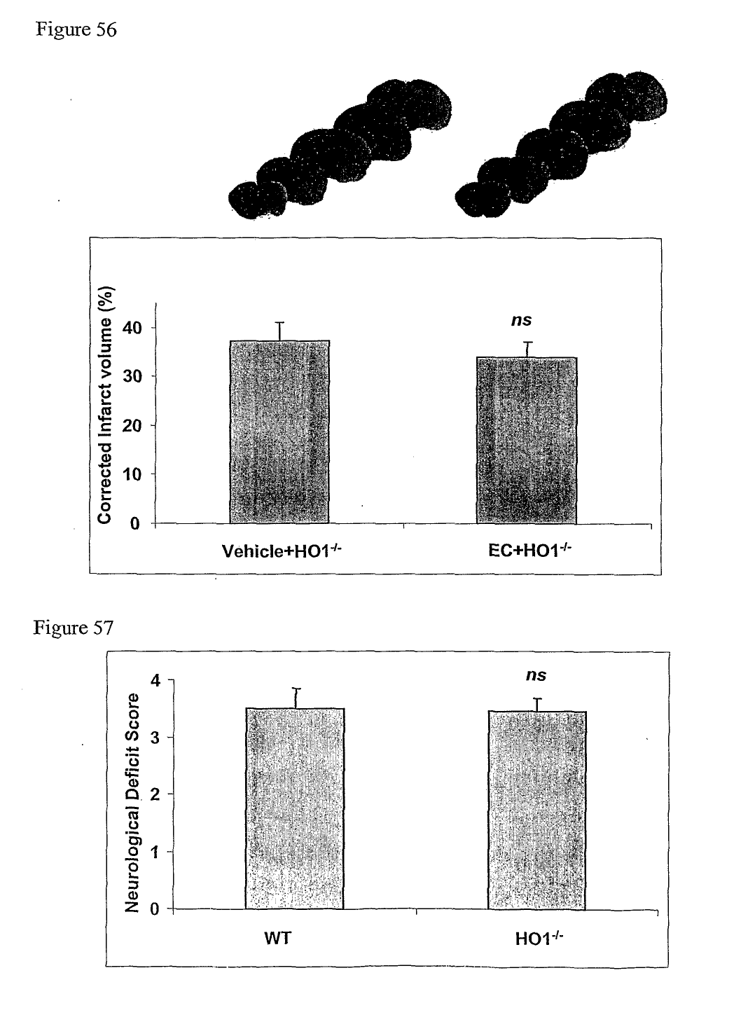

The preceding data demonstrated the dose dependent protection of EC in MCAO induced brain injury; however the mechanism involved was yet to be determined. Given the fact that in WT mice, HO1 may play a role in the protection, gene deleted HO1 mice were used to assess whether EC can protect or exacerbate the damage in these mice. Using the same protocol of EC treatment and MCAO, two groups of male Ho1^{-/-} mice (weighing 20-25 g; n=12) were selected and were treated with either normal saline or EC (30 mg/kg), 90 minutes before MCAO (90 minutes ischemia). After 24 h of reperfusion, animals were sacrificed and TTC was performed on brain sections. No significant difference in infarct volumes between the vehicle (37.1±3.9%) and EC treated HO1^{-/-} (33.8±3.2%) mice was observed, as shown in the graph in FIG. 56. Neurological deficit scores were also observed to have no significant differences between vehicle (3.5±0.5) and EC (3.4±0.2) treated HO1^{-/-} mice (FIG. 57). Taken together, The data presented here shows that EC could not restore the damage induced by MCAO in HO1^{-/-} mice. Thus, the protective mechanism of EC may be mediated through the up regulation of HO1 in WT mice, which then failed to induce the phase II enzyme in HO1^{-/-} because of lack of the responsible gene.

### Example 35

### EC Pre-Treatment in Nrf2 Knockout (Nrf2^{-/-}) and WT Mice

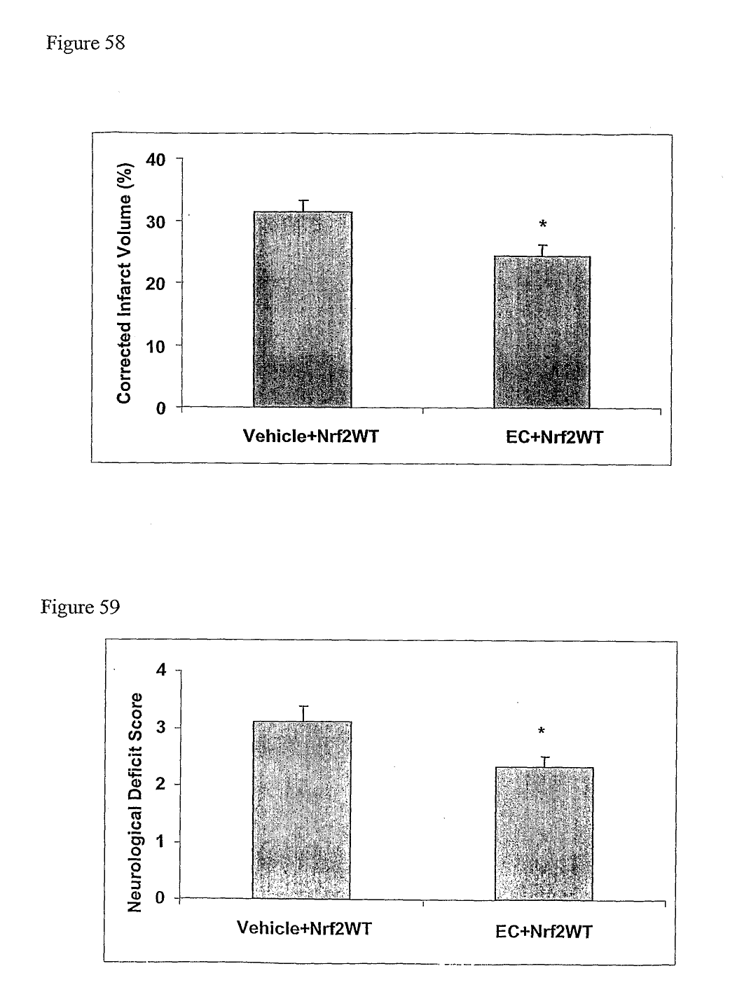

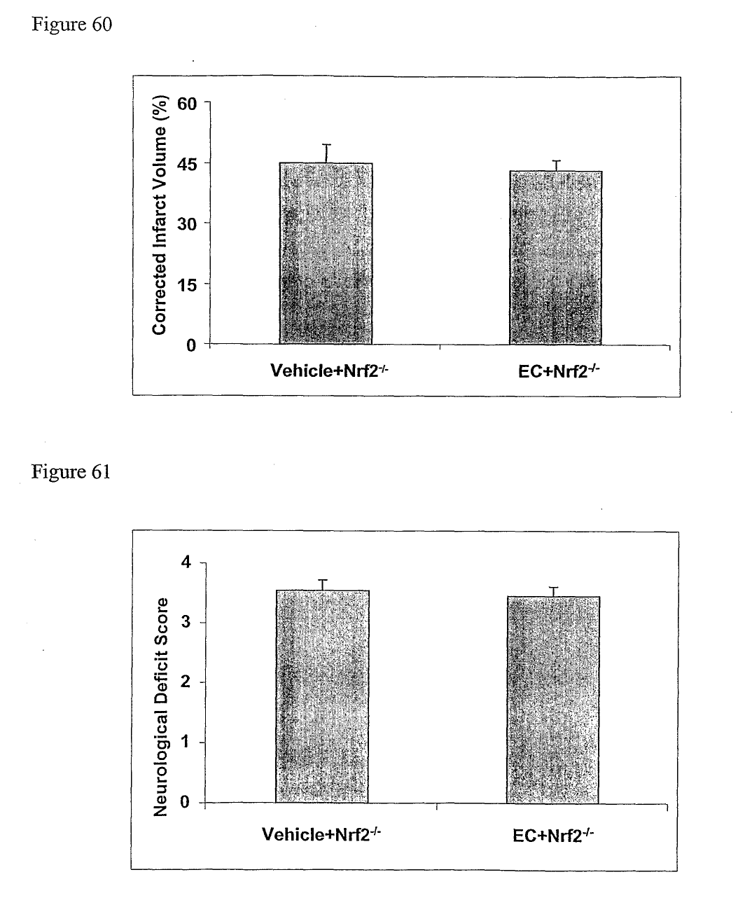

To further validate the pathway of HO1 upregulation, molecules upstream of HO1 were examined. There is ample evidence in the literature showing different molecules that up-regulate HO1 through keap1/ARE/Nrf2 mediation (Satoh et al. PNAS USA. 103: 768-772. 2006.; Shih et al. J Neurosci. 25: 10321-10335. 2005.). To determine whether EC works through that pathway, Nrf2 gene deleted and WT mice were used. In a separate cohort of experiments, 4 groups of male animals (weighing 20-25 g), 2 Nrf2^{-/-} and 2 WT (n=12 in each group) were treated with either single dose of EC (30 mg/kg) or vehicle, 90 minutes before MCAO (90 minutes). After 24 h of survival, animals were evaluated for NDS and sacrificed to obtain brain sections for TTC staining. Nrf2WT group mice treated with EC and vehicle demonstrated a significant difference (p<0.04) in infarct volumes between the EC (24.1±1.8%) and vehicle (31.3±1.9%) treatment groups (FIG. 58). Neurological deficit scores in Nrf2 WT mice were also observed to be significantly (p<0.02) less in EC (2.3±0.1) treated group as compared to the vehicle (3.1±0.26) group (FIG. 59). In the Nrf2^{-/-} group, mice treated with EC (43.0±2.4) were not observed to have significant protective effect as compared to the vehicle (44.8±4.6) treated group (FIG. 60). There was no significant difference observed in the NDS between EC (3.4±0.17) and vehicle (3.5±0.1) treated groups (FIG. 61). Therefore, significant protection of EC in Nrf2 WT, but not in Nrf2^{-/-}, is an indication that the protective mechanisms were brought through the activation of Nrf2 by EC, which after translocation to the nucleus activated phase II detoxification enzymes, likely through HO1.

### Example 36

### Screening Compounds

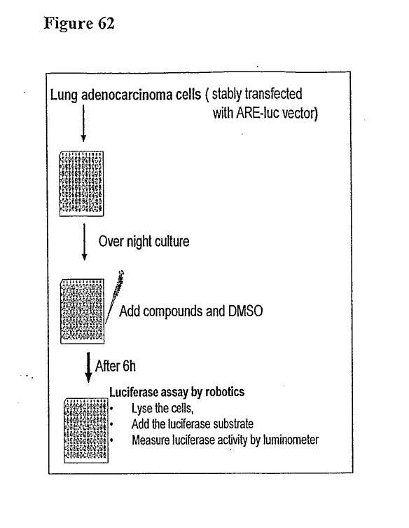

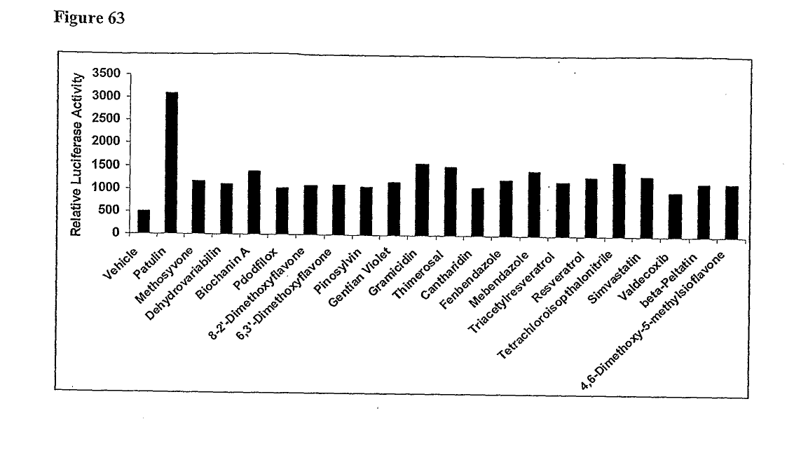

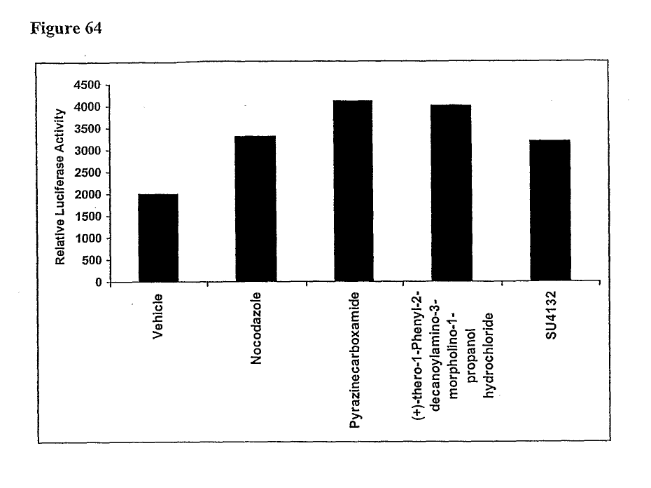

A high throughput approach is used to screen different chemicals for their potency to activate Nrf2. A cell based reporter assay approach is used for the identification agents that can activate Nrf2 mediated transcription. Briefly, lung adenocarcimona cells that are stably transfected with ARE-luciferase reporter vector are plated on to 96 well or 384 well plates. After overnight incubation, cells are pretreated for 12-16 h with different compounds. Luciferase activity is measured after 12 hours of treatment using luciferase assay system from Promega. The increase in luciferase activity reflects the degree of Nrf2 activation. FIG. 62 is a schematic depicting the method of screening for Nrf2 inhibitors by high throughput screening of chemical libraries. Chemical libraries that can be screened for Nrf2 modulatory compounds include CB01 (ChemBridge 1) and CB02 (ChemBridge 2), MSSP (Spectrum 1), Sigma LOPAC 1280, ChemBridge CNS-Set, ChemBridge Divert-SET, BIOMOL collection. FIGS. 63 and 64 are illustrate compounds that have been identified from these libraries as modulators of Nrf2 activity. Here, luciferase activity is an indication of Nrf2 activity, as described above.

### Methods of the Invention

The results reported herein were obtained using the following Materials and Methods:

### Animals and Care

Animal protocols were approved by the Institutional Animal Care and Use Committee of Johns Hopkins University. Nrf2 knockout (Nrf2^{-/-}) and wildtype (WT) CD1 mice were obtained and genotyped. Mice were fed with an AIN-76A diet, given water ad libitum, and housed under controlled conditions (23±2° C.; 12 hour light/dark periods). In some experiments animals were given Teklad Global 18% Protein Rodent Diet (Sterilizable) (Harlan Holding, Inc, Wilmington, Del., USA), formula 2018S, which is a fixed formula autoclavable pellet form chow containing no nitrosamines and a low level of natural phytoestrogens, with 18% protein (non-animal) and 5% fat for consistent growth, gestation, and lactation. The first rigid probe analysis used 45 of an original 98 WT mice. The remaining 53 mice were used for flexible probe analysis. In another probe analysis study, 17 WT and 17 HO-1^{-/-} mice were used. Of the 17 in each group, 10 were tested with a rigid probe and 7 with a flexible probe. All mice were male and weighed 20-25 g.

In some experiments, male WT and HO-1^{-/-} mice (8-10 weeks old) were orally administered 100 mg/kg EGb 761 (IPSEN Laboratories, Paris, France; WT, n=10; n=12) or vehicle [distilled water-PEG 400 (30:70), WT, 12=10; HO-1^{-/-}, n=11) once daily for 7 d before induction of ischemia.

Nrf2-Deficient ICR Mice

Nrf2-deficient ICR mice were generated as described (Itoh, K et al. Biochem. Biophy. Res. Comm. 236:313-322.1997). Nrf2-deficient mice were generated by replacing the b-ZIP region of Nrf2 gene with the SV40 nuclear localization signal (NLS) and β-galactosidase gene (Itoh K et al. Biochem Biophys Res Commun 236:313-322. 1997). Mice were genotyped for nrf2 status by PCR amplification of genomic DNA extracted from blood (Ramos-Gomez et al. PNAS U.S.A. 98:3410-3415.2001). PCR amplification was carried out using three different primers, 5′-TGGACGGGACTATTGAAGGCTG-3′ (sense for both genotypes), 5′-CGCCTTTTCAGTAGATGGAGG-3′ [anti-sense for wild-type nrf2 mice (nrf2+/+)], and 5′-GCGGATTGACCGTAATGGGATAGG-3′ (anti-sense for LacZ) (36). Mice were fed AIN-76A diet and water ad libidum and housed under controlled conditions (23±2° C.; 12/12 h light/dark periods.

### Antibodies and Reagents

The following antibodies were used: Anti-caspase 3 polyclonal antibody for immunohistochemistry (Idun Pharmaceuticals, La Jolla, Calif., USA); InnoGenex™ Iso-IHC DAB kit (InnoGenex, San Ramon, Calif., USA); biotinylated anti-mouse IgG and peroxidase-conjugated streptavidin, Vectashield HardSet mounting medium and Vector RTU HRP-avidin complex (Vector Laboratories, Burlingame, Calif., USA); rabbit anti-surfactant protein C (SpC) antibody (Chemicon International, Inc., Temecula, Calif., USA); rat anti-mouse Mac-3 antibody (BD Bioscience, Franklin Lakes, N.J., USA); anti-rabbit Texas red antibody, streptavidin-Texas red conjugated complex and DAPI (Molecular Probes Inc., Eugene, Oreg., USA); biotinylated rabbit anti-mouse secondary antibody (DakoCytomation, Carpinteria, Calif., USA); Fluorescein-FragEL DNA Fragmentation Detection Kit (Oncogene Research Products, San Diego, Calif., USA); Wright-Giemsa stain (Diff-Quik; Baxter Scientific Products, McGaw Park, Ill., USA); Octamer transcription factor 1 (OCT1) and CaspACE™ Assay kit (Promega Corporation, Madison, Wis., USA); halothane (Halocarbon Laboratories, River Edge, N.J., USA); QuickHyb solution (Stratagene, Carlsbad, Calif., USA); leupeptin, pepstatin A and normal mouse IgG1 (Sigma-Aldrich, St. Luis, Calif., USA); rat anti-mouse neutrophil antibody (Serotec, Raleigh, N.C., USA); actin and anti-mouse CD45R primary antibody (Santa Cruz Biotechnology Inc., Santa Cruz, Calif., USA); rabbit anti-caspase 3 antibody for Western blot (Cell Signaling technology, Inc., Beverly, Mass., USA); anti-CD34 and anti-lamin B1 antibody (Zymed Laboratories, Inc., South San Fransisco, Calif., USA); CH11 monoclonal antibody (Beckman Coulter, Inc., Fullerton, Calif., USA); ECL® Western blotting detection kit (Amersham Biosciences, Piscataway, N.J., U.S.A.); Bradford's reagent (Bio-Rad, Hercules, Calif., U.S.A.); PVDF membrane (Millipore, Bedford, Mass., USA).

Other antibodies used include anti-mouse CD3 and anti-mouse CD28 antibodies (Pharmingen, BD Biosciences, San Jose, Calif., USA); Mercury TransFactor ELISA kit (Clontech, BD Biosciences, Palo Alto, Calif., USA); biotinylated anti-IL-4 monoclonal antibody, anti-IL-13 polyclonal antibody, mouse IL-4, mouse IL-13, mouse eotaxin, human IL-4 and IL-13 ELISA Kits (R & D systems Inc., MN, USA); anti-NF-kB p65 and anti-NF-kB p50 polyclonal antibodies, rabbit anti-Nrf2 polyclonal antibody (Santa Cruz Biotechnology, Santa Cruz, Calif., USA); rabbit anti-rat IgG/HRP conjugate (DakoCytomation, Carpinteria, Calif., USA); BIOXYTECH GSH/GSSG-412 kit (Oxis International Inc., Portland, Oreg., USA); diaminobenzidine (Vector Laboratories, Burlingame, Calif., USA); Diff-Quick reagent (Baxter Dade, Dudingen, Switzerland); complete protease inhibitor cocktail tablets (Roche Pharmaceuticals, Nutley, N.J., USA); SuperScribe II reverse transcriptase, RNeasy mini kits, TOPO 2.1, KpnI, SacI and NotI restriction endonucleases (Invitrogen, Carsbad, Calif., USA); assay on demand kits, fluorogenic probes, TaqMan universal PCR master mix (Applied Biosystems, Foster City, Calif., USA); consensus sequence for the octamer transcription factor 1 (OCT1), PGL3 basic reporter construct and Dual-Luciferase® Reporter Assay system (Promega, Madison, Wis., USA); acetyl choline, 2,2′-azino-bis (3-ethylbenzothiazoline-6-sulfonic acid), bovine serum albumin, FCS, ketamine, ovalbumin, pepsin, normal rabbit serum, normal rabbit IgG₁, sodium pentobarbital, succinyl choline, xylazine, N-acetyl L-cysteine, collagenase IV, and bovine pancreatic DNase I (Sigma-Aldrich, St. Louis, Mo., USA); PMA and A23187 (Calbiochem, San Diego, Calif.); ECL chemiluminescence detection kit (Amersham Pharmacia Biotech, Piscataway, N.J., USA); PVDF membrane (Bio-Rad Laboratories, Hercules, Calif., USA); red cell lysis buffer (eBiosciences, San Diego, Calif., USA); CD4⁺ T cell isolation kit (Miltenyi Biotec, Album, Calif., USA); Cell stainer (Costar, Corning, N.Y., USA); anti-lamin B1 antibody (Zymed Laboratories Inc., South San Francisco, Calif., USA).

### Bronchoalveolar Lavage Fluid and Phenotyping

Mice (n=8) were anesthetized with 0.3 ml of pentobarbital (65 mg/ml) and the trachea was cannulated. Immediately following exposure to CS for 1.5 months or 6 months, mice (n=8 per group) were anesthetized with sodium pentobarbital. BAL fluid was collected with 1 ml followed by 2×1 ml of sterile PBS containing 5 mM EDTA, DTT (5 mM) and PMSF (5 mM). The BAL fluid was immediately centrifuged at 1500×g. The total cell count was measured, and cytospin preparation (Shandon Scientific Inc., Cheshire, UK) was performed. Cells were stained with Diff-Quick reagent, and a differential count of 300 cells was performed using standard morphological criteria (Saltini C et al. Am Rev Respir Dis 130:650-658.1984).

To examine endotoxin-mediated sepsis, the lungs were aspirated 3 times with 1 ml of sterile PBS to collect BAL fluid. Cells were counted by using a hemocytometer, and differential cell counts were performed on 300 cells from BAL fluid with Wright-Giemsa stain (Baxter Scientific Products, McGaw Park, Ill.).

### Histopathology and Immunohistochemistry.

Lungs were inflated with 10% buffered formalin through the trachea 24 h after the treatment and subsequently fixed for 24 h at 4° C. After paraffin embedding, 5-mm sections were cut and stained with H&E. For identification of neutrophils, lung sections were stained by using rat IgG anti-mouse neutrophil monoclonal antibody (Serotec, N.C.) followed by the secondary goat anti-rat IgG conjugated to horseradish peroxidase. Color development was performed with 3′,3′-diaminobenzidine, and the slides were counterstained with hematoxylin.

### Exposure to Cigarette Smoke

The CS machine for smoke exposure was similar to the one used by Witschi et al. (Carcinogenesis. 18:2035-2042.1997.); however, the exposure regimen in terms of chamber atmosphere and duration of CS exposure were considerably more intense. Mice 8 weeks of age were divided into four groups (n=40 per group): I, control nrf2 wild-type mice; II, experimental nrf2 wild-type mice; III, control nrf2-disrupted mice and IV, experimental nrf2 disrupted mice. Groups I and III were kept in a filtered air environment, and groups II and IV were subjected to CS for various time periods. CS exposure was carried out (7 h/day, 7 days/week for up to 6 months) by burning 2R4F reference cigarettes (2.45 mg nicotine per cigarette; purchased from the Tobacco Research Institute, University of Kentucky, Lexington, Ky., USA) using a smoking machine (Model TE-10, Teague Enterprises, Davis, Calif., USA). Each smoldering cigarette was puffed for 2 s, once every minute for a total of eight puffs, at a flow rate of 1.05 L/min, to provide a standard puff of 35 cm³. The smoke machine was adjusted to produce a mixture of sidestream smoke (89%) and mainstream smoke (11%) by burning five cigarettes at one time. Chamber atmosphere was monitored for total suspended particulates and carbon monoxide, with concentrations of 90 mg/m³ and 350 ppm, respectively.

### Treatment to Induce Endotoxic Shock

Endotoxic shock was induced in male mice (8 weeks old) of both genotypes by ip injection of LPS at doses of 0.75 or 1.5 mg per mouse (E. coli, serotype 055.B5; Sigma) as described in the literature. After LPS injection, the mice were monitored for 5 days. To induce non-lethal systemic inflammation, the mice were injected with LPS (ip, 60 μg per mouse) and or recombinant hTNF-α, (ip, 10 μg per mouse) (R & D systems). Control mice received an equivalent volume of vehicle. Intratracheal LPS instillation was used for induction of local inflammation in the lungs. Mice were first anesthetized by ip injection with 0.1 ml of a mixture of ketamine (10 mg/ml) and xylazine (1 mg/ml) in PBS. LPS was instilled intratracheally (10 μg in 50 μl sterile PBS) during inspiration. Control mice received an equivalent volume of vehicle.

### Morphologic and Morphometric Analyses

After exposing the mice to CS for various time periods (1.5, 3 and 6 months), the mice (n=5 per group) were anesthetized with halothane and the lungs were inflated with 0.5% low-melting agarose at a constant pressure of 25 cm as previously described (Kasahara et al. J Clin. Invest. 106:1311-1319. 2000). The inflated lungs were fixed in 10% buffered formalin and embedded in paraffin. Sections (5 μm) were stained with hematoxylin and eosin. Mean alveolar diameter, alveolar length, and mean linear intercepts were determined by computer-assisted morphometry with the Image Pro Plus software (Media Cybernetics, Silver Spring, Md., USA). The lung sections in each group were coded and representative images (15 per lung section) were acquired by an investigator masked to the identity of the slides, with a Nikon E800 microscope, 20× lens.

### TUNNEL Assay

Apoptotic cells in the tissue sections from the agarose-inflated lungs were detected by Fluorescein-FragEL DNA Fragmentation Detection Kit, according to the recommendations of the manufacturer. The lung sections (n=5 per group) were stained with the TdT labeling reaction mixture and mounted with Fluorescein-FragEL mounting medium. DAPI and flourescein were visualized at 330-380 nm and 465-495 nm, respectively. Overlapping DAPI in red and FITC in green create a yellow, apoptotic-positive signal. Images (15 per lung section) of the lung sections were acquired with a 20× lens. In each image, the number of DAPI-positive cells (red signal) and apoptotic cells (yellow) were counted manually. Apoptotic cells were normalized by the total number of DAPI-positive cells.

### Identification of Alveolar Apoptotic Cell Populations in the Lungs

To identify the different alveolar cell types undergoing apoptosis in the lungs, a fluorescent TUNEL labeling was performed in the lung sections from the air and CS-exposed (6 months) nrf2+/+ and nrf2−/− mice, using the Fluorescein-FragEL DNA Fragmentation Detection Kit by following the procedure described above. To identify the apoptotic type II epithelial cells in the lungs after TUNEL labeling, the lung sections were incubated first with an anti-mouse surfactant protein C (SpC) antibody, and then with an anti-rabbit Texas red antibody. Apoptotic endothelial cells were identified by incubating the fluorescent TUNEL labeled sections first with the anti-mouse CD 34 antibody and then with the biotinylated rabbit anti-mouse secondary antibody. The lung sections were rinsed in PBS and then incubated with the streptavidin-Texas red conjugated complex. The apoptotic macrophages in the lungs were identified by incubating the TUNEL labeled lung sections first with the rat anti-mouse Mac-3 antibody and then with the anti-rat Texas red antibody. Finally, DAPI was applied to all lung sections, incubated for 5 minutes, washed and mounted with Vectashield HardSet mounting medium. DAPI and flourescein were visualized at 330-380 nm and 465-495 nm, respectively. Images of the lung sections were acquired with the Nikon E800 microscope, 40× lens.

### Immunohistochemical Localization of Active Caspase-3

Immunohistochemical staining of active caspase-3 assay was performed using anti-active caspase-3 antibody (Kasahara Y et al. Am. J. Respir. Crit. Care. Med. 163:737-744.2001) and the active caspase-3-positive cells were counted with a macro using the Image Pro Plus program (Tudor, R M et al. Am. J. Respir. Cell. Mol. Bio. 29: 88-97.2003). The counts were normalized by the sum of the alveolar profiles herein named as alveolar length and expressed in μm or mm. Alveolar length correlates inversely with mean linear intercept, i.e., as the alveolar septa are destroyed, mean linear intercepts increases as total alveolar length, i.e., total alveolar septal length decreases.

### Caspase 3 Activity Assay

Caspase-3 activity was assessed by using a fluorometric CaspACE™ Assay commercial kit according to the manufacturer's instructions. Briefly, the frozen lung tissues were immediately homogenized with hypotonic lysis buffer [25 mM HEPES (pH 7.5), 5 mM MgCl₂, 5 mM EDTA, 5 mM DTT, 2 mM PMSF, 10 μg/ml pepstatin A and 10 μg/ml leupeptin] using a mechanical homogenizer on ice and centrifuged at 12, 000×g for 15 min at 4° C. The clear supernatant was collected and frozen in liquid nitrogen. The protein was quantified using Bradford's reagent. Lung supernatant containing 30 μg of protein was added to a reaction buffer (98 μl) containing 2 μl DMSO, 10 μl of 100 mM DTT and 32 μl of caspase assay buffer in a 96 well flat bottom microtitre plate (Corning-Costar Corp., Cambridge, Mass., USA). The reaction mixture was incubated at 30° C. for 30 min. Then, 2 ml of 2.5 mM caspase-3 substrate (Ac-DEVD-AMC) was added to the wells and incubated for 60 min at 30° C. The fluorescence of the reaction was measured at an excitation wavelength of 360 nm and an emission wavelength of 460 nm. 30 μg of proteins from anti-Fas antibody treated Jurkat cells (treated with 1 μg CH11 monoclonal antibody per ml RPMI containing 5×10⁵ cells for 16 h at 37° C.) were used as a positive control. Caspase-3 inhibitor (2 ml of 2.5 mM DEVD-CHO), a specific inhibitor of caspase-3, was used to show specificity of caspase-3 activity. The activity was below background levels after the addition of caspase-3 inhibitor. These experiments were performed in triplicate and repeated three times.

### Immunohistochemical Localization of 8-Oxo-dG

For the immunohistochemical localization and quantification of 8-oxo-dG, lung sections (n=5 per group) from the mice exposed to CS for 6 months were incubated with anti-8-oxo-dG antibody and stained using InnoGenex™ Iso-IHC DAB kit using mouse antibodies. Normal mouse-IgG1 antibody was used as a negative control. The 8-oxo-dG-positive cells were counted with a macro (using Image Pro Plus), and the counts were normalized by alveolar length as described (Tuder, R M et al. Am. J. Respir. Cell. Mol. Bio. 29: 88-97.2003).

### Immunohistochemical Localization of Inflammatory Cells in the Lungs

Macrophages were identified by the rat anti-mouse Mac-3 and secondary biotinylated anti-rat antibody immunostaining using the Vector RTU HRP-avidin complex with 3,3,-diaminobenzidine as the chromogenic substrate. The number of Mac-3 positive cells in the lung sections (n=3 per group and 10 fields/lung section) were counted manually and normalized by alveolar length.

### Electrophoretic Mobility Shift Assay (EMSA)

EMSA was carried out according to a procedure described earlier (Tirumalai R et al. Toxicol Lett 132:27-36.2002). For gel shift analysis, 10 μg of nuclear proteins that had been prepared from the lungs of mice exposed to air or to CS for 5 h was incubated with the labeled human NQO1 ARE, and the mixtures were analyzed on a 5% non-denaturing polyacrylamide gel. To determine the specificity of protein(s) binding to the ARE sequence, 50-fold excess of unlabeled competitor oligo (ARE consensus sequence) was incubated with the nuclear extract for 10 min prior to the addition of radiolabeled probe. For super shift analysis, labeled NQO1 ARE was first incubated for 30 min with 10 μg of nuclear proteins and then with 4 μg of anti-Nrf2 antibody for 2 h. Normal rabbit IgG₁ (4 μg) was used as a control for supershift assay. The mixtures were separated on native polyacrylamide gel and developed by autoradiography. The P³² labeled consensus sequence for the octamer transcription factor1 (OCT1) was used as a control for gel loading. The EMSA was performed three times with the nuclear proteins isolated from three different air or CS exposed nrf2+/+ and −/− mice.

### Western Blot Analysis

Western blot analysis was performed according to previously published procedures (Tirumalai R et al. Toxicol Lett 132:27-36.2002). To determine the nuclear accumulation of Nrf2, 50 μg of the nuclear proteins isolated from the lungs of air or CS-exposed (5 h) nrf2+/+ and −/− mice were separated by 10% sodium dodecyl sulfate polyacrylamide gel electrophoresis (SDS-PAGE), and electrophoretically transferred on to a PVDF membrane. The membranes were blocked with 5% (w/v) BSA in Tris-buffered saline[20 mM Tris/HCl (pH 7.6) and 150 mM NaCl] with 0.1% (v/v) Tween-20 for 2 h at room temperature, and then incubated overnight at 4° C. with polyclonal rabbit anti-Nrf2 antibody followed by incubation with HRP-conjugated secondary antibody. The blots were developed using an enhanced chemiluminescence Western blotting detection kit. Subsequently, the blots were stripped and reprobed with anti-lamin B1 antibody.

To identify the active caspase 3, the lung tissues (n=3) were homogenized with the lysis buffer [containing 50 mM Tris/HCl (pH 8.0), 150 mM NaCl, 0.5% (v/v) Nonidet P40, 2 mM EDTA and a protease inhibitor cocktail] on ice using a mechanical homogenizer. Following centrifugation at 12, 000×g for 15 min, the protein concentration of the supernatant was determined using Bradford's reagent. Equal amounts of protein (30 m) were resolved on 15% SDS-PAGE and transferred on to a PVDF membrane. The membranes were incubated with rabbit anti-caspase 3 antibody and then with secondary anti-rabbit antibody linked to HRP-conjugate. The blots were developed using the enhanced chemiluminescence Western blotting detection kit. Thereafter, blots were stripped and re-probed with antibodies to actin. Western blot was performed three times with protein extracts from three different air or CS exposed (6 months) nrf2+/+ and nrf2−/− mice. Band intensities of procaspase 3 and active caspase 3 of the three blots were determined using the NIH Image-Pro Plus software program. Values are represented as mean±SEM.

To determine the activation of NF-κB, nuclear extracts (15 μg) isolated from the lungs of saline or OVA challenged (1^{st} challenge) Nrf2^{+/+} and Nrf2^{-/-} mice were subjected to SDS-PAGE, as described above. NF-κB was detected by incubating the blots with anti-NT-κB p65 and anti-NF-κB p50 rabbit polyclonal antibodies. Then, the blots were stripped and reprobed with anti-lamin B1 antibody. Western blot was performed with protein extracts from 3 different saline or OVA challenged Nrf2^{+/+} and Nrf2^{-/-} mice, and band intensities of p65 and p50 subunits of NF-κB of the 3 blots were determined using NIH Image-Pro Plus software. Values are represented as mean±SEM.

Other antibodies used in Western analysis include antibodies specific for the p65, p50, IκB-α, α-tubulin (Santa Cruz Biotechnology, Santa Cruz, Calif.), P-IκB-α (Cell signaling Technology), TLR4 and CD14 (eBioscience)

### Transcriptional Profiling Using Oligonucleotide Microarrays

Lungs were excised from control (air-exposed) and CS-exposed (5 h) mice (n=3 per group) and processed for total RNA extraction using the TRIzol reagent. The isolated RNA was used for gene expression profiling with Murine Genome U74A version 2 arrays (Affymetrix, Santa Clara, Calif., US) using the procedures described (Thimmulappa, R. K. et al. Cancer Res 62:5196-5203. 2002). To identify the differentially expressed transcripts, pairwise comparison analyses were carried out with Data Mining Tool 3.0 program (Affymetrix). Only those differentially expressed genes that appeared in at least 6 of the 9 comparisons and showed a change of >1.4-fold were selected. In addition, the Mann-Whitney pairwise comparison test was performed to rank the results by concordance as an indication of the significance (P value≦0.05) of each identified change in gene expression. Genes which were upregulated only in the lungs of wild-type mice in response to CS were selected, and used for the identification of ARE(s) in their upstream sequence.

### Identification of ARE(s) in Nrf2 Regulated Genes

To identify the presence and location of ARE(s) in Nrf2-dependent genes, the murine homologs of human genes were employed (Human Genome build 34 version 1, the NCBI database). For every gene, a 10 kb sequence upstream from the transcription start site (TSS) was used to search for ARE (s) with the help of Genamics Expression 1.1 Pattern Finder tool software (Marcel Dinger, Hamilton, New Zealand) using the primary core sequence of ARE (RTGAYNNNGCR) (43) as the probe. TSS for all the genes was determined by following the Human Genome build 34, version 1 of the NCBI database.

### Northern Blotting

Northern blotting was performed according to the procedure described earlier (Thimmulappa, R. K. et al. Cancer Res 62:5196-5203. 2002). In brief, 10 μg of total RNA isolated from the lungs of air- and CS-exposed (5 h) mice (n=3) was separated on 1.2% agarose gel, transferred to nylon membranes (Nytran super charge, Schleicher & Schuell, Dassel, Germany), and ultraviolet-crosslinked. Full length probes for NQO1, γ-GCS (regulatory subunit), GST α1, HO-1, TrxR, Prx 1, GSR, G6PDH and β-actin were generated by PCR from the cDNA of murine liver. These PCR products were radiolabeled with [α-³²P] CTP and hybridized using QuickHyb solution according to the manufacturer's protocol. After the films were exposed to the phosphoimager screen for 24 h, hybridization signals were detected using a Bioimaging system (BAS1000, Fuji Photo Film, Tokyo, Japan). Quantification of mRNA was performed using Scion image analysis software (Scion Corporation, Frederick, Md., USA). Levels of RNA were quantified and normalized for RNA loading by stripping and reprobing the blots with a probe for β-actin.

### Enzyme Activity Assays

For measuring enzyme activity of selected genes, mice were exposed to CS for 5 h and sacrificed after 24 h. The lungs were excised (n=3 per group) and processed as described (Cho, H Y et al. Am J Respir Cell Mol Biol 26:175-182. 2002) to measure the activities of NQO1, G6PDH, GPx, Prx and GSR. Glutathione peroxidase activity was measured according to the procedure of Flohe and Gunzler (Assays of glutathione peroxidase. Methods Enzymol 105:114-121. 1984). NQO1 activity was determined using menadione as a substrate (Prochaska H J et al. Anal Biochem 169:328-336. 1998). The peroxidase activity of Prx was measured by monitoring the oxidation of NADPH as described (Chae H Z et al. Methods Enzymol 300:219-226. 1999). G6PDH activity was determined from the rate of glucose 6-phosphate dependent reduction of NADP⁺ (Lee CY. Glucose-6-phosphate dehydrogenase from mouse. Methods Enzymol 89 Pt D:252-257. 1982). GSR activity was determined from the rate of oxidation of NADPH by using oxidized glutathione as substrate (Carlberg I et al. Glutathione reductase. Methods Enzymol 113:484-490. 1985). Protein concentration was determined by using the Biorad DC reagent, with bovine serum albumin as the standard. The values for enzyme-specific activities are given as means±SE. Student's t-test was used to determine statistical significance.

### GSH and GSSG Analysis

The concentrations of GSH and GSSG in the lung tissues were measured using a BIOXYTECH GSH/GSSG-412 kit. To measure GSSG, 10 mg of lung tissue was homogenized with a solution (300 μl) containing 1-methyl-2-vinyl-pyridium trifluoromethane sulfonate (10 μl) and 5% cold metaphosphoric acid (290 μl) and centrifuged for 10 min at 1000×g. The supernatant was diluted (1/15) with GSSG buffer. Two hundred microliter of the diluted supernatant was mixed with an equal volume of chromogen, glutathione reductase enzyme solution and incubated at room temperature for 5 min. To this, 200 μl of NADPH was added and the change in absorbance was recorded at 412 nm for 3 min. To measure GSH, the lung tissue (10 mg) was homogenized with 5% cold metaphosphoric acid (350 μl) solution and centrifuged for 5 min at 1000×g. The remaining procedure was similar to the one described above for measuring GSSG. Different concentrations of GSSG were used as the standard.

Isolation of CD4⁺ T Cells and Macrophages from the Lungs

To isolate lung CD4⁺ T cells, mice were euthanatized and the pulmonary cavities were opened. The blood circulatory system in the lungs was cleared by perfusion through the right ventricle with 3 ml of saline containing 50 U of heparin per ml. Lungs were aseptically removed and cut into small pieces in cold PBS. The dissected tissue was then incubated in PBS containing collagenase IV (150 U/ml) and bovine pancreatic DNase I (50 U/ml) for 1 h at 37° C. The digested lungs were further disrupted by gently pushing the tissue through a nylon screen. The single-cell suspension was then washed and centrifuged at 500 g for 5 min. The pellet was resuspended in PBS and passed through a cell stainer to remove the coagulated proteins and centrifuged for 5 min at 500 g. To lyse the contaminating red blood cells, the cell pellet was incubated for 5 min at room with red cell lysis buffer. Cells were then washed with PBS containing 2% FBS and counted.

CD4⁺ T cells were isolated by negative selection using CD4⁺ T cell isolation kit. Cells (10⁷ cells) isolated from the lungs were first incubated with biotin-antibody cocktail containing anti-CD8 alpha, anti-CD11b, anti-CD45R, anti-DX5, and anti-Ter119 for 10 min and then with anti-biotin microbeads for 15 min at 4° C. The cells were then washed with 20 volumes of buffer and passed through MACS MS column. The magnetically labeled non-CD4⁺ T cells were depleted by retaining them on MACS MS column, while the eluents containing the unlabeled CD4⁺ T cells were collected. An aliquot of cells was analyzed by immunofluorescence and flow cytometry using anti-CD4 antibodies. After gating on scatter characteristics to exclude dead cells and debris, the purity of cells was 90-92% CD4⁺ T lymphocytes. RNA was isolated from the purified CD4⁺ T cells using RNeasy mini columns.

Alveolar macrophages were obtained from the OVA challenged (24 h after 1^{st} OVA challenge) Nrf2^{+/+} and Nrf2^{-/-} mice (15 mice in each group) by saline lavage (3×1 ml). The BAL fluid collected from each group was pooled separately and centrifuged at 500 g for 5 min at 4° C. The cell pellets were suspended in RPMI 1640 medium and cultured (in 6 well plates) for 2 hours in CO₂ incubator. The nonadherent cells were removed with the supernatant. The wells were washed 2 times with sterile PBS. The adherent macrophages were then lysed with RLT buffer and the RNA was isolated using RNeasy mini columns. Real Time RT P-CR was used to determine the expression of three well-characterized Nrf2-regulated genes (GCLm, GCLc and HO-1) in the isolated CD4+ T cells and macrophages by following the procedure described above. The fold change was obtained by comparing the message level of antioxidant genes in the CD4⁺ T and macrophages of wild-type mice over their levels in the knock out counterparts

The expression of Nrf2 mRNA in the lung CD4⁺ T cells and macrophages was determined by RT-PCR using the mouse Nrf2 5′-TCTCCTCGCTGGAAAAAGAA-3′ and 3′-AATGTGCTGGCTGTGCTTTA-5′ primers. Total RNA (500 ng) was reverse transcribed into cDNA in a volume of 50 μl, containing 1×PCR buffer [50 mM KCl and 10 mM Tris (pH 8.3)], 5 mM MgCl₂, 1 mM each dNTPs, 125 ng oligo (dT)₁₅, and 50 U of Moloney Murine Leukemia Virus reverse transcriptase (Life Technologies), at 45° C. for 15 min and 95° C. for 5 min using gene amp PCR System 9700 (Perkin Elmer Applied Biosystems, Foster City, Calif.). Separate but simultaneous PCR amplifications were performed with aliquots of cDNA (1 μl) at a final concentration of 1×PCR buffer, 4 mM MgCl₂, 400 μM dNTPs, and 1.25 U Taq Polymerase (Life Technologies) in a total volume of 50 μl using 240 nM each of forward and reverse primers.

### Assay of T Lymphocyte Activation

Spleens were asceptically removed from OVA challenged (48 h after 2^{nd} challenge) Nrf2^{+/+} and Nrf2^{-/-} mice and mechanically dissociated in cold PBS, followed by depletion of erythrocytes with lysis buffer containing NH₄Cl. Splenocytes were suspended in RPMI 1640 containing 10% FCS, 2 mM L-glutamine, 100 U/ml penicillin, 100 μg/ml streptomycin, 10 mM HEPES, and 20 μM 2-ME. Splenocytes (10⁶/ml) were incubated at 37° C. in a 5% CO₂ atmosphere and stimulated for 24 h with OVA (5 μg/ml) or anti-mouse CD3 plus anti-mouse CD28 antibodies (0.5 μg/ml each). After 24 h of incubation, cell-free culture supernatants were collected and stored at −70° C. until cytokine analyses were performed.

In order to determine whether Nrf2 μlayed a T cell intrinsic role in regulating Th2 cytokine gene expression, we isolated CD4⁺ T cells by negative immunomagnetic selection (see above) from single cell spleen suspensions of control wildtype and Nrf2^{-/-} mice. Equal numbers of viable cells (1×10⁶ million/ml) were incubated for 24 h in complete medium alone, or stimulated with plate bound anti-CD3 (2 μg/ml) plus soluble anti-CD28 (2 μg/ml) or calcium ionophore A23187 (1 μM) plus PMA (20 ng/ml). Cell supernatants were collected and analyzed for IL-4 or IL-13 secretion by ELISA.

### Construction of Nrf2 Expression Vector and IL-4 and IL-13 Promoter Constructs

An Nrf2 overexpressing construct was made with the ubiquitin C (pUbC) promoter. Nrf2 cDNA lacking a stop codon was cloned in TOPO 2.1 vector and sequenced. The Nrf2-Topo construct was digested with KpnI and NotI to release the Nrf2 cDNA. The cDNA was purified and ligated with pUB6/V5-His vector digested with KpnI and NotI. The recombinant clones were further screened and confirmed by sequencing. To test whether Nrf2 is able to bind to ARE and activate luciferase activity, the Nrf2 construct was transfected into Hepa cells stably transfected with heme oxygenase-1 ARE. Luciferase activity was measured after 36 h. For the IL-4 and IL-13 promoter constructs, human genomic DNA was used as a template with PCR primers designed to amplify sequences 270 and 312 basepairs upstream respectively, and 65 basepairs downstream of the transcription start sites. PCR primers contained restriction sites for KpnI and SacI to facilitate subsequent ligation. After sequencing to ensure accurate replication, PCR products were ligated into the KpnI and SacI sites of the luciferase-based reporter construct pGL3 Basic.

### Transfection in Jurkat Cell Line

To test the possibility that Nrf2 might act as a transcriptional repressor of Th2 cytokines, we first electroporated the Jurkat T cell line (20 million cells/0.5 ml of OPT-MEMI) with Nrf2 overexpressing vector (20 μg/20 million cells) or pUB6 control vector (20 μg/20 million cells) using a BioRad electrophorator (at 300V and 1050 capacity), and analyzed effects of Nrf2 overexpression on endogenous IL-13 gene expression. The cells were then mixed with OPT-MEMI (2 million cells/2 ml/well of 6 well plate) and incubated for 4 h at 37° C. in a CO₂ incubator. FBS (final concentration 10%) was added to each well and incubated for 14 h. Cells were centrifuged, resuspended in OPTI-MEMI (1×10⁶ cells/nil) with or without the calcium ionophore A23187 (0.5 μg/ml final) and PMA (10 ng/ml final) and cultured at 37° C. for 18 h in a CO₂ incubator. The cultures were centrifuged at 500 g for 5 min at 4° C. The supernatants were collected and IL-4 and IL-13 cytokines were assayed using the human Quantikine ELISA kits. The Jurkat T cells used in these experiments do not secrete abundant IL-4 protein due to poorly understood post-transcriptional defects. To ensure that Nrf2 was overexpressed and activate downstream target genes, cell pellets were homogenized with RLT buffer and the RNA was isolated using the RNeasy mini columns. The levels of Nrf2 and the classical Nrf2 regulated genes NQO1 and GCLm mRNA were analyzed using real time RT-PCR using the assay on demand kits containing the respective primers for human Nrf2, GCLc and NQO1 genes.

To test the possibility that Nrf2 was acting to repress Th2 cytokine gene transcription, Nrf2 or empty expression vectors were co-transfected into Jurkat T cells together with reporter constructs containing the human IL-4 or IL-13 promoters driving the firefly luciferase gene. Cells were transfected and stimulated as above although in a scaled down version (5 million cells, 5 μg reporter construct, up to 5 μg expression vector or control). Both approaches yielded similar transfection efficiencies. Eighteen hours after transfection, cells were lysed and firefly luciferase gene expression was analyzed by luminometry using a Monolight 3010 Luminometer and assay buffers according to the manufacturer's instructions (Promega).

### Sensitization and Challenge Protocols

Mice (male, 8 weeks old) were sensitized on day 0 by i.p. injection (100 μl/mouse) with 20 μg of ovalbumin complexed with aluminum potassium sulfate. On day 14, mice were sensitized a second time with 100 μg OVA. On days 24, 26 and 28, the mice were anesthetized by i.p. injection of 0.1 ml of a mixture of ketamine (10 mg/ml) and xylazine (1 mg/ml) diluted in sterile PBS and challenged with 200 μg of OVA (in 100 μl sterile PBS) by intratracheal instillation. The control groups received sterile PBS with aluminum potassium sulfate by i.p. route on day 0 and 14, and 0.1 ml of sterile PBS on day 24, 26 and 28. Mice were euthanized at different time points after OVA challenge for BAL, RNA isolation, histopathology, and for AHR measurements.

### Histochemistry

The lungs were inflated with 0.6 ml of buffered formalin (10%), fixed for 24 h at 4° C., prior to histochemical processing. The whole lung was embedded in paraffin, sectioned at a thickness of 5 μm, and stained with H&E (n=6) for routine histopathology. Tissue sections were also stained with PAS for the identification of stored mucosubstances within the mucus goblet cells lining the main axial airways (proximal), as previously described (Steiger D J et al. Am J Respir Cell Mol Biol 12:307-314.1995). The number of PAS positive cells was counted on longitudinal lung sections of the proximal airways. The percent PAS positive cells was determined by counting the mucus positive cells and unstained epithelial cells in the proximal airways under the microscope with a grid at 100× magnification. Six animals were used for each treatment. The sum of the values of five fields/slide, for five slides is provided for each animal. The data are expressed as mean±SEM.

### Immunohistochemical Staining of Eosinophils in the Lungs

For detection of eosinophils in tissues, the lung sections from the saline and OVA challenged (72 h after 3^{rd} challenged) mice (n=6) were deparaffinized and dehydrated in benzene and alcohol respectively, and the endogenous peroxidase activity was quenched with 0.6% H₂O₂ in 80% methanol for 20 minutes. Sections were then digested with pepsin for 10 min prior to blocking with 5% normal rabbit serum for 30 min at room temperature. Rat anti-mouse major basophilic protein −1 (MBP) antibody [kindly provided by James J. Lee, Mayo clinic, Arizona, USA was then applied for 60 min, followed by incubation with rabbit anti-rat IgG/HRP conjugate for 60 minutes. HRP was visualized with diaminobenzidine. Nuclei were stained by application of purified 2% methyl green for 2 min.

### Intervention With N-Acetyl Cysteine (NAC)

Nrf2^{+/+} and Nrf2^{-/-} mice (6 mice in each group) were sensitized with OVA by following the procedure as already described. Sensitized animals were randomly distributed into positive control (saline plus OVA), negative control (saline) and N-Acetyl Cysteine (NAC; Sigma) treated (NAC plus saline or antigen) groups. NAC was dissolved in distilled water (3 mmol/kg body weight, pH 7.0) and administered orally by gavage (Blesa S J et al. Eur Respir J 21:394-400. 2003) as a single daily dose for 7 days before challenge with the last dose being given 2 h before OVA challenge. Twenty-four hours after challenge, BAL fluids and lung tissues were harvested and analyzed as above. The experiment was repeated two times.

To investigate the effect of replenishing antioxidant in nrf2−/− mice on lung inflammation induced by non-lethal dose of LPS (60 μg per mouse), mice were pretreated with NAC (500 mg/kg body weight) three times, 4 h apart. After 1 h of the last dose of NAC, LPS was injected and BAL fluid analysis and expression of inflammatory genes were performed as described above. To determine the effect of replenishing antioxidant in nrf2−/− mice on LPS induced septic shock, NAC (500 mg/kg body weight) was administered (ip) every day for 4 days. After 1 h of the last dose of NAC, a lethal dose of LPS (1.5 mg per mouse) was injected. Mortality was observed as described above.

### Determination of Lipid Hydroperoxides and Protein Carbonyls in the Lungs

To quantify lipid hydroperoxides, lung tissues were homogenized in PBS (10 mM, containing 10 μM cupric sulfate) and incubated for 30 min at 37° C. in a shaking water bath. Five volumes of methanol were added to the lung homogenate, vortexed vigorously for 2 min and centrifuged at 8000 g for 5 min. 0.9 ml of Fox reagent was added to 0.1 ml of methanol extract, and incubated for 30 min at room temperature. The absorbance was read at 560 nm using a spectrophotometer. Hydrogen peroxide was used as the standard. Data were expressed as micromoles of lipid hydroperoxide per milligram of protein using the molar extinction coefficient of 43, 000 for hydroperoxides (Jiang, Z Y et al. Anal Biochem 202:384-389. 2003).

To determine the protein carbonyls, the lungs were homogenized in 10 mM HEPES buffer [containing 137 mM NaCl, 4.6 mM KCl, 1.1 mM KH₂PO₄, 0.6 mM MgSO₄, 1.1 mM EDTA, Tween 20 (5 mg/1), butylated hydroxytoluene (1 uM), leupeptin (0.5 μg/ml), pepstatin (0.7 μg/ml), aprotinin (0.5 μ/ml) and PMSF (40 μg/ml)] and centrifuged at 8000 g for 10 min at 4° C. Supernatant fractions were divided into two equal aliquots containing 0.7 to 1 mg protein each, precipitated with 10% TCA and centrifuged at 8000 g for 5 mM at room temperature. One pellet was treated with 2.5 M HCl, and the other was treated with an equal volume of dinitrophenyl hydrazine (10 mM) in HCl (2.5 M) at room temperature for 1 h. Samples were re-precipitated with TCA (10%) and subsequently with ethanol and ethyl acetate (1:1, v/v), and again re-precipitated with 10% TCA. The pellets were dissolved in phosphate buffer (20 mM, pH 6.5, containing 6 M guanidine hydrochloride) and left for 10 min at 37° C. with general vortex mixing. Samples were centrifuged at 6000 g for 5 min and the clear supernatants were collected. The difference in absorbance between DNPH-treated and the HCl control was determined at 370 nm. Data were expressed as nanomoles of carbonyl groups per milligram of protein using the molar extinction coefficient of 21, 000 for NADPH derivatives (Oliver Conn. et al. J Biol Chem 262:5488-5491. 1987)

### Measurement of Airway Responsiveness

On day 31 (96 h after the 3^{rd} OVA challenge), mice (n=7) were anesthetized with sodium pentobarbital, and their tracheas cannulated via tracheostomy. The animals were ventilated as previously described (Ewart S R et al. 79:560-566. 1995) with a tidal volume of 0.2 ml at 2 Hz. Succinylcholine was given (0.5 mg/kg body weight) intraperitoneally to eliminate all respiratory efforts. Aerosol acetylcholine challenges were administered by nebulization with an Aeroneb Pro (Aerogen, Inc., Mountain View, Calif., USA) nebulizer modified to decrease the dead space to 1 ml. Data were plotted as lung resistance and compliance at baseline and in response to a 10 s challenge of 0.3 mg/ml acetylcholine.

### Assay of T Lymphocyte Activation

In order to determine whether Nrf2 μlayed a T cell intrinsic role in regulating Th2 cytokine gene expression, CD4⁺ T cells and splenocytes from the spleen of saline and OVA challenged Nrf2^{+/+} and Nrf2^{-/-} mice were isolated and stimulated for 24 h in the absence or presence of anti-CD3 plus anti-CD28 antibodies, or the calcium ionophore A21387 plus the phorbol ester PMA, followed by analysis of cytokine secretion by ELISA.

### Luciferase Promoter Assay and Nrf2 Overexpression

Reporter constructs containing the human IL-4 and IL-13 promoter regions linked to the firefly luciferase gene were synthesized using standard techniques (pGL3 Basic, Promega). Promoter reporter constructs were co-transfected with an Nrf2-expression vector into Jurkat T cells followed by analysis of reporter gene expression using luminometry, or endogenous gene expression by real time RT-PCR and ELISA.

### ELISA Measurements of IL-4, IL-13 and Eotaxin

To measure the cytokine levels, the BAL fluid was collected from the lungs of each mouse (n=8) with 0.7 ml of PBS containing a cocktail of protease inhibitors and immediately centrifuged at 4° C. for 5 min at 1500×g. The supernatant was collected, aliquoted and frozen in liquid nitrogen. The levels of IL-4 and IL-3 in BAL fluid as well as in the supernatants from the splenocyte culture were determined by ELISA using IL-4 and IL-13 quantikine ELISA kits. Eotaxin level in BAL fluid was analyzed using mouse eotaxin ELISA kit.

### Quantification of GSH and GSSG in Lung Tissue

The concentrations of reduced and oxidized glutathiones in the lung tissues were measured using BIOXYTECH GSH/GSSG-412 kit (Oxis, International, Foster City Calif.).

### P65/Rel a DNA Binding Activity

DNA binding activity of the p65/Rel A subunit of NF-kB was determined using Mercury TrasFactor Kit (BD Biosciences). An equal amount of nuclear extracts isolated from the lungs were added to incubation wells precoated with the DNA-binding consensus sequence. The presence of translocated p65/Rel A subunit was then assessed by using Mercury TransFactor kit according to manufacturer instructions. Plates were read at 655 nm, and results were expressed as OD.

### Quantitative Real-Time RT-PCR

Total RNA was extracted from the lung tissues (n=3) with TRIZOL reagent and then used for first-strand cDNA synthesis. Reverse transcription was performed with random hexamer primers and SuperScribe II reverse transcriptase. Using 100 ng of cDNA as a template, quantification was performed by an ABI Prism 7000 Sequence Detector (Applied Biosystems, Foster City, Calif.) using the TaqMan 5′ nuclease activity from the TaqMan Universal PCR Master Mix, fluorogenic probes, and oligonucleotide primers. The copy numbers of cDNA targets were quantified according to the point during cycling when the PCR product was first detected. The PCR primers and probes detecting GST α3 (Accession No: X65021) were designed based on the sequences reported in GeneBank with the Primer Express software version 2.0 (Applied Biosystems, Foster City, Calif., USA) as follows: GST α3 forward primer 5′-CCTGGCAAGGTTACGAAGTGA-3′; GST α3 reverse primer 5′-CAGTTTCATCCC GTCGATCTC-3′; GST α3 probe FAM 5′-CTGATGTTCCAGCAAGTGCCC-3′ TAMRA. For the rest of the genes including GAPDH control, the assay on demand kits containing the respective primers were used. TaqMan assays were repeated in triplicate samples for each of nine selected antioxidant genes (GCLm, GCLc, GSR, GST α3, GST p2, G6PD, SOD2, SOD3, and HO-1) in each lung sample. The mRNA expression levels for all samples were normalized to the level of the housekeeping gene GAPDH.

In other studies, the NF-κB probe [5′-GTTGAGGGGACTTTCCCAGGC-3′] (Promega, Madison, Wis.) was end-labeled by T4 polynucleotide kinase in the presence of [³²P] ATP gamma. For EMSA, 5 μg of nuclear proteins was incubated with the labeled NF-κB probe in the presence of poly(dI-dC) in binding buffer (Promega) at 4° C. for 20 min. The mixture was then resolved by electrophoresis on a 5% nondenaturing polyacrylamide gel and developed by autoradiography. For supershift analysis, nuclear proteins were incubated with 1 to 2 μg of polyclonal antibody to either p65 and or p50 subunit of NF-κB (Santa Cruz Biotechnology) for 30 min after incubation with the labeled probe.

### Cecal Ligation and Puncture

Polymicrobial sepsis was induced by CLP. Briefly, a midline laparotomy was performed on the anesthetized mice and the cecum was identified. The distal 50% of exposed cecum was ligated with 3-0 silk suture and punctured with one pass of an 18-gauge needle. The cecum was replaced in the abdomen and the incision was closed with 3-0 suture. Another set of mice was subjected to midline laparotomy and manipulation of cecum without ligation and puncture (sham operation). Postoperatively, the animals were resuscitated with 1 ml subcutaneous injection of sterile 0.9% NaCl. Mice were monitored regularly and survival was recorded over a period of 5 days.

### Measurement of Lung Edema

Five animals per group were treated with LPS for 24 h. Mice were sacrificed by ip injection of sodium pentobarbital, and the lungs were excised. All extrapulmonary tissue was cleared, weighed (wet weight), dried for 48 h at 60° C., and then weighed again (dry weight). Lung edema was expressed as the ratio of wet weight to dry weight.

ELISA. Levels of TNF-α, TNFRI (p55) and TNFRII (p75) were measured by enzyme immunoassays by using murine ELISA kits (R&D Systems, Minneapolis, Minn.).

### Measurement of Myeloperoxidase

The activity of myeloperoxidase, an indicator of neutrophil accumulation, was measured in the supernatant fluid obtained from whole lung homogenates as described (Speyer C L, et al. Am J Pathol 163:2319-2328. 2003.)

### Microarray

Mice of both genotypes were subjected to systemic inflammation by ip injection of LPS (60 μg per mouse). Lungs were isolated at 30 min, 1 h, 6 h, 12 h, and 24 h after LPS challenge. Total RNA from the lungs was extracted by using TRIzol reagent (Gibco BRL, Life Technologies, Grand Island, N.Y.). The isolatedRNA was applied to Murine Genome MOE 430A GeneChip arrays (Affymetrix, Santa Clara, Calif.) according to procedures described previously (5). This array contains probes for detecting ˜44,500 well-characterized genes and 4371 expressed sequence tags.

Scanned output files were analyzed by using Affymetrix GeneChip Operating Software and were independently normalized to an average intensity of 500. Further analyses was done as described previously (5) by performing 9 pair-wise comparisons for each group (nrf2+/+LPS, n=3, vs. nrf2+/+ vehicle, n=3, and nrf2−/−LPS, n=3, vs. nrf2−/− vehicle, n=3). To limit the number of false positives, only those altered genes that showed a change of more than 1.5 fold and appeared in at least 6 of the 9 comparisons were selected. In addition, the Mann-Whitney pairwise comparison test was performed to rank the results by concordance as an indication of the significance (P≦50.05) of each identified change in gene expression.

### Isolation of Resident Peritoneal Macrophages and Treatment

Resident peritoneal macrophages were harvested from 4 mice of each genotype by peritoneal lavage with 5 ml of cold RPMI-1640 medium supplemented with 10% FBS. Isolated peritoneal macrophages from all mice of the same genotype were pooled and plated into 24-well plates at a density of 1×10⁶ cells/ml. Adherent cells were maintained in RPMI 1640 medium supplemented with 10% FBS, 1% penicillin, and 1% streptomycin for 16 h at 37° C. in a CO₂ incubator. Cells were then stimulated with LPS (1 ng/ml) in serum-free medium.

In Vitro IKK Kinase activity

Cytoplasmic extracts were isolated from cells using cell lysis buffer (Cell Signaling Technology) and protein was measured by BCA protein assay kit (Pierce). Cytoplasmic extracts (250 μg) were incubated with 1 μg IKKα monoclonal antibody (Santa Cruz Biotechnology) for 2 hr at 4° C., and then with protein A/G-conjugated Sepharose beads (Pierce) for 2 h at 4° C. After washing with cell lysis buffer for five times and once with the kinase buffer (Cell Signaling Technology), the beads were incubated with 20 μl kinase buffer containing 20 μM adenosine 5′-triphosphate (ATP), 5 μCi [³²P] ATP, and 1 μg GST-IκBα (1-317) substrate (Santa Cruz Biotechnology) at 30° C. for 30 min. The reaction was terminated by boiling the reaction mixture in 5× sodium dodecyl sulfate (SDS) sample buffer. Proteins were resolved on a 10% polyacrylamide gel under reducing conditions, the gel was dried, and the radiolabeled bands were visualized using autoradiography. To determine the total amounts of IKKα in each sample, immunoblotting was performed. Proteins (30 μg) from whole cell extract were resolved on a 12% SDS-acrylamide gel then electrotransferred to a PVDF and probed for IKKα (Santa Cruz Biotechnologies).

### Transfection and Luciferase Assay

MEFs from mice of both genotypes were prepared from 13.5-day embryos as described (44) and grown in Iscove's modified Dulbecco's medium supplemented with 10% FBS, 0.5% penicillin, and 0.5% streptomycin. MEFs (60-80% confluence) were transfected with luciferase reporter genes (pNF-κB-luc or ISRE-Tk-Luc vector) by using Lipofectamine2000 (Invitrogen). The Renilla-luciferase reporter gene (pRL-TK) was co-transfected for normalization. After the treatments, the reporter gene activity was measured using the Dual Luciferase Assay System (Promega). All transfection experiments were carried out in triplicate wells and were repeated separately at least 3 times.

### Reduced and Oxidized Glutathione

A Bioxytech GSH/GSSG-412 kit (Oxis Health Products, Portland, Oreg.) was used to measure reduced and oxidized glutathione in the lungs. Briefly, lung tissue was homogenized in cold 5% metaphosphoric acid. For measuring GSSG, 2-methyl-2-vinyl-pyridinium trifluoromethane sulfonate, a scavenger of reduced glutathione, was added to an aliquot of lung homogenate. The homogenates were centrifuged at 5000-×g for 5 min at 4° C., and the supernatant fluid was used to measure GSH and GSSG as per the manufacturer's instructions. Total GSH in MEFs were measured as previously described (Tirumalai R et al. Toxicol Lett 132:27-36.2002).

### Statistical Analysis

Statistical analysis was performed by analysis of variance (ANOVA), with the selection of the most conservative pairwise multiple comparison method, using the program SigmaStat and differences between groups were determined by Student's t test using the InStat program.

### Filament Models

Two different filaments (15 mm in length) were used to occlude the MCA: the rigid probe: 6-0 Ethilon monofilament (Ethicon, Inc., Somerville, N.J.), and the flexible probe: 8-0 Ethilon monofilament (Ethicon, Inc.). Rigid probes were prepared by briefly heating the tip of a 6.0 monofilament until the tip was swollen in proportion to form a bulb with diameter ranging from 180-200 μm. The swollen tip was dipped into methyl methacrylate glue (Super Glue, Ross Products. Inc., Columbus, Ohio) and left to dry overnight. Filaments were monitored under the microscope to ensure consistency in size and diameter.

To prepare the flexible monofilament, a small amount of silicone (CutterSil Light, Heraeus Kulzer, GmbH, Hanau, Germany) and hardener (CutterSil Universal, Heraeus Kulzer, Dormagen, Germany) were blended in a 3-to-1 ratio, and 5 mm of an 8-0 suture was briefly run through the mixture. The procedure was carried out under a microscope, and the monofilaments were evaluated for size and appearance. Efforts were made to ensure that the silicone coated only 5 mm at the tip. The filaments were allowed to dry overnight and used in surgeries the next day. The diameters of the 5-mm silicone-coated tip of the flexible filaments were consistently within the range of 180 to 200 μm. It is recommended that one person make the filaments to maintain consistency.

### Properties of Methyl Methacrylate and Silicone

Methyl methacrylate glue is a viscous liquid with a boiling point of 100° C. It is slightly soluble in water, and when dry has a hard and rigid surface. It has not been widely used in medical and dental procedures because it is toxic and chemically unstable. Silicone has a boiling point of 110° C., is nontoxic, and is immiscible in water. When dry, it has a smooth surface that reduces the coefficient of friction. Silicone has been used clinically for decades for shunts and catheters and is favored by surgeons for its biocompatibility and chemical stability.

### Transient Occlusion of the MCA

Each mouse was anesthetized with halothane (3% initial, 1% to 1.5% maintenance) in O₂ and air (80%: 20%). Under an operating microscope, a microfiber was attached to the skull for Laser-Doppler flowmetry (DRT4, Moor Instruments Ltd, Devon, England) measurement of relative cerebral blood flow (CBF). The MCA was occluded with a silicone-coated filament as previously described (Shah Z A et al. J Stroke Cerebrovasc Dis. in press, 2006). During occlusion, mice were kept in a humidity-controlled, 30° C.-chamber to help maintain a body core temperature of 37° C. After reperfusion, mice were again placed in the chamber for 2 hours and finally returned to their respective cages for survival up to 24 hours. Before the mice were sacrificed, neurological deficits were assessed with a 5-point neurological severity score.¹¹ Neurological deficits were graded by the following scale: 0, no deficit; 1, forelimb weakness; 2, circling to affected side; 3, inability to bear weight on the effected side; 4, no spontaneous motor activity. The brains were removed and cut into 2-mm coronal sections that were stained with 2, 3,5-triphenyltetrazolium chloride (TTC, Sigma, St. Louis, Mo.). Brain slices were scanned individually, and the unstained area was analyzed by a video image analyzing system (SigmaScan pro 5, Systat, Inc., Point Richmond, Calif.). Infarct volume was calculated as the percentage of infarct area to the total hemispheric area for each slice.

In experiments involving measurement of the relative cerebral blood flow (CBF), mice were placed in a prone position under an operating microscope, and the head was fixed in the anesthesia tube. A 0.5-mm diameter microfiber was glued to the skull with cyanoacrylate glue (Super Glue Gel, Ross Products, Inc.) over the area of the parietal cortex supplied mainly by the MCA (6 mm lateral and 1 mm posterior of bregma) and connected to a laser-Doppler flowmeter (DRT4, Moor Instruments Ltd, Devon, England). After turning the mice to the supine position, a midline-incision was made in the neck, and the right common carotid artery (CCA), external carotid artery (ECA), and internal carotid arteries (ICA) were isolated from the vagus nerve. The superior thyroid, lingual and maxillary arteries were cauterized and cut. The CCA was ligated and two closely spaced knots were placed on the distal part of the ECA with silk suture. The ECA was cut between the knots and the tied section, or stump, attached proximal to the CCA junction, was straightened to allow the filament to enter the ICA and block the MCA or circle of Willis. The ICA and the pterygopalatine artery were cleared and visualized. A microvascular clip was applied temporarily to the ICA proximal to the CCA bifurcation to stop the blood supply, and the ECA stump was incised to insert the filament. Once the tip of the inserted filament (6-0 or 8-0) reached the clip, a knot was tied on the ECA stump to prevent bleeding through the arteriotomy. The clip was then removed permanently, and the filament was carefully advanced up to 11 mm from the carotid artery bifurcation or until resistance was felt, confirming the filament was not in the pterygopalatine artery. A schematic depiction of the procedure is provided in FIG. 34. A drop in relative CBF by 80% or more, as measured by the laser-Doppler flowmeter, was considered a successful occlusion and was monitored constantly for up to 5 minutes. Mice not attaining the required decrease were excluded from the study. Cortical perfusion values were expressed as a percentage relative to baseline.

### Evaluation of Neurological Deficits

Motor deficits were graded by sensorimotor performance or neurological score by the method of Longa et al. (Stroke. 20:84-91.1989). Mice were evaluated at 1, 2, and 22 hours after occlusion with a 4-point neurological severity score with the following point scale: (1) no deficit, (2) forelimb weakness, (3) inability to bear weight on the affected side, (4) no spontaneous motor activity.

### Infarct Size and Volume

After 24 hours of reperfusion, mice were anesthetized, and their brains were frozen at −80° C. for a brief period, cut into five 2-mm coronal sections, and incubated in 2% 2,3,5-triphenyltetrazolium chloride (TTC, Sigma Co, St. Louis, Mo.) solution for 15-20 minutes at 37° C. The stained slices were transferred into 10% formaldehyde solution for fixation. Images of the five sections of each brain were captured with a digital camera using Matrox Intellicam software, version 2.0 (Dorval, QC, Canada). Brain slices were scanned individually, and the unstained area was analyzed by a video image analyzing system (SigmaScan pro 4 and 5, Systat, Inc., Point Richmond, Calif.). Intact volumes of ischemic ipsilateral and normal contralateral brain hemispheres were calculated by multiplying the sum of the areas by the distance between sections. Volumes of the infarct were measured indirectly by subtracting the nonischemic tissue area in the ipsilateral hemisphere from that of the normal contralateral hemisphere. Infarct size and volume were calculated and expressed as a percentage of infarct area to total hemispheric area for each slice.

### Blood Gas Measurements

In a separate cohort of mice (5 WT; 5 Nrf2^{-/-}) that underwent an identical stroke protocol, including CBF monitoring, blood samples were collected through a PE-10 femoral artery catheter (Intramedic; BD Diagnostic Systems, Sparks, Md.) 30 minutes before MCAO, 1 hour after initiation of MCAO, and 1 hour after reperfusion. The blood was evaluated for pH, PaO₂, and PaCO₂ via blood gas analysis (Rapidlab 248; Chiron Diagnostic Corporation, Norwood, Mass.). In some experiments, blood was drawn intermittently at different intervals of time; 30 minutes before MCAO, 1 h after the initiation of MCAO, and 1 h after reperfusion.

### Primary Neuronal Cell Analysis: Western Blots and Cell Survival Assays

Cortical neuronal cells were isolated from 17-day embryos of timed-pregnant mice and cultured in serum-free conditions. Neurons were plated onto poly-D-lysine-coated 24-well dishes at a density of 0.5×10⁶ cells/well in HEPES-buffered, high glucose Neurobasal medium with B27 supplement, and cultured at 37° C. in a 95% air/5% CO₂ humidified atmosphere. As previously described (Echeverria V et al. Eur J Neurosci. 22:2199-2206. 2005) all experiments were performed after 14 days in vitro, using cortical cell cultures enriched with more than 95% neurons. Cells were first incubated in medium containing B27 minus antioxidant (B27-AO™, Sigma) 2 hours before each experiment, as this medium does not contain antioxidants that could interfere with the analysis of free-radical damage to neurons. Neurons were exposed to the various drugs for 24 hours and assessed with the 3-(4,5-dimethylthiazol-2-yl)-2,5-diphenyltetrazolium bromide (MTT, Sigma) colorimetric assay, an indicator of the mitochondrial activity of living cells. After 2 hours incubation at 37° C. with 0.5 mg/mL of MTT, living cells containing MTT formazan crystals were solubilized in a solution of anhydrous isopropanol, 0.1 N HCl, and 0.1% Triton X-100. The optical density was measured at 570 nm. All experiments were repeated with at least three separate batches of cultures.

Caspase-3/7 assay was performed on cells treated for 8 hours at 37° C. in the presence of the appropriate agents following the manufacturer's protocol (Promega, Madison, Wis.). For Western blot analysis, neurons were exposed to 60 μM t-BuOOH, 300 μM glutamate, or 100 μM NMDA for 6 h. Experiments were terminated by application of sample buffer. Equivalent amounts of protein per sample were separated via SDS-polyacrylamide gel electrophoresis on 10% gels.

### Isolation of Cytosolic/Nuclear Fractions

Primary mouse cortical neurons were scraped from culture dishes, resuspended in cold Buffer A [10 mM HEPES-KOH (pH 7.9), 1.5 mM MgCl₂, 10 mM KCl, 0.5 mM dithiothreitol (DTT), and 0.2 mM phenylmethylsulfonyl fluoride (PMSF)], and kept on ice for 10 minutes. Then, 25 μL of 10% v/v Nonidet P40 was added to the cell suspension. Samples were then centrifuged at 12,000 g for 5 minutes at 4° C. The resultant supernatant was removed as the cytosolic fraction. Pellets were resuspended in 80 μL of Buffer B [20 mM HEPES-KOH (pH 7.9), 25% glycerol, 420 mM NaCl, 1.5 mM MgCl₂, 0.2 mM EDTA, 0.5 mM DTT and 0.2 mM PMSF] and kept on ice for 20 minutes for high salt extraction. After a final 2-minute centrifugation at 4° C., the supernatant, which contained the nuclear fraction, was collected and stored at −70° C. Samples were analyzed on 10% polyacrylamide gels as described as above.

### MCAO and Reperfusion.

Transient focal cerebral ischemia was induced by MCAO with an intraluminal filament technique as described previously (Shah et al., 2006). Relative CBF was measured by laser-Doppler flowmetry (Moor instruments, Devon, England) with a flexible probe affixed to the skull over the parietal cortex supplied by the MCA (2 mm posterior and 6 mm lateral to bregma). MCAO was maintained for 120 min during which the neck was closed with sutures, anesthesia was discontinued, and the animals were transferred to a temperature-controlled chamber to maintain body temperature at 37.0±0.5° C. After 120 min, the mice were briefly anesthetized with halothane, and reperfusion was achieved by withdrawing the filament and reopening the MCA. The neck was sutured, and the mice were returned to the temperature-controlled chamber for 6 h.

### Assessment of Neurological Score

Twenty-two hours after reperfusion, mice were scored for neurological function as described previously (Li, 2004 #11362). Mice were graded as follows: 0=no deficit; 1=forelimb weakness and torso turning to the ipsilateral side when held by tail; 2=circling to affected side; 3=unable to bear weight on affected side; and 4=no spontaneous locomotor activity or barrel rolling.

### Quantification of Infarct Volume

After the neurological assessment, mice were deeply anesthetized and their brains removed. The brains were sliced coronally into five 2-mm thick sections and incubated with 1% TTC in saline for 30 min at 37° C. The area of brain infarct, identified by the lack of TTC staining, was measured on the rostral and caudal surfaces of each slice and numerically integrated across the thickness of the slice to obtain an estimate of infarct volume (Sigma Scan Pro, Systat, Port Richmond, Calif.). Volumes from all five slices were summed to calculate total infarct volume over the entire hemisphere, expressed as a percentage of the volume of the contralateral hemisphere. Infarct volume was corrected for swelling by comparing the volumes of the ipsilateral and contralateral hemispheres. The corrected volume was calculated as: volume of contralateral hemisphere−(volume of ipsilateral hemisphere−volume of infarct).

### Regional CBF Assessment

Regional CBF was measured at end-ischemia in a separate cohort of mice (n=5) via [¹⁴C]-IAP autoradiography (Jay, 1988 #204), as previously described for rats and mice (Alkayed, 1998 #8150; Sawada, 2000 #6175). Mice anesthetized with halothane were subjected to MCAO and catheterized via the femoral artery and vein. At 60 min of ischemia, 4 μCi of [¹⁴C]-IAP was infused intravenously at a constant rate of 108 μl/min for 45 s. Arterial blood was sampled at 5-s intervals to obtain the arterial input function as described (Sampei, 2000 #8586). The total volume of blood withdrawn was 100-160 μl. At 45 s of infusion, the anesthetized mouse was decapitated. The brain was quickly removed, frozen in 2-methylbutane on dry ice, and stored at −80° C. The brain was later sliced into 20-μm-thick coronal sections on a cryostat, thaw mounted onto glass cover slips, and apposed to Kodak SB-5 film (Eastern Kodak Company, Rochester, N.Y.) for 1 week with ¹⁴C standards. Nine autoradiographic images at each of six coronal levels (+2, +1, 0, −1, −2, −3 mm from the bregma) were digitized, and regional blood flow was calculated with image analysis software (Inquiry, Loats Associates, Westminster, Md.).

### Primary Neuronal Cell Culture

All materials used for cell culture were obtained from Invitrogen (Carlsbad, Calif.). Cortical neuronal cells were isolated from 17-day embryos of timed-pregnant mice. Neurons were cultured in serum-free conditions and plated onto poly-D-lysine-coated 24-well dishes at a density of 0.5×10⁶ cells/well in HEPES-buffered, high glucose Neurobasal medium with B27 supplement (Invitrogen, Carlsbad, Calif.), as previously described (Doré et al. 1999). Cells were incubated in growth medium at 37° C. in a 95% air/5% CO₂ humidified atmosphere until the day of experiment. Half of the initial medium was removed at day 4 and replaced with fresh medium.

### H₂O₂-Induced Cytotoxicity

After 10 d in culture, mouse primary neurons were pre-treated with EGb 761 (10, 50, or 100 μg/ml) for 6 h, and then treated for 18 h with H₂O₂ (20 μM) or vehicle (control) with or without 5 μM HO inhibitor (SnPPIX, Porphyrin Products, Inc., (Logan, Utah). Cell survival was evaluated by the 3-(4,5-dimethylthiazol-2-yl)-2,5-diphenyl tetrazolium bromide (MTT) colorimetric assay.

### Glutamate-Induced Cytotoxicity

Mouse primary neurons cultured for 14 d were pre-treated for 6 h with EGb 761 (100 μg/ml). Then the cells were rinsed with PBS and incubated with fresh medium containing glutamate (30 μM) or vehicle (control) with or without 5 μM SnPPIX. Neurons were incubated for an additional 18 h, and the MTT assay was used to estimate the cell survival. Experimental conditions were conducted in quadruplicate and repeated four times with different batches of primary cultures.

### Assessment of Cell Survival

Neuronal survival was assessed and quantified with the MTT colorimetric assay. After a 2-h incubation at 37° C. with 0.5 mg/ml MTT, living cells containing MTT formazan crystals were solubilized in a solution of anhydrous isopropanol, 0.1 N HCl, and 0.1% Triton X-100. The optical density was determined at 570 nm. Cell viability of the vehicle-treated control group was defined as 100%, and MTT optical density in the treated groups was expressed as a percent of control. Experiments were repeated with at least three separate batches of cultures.

### Effect of Gingko Biloba Extracts on Protein Expression

To determine the effect of EGb 761 on HO-1 protein expression, mouse neuronal cultures were treated with 0 (vehicle-control), 10, 50, 100, or 500 for 8 h or with 100 μg/ml EGb 761 for 0, 1, 2, 4, 8, or 24 h, before being harvested for Western blot analysis. To determine whether inhibition of protein synthesis or mRNA synthesis can counter the effect of EGb 761 on HO-1 expression, neuronal cells were treated for 8 h with vehicle (control), EGb 761 (100 μg/ml), or EGb 761 components bilobalide (10 or 100) or ginkgolides (10 or 100 μg/ml) (each generously provided by IPSEN Laboratories (Paris, France) alone or together with the protein synthesis inhibitor CHX (Sigma) or the mRNA synthesis inhibitor ATD (Sigma). Cells were then harvested and homogenized for Western blot analysis.

### Western Blot Analysis

Neuronal cultures were solubilized with 250 μl of lysis buffer (50 mM Tris-HCl, pH 7.4; 150 mM NaCl; 0.5% Triton X-100), including protease inhibitor cocktail (Roche Diagnostics, Mannheim, Germany), on ice for 30 min and centrifuged for 10 min at 12,000 g. The supernatant was then collected, and protein concentration was quantified with the BCA assay (Pierce, Rockford, Ill.). Proteins were separated by SDS-PAGE on 12% gels (Invitrogen) and then transferred to nitrocellulose membranes (BIO-RAD, Hercules, Calif.)(Doré et al. 1999). Blots were stained with Ponceau S Solution (Sigma) to verify that equal amounts of protein were loaded in each lane. Membranes were blocked for 1 h at room temperature with 5% skim milk in PBS with 0.1% Tween 20 before incubation at 4° C. overnight with polyclonal antibodies to HO-1, HO-2, CP₄₅₀R (StressGen Inc., Victoria, BC), and anti-actin (Sigma) at dilutions of 1:2,000, 1:2,000, 1:2,000, and 1:5000 respectively. Blots were washed and incubated with secondary antibodies for 1 h at room temperature and developed by enhanced chemiluminescence (Amersham Biosciences, Piscataway, N.J.).

### Luminescence Analysis

Mouse hepatoma cells stably transformed with pARE-Luc (hepa pARE-luc) were used. pARE-luc is an antioxidant/electrophilic response element (ARE)-dependent reporter plasmids that uses the firefly luciferase gene as a reporter under the control of a minimal promoter of mouse HO1 gene with three copies of ARE. Hepa pARE-luc were plated at 10,000 cells/well in 96-well plates and maintained in DMEM containing 10% fetal bovine serum, 10 mg/ml gentamicin (Sigma), and 100 mg/ml genetisin (Invitrogen). On the second day after plating, cells were washed twice with PBS, lysed in 30 μl passive lysis buffer, and shaken for 20 min at room temperature. Luciferase assay reagent (50 μl; Promega, Madison, Wis.) was mixed with 10 μl of cell lysate, and fluorescence was read with a luminometer (EG & G Berthold, Nashua, N.H.).

### If Size and Infarct Volume

After 24 or 72 h of reperfusion, mice were anesthetized, and their brains dissected out and cut into 2-mm coronal sections. Brain slices were stained with 2,3,5-triphenyltetrazolium chloride (TTC, Sigma Co, St. Louis, Mo.) and fixed in 10% buffered normal saline for 24 h. 2-mm brain slices were scanned individually by a video image analyzing system and the necrotic lesions were measured and analyzed using image analysis software (SigmaScan pro 4 and 5, Systat, Inc., Point Richmond, Calif.). Cerebral cortex and striatum volumes in ipsilateral necrotic lesion and contralateral normal side of the brain were measured multiplying the sum of the areas by the distance between sections. Infarct volume was indirectly calculated by subtracting the volume of intact tissue in the ipsilateral hemisphere from that of the contralateral hemisphere and expressed as the percentage of infarct area to the total hemispheric area for each slice.

Drug administration {(−)-Epicatechin}

Epicatechin (EC) was given orally (per kilogram of body weight) through gavage and precautions were taken not only to minimize the stress to animals but also careful administration of the drug solution. For pre-treatment studies, a single dose of EC was given 90 minutes before middle cerebral artery occlusion (MCAO). In post-treatment experiments, animals were given EC 3.5 and 6 h after MCAO.

### Transient Occlusion of the MCA (MCAO)

MCAO procedure was slightly modified from the methods previously published by Shah et al. (Shah, et al. in press, 2006). Mice were anesthetized with halothane (3% initial, 1 to 1.5% maintenance) in O₂ and air (80%:20%). To measure relative cerebral blood flow (CBF), mice were placed in a porcine posture on a temperature controlled heat blanket (37° C.). Under an operating microscope, a 0.5-mm diameter microfiber was glued to the skull (over the area of parietal cortex) with cyanoacrylate glue (Super Glue Gel, Ross Products, Inc.) approximately 6 mm lateral and 1 mm posterior of bregma and connected to a laser-Doppler flowmeter (DRT4, Moor Instruments Ltd, Devon, England). Mice were allowed to return to supine position and a neck midline-incision was made to expose the right common carotid artery (CCA), external carotid artery (ECA), and internal carotid arteries (ICA) after dissecting in through out thyroid glands. All the arteries were separated from the vagus nerve. A specially devised method for making 7-0 Ethilon nylon filament (Ethicon, Inc., Somarville, N.J.) with 5 mm of the tip coated with silicone (Cutter Sil Light and Universal Hardener, Heraeus Kulzer, GmbH, Hanau, Germany) was employed and the filament was introduced into the ICA through the ECA stump to block the blood circulation to MCA or circle of Willis. The filament was carefully advanced up to 11 mm from the carotid artery bifurcation or until resistance was felt. The path of the filament was also monitored carefully to make sure filament does not enter the pterigoplatine bifurcation. A drop in cerebral blood flow by 80% or more, as measured by the laser-Doppler flowmeter, was considered to be a successful occlusion. CFB was monitored for up to 5 minutes and mice not attaining the required drop were terminated from the study. Cortical perfusion values were expressed as a percentage relative to baseline. Animals were shifted to a humidity/temperature-controlled chamber at 32° C. to maintain the body temperature during the 90 minutes of MCA occlusion, at 37° C. For reperfusion mice were briefly anesthetizing and filament was withdrawn. After suturing the neck, midline wound mice were again returned to a humidity/temperature-controlled chamber for 2 h to maintain the body temperature at 37° C. and then later shifted to their respective cages. A stroke was considered 100% successful only when no subarachnoid hemorrhage was observed, lesion was produced, and mouse survived up to requirement of the procedure.

### Measurement of Relative Cerebral Blood Flow (CBF)

Laser-Doppler flowmetry (DRT4, Moor Instruments Ltd, Devon, England) was used to measure CBF. An incision was given between the eye and ear exposing parietal cortex area (area supplied by MCA), a 0.5-mm diameter microfiber was attached with cyanoacrylate glue (6 mm lateral and 1 mm posterior of bregma). CBF was monitored at baseline and continued for 5 to 10 minutes after blocking the MCA. Animals not attaining the desired 80% drop in CBF were disqualified from the study.

### Statistical Analysis

Analysis of variance (ANOVA) was used to determine and compare the statistical significance of the differences between infarct volumes produced by rigid and flexible probes. Statistical significance was set at P<0.05. All values are expressed as mean±SEM, except where otherwise noted.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcornbination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Owuor E D, Kong A N. Antioxidants and oxidants regulated signal transduction pathways. Biochem Pharmacol. 2002; 64:765-770
Zhang X, Chen X, Song H, Chen H Z, Rovin B H. Activation of the Nrf2/antioxidant response pathway increases IL-8 expression. Eur J Immunol. 2005; 35:3258-3267
Wakabayashi N, Itoh K, Wakabayashi J, Motohashi H, Noda S, Takahashi S, Imakado S, Kotsuji T, Otsuka F, Roop D R, Harada T, Engel J D, Yamamoto M. Keap1-null mutation leads to postnatal lethality due to constitutive Nrf2 activation. Nat Genet. 2003; 35:238-245
Li N, Alam J, Venkatesan M I, Eiguren-Fernandez A, Schmitz D, Di Stefano E, Slaughter N, Killeen E, Wang X, Huang A, Wang M, Miguel A H, Cho A, Sioutas C, Nel A E. Nrf2 is a key transcription factor that regulates antioxidant defense in macrophages and epithelial cells: protecting against the proinflammatory and oxidizing effects of diesel exhaust chemicals. J Immunol. 2004; 173:3467-3481
Lee J M, Li J, Johnson D A, Stein T D, Kraft A D, Calkins M J, Jakel R J, Johnson J A. Nrf2, a multi-organ protector? Faseb J. 2005; 19:1061-1066
Zhao J, Kobori N, Aronowski J, Dash P K. Sulforaphane reduces infarct volume following focal cerebral ischemia in rodents. Neurosci Lett. 2006; 393:108-112
Kraft A D, Johnson D A, Johnson J A. Nuclear factor E2-related factor 2-dependent antioxidant response element activation by tert-butylhydroquinone and sulforaphane occurring preferentially in astrocytes conditions neurons against oxidative insult. J Neurosci. 2004; 24:1101-1112
Lee J M, Johnson J A: An important role of Nrf2-ARE pathway in the cellular defense mechanism. J Biochem Mol Biol. 2004; 37:139-143
Dhakshinamoorthy S, Porter AG. Nitric oxide-induced transcriptional up-regulation of protective genes by Nrf2 via the antioxidant response element counteracts apoptosis of neuroblastoma cells. J Biol Chem. 2004; 279:20096-20107
Rangasamy T, Guo J, Mitzner W A, Roman J, Singh A, Fryer A D, Yamamoto M, Kensler T W, Tuder R M, Georas S N, Biswal S. Disruption of Nrf2 enhances susceptibility to severe airway inflammation and asthma in mice. J Exp Med. 2005; 202:47-59
Shah Z A, Namiranian K, Klaus J, Kibler K, Doré S. Use of an optimized transient occlusion of the middle cerebral artery protocol for the mouse stroke model. J Stroke Cerebrovasc Dis. in press, 2006
Echeverria V, Clerman A, Doré S. Stimulation of PGE2 receptors EP2 and EP4 protects cultured neurons against oxidative stress and cell death following b-amyloid exposure. Eur J Neurosci. 2005; 22:2199-2206
Noto T, Ishiye M, Furuich Y, Keida Y, Katsuta K, Moriguchi A, Matsuoka N, Aramori I, Goto T, Yanagihara T. Neuroprotective effect of tacrolimus (FK506) on ischemic brain damage following permanent focal cerebral ischemia in the rat. Brain Res Mol Brain Res. 2004; 128:30-38
Frykholm P, Hillered L, Langstrom B, Persson L, Valtysson J, Enblad P. Relationship between cerebral blood flow and oxygen metabolism, and extracellular glucose and lactate concentrations during middle cerebral artery occlusion and reperfusion: a microdialysis and positron emission tomography study in nonhuman primates. J Neurosurg. 2005; 102:1076-1084
Namiranian K, Koehler R C, Sapirstein A, Doré S. Stroke outcomes in mice lacking the genes for neuronal heme oxygenase-2 and nitric oxide synthase. Curr Neurovasc Res. 2005; 2:23-27
Shih A Y, Li P, Murphy T H. A small-molecule-inducible Nrf2-mediated antioxidant response provides effective prophylaxis against cerebral ischemia in vivo. J Neurosci. 2005; 25:10321-10335
Shih A Y, Johnson D A, Wong G, Kraft A D, Jiang L, Erb H, Johnson J A, Murphy T H. Coordinate regulation of glutathione biosynthesis and release by Nrf2-expressing glia potently protects neurons from oxidative stress. J Neurosci. 2003; 23:3394-3406
Lee J M, Shih A Y, Murphy T H, Johnson J A. NF-E2-related factor-2 mediates neuroprotection against mitochondrial complex I inhibitors and increased concentrations of intracellular calcium in primary cortical neurons. J Biol Chem. 2003; 278:37948-37956
Bedogni B, Pani G, Colavitti R, Riccio A, Borrello S, Murphy M, Smith R, Eboli M L, Galeotti T. Redox regulation of cAMP-responsive element-binding protein and induction of manganous superoxide dismutase in nerve growth factor-dependent cell survival. J Biol Chem. 2003; 278:16510-16519
Aoki Y, Sato H, Nishimura N, Takahashi S, Itoh K, Yamamoto M. Accelerated DNA adduct formation in the lung of the Nrf2 knockout mouse exposed to diesel exhaust. Toxicol Appl Pharmacol. 2001; 173:154-160
Chan K, Han X D, Kan Y W. An important function of Nrf2 in combating oxidative stress: detoxification of acetaminophen. Proc Natl Acad Sci USA. 2001; 98:4611-4616
Cho H Y, Jedlicka A E, Reddy S P, Kensler T W, Yamamoto M, Zhang L Y, Kleeberger S R. Role of NRF2 in protection against hyperoxic lung injury in mice. Am J Respir Cell Mol Biol. 2002; 26:175-182
Szatkowski M, Attwell D. Triggering and execution of neuronal death in brain ischaemia: two phases of glutamate release by different mechanisms. Trends Neurosci. 1994; 17:359-365
Parfenova H, Basuroy S, Bhattacharya S, Tcheranova D, Qu Y, Regan R F, Leffler C W. Glutamate induces oxidative stress and apoptosis in cerebral vascular endothelial cells: contributions of HO-1 and HO-2 to cytoprotection. Am J Physiol Cell Physiol. 2005; 290:01399-1410
Love S. Oxidative stress in brain ischemia. Brain Pathol. 1999; 9:119-131 Association AH: Heart disease and stroke statistics—2005 update. Edited by. Dallas, Tex.: American Heart Association; 2005:1-63.
Bonventre J V, Huang Z, Taheri M R, et al. Reduced fertility and postischaemic brain injury in mice deficient in cytosolic phospholipase A2. Nature 1997; 390:622-625.
Sharp F R, Lu A, Tang Y, et al. Multiple molecular penumbras after focal cerebral ischemia. J Cereb Blood Flow Metab 2000; 20:1011-1032.
Chen J F, Huang Z, Ma J, et al. A(2A) adenosine receptor deficiency attenuates brain injury induced by transient focal ischemia in mice. J Neurosci 1999; 19:9192-9200.
Pan Y, Zhang H, Acharya A B, et al. Effect of testosterone on functional recovery in a castrate male rat stroke model. Brain Res 2005; 1043:195-204.
Shah Z A, Vohora S B. Antioxidant/restorative effects of calcined gold preparations used in Indian systems of medicine against global and focal models of ischaemia. Pharmacol Toxicol 2002; 90:254-259.
Namiranian K, Koehler R C, Sapirstein A, et al. Stroke outcomes in mice lacking the genes for neuronal heme oxygenase-2 and nitric oxide synthase. Curr Neurovasc Res 2005; 2:23-27.
DeBow S B, Clark D L, MacLellan C L, et al. Incomplete assessment of experimental cytoprotectants in rodent ischemia studies. Can J Neurol Sci 2003; 30:368-374.
Gerriets T, Stolz E, Walberer M, et al. Neuroprotective effects of MK-801 in different rat stroke models for permanent middle cerebral artery occlusion: adverse effects of hypothalamic damage and strategies for its avoidance. Stroke 2003; 34:2234-2239.
Furuya K, Kawahara N, Kawai K, et al. Proximal occlusion of the middle cerebral artery in C57Black6 mice: relationship of patency of the posterior communicating artery, infarct evolution, and animal survival. J Neurosurg 2004; 100:97-105.
Longa E Z, Weinstein P R, Carlson S, et al. Reversible middle cerebral artery occlusion without craniectomy in rats. Stroke 1989; 20:84-91.
Kawamura S, Li Y, Shirasawa M, et al. Reversible middle cerebral artery occlusion in rats using an intraluminal thread technique. Surg Neurol 1994; 41:368-373.
Oliff H S, Weber E, Eilon G, et al. The role of strain/vendor differences on the outcome of focal ischemia induced by intraluminal middle cerebral artery occlusion in the rat. Brain Res 1995; 675:20-26.
Belayev L, Alonso O F, Busto R, et al. Middle cerebral artery occlusion in the rat by intraluminal suture. Neurological and pathological evaluation of an improved model. Stroke 1996; 27:1616-1622; discussion 1623.
Dogan A, Baskaya M K, Rao V L, et al. Intraluminal suture occlusion of the middle cerebral artery in Spontaneously Hypertensive rats. Neurol Res 1998; 20:265-270.
Aspey B S, Taylor F L, Terruli M, et al. Temporary middle cerebral artery occlusion in the rat: consistent protocol for a model of stroke and reperfusion. Neuropathol Appl Neurobiol 2000; 26:232-242.
Barber P A, Hoyte L, Kirk D, et al. Early T1- and T2-weighted MRI signatures of transient and permanent middle cerebral artery occlusion in a murine stroke model studied at 9.4T. Neurosci Lett 2005; 388:54-59.
Belayev L, Busto R, Zhao W, et al. Middle cerebral artery occlusion in the mouse by intraluminal suture coated with poly-L-lysine: neurological and histological validation. Brain Res 1999; 833:181-190.
Tsuchiya D, Hong 5, Kayama T, et al. Effect of suture size and carotid clip application upon blood flow and infarct volume after permanent and temporary middle cerebral artery occlusion in mice. Brain Res 2003; 970:131-139.
Tureyen K, Vemuganti R, Sailor K A, et al. Ideal suture diameter is critical for consistent middle cerebral artery occlusion in mice. Neurosurgery 2005; 56:196-200; discussion 196-200.
Kitagawa K, Matsumoto M, Yang G, et al. Cerebral ischemia after bilateral carotid artery occlusion and intraluminal suture occlusion in mice: evaluation of the patency of the posterior communicating artery. J Cereb Blood Flow Metab 1998; 18:570-579.
Connolly E S, Jr., Winfree C J, Stern D M, et al. Procedural and strain-related variables significantly affect outcome in a murine model of focal cerebral ischemia. Neurosurgery 1996; 38:523-531; discussion 532.
Galati, G., Lin, A., Sultan, A. M. & O'Brien, P. J. Cellular and in vivo hepatotoxicity caused by green tea phenolic acids and catechins. Free Radic Biol Med 40, 570-80 (2006).
Satoh, T. et al. Activation of the Keap1/Nrf2 pathway for neuroprotection by electrophilic phase II inducers. Proc Natl Acad Sci USA 103, 768-73 (2006).
Shih, A. Y., Li, P. & Murphy, T. H. A small-molecule-inducible Nrf2-mediated antioxidant response provides effective prophylaxis against cerebral ischemia in vivo. J Neurosci 25, 10321-35 (2005).

1-8. (canceled)

9. A method of preventing or ameliorating in a subject in need thereof a neurodegenerative disease selected from the group consisting of Alzheimer's disease (AD) Creutzfeldt-Jakob disease, Huntington's disease, Lewy body disease, Pick's disease, Parkinson's disease, amyotrophic lateral sclerosis (ALS), neurofibromatosis and cognitive deficits, the method comprising contacting a neuronal cell with an agent listed in Table 1A, wherein the agent increases by at least 10% an Nrf2 biological activity in the cell, and the agent is not a triterpenoid, thereby preventing or ameliorating the neurodegenerative disease.

10. (canceled)

11. The method of claim 9, wherein the method reduces cell death in a neural tissue of the subject.

12. The method of claim 9, wherein the method increases Nrf2 transcription or translation.

13. The method of claim 9, wherein the agent increases a Nrf2 biological activity selected from the group consisting of binding to an antioxidant-response element (ARE), nuclear accumulation, or the transcriptional induction of target genes.

14. The method of claim 13, wherein the Nrf2 target gene is selected from the group consisting of HO-1, NQO1, GCLm, GST α1, TrxR, Pxr 1, GSR, G6PDH, γGCLm, GCLc, G6PD, GST α3, GST p2, SOD2, SOD 3 and GSR.

15-17. (canceled)

18. The method of claim 14, wherein the agent disrupts Keap1 binding to Nrf2.

19. The method of claim 18, wherein the agent is an antibody or peptide.

20. (canceled)

21. A method for protecting a neuronal cell from ischemic injury, the method comprising contacting the neuronal cell with a Keap1 inhibitor, thereby protecting the neuronal cell from ischemic injury.

22. The method of claim 21, wherein the method decreases sensitivity to an oxidative stress.

23. The method of claim 21, wherein the method decreases cell death.

24. The method of claim 23, wherein the method reduces caspase-3.

25. The method of claim 21, wherein the cell is a pulmonary cell, endothelial cell, pulmonary endothelial cell, glial cell, smooth muscle cell, epithelial cell, alveolar cell or neuronal cell.

26. The method of claim 21, wherein the agent is a compound listed in Table 1A.

27. The method of claim 21, wherein the compound is Triterpenoid-155, Triterpenoid-156, Triterpenoid-162, Triterpenoid-225, a tricyclic bis-enone, is a flavonoid, epicatechin, Egb-761, bilobalide, ginkgolide, or tert-butyl hydroperoxide.

28. The method of claim 21, wherein the agent reduces Keap1 inhibition of Nrf2.

29-30. (canceled)

31. The method of claim 21, wherein the agent disrupts Keap1 binding to Nrf2.

32. The method of claim 21, wherein the agent is an antibody or peptide.

33-41. (canceled)

42. A pharmaceutical composition for the treatment or prevention of a condition selected from the group consisting of pulmonary inflammatory condition, pulmonary fibrosis, asthma, chronic obstructive pulmonary disease, emphysema, sepsis, septic shock, hemorrhage, hearth ischemia, cerebral ischemia, cognitive deficits, and a neurodegenerative disorder, comprising a therapeutically effective amount of an agent that increases a Nrf2 biological activity or Nrf2 expression, or a therapeutically effective amount of an agent that inhibits a Keap1 biological activity or Keap1 expression.

43. The pharmaceutical composition of claim 42, wherein the agent is a compound listed in Table 1A.

44. The pharmaceutical composition of claim 42, wherein the compound is Triterpenoid-155, Triterpenoid-156, Triterpenoid-162, Triterpenoid-225, tricyclic bis-enones, is a flavonoid, is epicatechin, Egb-761, bilobalide, tert-butyl hydroperoxide, or ginkgolide.

45-70. (canceled)
